SEQUENCE LISTING (206 sequences)

SEQ ID NO: 1 - protein, 4 aa, synthetic construct
SGGS

SEQ ID NO: 2 - protein, 529 aa, unidentified
  source 1..529: note = DPANN super-phylum
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 3 - protein, 507 aa, synthetic construct
MEEAKTVSKTLSLRILRPLYSAEIEKEIKEEKERRKQGGKSGELDSGFYKKLEKKHTQMFGWDKLNLMLSQLQRQIARVFNQSISELYIETVIQGKKSNKHYTSKIVYNRAYSVFYNAYLALGITSKVEANFRSTELLMQKSSLPTAKSDNFPILLHKQKGVEGEEGGFKISADGNDLIFEIPIPFYEYDSANKKEPFKWIKKGGQKPTIKLILSTFRRQRNKGWAKDEGTDAEIRKVIEGKYQVSHIEINRGKKLGDHQKWFVNFTIEQPIYERKLDKNIIGGIDVGIKSPLVCAVNNSFARYSVDSNDVLKFSKQAFAFRRRLLSKNSLKRSGHGSKNKLDPITRMTEKNDRFRKKIIERWAKEVTNFFIKNQVGTVQIEDLSTMKDRQDNFFNQYLRGFWPYYQMQNLIENKLKEYGIETKRIKARYTSQLCSNPSCRHWNSYFSFDHRKTNNFPKFKCEKCALEISADYNAARNISTPDIEKFVAKATKGINLPDKNENVILE

SEQ ID NO: 4 - protein, 500 aa, synthetic construct
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 5 - protein, 726 aa, synthetic construct
MERQKVPQIRKIVRVVPLRILRPKYSDVIENALKKFKEKGDDTNTNDFWRAIRDRDTEFFRKELNFSEDEINQLERDTLFRVGLDNRVLFSYFDFLQEKLMKDYNKIISKLFINRQSKSSFENDLTDEEVEELIEKDVTPFYGAYIGKGIKSVIKSNLGGKFIKSVKIDRETKKVTKLTAINIGLMGLPVAKSDTFPIKIIKTNPDYITFQKSTKENLQKIEDYETGIEYGDLLVQITIPWFKNENKDFSLIKTKEAIEYYKLNGVGKKDLLNINLVLTTYHIRKKKSWQIDGSSQSLVREMANGELEEKWKSFFDTFIKKYGDEGKSALVKRRVNKKSRAKGEKGRELNLDERIKRLYDSIKAKSFPSEINLIPENYKWKLHFSIEIPPMVNDIDSNLYGGIDFGEQNIATLCVKNIEKDDYDFLTIYGNDLLKHAQASYARRRIMRVQDEYKARGHGKSRKTKAQEDYSERMQKLRQKITERLVKQISDFFLWRNKFHMAVCSLRYEDLNTLYKGESVKAKRMRQFINKQQLFNGIERKLKDYNSEIYVNSRYPHYTSRLCSKCGKLNLYFDFLKFRTKNIIIRKNPDGSEIKYMPFFICEFCGWKQAGDKNASANIADKDYQDKLNKEKEFCNIRKPKSKKEDIGEENEEERDYSRRFNRNSFIYNSLKKDNKLNQEKLFDEWKNQLKRKIDGRNKFEPKEYKDRFSYLFAYYQEIIKNESES

SEQ ID NO: 6 - protein, 517 aa, synthetic construct
MVPTELITKTLQLRVIRPLYFEEIEKELAELKEQKEKEFEETNSLLLESKKIDAKSLKKLKRKARSSAAVEFWKIAKEKYPDILTKPEMEFIFSEMQKMMARFYNKSMTNIFIEMNNDEKVNPLSLISKASTEANQVIKCSSISSGLNRKIAGSINKTKFKQVRDGLISLPTARTETFPISFYKSTANKDEIPISKINLPSEEEADLTITLPFPFFEIKKEKKGQKAYSYFNIIEKSGRSNNKIDLLLSTHRRQRRKGWKEEGGTSAEIRRLMEGEFDKEWEIYLGEAEKSEKAKNDLIKNMTRGKLSKDIKEQLEDIQVKYFSDNNVESWNDLSKEQKQELSKLRKKKVEELKDWKHVKEILKTRAKIGWVELKRGKRQRDRNKWFVNITITRPPFINKELDDTKFGGIDLGVKVPFVCAVHGSPARLIIKENEILQFNKMVSARNRQITKDSEQRKGRGKKNKFIKKEIFNERNELFRKKIIERWANQIVKFFEDQKCATVQIENLESFDRTSYK

SEQ ID NO: 7 - protein, 534 aa, synthetic construct
MKLSEQENITTGVKFKLKLDKETSEGLNDYFDEYGKAINFAIKVIQKELAEDRFAGKVRLDENKKPLLNEDGKKIWDFPNEFCSCGKQVNRYVNGKSLCQECYKNKFTEYGIRKRMYSAKGRKAEQDINIKNSTNKISKTHFNYAIREAFILDKSIKKQRKERFRRLREMKKKLQEFIEIRDGNKILCPKIEKQRVERYIHPSWINKEKKLEDFRGYSMSNVLGKIKILDRNIKREEKSLKEKGQINFKARRLMLDKSVKFLNDNKISFTISKNLPKEYELDLPEKEKRLNWLKEKIKIIKNQKPKYAYLLRKDDNFYLQYTLETEFNLKEDYSGIVGIDRGVSHIAVYTFVHNNGKNERPLFLNSSEILRLKNLQKERDRFLRRKHNKKRKKSNMRNIEKKIQLILHNYSKQIVDFAKNKNAFIVFEKLEKPKKNRSKMSKKSQYKLSQFTFKKLSDLVDYKAKREGIKVLYISPEYTSKECSHCGEKVNTQRPFNGNSSLFKCNKCGVELNADYNASINIAKKGLNILNSTN

SEQ ID NO: 8 - protein, 537 aa, synthetic construct
MEESIITGVKFKLRIDKETTKKLNEYFDEYGKAINFAVKIIQKELADDRFAGKAKLDQNKNPILDENGKKIYEFPDEFCSCGKQVNKYVNNKPFCQECYKIRFTENGIRKRMYSAKGRKAEHKINILNSTNKISKTHFNYAIREAFILDKSIKKQRKKRNERLRESKKRLQQFIDMRDGKREICPTIKGQKVDRFIHPSWITKDKKLEDFRGYTLSIINSKIKILDRNIKREEKSLKEKGQIIFKAKRLMLDKSIRFVGDRKVLFTISKTLPKEYELDLPSKEKRLNWLKEKIEIIKNQKPKYAYLLRKNIESEKKPNYEYYLQYTLEIKPELKDFYDGAIGIDRGINHIAVCTFISNDGKVTPPKFFSSGEILRLKNLQKERDRFLLRKHNKNRKKGNMRVIENKINLILHRYSKQIVDMAKKLNASIVFEELGRIGKSRTKMKKSQRYKLSLFIFKKLSDLVDYKSRREGIRVTYVPPEYTSKECSHCGEKVNTQRPFNGNYSLFKCNKCGIQLNSDYNASINIAKKGLKIPNST

SEQ ID NO: 9 - protein, 541 aa, synthetic construct
MWTIVIGDFIEMPKQDLVTTGIKFKLDVDKETRKKLDDYFDEYGKAINFAVKIIQKNLKEDRFAGKIALGEDKKPLLDKDGKKIYNYPNESCSCGNQVRRYVNAKPFCVDCYKLKFTENGIRKRMYSARGRKADSDINIKNSTNKISKTHFNYAIREGFILDKSLKKQRSKRIKKLLELKRKLQEFIDIRQGQMVLCPKIKNQRVDKFIHPSWLKRDKKLEEFRGYSLSVVEGKIKIFNRNILREEDSLRQRGHVNFKANRIMLDKSVRFLDGGKVNFNLNKGLPKEYLLDLPKKENKLSWLNEKISLIKLQKPKYAYLLRREGSFFIQYTIENVPKTFSDYLGAIGIDRGISHIAVCTFVSKNGVNKAPVFFSSGEILKLKSLQKQRDLFLRGKHNKIRKKSNMRNIDNKINLILHKYSRNIVNLAKSEKAFIVFEKLEKIKKSRFKMSKSLQYKLSQFTFKKLSDLVEYKAKIEGIKVDYVPPEYTSKECSHCGEKVDTQRPFNGNSSLFKCNKCRVQLNADYNASINIAKKSLNISNN

SEQ ID NO: 10 - protein, 544 aa, synthetic construct
MNMSKTTISVKLKIIDLSSEKKEFLDNYFNEYAKATTFCQLRIRRLLRNTHWLGKKEKSSKKWIFESGICDLCGENKELVNEDRNSGEPAKICKRCYNGRYGNQMIRKLFVSTKKREVQENMDIRRVAKLNNTHYHRIPEEAFDMIKAADTAEKRRKKNVEYDKKRQMEFIEMFNDEKKRAARPKKPNERETRYVHISKLESPSKGYTLNGIKRKIDGMGKKIERAEKGLSRKKIFGYQGNRIKLDSNWVRFDLAESEITIPSLFKEMKLRITGPTNVHSKSGQIYFAEWFERINKQPNNYCYLIRKTSSNGKYEYYLQYTYEAEVEANKEYAGCLGVDIGCSKLAAAVYYDSKNKKAQKPIEIFTNPIKKIKMRREKLIKLLSRVKVRHRRRKLMQLSKTEPIIDYTCHKTARKIVEMANTAKAFISMENLETGIKQKQQARETKKQKFYRNMFLFRKLSKLIEYKALLKGIKIVYVKPDYTSQTCSSCGADKEKTERPSQAIFRCLNPTCRYYQRDINADFNAAVNIAKKALNNTEVVTTLL

SEQ ID NO: 11 - protein, 564 aa, synthetic construct
MARAKNQPYQKLTTTTGIKFKLDLSEEEGKRFDEYFSEYAKAVNFCAKVIYQLRKNLKFAGKKELAAKEWKFEISNCDFCNKQKEIYYKNIANGQKVCKGCHRTNFSDNAIRKKMIPVKGRKVESKFNIHNTTKKISGTHRHWAFEDAADIIESMDKQRKEKQKRLRREKRKLSYFFELFGDPAKRYELPKVGKQRVPRYLHKIIDKDSLTKKRGYSLSYIKNKIKISERNIERDEKSLRKASPIAFGARKIKMSKLDPKRAFDLENNVFKIPGKVIKGQYKFFGTNVANEHGKKFYKDRISKILAGKPKYFYLLRKKVAESDGNPIFEYYVQWSIDTETPAITSYDNILGIDAGITNLATTVLIPKNLSAEHCSHCGNNHVKPIFTKFFSGKELKAIKIKSRKQKYFLRGKHNKLVKIKRIRPIEQKVDGYCHVVSKQIVEMAKERNSCIALEKLEKPKKSKFRQRRREKYAVSMFVFKKLATFIKYKAAREGIEIIPVEPEGTSYTCSHCKNAQNNQRPYFKPNSKKSWTSMFKCGKCGIELNSDYNAAFNIAQKALNMTSA

SEQ ID NO: 12 - protein, 610 aa, synthetic construct
MDEKHFFCSYCNKELKISKNLINKISKGSIREDEAVSKAISIHNKKEHSLILGIKFKLFIENKLDKKKLNEYFDNYSKAVTFAARIFDKIRSPYKFIGLKDKNTKKWTFPKAKCVFCLEEKEVAYANEKDNSKICTECYLKEFGENGIRKKIYSTRGRKVEPKYNIFNSTKELSSTHYNYAIRDAFQLLDALKKQRQKKLKSIFNQKLRLKEFEDIFSDPQKRIELSLKPHQREKRYIHLSKSGQESINRGYTLRFVRGKIKSLTRNIEREEKSLRKKTPIHFKGNRLMIFPAGIKFDFASNKVKISISKNLPNEFNFSGTNVKNEHGKSFFKSRIELIKTQKPKYAYVLRKIKREYSKLRNYEIEKIRLENPNADLCDFYLQYTIETESRNNEEINGIIGIDRGITNLACLVLLKKGDKKPSGVKFYKGNKILGMKIAYRKHLYLLKGKRNKLRKQRQIRAIEPKINLILHQISKDIVKIAKEKNFAIALEQLEKPKKARFAQRKKEKYKLALFTFKNLSTLIEYKSKREGIPVIYVPPEKTSQMCSHCAINGDEHVDTQRPYKKPNAQKPSYSLFKCNKCGIELNADYNAAFNIAQKGLKTLMLNHSH

SEQ ID NO: 13 - protein, 613 aa, synthetic construct
MISLKLKLLPDEEQKKLLDEMFWKWASICTRVGFGRADKEDLKPPKDAEGVWFSLTQLNQANTDINDLREAMKHQKHRLEYEKNRLEAQRDDTQDALKNPDRREISTKRKDLFRPKASVEKGFLKLKYHQERYWVRRLKEINKLIERKTKTLIKIEKGRIKFKATRITLHQGSFKIRFGDKPAFLIKALSGKNQIDAPFVVVPEQPICGSVVNSKKYLDEITTNFLAYSVNAMLFGLSRSEEMLLKAKRPEKIKKKEEKLAKKQSAFENKKKELQKLLGRELTQQEEAIIEETRNQFFQDFEVKITKQYSELLSKIANELKQKNDFLKVNKYPILLRKPLKKAKSKKINNLSPSEWKYYLQFGVKPLLKQKSRRKSRNVLGIDRGLKHLLAVTVLEPDKKTFVWNKLYPNPITGWKWRRRKLLRSLKRLKRRIKSQKHETIHENQTRKKLKSLQGRIDDLLHNISRKIVETAKEYDAVIVVEDLQSMRQHGRSKGNRLKTLNYALSLFDYANVMQLIKYKAGIEGIQIYDVKPAGTSQNCAYCLLAQRDSHEYKRSQENSKIGVCLNPNCQNHKKQIDADLNAARVIASCYALKINDSQPFGTRKRFKKRTTN

SEQ ID NO: 14 - protein, 615 aa, synthetic construct
METLSLKLKLNPSKEQLLVLDKMFWKWASICTRLGLKKAEMSDLEPPKDAEGVWFSKTQLNQANTDVNDLRKAMQHQGKRIEYELDKVENRRNEIQEMLEKPDRRDISPNRKDLFRPKAAVEKGYLKLKYHKLGYWSKELKTANKLIERKRKTLAKIDAGKMKFKPTRISLHTNSFRIKFGEEPKIALSTTSKHEKIELPLITSLQRPLKTSCAKKSKTYLDAAILNFLAYSTNAALFGLSRSEEMLLKAKKPEKIEKRDRKLATKRESFDKKLKTLEKLLERKLSEKEKSVFKRKQTEFFDKFCITLDETYVEALHRIAEELVSKNKYLEIKKYPVLLRKPESRLRSKKLKNLKPEDWTYYIQFGFQPLLDTPKPIKTKTVLGIDRGVRHLLAVSIFDPRTKTFTFNRLYSNPIVDWKWRRRKLLRSIKRLKRRLKSEKHVHLHENQFKAKLRSLEGRIEDHFHNLSKEIVDLAKENNSVIVVENLGGMRQHGRGRGKWLKALNYALSHFDYAKVMQLIKYKAELAGVFVYDVAPAGTSINCAYCLLNDKDASNYTRGKVINGKKNTKIGECKTCKKEFDADLNAARVIALCYEKRLNDPQPFGTRKQFKPKKP

SEQ ID NO: 15 - protein, 775 aa, synthetic construct
MKALKLQLIPTRKQYKILDEMFWKWASLANRVSQKGESKETLAPKKDIQKIQFNATQLNQIEKDIKDLRGAMKEQQKQKERLLLQIQERRSTISEMLNDDNNKERDPHRPLNFRPKGWRKFHTSKHWVGELSKILRQEDRVKKTIERIVAGKISFKPKRIGIWSSNYKINFFKRKISINPLNSKGFELTLMTEPTQDLIGKNGGKSVLNNKRYLDDSIKSLLMFALHSRFFGLNNTDTYLLGGKINPSLVKYYKKNQDMGEFGREIVEKFERKLKQEINEQQKKIIMSQIKEQYSNRDSAFNKDYLGLINEFSEVFNQRKSERAEYLLDSFEDKIKQIKQEIGESLNISDWDFLIDEAKKAYGYEEGFTEYVYSKRYLEILNKIVKAVLITDIYFDLRKYPILLRKPLDKIKKISNLKPDEWSYYIQFGYDSINPVQLMSTDKFLGIDRGLTHLLAYSVFDKEKKEFIINQLEPNPIMGWKWKLRKVKRSLQHLERRIRAQKMVKLPENQMKKKLKSIEPKIEVHYHNISRKIVNLAKDYNASIVVESLEGGGLKQHGRKKNARNRSLNYALSLFDYGKIASLIKYKADLEGVPMYEVLPAYTSQQCAKCVLEKGSFVDPEIIGYVEDIGIKGSLLDSLFEGTELSSIQVLKKIKNKIELSARDNHNKEINLILKYNFKGLVIVRGQDKEEIAEHPIKEINGKFAILDFVYKRGKEKVGKKGNQKVRYTGNKKVGYCSKHGQVDADLNASRVIALCKYLDINDPILFGEQRKSFK

SEQ ID NO: 16 - protein, 777 aa, synthetic construct
MVTRAIKLKLDPTKNQYKLLNEMFWKWASLANRFSQKGASKETLAPKDGTQKIQFNATQLNQIKKDVDDLRGAMEKQGKQKERLLIQIQERLLTISEILRDDSKKEKDPHRPQNFRPFGWRRFHTSAYWSSEASKLTRQVDRVRRTIERIKAGKINFKPKRIGLWSSTYKINFLKKKINISPLKSKSFELDLITEPQQKIIGKEGGKSVANSKKYLDDSIKSLLIFAIKSRLFGLNNKDKPLFENIITPNLVRYHKKGQEQENFKKEVIKKFENKLKKEISQKQKEIIFSQIERQYENRDATFSEDYLRAISEFSEIFNQRKKERAKELLNSFNEKIRQLKKEVNGNISEEDLKILEVEAEKAYNYENGFIEWEYSEQFLGVLEKIARAVLISDNYFDLKKYPILIRKPTNKSKKITNLKPEEWDYYIQFGYGLINSPMKIETKNFMGIDRGLTHLLAYSIFDRDSEKFTINQLELNPIKGWKWKLRKVKRSLQHLERRMRAQKGVKLPENQMKKRLKSIEPKIESYYHNLSRKIVNLAKANNASIVVESLEGGGLKQHGRKKNSRHRALNYALSLFDYGKIASLIKYKSDLEGVPMYEVLPAYTSQQCAKCVLKKGSFVEPEIIGYIEEIGFKENLLTLLFEDTGLSSVQVLKKSKNKMTLSARDKEGKMVDLVLKYNFKGLVISQEKKKEEIVEFPIKEIDGKFAVLDSAYKRGKERISKKGNQKLVYTGNKKVGYCSVHGQVDADLNASRVIALCKYLGINEPIVFGEQRKSFK

SEQ ID NO: 17 - protein, 610 aa, synthetic construct
MDLITEPIQPHKSSSLRSKEFLEYQISDFLNFSLHSLFFGLASNEGPLVDFKIYDKIVIPKPEERFPKKESEEGKKLDSFDKRVEEYYSDKLEKKIERKLNTEEKNVIDREKTRIWGEVNKLEEIRSIIDEINEIKKQKHISEKSKLLGEKWKKVNNIQETLLSQEYVSLISNLSDELTNKKKELLAKKYSKFDDKIKKIKEDYGLEFDENTIKKEGEKAFLNPDKFSKYQFSSSYLKLIGEIARSLITYKGFLDLNKYPIIFRKPINKVKKIHNLEPDEWKYYIQFGYEQINNPKLETENILGIDRGLTHILAYSVFEPRSSKFILNKLEPNPIEGWKWKLRKLRRSIQNLERRWRAQDNVKLPENQMKKNLRSIEDKVENLYHNLSRKIVDLAKEKNACIVFEKLEGQGMKQHGRKKSDRLRGLNYKLSLFDYGKIAKLIKYKAEIEGIPIYRIDSAYTSQNCAKCVLESRRFAQPEEISCLDDFKEGDNLDKRILEGTGLVEAKIYKKLLKEKKEDFEIEEDIAMFDTKKVIKENKEKTVILDYVYTRRKEIIGTNHKKNIKGIAKYTGNTKIGYCMKHGQVDADLNASRTIALCKNFDINNPEIWK

SEQ ID NO: 18 - protein, 518 aa, synthetic construct
MVINLFGYKFALYPNKTQEELLNKHLGECGWLYNKAIEQNEYYKADSNIEEAQKKFELLPDKNSDEAKVLRGNISKDNYVYRTLVKKKKSEINVQIRKAVVLRPAETIRNLAKVKKKGLSVGRLKFIPIREWDVLPFKQSDQIRLEENYLILEPYGRLKFKMHRPLLGKPKTFCIKRTATDRWTISFSTEYDDSNMRKNDGGQVGIDVGLKTHLRLSNENPDEDPRYPNPKIWKRYDRRLTILQRRISKSKKLGKNRTRLRLRLSRLWEKIRNSRADLIQNETYEILSENKLIAIEDLNVKGMQEKKDKKGRKGRTRAQEKGLHRSISDAAFSEFRRVLEYKAKRFGSEVKPVSAIDSSKECHNCGNKKGMPLESRIYECPKCGLKIDRDLNSAKVILARATGVRPGSNARADTKISATAGASVQTEGTVSEDFRQQMETSDQKPMQGEGSKEPPMNPEHKSSGRGSKHVNIGCKNKVGLYNEDENSRSTEKQIMDENRSTTEDMVEIGALHSPVLTT

SEQ ID NO: 19 - protein, 602 aa, synthetic construct
MSQSLLKWHDMAGRDKDASRSLQKSAVEGVLLHLTASHRVALEMLEKSVSQTVAVTMEAAQQRLVIVLEDDPTKATSRKRVISADLQFTREEFGSLPNWAQKLASTCPEIATKYADKHINSIRIAWGVAKESTNGDAVEQKLQWQIRLLDVTMFLQQLVLQLADKALLEQIPSSIRGGIGQEVAQQVTSHIQLLDSGTVLKAELPTISDRNSELARKQWEDAIQTVCTYALPFSRERARILDPGKYAAEDPRGDRLINIDPMWARVLKGPTVKSLPLLFVSGSSIRIVKLTLPRKHAAGHKHTFTATYLVLPVSREWINSLPGTVQEKVQWWKKPDVLATQELLVGKGALKKSANTLVIPISAGKKRFFNHILPALQRGFPLQWQRIVGRSYRRPATHRKWFAQLTIGYTNPSSLPEMALGIHFGMKDILWWALADKQGNILKDGSIPGNSILDFSLQEKGKIERQQKAGKNVAGKKYGKSLLNATYRVVNGVLEFSKGISAEHASQPIGLGLETIRFVDKASGSSPVNARHSNWNYGQLSGIFANKAGPAGFSVTEITLKKAQRDLSDAEQARVLAIEATKRFASRIKRLATKRKDDTLFV

SEQ ID NO: 20 - protein, 494 aa, synthetic construct
MEPVEKERFYYRTYTFRLDGQPRTQNLTTQSGWGLLTKAVLDNTKHYWEIVHHARIANQPIVFENPVIDEQGNPKLNKLGQPRFWKRPISDIVNQLRALFENQNPYQLGSSLIQGTYWDVAENLASWYALNKEYLAGTATWGEPSFPEPHPLTEINQWMPLTFSSGKVVRLLKNASGRYFIGLPILGENNPCYRMRTIEKLIPCDGKGRVTSGSLILFPLVGIYAQQHRRMTDICESIRTEKGKLAWAQVSIDYVREVDKRRRMRRTRKSQGWIQGPWQEVFILRLVLAHKAPKLYKPRCFAGISLGPKTLASCVILDQDERVVEKQQWSGSELLSLIHQGEERLRSLREQSKPTWNAAYRKQLKSLINTQVFTIVTFLRERGAAVRLESIARVRKSTPAPPVNFLLSHWAYRQITERLKDLAIRNGMPLTHSNGSYGVRFTCSQCGATNQGIKDPTKYKVDIESETFLCSICSHREIAAVNTATNLAKQLLDE

SEQ ID NO: 21 - protein, 649 aa, synthetic construct
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGRHGHTRTDRLPAGNTLWRADFATSAEVAAPKWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANRRFSMWNYSRLATLIEWLGTDIATRDCGTAAPLAHKVSDYLTHFTCPECGACRKAGQKKEIADTVRAGDILTCRKCGFSGPIPDNFIAEFVAKKALERMLKKKPV

SEQ ID NO: 22 - protein, 414 aa, synthetic construct
MAKRNFGEKSEALYRAVRFEVRPSKEELSILLAVSEVLRMLFNSALAERQQVFTEFIASLYAELKSASVPEEISEIRKKLREAYKEHSISLFDQINALTARRVEDEAFASVTRNWQEETLDALDGAYKSFLSLRRKGDYDAHSPRSRDSGFFQKIPGRSGFKIGEGRIALSCGAGRKLSFPIPDYQQGRLAETTKLKKFELYRDQPNLAKSGRFWISVVYELPKPEATTCQSEQVAFVALGASSIGVVSQRGEEVIALWRSDKHWVPKIEAVEERMKRRVKGSRGWLRLLNSGKRRMHMISSRQHVQDEREIVDYLVRNHGSHFVVTELVVRSKEGKLADSSKPERGGSLGLNWAAQNTGSLSRLVRQLEEKVKEHGGSVRKHKLTLTEAPPARGAENKLWMARKLRESFLKEV

SEQ ID NO: 23 - protein, 449 aa, synthetic construct
MAKGKKKEGKPLYRAVRFEIFPTSDQITLFLRVSKNLQQVWNEAWQERQSCYEQFFGSIYERIGQAKKRAQEAGFSEVWENEAKKGLNKKLRQQEISMQLVSEKESLLQELSIAFQEHGVTLYDQINGLTARRIIGEFALIPRNWQEETLDSLDGSFKSFLALRKNGDPDAKPPRQRVSENSFYKIPGRSGFKVSNGQIYLSFGKIGQTLTSVIPEFQLKRLETAIKLKKFELCRDERDMAKPGRFWISVAYEIPKPEKVPVVSKQITYLAIGASRLGVVSPKGEFCLNLPRSDYHWKPQINALQERLEGVVKGSRKWKKRMAACTRMFAKLGHQQKQHGQYEVVKKLLRHGVHFVVTELKVRSKPGALADASKSDRKGSPTGPNWSAQNTGNIARLIQKLTDKASEHGGTVIKRNPPLLSLEERQLPDAQRKIFIAKKLREEFLADQK

SEQ ID NO: 24 - protein, 711 aa, synthetic construct
MAKREKKDDVVLRGTKMRIYPTDRQVTLMDMWRRRCISLWNLLLNLETAAYGAKNTRSKLGWRSIWARVVEENHAKALIVYQHGKCKKDGSFVLKRDGTVKHPPRERFPGDRKILLGLFDALRHTLDKGAKCKCNVNQPYALTRAWLDETGHGARTADIIAWLKDFKGECDCTAISTAAKYCPAPPTAELLTKIKRAAPADDLPVDQAILLDLFGALRGGLKQKECDHTHARTVAYFEKHELAGRAEDILAWLIAHGGTCDCKIVEEAANHCPGPRLFIWEHELAMIMARLKAEPRTEWIGDLPSHAAQTVVKDLVKALQTMLKERAKAAAGDESARKTGFPKFKKQAYAAGSVYFPNTTMFFDVAAGRVQLPNGCGSMRCEIPRQLVAELLERNLKPGLVIGAQLGLLGGRIWRQGDRWYLSCQWERPQPTLLPKTGRTAGVKIAASIVFTTYDNRGQTKEYPMPPADKKLTAVHLVAGKQNSRALEAQKEKEKKLKARKERLRLGKLEKGHDPNALKPLKRPRVRRSKLFYKSAARLAACEAIERDRRDGFLHRVTNEIVHKFDAVSVQKMSVAPMMRRQKQKEKQIESKKNEAKKEDNGAAKKPRNLKPVRKLLRHVAMARGRQFLEYKYNDLRGPGSVLIADRLEPEVQECSRCGTKNPQMKDGRRLLRCIGVLPDGTDCDAVLPRNRNAARNAEKRLRKHREAHNA

SEQ ID NO: 25 - protein, 743 aa, synthetic construct
MSVRSFQARVECDKQTMEHLWRTHKVFNERLPEIIKILFKMKRGECGQNDKQKSLYKSISQSILEANAQNADYLLNSVSIKGWKPGTAKKYRNASFTWADDAAKLSSQGIHVYDKKQVLGDLPGMMSQMVCRQSVEAISGHIELTKKWEKEHNEWLKEKEKWESEDEHKKYLDLREKFEQFEQSIGGKITKRRGRWHLYLKWLSDNPDFAAWRGNKAVINPLSEKAQIRINKAKPNKKNSVERDEFFKANPEMKALDNLHGYYERNFVRRRKTKKNPDGFDHKPTFTLPHPTIHPRWFVFNKPKTNPEGYRKLILPKKAGDLGSLEMRLLTGEKNKGNYPDDWISVKFKADPRLSLIRPVKGRRVVRKGKEQGQTKETDSYEFFDKHLKKWRPAKLSGVKLIFPDKTPKAAYLYFTCDIPDEPLTETAKKIQWLETGDVTKKGKKRKKKVLPHGLVSCAVDLSMRRGTTGFATLCRYENGKIHILRSRNLWVGYKEGKGCHPYRWTEGPDLGHIAKHKREIRILRSKRGKPVKGEESHIDLQKHIDYMGEDRFKKAARTIVNFALNTENAASKNGFYPRADVLLLENLEGLIPDAEKERGINRALAGWNRRHLVERVIEMAKDAGFKRRVFEIPPYGTSQVCSKCGALGRRYSIIRENNRREIRFGYVEKLFACPNCGYCANADHNASVNLNRRFLIEDSFKSYYDWKRLSEKKQKEEIETIESKLMDKLCAMHKISRGSISK

SEQ ID NO: 26 - protein, 486 aa, synthetic construct
MAEENTLHLTLAMSLPLNDLPENRTRSELWRRQWLPQKKLSLLLGVNQSVRKAAADCLRWFEPYQELLWWEPTDPDGKKLLDKEGRPIKRTAGHMRVLRKLEEIAPFRGYQLGSAVKNGLRHKVADLLLSYAKRKLDPQFTDKTSYPSIGDQFPIVWTGAFVCYEQSITGQLYLYLPLFPRGSHQEDITNNYDPDRGPALQVFGEKEIARLSRSTSGLLLPLQFDKWGEATFIRGENNPPTWKATHRRSDKKWLSEVLLREKDFQPKRVELLVRNGRIFVNVACEIPTKPLLEVENFMGVSFGLEHLVTVVVINRDGNVVHQRQEPARRYEKTYFARLERLRRRGGPFSQELETFHYRQVAQIVEEALRFKSVPAVEQVGNIPKGRYNPRLNLRLSYWPFGKLADLTSYKAVKEGLPKPYSVYSATAKMLCSTCGAANKEGDQPISLKGPTVYCGNCGTRHNTGFNTALNLARRAQELFVKGVVAR

SEQ ID NO: 27 - protein, 400 aa, synthetic construct
MTTQKTYNFCFYDQRFFELSKEAGEVYSRSLEEFWKIYDETGVWLSKFDLQKHMRNKLERKLLHSDSFLGAMQQVHANLASWKQAKKVVPDACPPRKPKFLQAILFKKSQIKYKNGFLRLTLGTEKEFLYLKWDINIPLPIYGSVTYSKTRGWKINLCLETEVEQKNLSENKYLSIDLGVKRVATIFDGENTITLSGKKFMGLMHYRNKLNGKTQSRLSHKKKGSNNYKKIQRAKRKTTDRLLNIQKEMLHKYSSFIVNYAIRNDIGNIIIGDNSSTHDSPNMRGKTNQKISQNPEQKLKNYIKYKFESISGRVDIVPEPYTSRKCPHCKNIKKSSPKGRTYKCKKCGFIFDRDGVGAINIYNENVSFGQIISPGRIRSLTEPIGMKFHNEIYFKSYVAA

SEQ ID NO: 28 - protein, 84 aa, unidentified
  source 1..84: note = Bacteriophage PBS2
MTNLSDIIEKETGKQLVIQESILMLPEEVEEVIGNKPESDILVHTAYDESTDENVMLLTSDAPEYKPWALVIQDSNGENKIKML

SEQ ID NO: 29 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 30-32 - skipped (3 entries)

SEQ ID NO: 33 - protein, 198 aa, Homo sapiens [sequence omitted]

SEQ ID NO: 34 - protein, 198 aa, Mus musculus
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 35 - protein, 198 aa, unidentified
  source 1..198: note = Canis familiaris
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 36 - protein, 199 aa, Bos taurus
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 37 - protein, 429 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYIPVPSSSSSTLSNICLTKGLPETRFCVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 38 - protein, 429 aa, Rattus norvegicus
MGPFCLGCSHRKCYSPIRNLISQETFKFHFKNLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVERRRVHLLSEEEFYSQFYNQRVKHLCYYHGVKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 39 - protein, 370 aa, Macaca mulatta
MVEPMDPRTFVSNFNNRPILSGLNTVWLCCEVKTKDPSGPPLDAKIFQGKVYSKAKYHPEMRFLRWFHKWRQLHHDQEYKVTWYVSWSPCTRCANSVATFLAKDPKVTLTIFVARLYYFWKPDYQQALRILCQKRGGPHATMKIMNYNEFQDCWNKFVDGRGKPFKPRNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRQGRPFQPWDGLDEHSQALSGRLRAI

SEQ ID NO: 40 - protein, 384 aa, Pan troglodytes
MKPHFRNPVERMYQDTFSDNFYNRPILSHRNTVWLCYEVKTKGPSRPPLDAKIFRGQVYSKLKYHPEMRFFHWFSKWRKLHRDQEYEVTWYISWSPCTKCTRDVATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTSNFNNELWVRGRHETYLCYEVERLHNDTWVLLNQRRGFLCNQAPHKHGFLEGRHAELCFLDVIPFWKLDLHQDYRVTCFTSWSPCFSCAQEMAKFISNNKHVSLCIFAARIYDDQGRCQEGLRTLAKAGAKISIMTYSEFKHCWDTFVDHQGCPFQPWDGLEEHSQALSGRLRAILQNQGN

SEQ ID NO: 41 - protein, 377 aa, Chlorocebus sabaeus
MNPQIRNMVEQMEPDIFVYYFNNRPILSGRNTVWLCYEVKTKDPSGPPLDANIFQGKLYPEAKDHPEMKFLHWFRKWRQLHRDQEYEVTWYVSWSPCTRCANSVATFLAEDPKVTLTIFVARLYYFWKPDYQQALRILCQERGGPHATMKIMNYNEFQHCWNEFVDGQGKPFKPRKNLPKHYTLLHATLGELLRHVMDPGTFTSNFNNKPWVSGQRETYLCYKVERSHNDTWVLLNQHRGFLRNQAPDRHGFPKGRHAELCFLDLIPFWKLDDQQYRVTCFTSWSPCFSCAQKMAKFISNNKHVSLCIFAARIYDDQGRCQEGLRTLHRDGAKIAVMNYSEFEYCWDTFVDRQGRPFQPWDGLDEHSQALSGRLRAI

SEQ ID NO: 42 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 43 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 44 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 45 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 46 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 47 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 48 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 49 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 50 - protein, 229 aa, Mus musculus
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 51 - protein, 229 aa, Rattus norvegicus [sequence omitted]

SEQ ID NO: 52 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 53 - protein, 208 aa, synthetic construct
MTDAEYVRIHEKLDIYTFKKQFSNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWVCKLYYEKNARNQIGLWNLRDNGVGLNVMVSEHYQCCRKIFIQSSHNQLNENRWLEKTLKRAEKRRSELSIMFQVKILHTTKSPAV

SEQ ID NO: 54 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISIMTYSEFKHCWDTFVDHQGCPFQPWDGLDEHSQDLSGRLRAILQNQEN

SEQ ID NO: 55 - protein, 184 aa, Homo sapiens
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQ

SEQ ID NO: 56 - protein, 184 aa, Homo sapiens
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISIMTYSEFKHCWDTFVDHQGCPFQPWDGLDEHSQDLSGRLRAILQ

SEQ ID NO: 57 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 58 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARNAKTGAAGSLMDVLHHPGMNHRVEITEGILADECAALLSDFFRMRRQEIKAQKKAQSSTD

SEQ ID NO: 59 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARGAKTGAAGSLMDVLHHPGMNHRVEITEGILADECAALLSDFFRMRRQEIKAQKKAQSSTD

SEQ ID NO: 60 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARVAKTGAAGSLMDVLHHPGMNHRVEITEGILADECAALLSDFFRMRRQEIKAQKKAQSSTD

SEQ ID NO: 61 - protein, 166 aa, synthetic construct
SEVEFSYEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARNAKTGAAGSLMDVLHHPGMSHRVEITEGILADECAALLSDFFRMRRQEIKAQKKAQSSTD

SEQ ID NO: 62 - protein, 166 aa, synthetic construct
SEVEFSYEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARNAKTGAAGSLMDVLHHPGMSHRVEITEGILADECAALLSDFFRMRRQDIKAQKKAQSSTD

SEQ ID NO: 63 - protein, 166 aa, synthetic construct
SEVEFSYEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARNAKTGAAGSLMDVLHHPGMSHRVEITEGILADECAALLSDFFRMRRQGIKAQKKAQSSTD

SEQ ID NO: 64 - protein, 166 aa, synthetic construct
SEVEFSYEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARNAKTGAAGSLMDVLHHPGMSHRVEITEGILADECAALLSDFFRMRRQVIKAQKKAQSSTD

SEQ ID NO: 65 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGVRNAKTGAAGSLMDVLHHPGMNHRVEITEGILADECAALLSYFFRMRRQVIKAQKKAQSSTD

SEQ ID NO: 66 - protein, 166 aa, synthetic construct
AEVEFSHEYWMRHALTLAKRAWDEREVPVGAVLVHNNRVIGEGWNRPFGRHDPTAHAEIMALRQGGLVMQNYRLIDATLYVTLEPCVMCAGAMIHSRIGRVVFGVRNAKTGAAGSLMDVLHHPGMNHRVEITEGILADECAALLSYFFRMRRQVIKAQKKAQSSTD

SEQ ID NO: 67 - protein, 166 aa, synthetic construct
SEVEFSYEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGTRNAKTGAAGSLMDVLHHPGMSHRVEITEGILADECAALLSDFFRMRRQEIKAQSKAQSSTD

SEQ ID NO: 68 - protein, 166 aa, synthetic construct
SEVEFSHEYWMRHALTLAKRAWDEGEVPVGAVLVHNNRVIGEGWNRPIGRHDPTAHAEIMALRQGGLVMQNYRLIDATLYVTFEPCVMCAGAMIHSRIGRVVFGVHNAKTGAAGSLMDVLHYPGMNHRVEITEGILADECNDLLSYFFRMRRQVFKAQKKAQSSTD

SEQ ID NO: 69 - protein, 166 aa, synthetic construct
SEVEFSHEYWMRHALTLAKRAWDGGEVPVGAVLVHNNRVIGEGWNRPIGRHDPTAHAEIMALRQGGLVMQNYRLIDATLYVTFEPCVMCAGAMIHSRIGRVVFGVHNAKTGAAGSLMDVLHYPGMNHRVEITEGILADECNDLLSYFFRMRRQVFKAQKKAQSSTD

SEQ ID NO: 70 - protein, 166 aa, synthetic construct
SEVEFSHEYWMRHALTLAKRAWDDGEVPVGAVLVHNNRVIGEGWNRPIGRHDPTAHAEIMALRQGGLVMQNYRLIDATLYVTFEPCVMCAGAMIHSRIGRVVFGVKNAKTGAAGSLMDVLHYPGMNHRVEITEGILADECNGLLSYFFRMRRQVFKAQKKAQSSTD

SEQ ID NO: 71 - protein, 166 aa, synthetic construct
SEVEFSHEYWMRHALTLAKRAWDEQEVPVGAVLVHNNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECNALLSYFFRMRRQVFKAQKKAQSSTD

SEQ ID NO: 72 - protein, 166 aa, synthetic construct
SEVEFSHEYWMRHALTLAKRAWDMGEVPVGAVLVHNNRVIGEGWNRPIGRHDPTAHAEIMALRQGGLVMQNYRLIDATLYVTFEPCVMCAGAMIHSRIGRVVFGVPNAKTGAAGSLMDVLHYPGMNHRVEITEGILADECNDLLSYFFRMRRQVFKAQKKAQSSTD

SEQ ID NO: 73 - protein, 166 aa, synthetic construct
SEVEFSHEYWMRHALTLAKRAWDECEVPVGAVLVHNNRVIGEGWNRPIGRHDPTAHAEIMALRQGGLVMQNYRLIDATLYVTFEPCVMCAGAMIHSRIGRVVFGVHNAKTGAAGSLMDVLHYPGMNHRVEITEGILADECNALLSYFFRMRRQVFKAQKKAQSSTD

SEQ ID NO: 74 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECNLLLSYFFRMRRQVFKAQKKAQSSTD

SEQ ID NO: 75 - protein, 166 aa, synthetic construct
SEVEFSHEYWMRHALTLAKRAWDEGEVPVGAVLVHNNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECNALLSYFFRMRRQVFKAQKKAQSSTD

SEQ ID NO: 76 - protein, 166 aa, synthetic construct
SEVEFSHEYWMRHALTLAKRAWDEREVPVGAVLVHNNRVIGEGWNRPIGHHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFNAQKKAQSSTD

SEQ ID NO: 77 - protein, 166 aa, synthetic construct
SEVEFSHEYWMRHALTLAKRAWDAGEVPVGAVLVHNNRVIGEGWNRPIGRHDPTAHAEIMALRQGGLVMQNYRLIDATLYVTFEPCVMCAGAMIHSRIGRVVFGVNNAKTGAAGSLMDVLHYPGMNHRVEITEGILADECNELLSYFFRMRRQVFKAQKKAQSSTD

SEQ ID NO: 78 - protein, 166 aa, synthetic construct
SEVEFSHEYWMRHALTLAKRAWDEREVPVGAVLVHTNRVIGEGWNRTIGRHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFKAQKKAQSSTD

SEQ ID NO: 79 - protein, 166 aa, synthetic construct
SEVEFSHEYWMRHALTLAKRAWDEREVPVGAVLVHSNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFKAQKKAQSSTD

SEQ ID NO: 80 - protein, 166 aa, synthetic construct
SEVEFSHEYWMRHALTLAKRAWDEREVPVGAVLVLNNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFKAQKKAQSSTD

SEQ ID NO: 81 - protein, 166 aa, synthetic construct
SEVEFSHEYWMRHALTLAKRAWDEREVPVGAVLVLNNRVIGEGWNRLIGRHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFKAQKKAQSSTD

SEQ ID NO: 82 - protein, 166 aa, synthetic construct
SEVEFSHEYWMRHALTLAKRAWDEREVPVGAVLVLNNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFNAQKKAQSSTD

SEQ ID NO: 83 - protein, 166 aa, synthetic construct
SEVEFSHEYWMRHALTLAKRAWDEREVPVGAVLVLNNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFKAQKKAQSSTD

SEQ ID NO: 84 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECAALLRYFFRMRRQVFKAQKKAQSSTD

SEQ ID NO: 85 - protein, 166 aa, synthetic construct
SEVEFSHEYWMRHALTLAKRAWDEREVPVGAVLVHSNRVIGEGWNRPIGHHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFKAQKKAQSSTD

SEQ ID NO: 86 - protein, 166 aa, synthetic construct
SEVEFSHEYWMRHALTLAKRAWDEREVPVGAVLVHNNRVIGEGWNRPIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFNAQKKAQSSTD

SEQ ID NO: 87 - protein, 159 aa, synthetic construct
GSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADNPKGGCSGSLMNLLQQSNFNHRAIVDKGVLKEACSTLLTTFFKNLRANKKSTN

SEQ ID NO: 88 - protein, 159 aa, synthetic construct
GSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGAANPKGGCSGSLMNLLQQSNFNHRAIVDKGVLKEACSTLLTTFFKNLRANKKSTN

SEQ ID NO: 89 - protein, 159 aa, synthetic construct
GSHMTNDIYFMTLAIEEAKKAAQLPEVPIGAIITKDDEVIARAHNLRETLQQPTAHAEHIAIERAAKVLGSWRLEGCTLYVTLEPCVMCAGTIVMSRIPRVVYGAANPKGGCSGSLMNLLQQSNFNHRAIVDKGVLKEACSTLLTTFFKNLRANKKSTN

SEQ ID NO: 90 - protein, 159 aa, synthetic construct
GSHMTNDIYFMTLAIEEAKKAAQLPEVPIGAIITKDDEVIARAHNLRETLQQPTAHAEHIAIERAAKVLGSWRLEGCTLYVTLEPCVMCAGTIVMSRIPRVVYGAANPKGGCSGSLMNLLQQSNFNHRAIVDKGVLKEACATLLTTFFKNLRANKKSTN

SEQ ID NO: 91 - protein, 159 aa, synthetic construct
GSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGAANPKGGCSGSLMNLLQQSNFNHRAIVDKGVLKEACATLLTTFFKNLRANKKSTN

SEQ ID NO: 92 - protein, 166 aa, synthetic construct
SEVEFSHEYWMRHALTLAKRAWDEREVPVGAVLVHNNRVIGEGWNRSIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 93 - protein, 166 aa, synthetic construct
SEVEFSHEYWMRHALTLAKRAWDEREVPVGAVLVHNNRVIGEGWNRTIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 94 - protein, 166 aa, synthetic construct
SEVEFSHEYWMRHALTLAKRAWDEREVPVGAVLVHNNRVIGEGWNRAIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 95 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECNALLSDFFRMRRQEIKAQKKAQSSTD

SEQ ID NO: 96 - protein, 166 aa, synthetic construct
SEVEFSHEYWMRHALTLAKRARDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 97 - protein, 166 aa, synthetic construct
SEVEFSHEYWMRHALTLAKRALDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 98 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMPRQEIKAQKKAQSSTD

SEQ ID NO: 99 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMHRQEIKAQKKAQSSTD

SEQ ID NO: 100 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFKAQKKAQSSTD

SEQ ID NO: 101 - protein, 166 aa, synthetic construct
SEVEFSHEYWMRHALTLAKRAWDEREVPVGAVLVLNNRVIGEGWNRPIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 102 - protein, 166 aa, synthetic construct
SEVEFSHEYWMRHALTLAKRAWDEREVPVGAVLVLNNRVIGEGWNRSIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 103 - protein, 166 aa, synthetic construct
SEVEFSHEYWMRHALTLAKRAWDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 104 - protein, 166 aa, synthetic construct
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 105 - protein, 160 aa, Staphylococcus aureus
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 106 - protein, 161 aa, Bacillus subtilis
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 107 - protein, 183 aa, unidentified
  source 1..183: note = Salmonella typhimurium
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 108 - protein, 164 aa, Shewanella putrefaciens
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 109 - protein, 173 aa, Haemophilus influenzae
  source 1..173: note = strain F3031
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 110 - protein, 161 aa, unidentified
  source 1..161: note = Caulobacter crescentus
MRTDESEDQDHRMMRLALDAARAAAEAGETPVGAVILDPSTGEVIATAGNGPIAAHDPTAHAEIAAMRAAAAKLGNYRLTDLTLVVTLEPCAMCAGAISHARIGRVVFGADDPKGGAVVHGPKFFAQPTCHWRPEVTGGVLADESADLLRGFFRARRKAKI

SEQ ID NO: 111 - protein, 179 aa, Geobacter sulfurreducens
MSSLKKTPIRDDAYWMGKAIREAAKAAARDEVPIGAVIVRDGAVIGRGHNLREGSNDPSAHAEMIAIRQAARRSANWRLTGATLYVTLEPCLMCMGAIILARLERVVFGCYDPKGGAAGSLYDLSADPRLNHQVRLSPGVCQEECGTMLSDFFRDLRRRKKAKATPALFIDERKVPPEP

SEQ ID NO: 112 - protein, 171 aa, Streptococcus pyogenes
MPYSLEEQTYFMQEALKEAEKSLQKAEIPIGCVIVKDGEIIGRGHNAREESNQAIMHAEIMAINEANAHEGNWRLLDTTLFVTIEPCVMCSGAIGLARIPHVIYGASNQKFGGADSLYQILTDERLNHRVQVERGLLAADCANIMQTFFRQGRERKKIAKHLIKEQSDPFD

SEQ ID NO: 113 - protein, 166 aa, synthetic construct; identical to SEQ ID NO: 29

SEQ ID NO: 114 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNAKTGAAGSLMDVLHYPGMNHRVEITEGILADECAALLCYFFRMPRQVFNAQKKAQSSTD

SEQ ID NO: 115 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 116 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSIN

SEQ ID NO: 117 - protein, 151 aa, unidentified
  source 1..151: note = Aquifex aeolicus
MGKEYFLKVALREAKRAFEKGEVPVGAIIVKEGEIISKAHNSVEELKDPTAHAEMLAIKEACRRLNTKYLEGCELYVTLEPCIMCSYALVLSRIEKVIFSALDKKHGGVVSVFNILDEPTLNHRVKWEYYPLEEASELLSEFFKKLRNNII

SEQ ID NO: 118 - protein, 166 aa, synthetic construct
SEVEFSHEYWMRHALTLAKRARDEGEVPVGAVLVLNNRVIGEGWNRAIGLYDPTAHAEIMALRQGGLVMQNYRLIDATLYVTFEPCVMCAGAMIHSRIGRVVFGVRNSKRGAAGSLMNVLNYPGMDHRVEITEGILADECAALLCDFYRMPRQVFNAQKKAQSSIN

SEQ ID NO: 119 - protein, 166 aa, synthetic construct
SEVEFSHEYWMRHALTLAKRARDEGEVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMDHRVEITEGILADECAALLCDFYRMPRQVFNAQKKAQSSIN

SEQ ID NO: 120 - protein, 166 aa, synthetic construct
SEVEFSHEYWMRHALTLAKRARDEREVPVGAVLVLNNRVIGEGWNRAIGLHDPTAHAEIMALRQGGLVMQNYGLIDATLYVTFEPCVMCAGAIIHSRIGRVVFGVRNSKRGAAGSLMNVLNYPGMNHRVEITEGILADECAALLCDFYRMPRQVFNAQKKAQSSIN

SEQ ID NO: 121 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMDHRVEITEGILADECAALLCDFYRMPRQVFNAQKKAQSSIN

SEQ ID NO: 122 - protein, 166 aa, synthetic construct
SEVEFSHEYWMRHALTLAKRARDEGEVPVGAVLVLNNRVIGEGWNRAIGLYDPTAHAEIMALRQGGLVMQNYGLIDATLYVTFEPCVMCAGAMIHSRIGRVVFGVRNSKRGAAGSLMNVLNYPGMDHRVEITEGILADECAALLCDFYRMPRQVFNAQKKAQSSIN

SEQ ID NO: 123 - protein, 166 aa, synthetic construct
SEVEFSHEYWMRHALTLAKRARDEGEVPVGAVLVLNNRVIGEGWNRAIGLYDPTAHAEIMALRQGGLVMQNYGLIDATLYSTFEPCVMCAGAMIHSRIGRVVFGVRNSKRGAAGSLMNVLNYPGMDHRVEITEGILADECAALLCDFYRMPRRVFNAQKKAQSSIN

SEQ ID NO: 124 - protein, 677 aa, unidentified
  source 1..677: note = Moloney murine leukemia virus
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 125 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 126 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 127 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 128 - protein, 677 aa, synthetic construct
TLNIEDEYRLHETSKEPDVSLGSTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 129 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 130 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSP

SEQ ID NO: 131 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 132 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 133 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSKARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 134 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 135 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 136 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 137 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGTAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 138 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSP

SEQ ID NO: 139 - protein, 677 aa, synthetic construct
TLNIEDEYRLHETSKEPDVSLGSTWLSDFPQAWAETGGMGLAVRQAPLIILLKATSTPVSIKQYPMKQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPALFDEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRKFLGTAGNCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAELIALTQALKMAEGKKLNVYTNSRYAFATAHIQGEIYRRRGLLTSEGKEIKNKDEILALLKALFLPKRLSIIHCPGHQKGHSAEARGNRMANQAARKAAITETPDTSTLLIENSSP

SEQ ID NO: 140 - protein, 677 aa, synthetic construct
TLNIEDEYRLHETSKEPDVSLGSTWLSDFPQAWAETGGMGLAVRQAPLIILLKATSTPVSIKQYPMKQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPALFNEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRKFLGTAGNCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAELIALTQALKMAEGKKLNVYTNSRYAFATAHIQGEIYRRRGLLTSEGKEIKNKDEILALLKALFLPKRLSIIHCPGHQKGHSAEARGNRMANQAARKAAITETPDTSTLLIENSSP

SEQ ID NO: 141 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 142 - protein, 7 aa, unidentified
  source 1..7: note = simian vacuolating virus 40
PKKKRKV

SEQ ID NO: 143 - protein, 19 aa, synthetic construct
MKRTADGSEFESPKKKRKV

SEQ ID NO: 144 - protein, 30 aa, synthetic construct
MDSLLMNRRKFLYQFKNVRWAKGRRETYLC

SEQ ID NO: 145 - protein, 20 aa, synthetic construct
AVKRPAATKKAGQAKKKKLD

SEQ ID NO: 146 - protein, 25 aa, synthetic construct
MSRRRKANPTKLSENAKKLAKEVEN

SEQ ID NO: 147 - protein, 9 aa, synthetic construct
PAAKRVKLD

SEQ ID NO: 148 - protein, 9 aa, synthetic construct
KLKIKRPVK

SEQ ID NO: 149 - protein, 8 aa, synthetic construct
VSRKRPRP

SEQ ID NO: 150 - protein, 10 aa, unidentified
  source 1..10: note = Hepatitis D virus
EGAPPAKRAR

SEQ ID NO: 151 - protein, 12 aa, Mus musculus
PPQPKKKPLDGE

SEQ ID NO: 152 - protein, 21 aa, synthetic construct
SGGSKRTADGSEFEPKKKRKV

SEQ ID NO: 153 - protein, 18 aa, unidentified
  source 1..18: note = simian vacuolating virus 40
KRTADGSEFESPKKKRKV

SEQ ID NO: 154 - protein, 16 aa, Xenopus laevis
KRXXXXXXXXXXKKKL

SEQ ID NO: 155 - protein, 17 aa, synthetic construct
KRTADGSEFEPKKKRKV

SEQ ID NO: 156 - protein, 150 aa, synthetic construct
  REGION 6..150: note = may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 157 - protein, 30 aa, synthetic construct
  REGION 2..30: note = may be absent
GGGGGGGGGGGGGGGGGGGGGGGGGGGGGG

SEQ ID NO: 158 - protein, 150 aa, synthetic construct
  REGION 6..150: note = may be absent
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 159 - protein, 90 aa, synthetic construct
  REGION 4..90: note = may be absent
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 160 - protein, 120 aa, synthetic construct
  REGION 5..120: note = may be absent
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 161 - protein, 60 aa, synthetic construct
  REGION 3..60: note = may be absent
XPXPXPXPXPXPXPXPXPXPXPXPXPXPXPXPXPXPXPXPXPXPXPXPXPXPXPXPXPXP

SEQ ID NO: 162 - protein, 16 aa, synthetic construct
SGSETPGTSESATPES

SEQ ID NO: 163 - protein, 12 aa, synthetic construct
SGSETPGTSESA

SEQ ID NO: 164 - protein, 21 aa, synthetic construct
SGSETPGTSESATPEGGSGGS

SEQ ID NO: 165 - protein, 32 aa, synthetic construct
SGGSSGGSSGSETPGTSESATPESSGGSSGGS

SEQ ID NO: 166 - protein, 10 aa, synthetic construct
SGGSGGSGGS

SEQ ID NO: 167 - protein, 60 aa, synthetic construct
SGGSSGGSSGSETPGTSESATPESAGSYPYDVPDYAGSAAPAAKKKKLDGSGSGGSSGGS

SEQ ID NO: 168 - protein, 6 aa, synthetic construct
GGSGGS

SEQ ID NO: 169 - protein, 9 aa, synthetic construct
GGSGGSGGS

SEQ ID NO: 170 - protein, 33 aa, synthetic construct
SGGSSGGSSGSETPGTSESATPESSGGSSGGSS

SEQ ID NO: 171 - protein, 60 aa, synthetic construct; identical to SEQ ID NO: 167

SEQ ID NO: 172 - RNA, 202 nt, unidentified
  source 1..202: mol_type = genomic RNA; note = DPANN super-phylum
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaac

SEQ ID NO: 173 - RNA, 198 nt, synthetic construct
  source 1..198: mol_type = other RNA
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttcctctccaattctgcacaagaaagttgcagaacccgaatagaaatgaaggaatgcaac

SEQ ID NO: 174 - RNA, 202 nt, synthetic construct
  source 1..202: mol_type = other RNA
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcattcttcctctccaattctgcacaagaaagttgcagaacccgaatagacgaatgaaggaatgcaac

SEQ ID NO: 175 - RNA, 202 nt, synthetic construct
  source 1..202: mol_type = other RNA
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatatttcctctccaattctgcacaagaaagttgcagaacccgaatagacgtatgaaggaatgcaac

SEQ ID NO: 176 - RNA, 166 nt, synthetic construct
  source 1..166: mol_type = other RNA
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttcaagaaagtgaatgaaggaatgcaac

SEQ ID NO: 177 - protein, 229 aa, synthetic construct
MSSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLK

SEQ ID NO: 178 - protein, 229 aa, synthetic construct
MSSETGPVAVDPTLRRRIEPHEFEVFFDPRELRKETCLLYEINWGGRHSIWRHTSQNTNKHVEVNFIEKFTTERYFCPNTRCSITWFLSYSPCGECSRAITEFLSRYPHVTLFIYIARLYHHADPENRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 179 - protein, 229 aa, synthetic construct
MSSETGPVAVDPTLRRRIEPHEFEVFFDPRELRKETCLLYEINWGGRHSIWRHTSQNTNKHVEVNFIEKFTTERYFCPNTRCSITWFLSYSPCGECSRAITEFLSRYPHVTLFIYIARLYHHADPRNRQGLEDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 180 - protein, 229 aa, synthetic construct
MSSETGPVAVDPTLRRRIEPHEFEVFFDPRELRKETCLLYEINWGGRHSIWRHTSQNTNKHVEVNFIEKFTTERYFCPNTRCSITWFLSYSPCGECSRAITEFLSRYPHVTLFIYIARLYHHADPENRQGLEDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 181 - protein, 229 aa, synthetic construct
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPENRQGLEDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 182 - protein, 229 aa, synthetic construct
MSSETGPVAVDPTLRRRIEPHEFEVFFDPRELAKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 183 - protein, 229 aa, synthetic construct
MSSETGPVAVDPTLRRRIEPHEFEVFFDPRELAAETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 184 - protein, 229 aa, synthetic construct
MSSETGPVAVDPTLRRRIEPHEFEVFFDPRELAAETCLLYEINWGGRHSIWRHTSQNTNKHVEVNFIEKFTTERYFCPNTRCSITWFLSWSPCGECSRAITEFLSRYPHVTLFIYIARLYHLANPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 185 - protein, 162 aa, synthetic construct
MFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYHEDERNRQGLRDLVNSGVTIRIMDLPDYNYCWKTFVSDQGGDEDYWPGHFAPWIKQYSLKL

SEQ ID NO: 186 - protein, 162 aa, synthetic construct
MFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKL

SEQ ID NO: 187 - DNA, 130 bp, unidentified
  source 1..130: mol_type = genomic DNA; note = DPANN super-phylum
ctttcttcggaaagtaaccctcgaaacaaattcatttttcctctccaattctgcacaagaaagttgcagaacccgaatagacgaatgaaggaatgcaaccagctattcaggctggcccgtttttttaagc

SEQ ID NO: 188 - DNA, 130 bp, unidentified
  source 1..130: mol_type = genomic DNA; note = DPANN super-phylum
gcttaaaaaaacgggccagcctgaatagctggttgcattccttcattcgtctattcgggttctgcaactttcttgtgcagaattggagaggaaaaatgaatttgtttcgagggttactttccgaagaaag

SEQ ID NO: 189 - DNA, 94 bp, unidentified
  source 1..94: mol_type = genomic DNA; note = DPANN super-phylum
tcagcctaatgtcgagaagtgctttcttcggaaagtaaccctcgaaacaaattcatttttcctctccaattctgcacaagaaagttgcagaacc

SEQ ID NO: 190 - DNA, 93 bp, synthetic construct
  source 1..93: mol_type = other DNA
tcagcctaatgtcgagaagtgctttcttcggaaagtaaccctcgaaacaaattcattttcctctccaattctgcacaagaaagttgcagaacc

SEQ ID NO: 191 - DNA, 94 bp, synthetic construct; identical to SEQ ID NO: 189
  source 1..94: mol_type = other DNA

SEQ ID NO: 192 - DNA, 94 bp, synthetic construct; identical to SEQ ID NO: 189
  source 1..94: mol_type = other DNA

SEQ ID NO: 193 - DNA, 94 bp, synthetic construct; identical to SEQ ID NO: 189
  source 1..94: mol_type = other DNA

SEQ ID NO: 194 - DNA, 94 bp, synthetic construct; identical to SEQ ID NO: 189
  source 1..94: mol_type = other DNA

SEQ ID NO: 195 - DNA, 93 bp, synthetic construct; identical to SEQ ID NO: 190
  source 1..93: mol_type = other DNA

SEQ ID NO: 196 - DNA, 94 bp, synthetic construct
  source 1..94: mol_type = other DNA
tcagcctaatgtcgagaagtgttttcttcggaaagtaaccctcgaaacaaattcatttttcctctccaattctgcacaagaaagttgcagaacc

SEQ ID NO: 197 - DNA, 94 bp, synthetic construct; identical to SEQ ID NO: 189
  source 1..94: mol_type = other DNA

SEQ ID NO: 198 - RNA, 65 nt, synthetic construct
  source 1..65: mol_type = other RNA
aaattcatttttcctctccaattctgcacaagaaagttgcagaacccgaatagacgaatgaagga

SEQ ID NO: 199 - RNA, 61 nt, synthetic construct
  source 1..61: mol_type = other RNA
aaattcatttcctctccaattctgcacaagaaagttgcagaacccgaatagaaatgaagga

SEQ ID NO: 200 - RNA, 65 nt, synthetic construct
  source 1..65: mol_type = other RNA
aaattcattcttcctctccaattctgcacaagaaagttgcagaacccgaatagacgaatgaagga

SEQ ID NO: 201 - RNA, 65 nt, synthetic construct
  source 1..65: mol_type = other RNA
aaattcatatttcctctccaattctgcacaagaaagttgcagaacccgaatagacgtatgaagga

SEQ ID NO: 202 - RNA, 29 nt, synthetic construct
  source 1..29: mol_type = other RNA
aaattcatttcaagaaagtgaatgaagga

SEQ ID NO: 203 - RNA, 29 nt, synthetic construct
  source 1..29: mol_type = other RNA
aaattcatttcaagaaagtgaatgaagga

SEQ ID NO: 204 - protein, 19 aa, synthetic construct
NLSKRPAAIKKAGQAKKKK

SEQ ID NO: 205 - protein, 11 aa, synthetic construct
RQRRNELKRSF

SEQ ID NO: 206 - protein, 38 aa, synthetic construct
NQSSNFGPMKGGNFGGRSSGPYGGGGQYFAKPRNQGGY

(19) United States
(12) Patent Application Publication
(10) Pub. No.: US 2025/0101395 A1
(43) Pub. Date: Mar. 27, 2025

(54) EVOLVED CAS14A1 VARIANTS, COMPOSITIONS, AND METHODS OF MAKING AND USING SAME IN GENOME EDITING

(71) Applicants: The Broad Institute, Inc., Cambridge, MA (US); President and Fellows of Harvard College, Cambridge, MA (US)

(72) Inventors: David R. Liu, Cambridge, MA (US); Aditya Raguram, Cambridge, MA (US)

(73) Assignees: The Broad Institute, Inc., Cambridge, MA (US); President and Fellows of Harvard College, Cambridge, MA (US)

(21) Appl. No.: 18/972,158

(22) Filed: Dec. 6, 2024

Related U.S. Application Data
(63) Continuation of application No. PCT/US2023/068064, filed on Jun. 7, 2023.
(60) Provisional application No. 63/350,242, filed on Jun. 8, 2022.

(51) Int. Cl. C12N 9/22; C12N 9/12; C12N 9/78; C12N 15/11; C12N 15/90

(52) U.S. Cl. CPC C12N 9/22; C12N 9/1276; C12N 9/78; C12N 15/11; C12N 15/907; C12Y 207/07049; C12Y 305/04001; C12Y 305/04004; C07K 2319/09; C12N 2310/20

(57) ABSTRACT

The present disclosure provides Cas protein variants comprising one or more amino acid substitutions relative to wild-type Cas14a1. Fusion proteins comprising the Cas protein variants described herein are also provided by the present disclosure. Further provided herein are methods for modifying a target nucleic acid using the Cas proteins and fusion proteins provided herein. The present disclosure also provides guide RNAs, complexes, polynucleotides, systems, cells, kits, and pharmaceutical compositions.

### RELATED APPLICATIONS

This application claims priority under 35 U.S.C. 119(e) to U.S. Provisional Application, 63/350,242, filed Jun. 8, 2022, the contents of which is incorporated by reference herein.

### FEDERALLY SPONSORED RESEARCH

This invention was made with government support under Grant Nos. U01 AI142756, RM1 HG009490, R01EB031172, and R35 GM118062, awarded by the National Institutes of Health. The government has certain rights in the invention.

### REFERENCE TO AN ELECTRONIC SEQUENCE LISTING

The contents of the electronic sequence listing (B119570152W000-SEQ-JQM.xml; Size: 231,271 bytes; and Date of Creation: Jun. 6, 2023) is herein incorporated by reference in its entirety.

### BACKGROUND OF THE INVENTION

CRISPR-Cas systems have been developed for use in genome modification (e.g., genome editing). In particular, several genome modifying agents using the CRISPR-Cas9 protein have been developed. These systems include, for example, base editors comprising a Cas protein fused to a deaminase, which facilitate the introduction of single nucleotide modifications to a genome without induction of a double-strand break. Such genome modifying systems also include prime editors comprising a Cas protein fused to a reverse transcriptase, which facilitate the insertion or deletion of a sequence of interest at a target site in a genome without the need for double strand breaks or a donor DNA template.

Current genome modifying agents, including base editors and prime editors, are quite large due, at least in part, to their reliance on Cas9 proteins. Staphylococcus aureus Cas9 (SpCas9), for example, is a 1368 amino acid-long protein, with a mass of over 158 kDa. Accordingly, there is a need for smaller-sized CRISPR-Cas proteins for use in genome modifying agents and systems to facilitate their delivery into cells, particularly in vivo.

### SUMMARY OF THE INVENTION

The present disclosure describes the use of multiple rounds of phage-assisted continuous evolution (PACE) and non-continuous evolution (PANCE) of Cas14a1 (a Cas protein from an archea of the DPANN super-phylum) to yield several variants with improved activity (e.g., improved base editing activity when fused to a deaminase) in bacterial and mammalian cells (e.g., human cells). Rational engineering of the Cas14a1 guide RNA was also performed, further enabling robust activity of the improved Cas14a1 variants provided herein in human cells. Because Cas14a1 is only 529 amino acids long (i.e., less than half the size of Cas9), and therefore small enough to enable single-AAV delivery of various CRISPR-based genome editing agents into cells, including base editors and prime editors, the evolved Cas variants described herein are useful in high-activity genome editing agents. Single-AAV delivery is discussed further in, e.g., International Patent Application No. PCT/US2023/066389, filed Apr. 28, 2023, which is incorporated herein by reference. AAV have a packaging capacity of ˜4.9 kilobases (kb), and the small size of Cas14a1 (and the evolved variants thereof described herein) therefore provides advantages for delivering Cas14a1-containing genome editing agents using AAV.

Accordingly, in one aspect, the present disclosure provides Cas proteins comprising an amino acid sequence that is at least 70%, at least 75%, at least 80%, at least 85%, at least 90%, at least 95%, or at least 99% identical to the amino acid sequence of a Cas protein of SEQ ID NO: 2, wherein the amino acid sequence of the Cas protein comprises at least 1, at least 2, at least 3, at least 4, at least 5, at least 6, at least 7, at least 8, at least 9, at least 10, at least 11, or at least 12 substitutions at positions selected from the group consisting of amino acid residues 1, 2, 11, 25, 32, 37, 41, 43, 44, 46, 58, 66, 76, 87, 118, 131, 134, 137, 138, 148, 157, 179, 201, 203, 206, 209, 210, 228, 260, 266, 268, 274, 282, 284, 296, 297, 298, 301, 303, 305, 309, 313, 320, 330, 341, 349, 352, 353, 366, 367, 372, 378, 392, 423, 425, 430, 461, 471, 477, 483, 486, 507, 508, 510, 513, 519, 528, and 529 of the amino acid sequence provided in SEQ ID NO: 2. In some embodiments, the Cas proteins comprise one or more substitutions selected from the group consisting of M1X, A2X, K11X, K25X, N32X, I37X, K41X, K43X, D44X, V46X, A58X, R66X, K76X, G87X, I118X, I131X, A134X, V137X, E138X, R148X, A157X, K179X, Q201X, T203X, E206X, N209X, H210X, E228X, K260X, S266X, D268X, E274X, D282X, Q284X, I296X, C297X, E298X, A301X, M303X, N305X, D309X, I313X, S320X, K330X, F341X, N349X, F352X, H353X, L366X, K367X, K372X, A378X, S392X, N423X, E425X, K430X, I461X, T471X, K477X, N483X, N486X, E507X, N508X, A510X, A513X, N519X, E528X, and P529X, relative to the amino acid sequence provided in SEQ ID NO: 2, wherein X represents any amino acid other than the wild type amino acid. In certain embodiments, the Cas proteins comprise one or more substitutions selected from the group consisting of M1R, A2S, K11T, K25R, N32D, I37V, K41E, K43R, D44G, V46G, A58T, R66S, K76E, K76T, G87E, I118F, I131T, A134T, V137A, E138A, R148K, A157T, K179T, Q201R, T203R, E206K, N209K, H210Y, E228D, K260R, S266I, D268A, E274D, D282E, Q284R, I296N, I296F, C297G, E298G, A301T, M303V, N305H, D309A, I313V, S320N, K330T, F341S, F341C, N349S, F352Y, H353Y, L366M, K367E, K372M, A378V, S392I, N423T, N423S, N423D, E425K, K430R, I461V, T471I, K477E, N483D, N486D, E507D, N508D, A510D, A513S, N519I, E528K, and P529S, relative to the amino acid sequence of SEQ ID NO: 2. In certain embodiments, a Cas protein comprises the amino acid substitutions A58T, K76E, Q201R, H210Y, E274D, A301T, F341C, E425K, N486D, and S524P relative to the amino acid sequence of SEQ ID NO: 2.

In some embodiments, the Cas proteins comprise at least 2, at least 3, at least 4, at least 5, at least 6, at least 7, at least 8, at least 9, at least 10, at least 11, or at least 12 substitutions at positions selected from the group consisting of amino acid residues 1, 79, 111, 121, 133, 135, 151, 179, 202, 213, 228, 232, 236, 244, 260, 261, 280, 285, 313, 344, 369, 374, 388, 392, 393, 423, 425, 429, 430, 448, 459, 460, 464, 497, 513, 516, 525, and 526 of the amino acid sequence provided in SEQ ID NO: 2. In some embodiments, the substitutions are selected from the group consisting of M1X, D79X, E111X, Y121X, N133X, S135X, E151X, K179X, Y202X, D213X, E228X, Y232X, E236X, Q244X, K260X, R261X, N280X, T285X, I313X, Y344X, N369X, A374X, L388X, S392X, E393X, N423X, K425X, R429X, K430X, M448X, Y459X, G460X, R464X, H497X, A513X, N516X, T525X, and K526X, relative to the amino acid sequence provided in SEQ ID NO: 2, wherein X represents any amino acid other than the wild type amino acid. In certain embodiments, the substitutions are selected from the group consisting of M1K, M1I, D79Y, E111K, Y121H, N133T, N133K, S135R, E151K, E151A, K179E, Y202D, Y202C, D213A, D213N, E228G, Y232C, Y232F, E236D, Q244K, Q244R, K260R, R261K, N280S, T285I, I313V, I313T, Y344C, N369D, A374V, L388R, S392I, E393K, N423T, N423D, K425E, R429L, K430R, M448I, Y459S, G460A, R464I, H497P, A513V, N516S, T525A, and K526R, relative to the amino acid sequence provided in SEQ ID NO: 2. In certain embodiments, a Cas protein further comprises the amino acid substitutions N133K, E228G, E236D, Q244K, K260R, T285I, A374V, and K425E relative to SEQ ID NO: 2.

In another aspect, the present disclosure provides fusion proteins. In some embodiments, the fusion proteins comprise (i) a Cas protein variant provided herein; and (ii) an effector domain. In some embodiments, an effector domain comprises nuclease activity, nickase activity, recombinase activity, deaminase activity, methyltransferase activity, methylase activity, acetylase activity, acetyltransferase activity, transcriptional activation activity, transcriptional repression activity, or polymerase activity. In certain embodiments, the effector domain is a nucleic acid editing domain, such as a deaminase domain (i.e., the fusion protein is a base editor, such as a cytosine base editor when the deaminase is a cytidine deaminase, or an adenine base editor when the deaminase is an adenosine deaminase). In some embodiments, the fusion proteins comprise (i) a Cas protein variant provided herein; and (ii) a domain comprising an RNA-dependent DNA polymerase activity. In certain embodiments, the domain comprising an RNA-dependent DNA polymerase activity is a reverse transcriptase (i.e., the fusion protein is a prime editor).

In another aspect, the present disclosure provides guide RNAs (gRNAs) created by rational engineering. In some embodiments, the gRNAs provided herein comprise mutations in a poly-U tract of the wild type Cas14a1 gRNA backbone sequence. In some embodiments, the gRNAs provided herein comprise a nucleic acid sequence of any one of SEQ ID NOs: 173-176, or a nucleic acid sequence that is at least 70%, at least 75%, at least 80%, at least 85%, at least 90%, at least 95%, or at least 99% identical to the nucleic acid sequence of any one of SEQ ID NOs: 173-176. In some embodiments, the gRNAs comprise a nucleic acid sequence that is 100% identical to the nucleic acid sequence of any one of SEQ ID NOs: 173-176 (e.g., the nucleic acid sequence 5′-CUUCACUGAUAAAGUGGAGAACCGCUUCACCAAAAGCUGUCCCUUAGGGGAUUA GAACUUGAGUGAAGGUGGGCUGCUUGCAUCAGCCUAAUGUCGAGAAGUGCUUUC UUCGGAAAGUAACCCUCGAAACAAAUUCAUUUCAAGAAAGUGAAUGAAGGAAUG CAAC-3′ (SEQ ID NO: 176)). In some embodiments, the gRNAs provided herein comprise a backbone sequence with one or more substitutions relative to a wild-type Cas14a1 gRNA, and the portions of the gRNA other than the backbone sequence do not comprise any substitutions relative to a wild-type Cas14a1 gRNA.

In another aspect, the present disclosure provides complexes comprising a fusion protein (e.g., any of the fusion proteins provided herein) and a gRNA (e.g., any of the gRNAs provided herein). In some embodiments, a complex comprises any of the fusion proteins provided herein and a wild type Cas14a1 gRNA. In some embodiments, a complex comprises any of the engineered gRNAs provided herein and a fusion protein comprising wild type Cas14a1.

In another aspect, the present disclosure provides methods of modifying (e.g., editing, cutting, nicking, recombining, or making epigenetic changes such as methylation or acetylation) a target nucleic acid molecule comprising contacting the target nucleic acid molecule with any of the fusion proteins provided herein and a guide RNA, or with any of the complexes provided herein. In some embodiments, the target sequence comprises a sequence associated with a disease or disorder (e.g., a point mutation, such as a T→C point mutation or an A→G point mutation).

In another aspect, the present disclosure provides polynucleotides encoding any of the Cas proteins, fusion proteins, guide RNAs, or complexes (e.g., each component of the complexes) provided herein. The present disclosure also provides vectors comprising any of the polynucleotides provided herein.

In another aspect, the present disclosure provides cells comprising any of the Cas proteins, fusion proteins, guide RNAs, complexes, polynucleotides, or vectors provided herein. In some embodiments, the cell is in a non-human animal.

In another aspect, the present disclosure provides kits comprising any of the Cas proteins, fusion proteins, guide RNAs, complexes, polynucleotides, or vectors provided herein.

In another aspect, the present disclosure provides pharmaceutical compositions comprising any of the Cas proteins, fusion proteins, guide RNAs, complexes, polynucleotides, vectors, or cells provided herein, and a pharmaceutically acceptable excipient.

In another aspect, the present disclosure provides AAVs comprising any of the Cas proteins, fusion proteins, guide RNAs, complexes, polynucleotides, vectors, or pharmaceutical compositions provided herein.

In another aspect, the present disclosure contemplates the use of any of the Cas proteins, fusion proteins, guide RNAs, complexes, polynucleotides, vectors, and pharmaceutical compositions provided herein in the manufacture of a medicament for the treatment of a disease or disorder. In some embodiments, any of the Cas proteins, fusion proteins, guide RNAs, complexes, polynucleotides, vectors, and pharmaceutical compositions provided herein are for use in medicine.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1B show that wild-type Cas14a1 is weakly active in a DNA-binding circuit. Initial experiments optimized the guide RNA binding site in order to establish that wild-type Cas14a1 was compatible with existing DNA-binding phage-assisted continuous evolution (PACE) circuits.

FIG. 2 shows that Cas14a1/sgRNA selection phage (SP) propagates on the DNA-binding circuit.

FIG. 3 shows a Cas14a1 evolution circuit that enables guide RNA coevolution.

FIG. 4 shows evolution of Cas14a1 in a limited-expression DNA-binding phage-assisted non-continuous evolution (PANCE).

FIG. 5 shows further data for evolution of Cas14a1 in a limited-expression DNA-binding PANCE for four different lagoons.

FIG. 6 provides a table showing mutants from the first round of Cas14a1 PACE.

FIGS. 7A-7C show that the wild-type Cas14a1 sgRNA is not compatible with expression from the U6 promoter (pU6), which is the most commonly used strategy for expressing guide RNAs in human cells.

FIG. 8 shows that evolved sgRNAs contain mutations in a particular poly-T region.

FIG. 9 shows that alternative non-U5 sgRNAs are compatible with Cas14a1.

FIG. 10 shows that evolved Cas14a1 variants are active in HEK293T cells with engineered guide RNAs. Engineered guide RNAs were found to enable adenine base editing with Cas14a1 variants in human cells.

FIG. 11 provides additional data showing that evolved Cas14a1 variants are active in HEK293T cells with engineered guide RNAs. The percentage of total sequencing reads with A·T converted to G·C at various edit sites are shown.

FIGS. 12A-12B provide additional data showing that evolved Cas14a1 variants are active in HEK293T cells with engineered guide RNAs. FIG. 12A shows the percentage of total sequencing reads with A·T converted to G·C at an edit site. FIG. 12B shows mutations in the Cas14a1 variants tested.

FIG. 13 shows progression of a Cas14a1 high-stringency DNA-binding PACE.

FIGS. 14A-14B show that evolved Cas14a1 variants from additional DNA-binding PACE exhibit further improved activity in HEK293T cells. FIG. 14A shows mutations in the Cas14a1 variants tested. FIG. 14B shows the percentage of total sequencing reads with A·T converted to G·C at various edit sites.

FIG. 15 provides a protein structure with mutations from the DNA-binding PACEs labeled and/or circled.

FIG. 16 shows a further round of adenosine base editor (ABE)-PANCE evolution.

FIG. 17 shows progression of a further round of ABE-PACE evolution.

FIG. 18 provides a table showing mutants from a further round of ABE-PACE evolution.

FIG. 19 provides a protein structure with mutations from ABE-PACE circled.

FIGS. 20A-20B show that the variants evolved using ABE-PACE exhibit improved adenine base editing efficiencies in HEK293T cells. FIG. 20A shows mutations in the Cas14a1 variants tested. FIG. 20B shows the percentage of total sequencing reads with A·T converted to G·C at various edit sites.

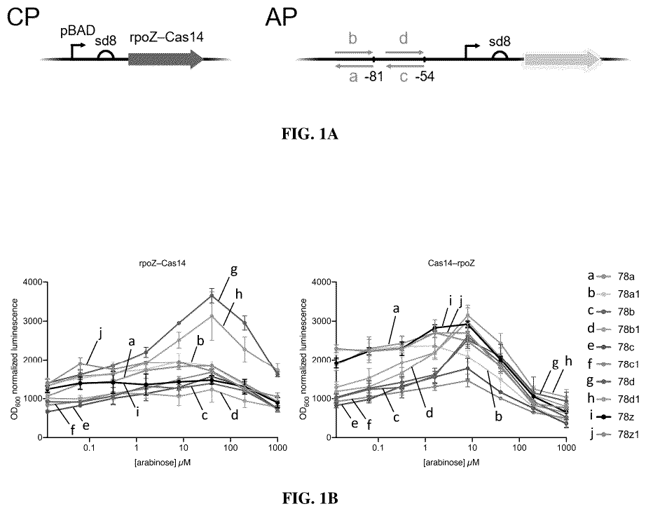

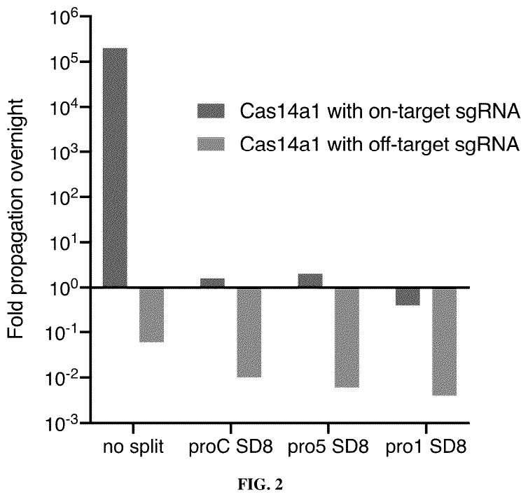

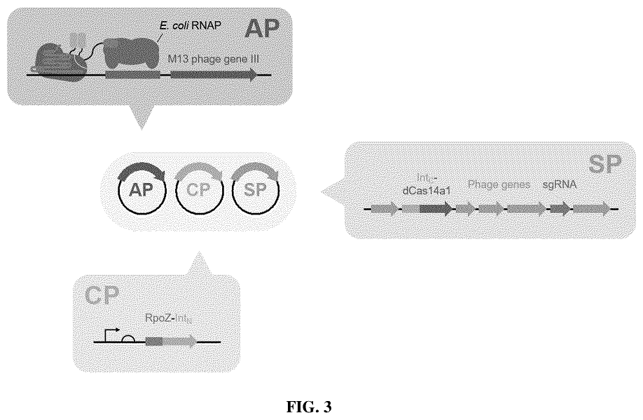

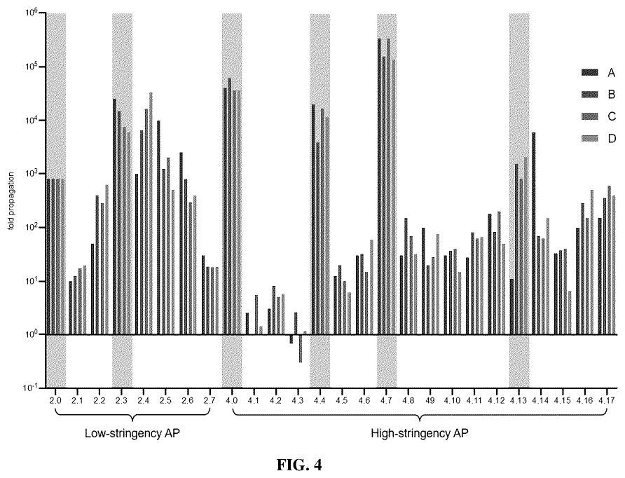

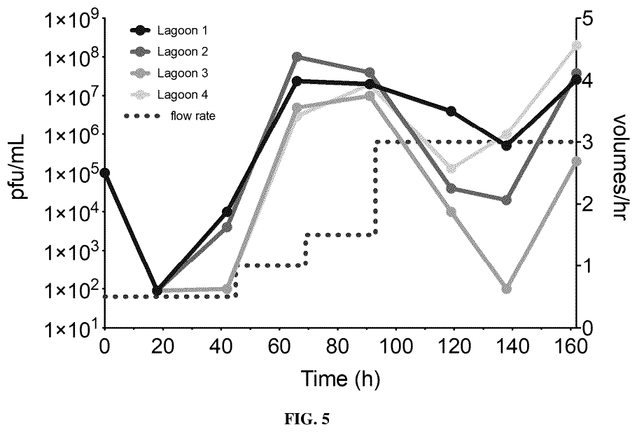

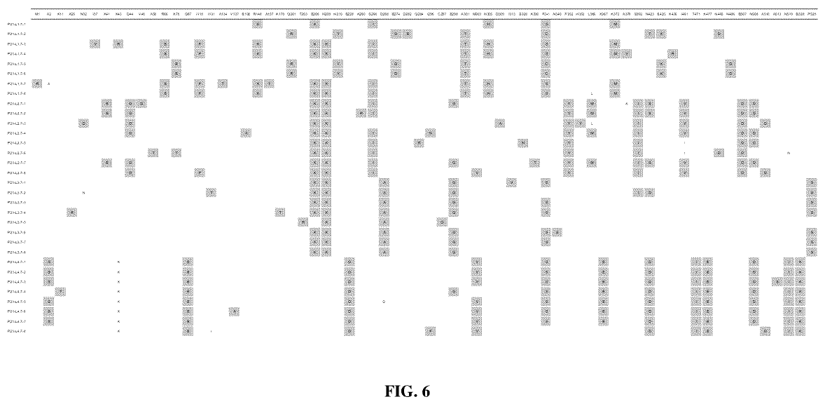

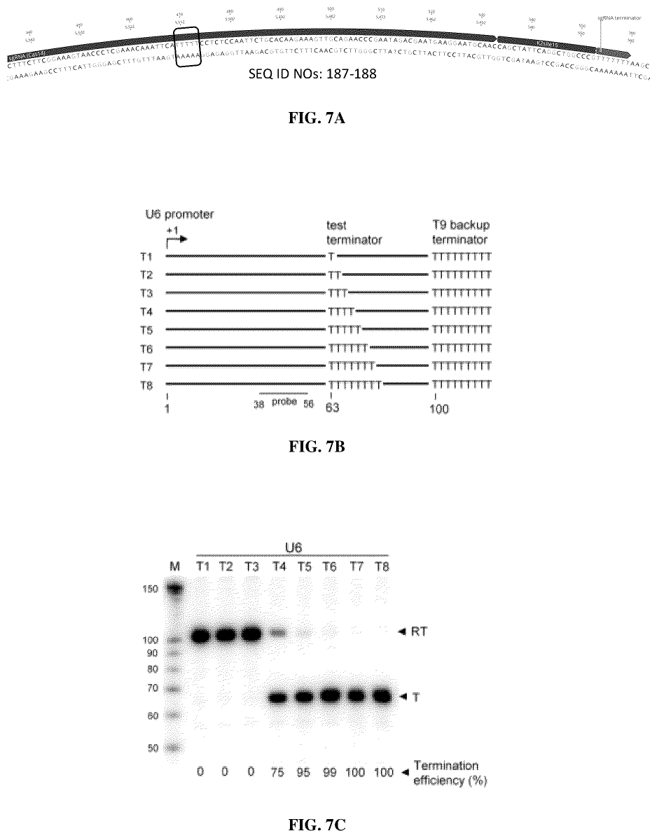

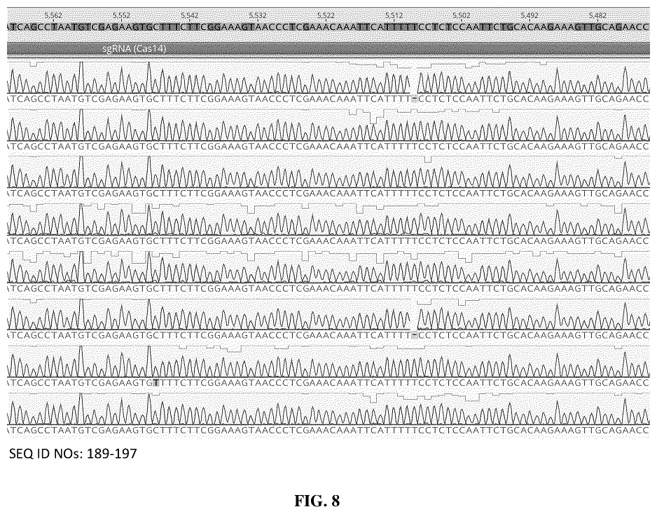

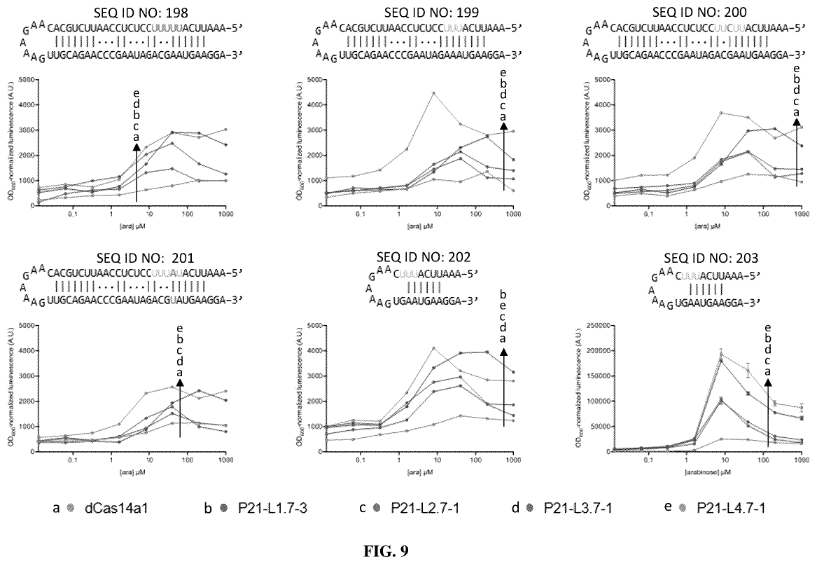

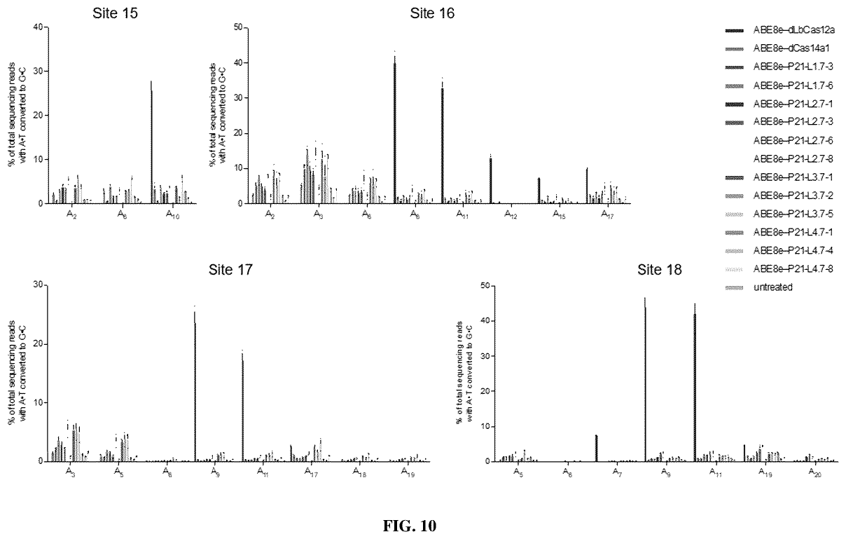

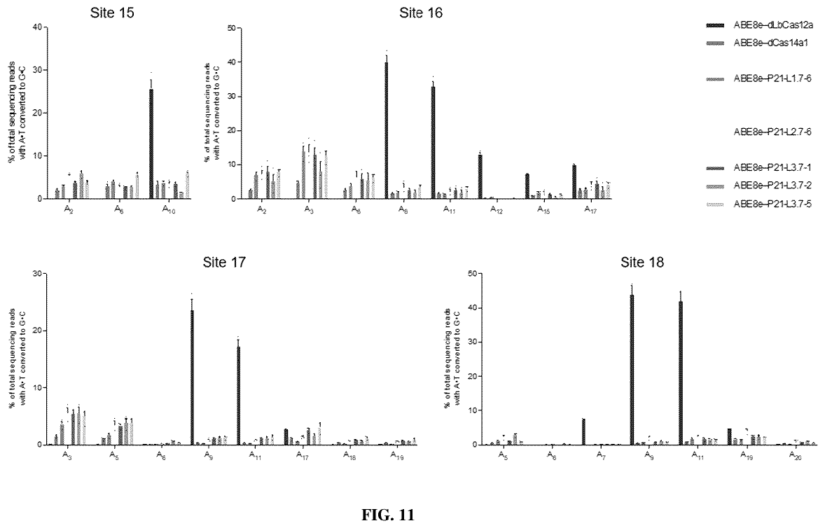

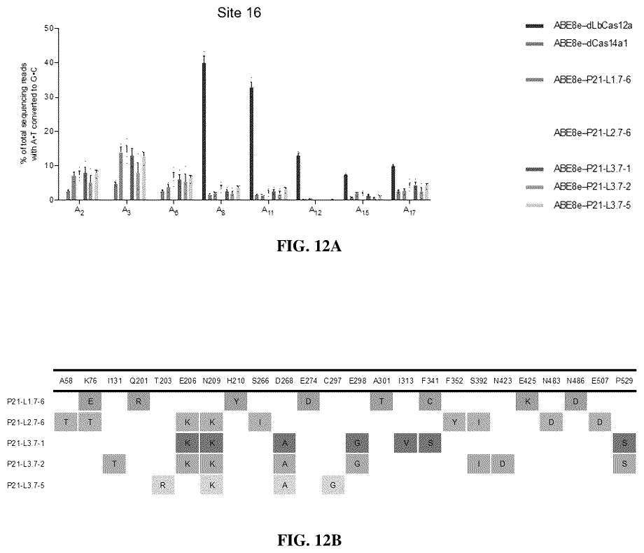

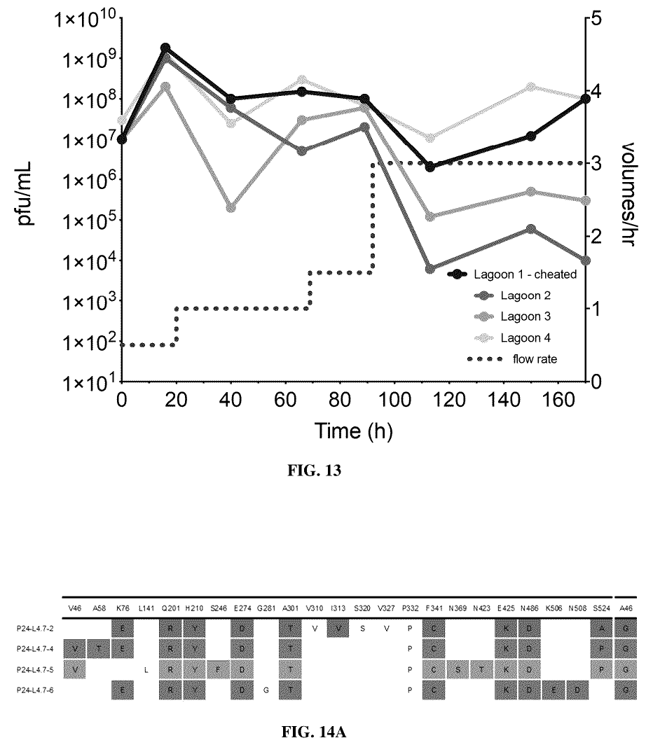

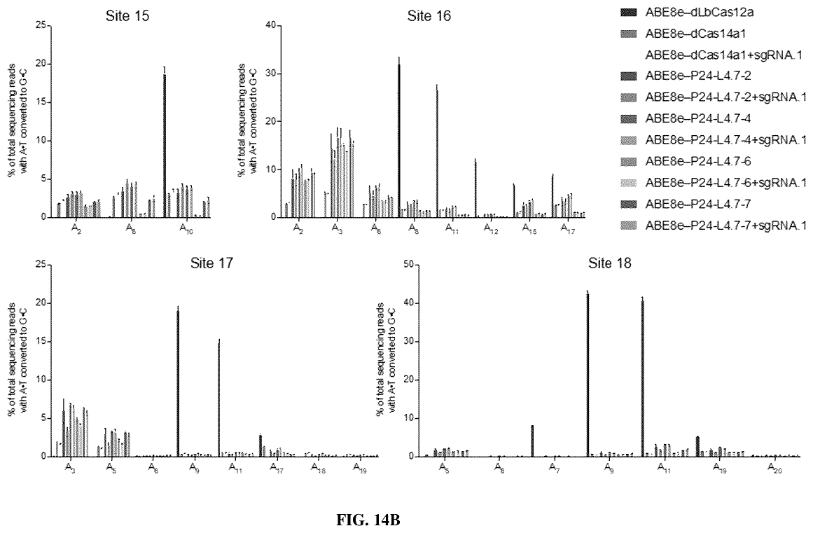

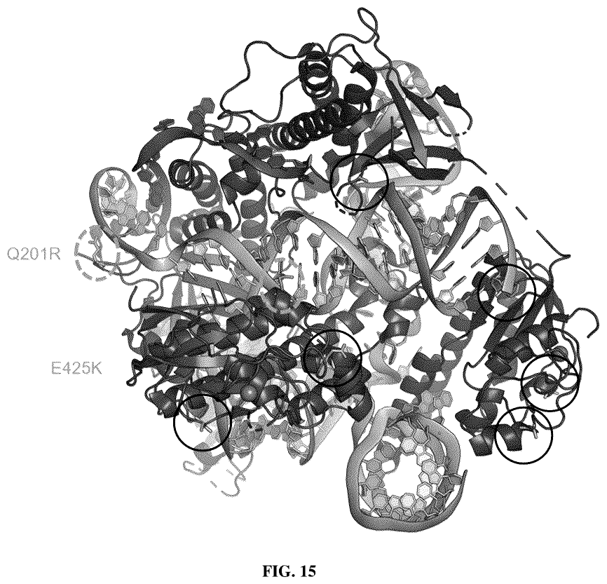

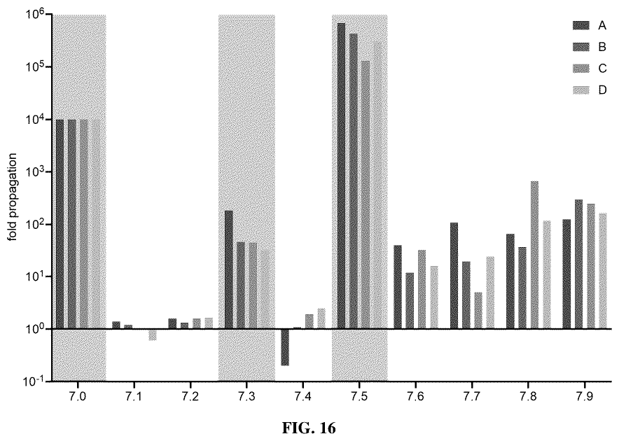

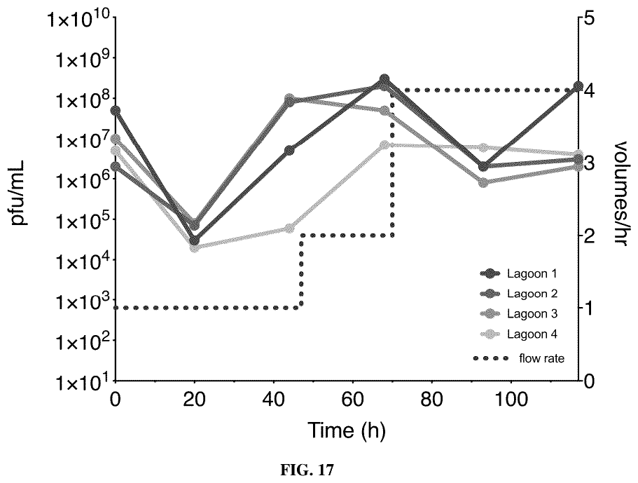

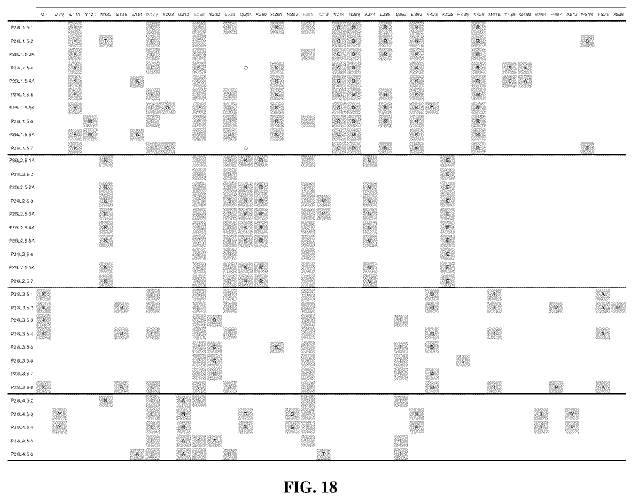

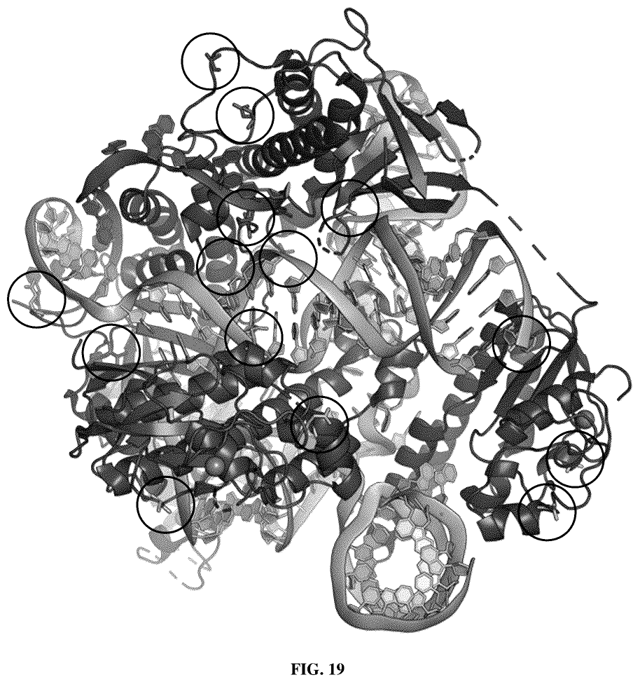

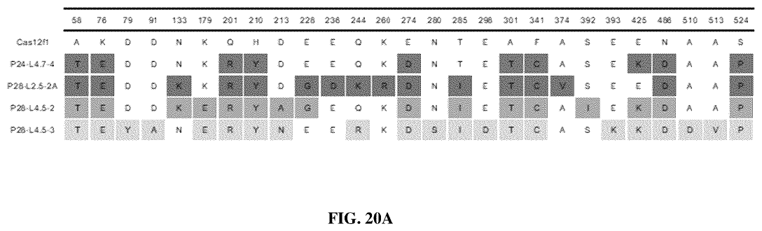

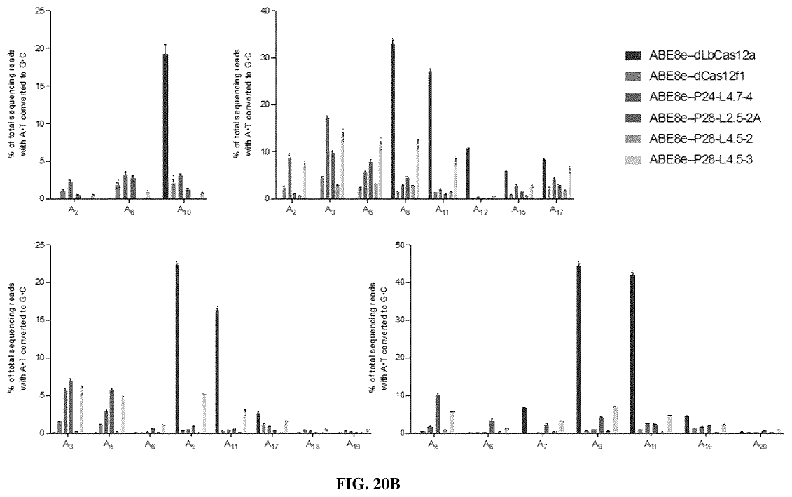

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

### Adenosine Deaminase

As used herein, the term “adenosine deaminase” or “adenosine deaminase domain” refers to a protein or enzyme that catalyzes a deamination reaction of an adenosine (or adenine). The terms are used interchangeably. In certain embodiments, the disclosure provides nucleobase editor fusion proteins comprising one or more adenosine deaminase domains (e.g., fused to any of the Cas14a1 variants disclosed herein). For instance, an adenosine deaminase domain may comprise a heterodimer of a first adenosine deaminase and a second deaminase domain, connected by a linker. Adenosine deaminases (e.g., engineered adenosine deaminases or evolved adenosine deaminases) provided herein may be enzymes that convert adenine (A) to inosine (I) in DNA or RNA. Such adenosine deaminases can lead to an A:T to G:C base pair conversion. In some embodiments, the deaminase is a variant of a naturally-occurring deaminase from an organism. In some embodiments, the deaminase does not occur in nature. For example, in some embodiments, the deaminase is at least 50%, at least 55%, at least 60%, at least 65%, at least 70%, at least 75%, at least 80%, at least 85%, at least 90%, at least 95%, at least 96%, at least 97%, at least 98%, at least 99%, or at least 99.5% identical to a naturally-occurring deaminase.

In some embodiments, the adenosine deaminase is derived from a bacterium, such as, E. coli, S. aureus, S. typhi, S. putrefaciens, H. influenzae, C. Jejuni, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. In some embodiments, the adenosine deaminase comprises ecTadA(8e) (i.e., as used in the base editor ABE8e) as described further herein. Reference is made to U.S. Patent Publication No. 2018/0073012, published Mar. 15, 2018, which is incorporated herein by reference.

### Base Editing

“Base editing” refers to genome editing technology that involves the conversion of a specific nucleic acid base into another at a targeted genomic locus. In certain embodiments, this can be achieved without requiring double-stranded DNA breaks (DSB), or single stranded breaks (i.e., nicking). To date, other genome editing techniques, including CRISPR-based systems, begin with the introduction of a DSB at a locus of interest. Subsequently, cellular DNA repair enzymes mend the break, commonly resulting in random insertions or deletions (indels) of bases at the site of the DSB. However, when the introduction or correction of a point mutation at a target locus is desired rather than stochastic disruption of the entire gene, these genome editing techniques are unsuitable, as correction rates are low (e.g., typically 0.1% to 5%), with the major genome editing products being indels. In order to increase the efficiency of gene correction without simultaneously introducing random indels, the CRISPR system is modified to directly convert one DNA base into another without DSB formation. See, Komor, A. C., et al., Programmable editing of a target base in genomic DNA without double-stranded DNA cleavage. Nature 533, 420-424 (2016), the entire contents of which is incorporated by reference herein. In some embodiments, base editing is accomplished using a fusion protein comprising a deaminase and any of the Cas14a1 variants provided herein.

In principle, there are 12 possible base-to-base changes that may occur via individual or sequential use of transition (i.e., a purine-to-purine change or pyrimidine-to-pyrimidine change) or transversion (i.e., a purine-to-pyrimidine or pyrimidine-to-purine) editors. These include transition base editors such as the cytosine base editor (“CBE”), also known as a C-to-T base editor (or “CTBE”). This type of editor converts a C:G Watson-Crick nucleobase pair to a T:A Watson-Crick nucleobase pair. Because the corresponding Watson-Crick paired bases are also interchanged as a result of the conversion, this category of base editor may also be referred to as a guanine base editor (“GBE”) or G-to-A base editor (or “GABE”). Other transition base editors include the adenine base editor (or “ABE”), also known as an A-to-G base editor (“AGBE”). This type of editor converts an A:T Watson-Crick nucleobase pair to a G:C Watson-Crick nucleobase pair. Because the corresponding Watson-Crick paired bases are also interchanged as a result of the conversion, this category of base editor may also be referred to as a thymine base editor (or “TBE”) or T-to-G base editor (“TGBE”).

### Base Editors

The terms “base editor (BE)” and “nucleobase editor,” which are used interchangeably herein, refer to an agent comprising a polypeptide that is capable of making a modification to a base (e.g., A, T, C, G, or U) within a nucleic acid sequence (e.g., DNA or RNA) that converts one base to another (e.g., A to G, A to C, A to T, C to T, C to G, C to A, G to A, G to C, G to T, T to A, T to C, or T to G). In some embodiments, the nucleobase editor is capable of deaminating a base within a nucleic acid, such as a base within a DNA molecule. In the case of an adenosine nucleobase editor, the nucleobase editor is capable of deaminating an adenine (A) in DNA. Such nucleobase editors may include a nucleic acid programmable DNA binding protein (napDNAbp) fused to an adenosine deaminase. Some nucleobase editors include CRISPR-mediated fusion proteins that are utilized in the base editing methods described herein. In some embodiments, the nucleobase editor comprises a Cas14a1 protein (e.g., any of the Cas14a1 variants described herein) fused to a deaminase that binds a nucleic acid in a guide RNA-programmed manner via the formation of an R-loop, but does not cleave the nucleic acid.

In some embodiments, a nucleobase editor is a macromolecule or macromolecular complex that results primarily (e.g., more than 80%, more than 85%, more than 90%, more than 95%, more than 99%, more than 99.9%, or 100%) in the conversion of a nucleobase in a polynucleotide sequence into another nucleobase (i.e., a transition or transversion) using a combination of 1) a nucleotide-, nucleoside-, or nucleobase-modifying enzyme and 2) a nucleic acid binding protein that can be programmed to bind to a specific nucleic acid sequence.

In some embodiments, the nucleobase editor comprises a DNA binding domain (e.g., a programmable DNA binding domain, such as any of the Cas14a1 variants described herein) that directs it to a target sequence. In some embodiments, the nucleobase editor comprises a nucleobase modification domain fused to a programmable DNA binding domain (e.g., a Cas14a1 variant). The terms “nucleobase modifying enzyme” and “nucleobase modification domain,” which are used interchangeably herein, refer to an enzyme that can modify a nucleobase and convert one nucleobase to another (e.g., a deaminase, such as a cytidine deaminase or an adenosine deaminase). The nucleobase modifying enzyme of the nucleobase editor may target cytosine (C) bases in a nucleic acid sequence and convert the C to a thymine (T) base. In some embodiments, C to T editing is carried out by a deaminase, e.g., a cytidine deaminase. In some embodiments, A to G editing is carried out by a deaminase, e.g., an adenosine deaminase. Nucleobase editors that can carry out other types of base conversions (e.g., C to G) are also contemplated.

In some embodiments, a nucleobase editor converts a C to a T. In some embodiments, the nucleobase editor comprises a cytosine deaminase. A “cytosine deaminase”, or “cytidine deaminase,” refers to an enzyme that catalyzes the chemical reaction “cytosine+H₂O→uracil+NH₃” or “5-methyl-cytosine+H₂O→thymine+NH₃.” As may be apparent from the reaction formula, such chemical reactions result in a C to U/T nucleobase change. In the context of a gene, such a nucleotide change, or mutation, may in turn lead to an amino acid change in the protein, which may affect the protein's function, e.g., loss-of-function or gain-of-function. In some embodiments, the C to T nucleobase editor comprises a Cas14a1 variant described herein fused to a cytidine deaminase. In some embodiments, the cytidine deaminase domain is fused to the N-terminus of the Cas14a1 variant. In some embodiments, the nucleobase editor further comprises a domain that inhibits uracil glycosylase, and/or a nuclear localization signal. Exemplary nucleobase editors have been described in the art, e.g., in Rees & Liu, Nat Rev Genet. 2018; 19(12):770-788 and Koblan et al., Nat Biotechnol. 2018; 36(9):843-846; as well as U.S. Patent Application Publication No. 2018/0073012, published Mar. 15, 2018, which issued as U.S. Pat. No. 10,113,163 on Oct. 30, 2018; U.S. Patent Application Publication No. 2017/0121693, published May 4, 2017, which issued as U.S. Pat. No. 10,167,457 on Jan. 1, 2019; PCT Application Publication No. WO 2017/070633, published Apr. 27, 2017; U.S. Patent Application Publication No. 2015/0166980, published Jun. 18, 2015; U.S. Pat. No. 9,840,699, issued Dec. 12, 2017; U.S. Pat. No. 10,077,453, issued Sep. 18, 2018; PCT Application Publication No. WO 2019/023680, published Jan. 31, 2019; PCT Application Publication No. WO 2018/0176009, published Sep. 27, 2018, International Patent Application No. PCT/US2019/033848, filed May 23, 2019, International Patent Application No. PCT/US2019/47996, filed Aug. 23, 2019; International Patent Application No. PCT/US2019/049793, filed Sep. 5, 2019; International Patent Application No. PCT/US2020/028568, filed Apr. 17, 2020; International Patent Application No. PCT/US2019/61685, filed Nov. 15, 2019; International Patent Application No. PCT/US2019/57956, filed Oct. 24, 2019; International Patent Application No. PCT/US2019/58678, filed Oct. 29, 2019, the contents of each of which are incorporated herein by reference.

In some embodiments, a nucleobase editor converts an A to a G. In some embodiments, the nucleobase editor comprises an adenosine deaminase. An “adenosine deaminase” is an enzyme involved in purine metabolism. It is needed for the breakdown of adenosine from food and for the turnover of nucleic acids in tissues. Its primary function in humans is the development and maintenance of the immune system. An adenosine deaminase catalyzes hydrolytic deamination of adenosine (forming inosine, which base pairs as G) in the context of DNA. There are no known natural adenosine deaminases that act on DNA. Instead, known adenosine deaminase enzymes only act on RNA (tRNA or mRNA). Evolved deoxyadenosine deaminase enzymes that accept DNA substrates and deaminate dA to deoxyinosine have been described, e.g., in International Patent Application No. PCT/US2017/045381, filed Aug. 3, 2017, which published as WO 2018/027078, International Patent Application No. PCT/US2019/033848, which published as WO 2019/226953, International Patent Application No PCT/US2019/033848, filed May 23, 2019, and International Patent Application No. PCT/US2020/028568, filed Apr. 17, 2020; each of which is incorporated herein by reference.

Exemplary adenosine and cytidine nucleobase editors are also described in Rees & Liu, Base editing: precision chemistry on the genome and transcriptome of living cells, Nat. Rev. Genet. 2018; 19(12):770-788; as well as U.S. Patent Application Publication No. 2018/0073012, published Mar. 15, 2018, which issued as U.S. Pat. No. 10,113,163 on Oct. 30, 2018; U.S. Patent Application Publication No. 2017/0121693, published May 4, 2017, which issued as U.S. Pat. No. 10,167,457 on Jan. 1, 2019; PCT Application Publication No. WO 2017/070633, published Apr. 27, 2017; U.S. Patent Application Publication No. 2015/0166980, published Jun. 18, 2015; U.S. Pat. No. 9,840,699, issued Dec. 12, 2017; and U.S. Pat. No. 10,077,453, issued Sep. 18, 2018, the contents of each of which are incorporated herein by reference in their entireties.

### Cytosine Deaminase

As used herein, a “cytosine deaminase” encoded by the CDA gene is an enzyme that catalyzes the removal of an amine group from cytidine (i.e., the base cytosine when attached to a ribose ring) to uridine (C to U) and deoxycytidine to deoxyuridine (C to U). A non-limiting example of a cytosine deaminase is APOBEC1 (“apolipoprotein B mRNA editing enzyme, catalytic polypeptide 1”). Another example is AID (“activation-induced cytosine deaminase”). Under standard Watson-Crick hydrogen bond pairing, a cytosine base hydrogen bonds to a guanine base. When cytidine is converted to uridine (or deoxycytidine is converted to deoxyuridine), the uridine (or the uracil base of uridine) undergoes hydrogen bond pairing with the base adenine. Thus, a conversion of “C” to uridine (“U”) by cytosine deaminase will cause the insertion of “A” instead of a “G” during cellular repair and/or replication processes. Since the adenine “A” pairs with thymine “T”, the cytosine deaminase in coordination with DNA replication causes the conversion of a C·G pairing to a T·A pairing in the double-stranded DNA molecule.

### CRISPR

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas14a1) and CRISPR-associated RNA, a prokaryotic immune defense system. In nature, CRISPR clusters are transcribed and processed into CRISPR RNA (crRNA). CRISPR biology is well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference).

In general, a “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. The tracrRNA of the system is complementary (fully or partially) to the tracr mate sequence present on the guide RNA.

CRISPR Cas proteins include, but are not limited to, Cas14 proteins, including Cas14a1, Cas14a2, Cas14a3, Cas14a4, Cas14a5, Cas14a6, Cas14b1, Cas14b2, Cas14b3, Cas14b4, Cas14b5, Cas14b6, Cas14b7, Cas14b8, Cas14b9, Cas14b10, Cas14b11, Cas14b12, Cas14b13, Cas14b14, Cas14b15, Cas14b16, Cas14c1, Cas14c2, Cas14d1, Cas14d2, Cas14d3, Cas14e1, Cas14e2, Cas14e3, Cas14f1, Cas14f2, Cas14g1, Cas14g2, Cas14h1, Cas14h2, Cas14h3, Cas14u1, Cas14u2, Cas14u3, Cas14u4, Cas14u5, Cas14u6, Cas14u7, and Cas14u8. Cas14 proteins, including, e.g., Cas14a1, are known in the art and described further in Harrington, L. B. et al., Programmed DNA destruction by miniature CRISPR-Cas14 enzymes. Science 362, 839-842 (2018); Karvelis, T. et al., PAM recognition by miniature CRISPR-Cas12f nucleases triggers programmable double-stranded DNA target cleavage. Nucleic Acids Res. 48, 5016-5023 (2020); and Xu, X. et al., Engineered miniature CRISPR-Cas system for mammalian genome regulation and editing. Mol. Cell 81, 4333-4345 (2021), each of which is incorporated herein by reference. Cas14 proteins may also be referred to as Cas12f, or C2c10, in the present disclosure, or in any of the references cited herein.

### Deaminase

The term “deaminase” or “deaminase domain” refers to a protein or enzyme that catalyzes a deamination reaction. In some embodiments, the deaminase is an adenosine (or adenine) deaminase, which catalyzes the hydrolytic deamination of adenine or adenosine. In some embodiments, the adenosine deaminase catalyzes the hydrolytic deamination of adenine or adenosine in deoxyribonucleic acid (DNA) to inosine. In other embodiments, the deaminase is a cytidine (or cytosine) deaminase, which catalyzes the hydrolytic deamination of cytidine or cytosine.

The deaminases provided herein may be from any organism, such as a bacterium. In some embodiments, the deaminase or deaminase domain is a variant of a naturally occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally occurring deaminase.

### DNA Synthesis Template

As used herein, the term “DNA synthesis template” refers to the region or portion of the extension arm of a PEgRNA that is utilized as a template strand by a polymerase of a prime editor to encode a 3′ single-strand DNA flap that contains the desired edit and that then, through the mechanism of prime editing, replaces the corresponding endogenous strand of DNA at the target site. The extension arm, including the DNA synthesis template, may be comprised of DNA or RNA. In the case of RNA, the polymerase of the prime editor can be an RNA-dependent DNA polymerase (e.g., a reverse transcriptase). In the case of DNA, the polymerase of the prime editor can be a DNA-dependent DNA polymerase. In various embodiments, the DNA synthesis template may comprise the “edit template” and the “homology arm,” and all or a portion of the optional 5′ end modifier region, e2. That is, depending on the nature of the e2 region (e.g., whether it includes a hairpin, toeloop, or stem/loop secondary structure), the polymerase may encode none, some, or all of the e2 region as well. Said another way, in the case of a 3′ extension arm, the DNA synthesis template can include the portion of the extension arm that spans from the 5′ end of the primer binding site (PBS) to 3′ end of the gRNA core that may operate as a template for the synthesis of a single-strand of DNA by a polymerase (e.g., a reverse transcriptase). In the case of a 5′ extension arm, the DNA synthesis template can include the portion of the extension arm that spans from the 5′ end of the PEgRNA molecule to the 3′ end of the edit template. Preferably, the DNA synthesis template excludes the primer binding site (PBS) of PEgRNAs either having a 3′ extension arm or a 5′ extension arm. Certain embodiments described here refer to an “an RT template,” which is inclusive of the edit template and the homology arm, i.e., the sequence of the PEgRNA extension arm that is actually used as a template during DNA synthesis. The term “RT template” is equivalent to the term “DNA synthesis template.”

### Edit Template

The term “edit template” refers to a portion of the extension arm of a PEgRNA that encodes the desired edit in the single strand 3′ DNA flap that is synthesized by the polymerase, e.g., a DNA-dependent DNA polymerase, RNA-dependent DNA polymerase (e.g., a reverse transcriptase). Certain embodiments described here refer to “an RT template,” which refers to both the edit template and the homology arm together, i.e., the sequence of the PEgRNA extension arm that is actually used as a template during DNA synthesis. The term “RT edit template” is also equivalent to the term “DNA synthesis template,” but wherein the RT edit template reflects the use of a prime editor having a polymerase that is a reverse transcriptase, and wherein the DNA synthesis template reflects more broadly the use of a prime editor having any polymerase.

### Effector Domain

As used herein, an “effector domain” refers to a molecule (e.g., a protein) that regulates a biological activity and/or is capable of modifying a biological molecule (e.g., a protein, or a nucleic acid such as DNA or RNA). In some embodiments, the effector domain is a protein. In some embodiments, the effector domain is capable of modifying a protein (e.g., a histone on a nucleic acid molecule). In some embodiments, the effector domain is capable of modifying DNA (e.g., genomic DNA). In some embodiments, the effector domain is capable of modifying RNA (e.g., mRNA). In some embodiments, the effector molecule is a nucleic acid editing domain. In some embodiments, the effector molecule is capable of regulating an activity of a nucleic acid (e.g., transcription, and/or translation). Exemplary effector domains include, without limitation, a deaminase, a nuclease, a nickase, a recombinase, a methyltransferase, a methylase, an acetylase, an acetyltransferase, a transcriptional activator, or a transcriptional repressor domain. In some embodiments, the effector domain is a nucleic acid editing domain. Some aspects of the disclosure provide fusion proteins comprising a Cas14a1 variant domain and a nucleic acid editing domain.

### Extension Arm

The term “extension arm” refers to a nucleotide sequence component of a PEgRNA that provides several functions, including a primer binding site and an edit template for reverse transcriptase. In some embodiments, the extension arm is located at the 3′ end of the guide RNA. In other embodiments, the extension arm is located at the 5′ end of the guide RNA. In some embodiments, the extension arm also includes a homology arm. In various embodiments, the extension arm comprises the following components in a 5′ to 3′ direction: the homology arm, the edit template, and the primer binding site. Since polymerization activity of the reverse transcriptase is in the 5′ to 3′ direction, the preferred arrangement of the homology arm, edit template, and primer binding site is in the 5′ to 3′ direction such that the reverse transcriptase, once primed by an annealed primer sequence, polymerizes a single strand of DNA using the edit template as a complementary template strand. Further details, such as the length of the extension arm, are described elsewhere herein.

The extension arm may also be described as comprising generally two regions: a primer binding site (PBS) and a DNA synthesis template, for instance. The primer binding site binds to the primer sequence that is formed from the endogenous DNA strand of the target site when it becomes nicked by the prime editor complex, thereby exposing a 3′ end on the endogenous nicked strand. As explained herein, the binding of the primer sequence to the primer binding site on the extension arm of the PEgRNA creates a duplex region with an exposed 3′ end (i.e., the 3′ of the primer sequence), which then provides a substrate for a polymerase to begin polymerizing a single strand of DNA from the exposed 3′ end along the length of the DNA synthesis template. The sequence of the single strand DNA product is the complement of the DNA synthesis template. Polymerization continues towards the 5′ of the DNA synthesis template (or extension arm) until polymerization terminates. Thus, the DNA synthesis template represents the portion of the extension arm that is encoded into a single strand DNA product (i.e., the 3′ single strand DNA flap containing the desired genetic edit information) by the polymerase of the prime editor complex and that ultimately replaces the corresponding endogenous DNA strand of the target site that sits immediately downstream of the PE-induced nick site. Without being bound by theory, polymerization of the DNA synthesis template continues towards the 5′ end of the extension arm until a termination event. Polymerization may terminate in a variety of ways, including, but not limited to, (a) reaching a 5′ terminus of the PEgRNA (e.g., in the case of the 5′ extension arm wherein the DNA polymerase simply runs out of template), (b) reaching an impassable RNA secondary structure (e.g., hairpin or stem/loop), or (c) reaching a replication termination signal, e.g., a specific nucleotide sequence that blocks or inhibits the polymerase, or a nucleic acid topological signal, such as, supercoiled DNA or RNA.

### Fusion Protein

The term “fusion protein” as used herein refers to a hybrid polypeptide that comprises protein domains from at least two different proteins. One protein may be located at the amino-terminal (N-terminal) portion of the fusion protein or at the carboxy-terminal (C-terminal) protein, thus forming an “amino-terminal fusion protein” or a “carboxy-terminal fusion protein,” respectively. A protein may comprise different domains, for example, a Cas14a1 variant disclosed herein fused to a deaminase (i.e., a base editor), or fused to a reverse transcriptase (i.e., a prime editor). Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which is incorporated herein by reference.

Guide RNA (“gRNA”)

As used herein, the term “guide RNA” is a particular type of guide nucleic acid which is commonly associated with a Cas protein (e.g., Cas14a1, or a variant thereof), directing the Cas protein to a specific sequence in a DNA molecule that includes complementarity to the protospacer sequence of the guide RNA. However, this term also embraces the equivalent guide nucleic acid molecules that associate with Cas protein equivalents, homologs, orthologs, or paralogs, whether naturally occurring or non-naturally occurring (e.g., engineered or recombinant), and which otherwise program the Cas protein equivalent to localize to a specific target nucleotide sequence. The Cas protein equivalents may include other napDNAbps from any type of CRISPR system (e.g., type II, V, VI), including Cpf1 (a type-V CRISPR-Cas system), C2c1 (a type V CRISPR-Cas system), C2c2 (a type VI CRISPR-Cas system), and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. Exemplary sequences and structures of guide RNAs are provided herein.

Functionally, guide RNAs associate with a Cas protein, directing (or programming) the Cas protein to a specific sequence in a DNA molecule that includes a sequence complementary to the protospacer sequence for the guide RNA. A gRNA is a component of the CRISPR/Cas system. The sequence specificity of a Cas DNA-binding protein is determined by gRNAs, which have nucleotide base-pairing complementarity to target DNA sequences. The native gRNA comprises a 20 nucleotide (nt) Specificity Determining Sequence (SDS), or spacer, which specifies the DNA sequence to be targeted, and is immediately followed by an 80 nt scaffold sequence, which associates the gRNA with the Cas protein. In some embodiments, an SDS of the present disclosure has a length of 15 to 100 nucleotides, or more. For example, an SDS may have a length of 15 to 90, 15 to 85, 15 to 80, 15 to 75, 15 to 70, 15 to 65, 15 to 60, 15 to 55, 15 to 50, 15 to 45, 15 to 40, 15 to 35, 15 to 30, or 15 to 20 nucleotides. In some embodiments, the SDS is 20 nucleotides long. For example, the SDS may be 15, 16, 17, 18, 19, 20, 21, 22, 23, 24, or 25 nucleotides long. At least a portion of the target DNA sequence is complementary to the SDS of the gRNA. For a Cas protein to successfully bind to the DNA target sequence, a region of the target sequence is complementary to the SDS of the gRNA sequence and is immediately followed by the correct protospacer adjacent motif (PAM) sequence. In some embodiments, an SDS is 100% complementary to its target sequence. In some embodiments, the SDS sequence is less than 100% complementary to its target sequence and is, thus, considered to be partially complementary to its target sequence. For example, a targeting sequence may be 99%, 98%, 97%, 96%, 95%, 94%, 93%, 92%, 91%, or 90% complementary to its target sequence. In some embodiments, the SDS of template DNA or target DNA may differ from a complementary region of a gRNA by 1, 2, 3, 4, or 5 nucleotides.

In some embodiments, the guide RNA is about 15-120 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 97, 98, 99, 100, 101, 102, 103, 104, 105, 106, 107, 108, 109, 110, 111, 112, 113, 114, 115, 116, 117, 118, 119, or 120 nucleotides long. In some embodiments, the guide RNA comprises a sequence of 10, 11, 12, 13, 14, 15, 16, 17, 18, 19, 20, or more contiguous nucleotides that is complementary to a target sequence. Sequence complementarity refers to distinct interactions between adenine and thymine (DNA) or uracil (RNA), and between guanine and cytosine.

### Linker

The term “linker,” as used herein, refers to a molecule linking two other molecules or moieties. The linker can be an amino acid sequence in the case of a linker joining two components of a fusion protein. For example, a Cas14a1 variant provided herein can be fused to a deaminase (e.g., an adenosine deaminase or a cytosine deaminase, in the context of a base editor), or to a polymerase (e.g., a reverse transcriptase, in the context of a prime editor) by an amino acid linker sequence. The linker can also be a nucleotide sequence in the case of joining two nucleotide sequences together (e.g., in a gRNA). In other embodiments, the linker is a non-peptidic linker. In some embodiments, the linker is an organic molecule, group, polymer, or chemical moiety. In some embodiments, the linker is 5-200 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

napDNAbp

As used herein, the term “nucleic acid programmable DNA binding protein” or “napDNAbp,” of which Cas proteins such as Cas14a1 and variants thereof are examples, refers to a protein that uses RNA:DNA hybridization to target and bind to specific sequences in a DNA molecule. Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas14a1, or a variant thereof as described herein) to localize and bind to a complementary sequence.

Without being bound by theory, the binding mechanism of a napDNAbp-guide RNA complex, in general, includes the step of forming an R-loop whereby the napDNAbp induces the unwinding of a double-strand DNA target, thereby separating the strands in the region bound by the napDNAbp. The guide RNA protospacer then hybridizes to the “target strand.” This displaces a “non-target strand” that is complementary to the target strand, which forms the single strand region of the R-loop. In some embodiments, the napDNAbp includes one or more nuclease activities, which then cut the DNA, leaving various types of lesions. For example, the napDNAbp may comprise a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand.

### Nuclear Localization Sequence (NLS)

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO/2001/038547 on May 31, 2001, the contents of which are incorporated herein by reference for its disclosure of exemplary nuclear localization sequences. In some embodiments, an NLS is fused to the N-terminus of any of the Cas14a1 variants provided herein.

### Nucleic Acid Editing Domain

The term “nucleic acid editing domain,” as used herein refers to a protein or enzyme capable of making one or more modifications (e.g., deamination of a cytidine residue) to a nucleic acid (e.g., DNA or RNA). Exemplary nucleic acid editing domains include, but are not limited to a deaminase, a nuclease, a nickase, a recombinase, a methyltransferase, a methylase, an acetylase, an acetyltransferase, a transcriptional activator, or a transcriptional repressor domain. In some embodiments, the nucleic acid editing domain is a deaminase, a nuclease, a nickase, a recombinase, a methyltransferase, a methylase, an acetylase, an acetyltransferase, a transcriptional activator, or a transcriptional repressor domain. In some embodiments, the nucleic acid editing domain is a deaminase domain (e.g., a cytidine deaminase, such as an APOBEC or an AID deaminase, or an adenosine deaminase, such as ecTadA). In some embodiments, the nucleic acid editing domain is a cytidine deaminase domain (e.g., an APOBEC or an AID deaminase). In some embodiments, the nucleic acid editing domain is an adenosine deaminase domain (e.g., an ecTadA).

### Nucleic Acid Molecule

The term “nucleic acid,” as used herein, (also referred to as a “polynucleotide”) refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, O(6) methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages).

### PEgRNA

As used herein, the terms “prime editing guide RNA” or “PEgRNA” or “extended guide RNA” refer to a specialized form of a guide RNA that has been modified to include one or more additional sequences for implementing the prime editing methods and compositions described herein. As described herein, the prime editing guide RNAs comprise one or more “extended regions” of nucleic acid sequence. The extended regions may comprise, but are not limited to, single-stranded RNA or DNA. Further, the extended regions may occur at the 3′ end of a traditional guide RNA. In other arrangements, the extended regions may occur at the 5′ end of a traditional guide RNA. In still other arrangements, the extended region may occur at an intramolecular region of the traditional guide RNA, for example, in the gRNA core region which associates and/or binds to the napDNAbp. The extended region comprises a “DNA synthesis template” that encodes (by the polymerase of the prime editor) a single-stranded DNA which, in turn, has been designed to be (a) homologous with the endogenous target DNA to be edited, and (b) which comprises at least one desired nucleotide change (e.g., a transition, a transversion, a deletion, or an insertion) to be introduced or integrated into the endogenous target DNA. The extended region may also comprise other functional sequence elements, such as, but not limited to, a “primer binding site” and a “spacer or linker” sequence, or other structural elements, such as, but not limited to, aptamers, stem loops, hairpins, toe loops (e.g., a 3′ toeloop), or an RNA-protein recruitment domain (e.g., MS2 hairpin). As used herein, the “primer binding site” comprises a sequence that hybridizes to a single-strand DNA sequence having a 3′ end generated from the nicked DNA of the R-loop.

In certain embodiments, the PEgRNAs have a 5′ extension arm, a spacer, and a gRNA core. The 5′ extension further comprises in the 5′ to 3′ direction a reverse transcriptase template, a primer binding site, and a linker. The reverse transcriptase template may also be referred to more broadly as the “DNA synthesis template” where the polymerase of a prime editor described herein is not an RT, but another type of polymerase.

In still other embodiments, the PEgRNAs have in the 5′ to 3′ direction a spacer (1), a gRNA core (2), and an extension arm (3). The extension arm (3) is at the 3′ end of the PEgRNA. The extension arm (3) further comprises in the 5′ to 3′ direction a “primer binding site” (A), an “edit template” (B), and a “homology arm” (C). The extension arm (3) may also comprise an optional modifier region at the 3′ and 5′ ends, which may be the same sequences or different sequences. In addition, the 3′ end of the PEgRNA may comprise a transcriptional terminator sequence. These sequence elements of the PEgRNAs are further described and defined herein.

In still other embodiments, the PEgRNAs have in the 5′ to 3′ direction an extension arm (3), a spacer (1), and a gRNA core (2). The extension arm (3) is at the 5′ end of the PEgRNA. The extension arm (3) further comprises in the 3′ to 5′ direction a “primer binding site” (A), an “edit template” (B), and a “homology arm” (C). The extension arm (3) may also comprise an optional modifier region at the 3′ and 5′ ends, which may be the same sequences or different sequences. The PEgRNAs may also comprise a transcriptional terminator sequence at the 3′ end. These sequence elements of the PEgRNAs are further described and defined herein.

### Polymerase

As used herein, the term “polymerase” refers to an enzyme that synthesizes a nucleotide strand and that may be used in connection with the prime editor fusion proteins described herein. The polymerase can be a “template-dependent” polymerase (i.e., a polymerase that synthesizes a nucleotide strand based on the order of nucleotide bases of a template strand). The polymerase can also be a “template-independent” polymerase (i.e., a polymerase that synthesizes a nucleotide strand without the requirement of a template strand). A polymerase may also be further categorized as a “DNA polymerase” or an “RNA polymerase.” In various embodiments, the prime editors comprise a DNA polymerase. In various embodiments, the DNA polymerase can be a “DNA-dependent DNA polymerase” (i.e., whereby the template molecule is a strand of DNA). In such cases, the DNA template molecule can be a PEgRNA, wherein the extension arm comprises a strand of DNA. In such cases, the PEgRNA may be referred to as a chimeric or hybrid PEgRNA which comprises an RNA portion (i.e., the guide RNA components, including the spacer and the gRNA core) and a DNA portion (i.e., the extension arm). In various other embodiments, the DNA polymerase can be an “RNA-dependent DNA polymerase” (i.e., whereby the template molecule is a strand of RNA). In such cases, the PEgRNA is RNA, i.e., including an RNA extension. The term “polymerase” may also refer to an enzyme that catalyzes the polymerization of nucleotides (i.e., the polymerase activity). Generally, the enzyme will initiate synthesis at the 3′-end of a primer annealed to a polynucleotide template sequence (e.g., such as a primer sequence annealed to the primer binding site of a PEgRNA) and will proceed toward the 5′ end of the template strand. A “DNA polymerase” catalyzes the polymerization of deoxynucleotides. As used herein in reference to a DNA polymerase, the term DNA polymerase includes a “functional fragment thereof.” A “functional fragment thereof” refers to any portion of a wild-type or mutant DNA polymerase that encompasses less than the entire amino acid sequence of the polymerase and that retains the ability, under at least one set of conditions, to catalyze the polymerization of a polynucleotide. Such a functional fragment may exist as a separate entity, or it may be a constituent of a larger polypeptide, such as a fusion protein.

### Prime Editing

As used herein, the term “prime editing” refers to an approach for gene editing using napDNAbps (e.g., a Cas14a1 variant described herein), a polymerase (e.g., a reverse transcriptase), and specialized guide RNAs that include a DNA synthesis template for encoding desired new genetic information (or deleting genetic information) that is then incorporated into a target DNA sequence. Prime editing is described in Anzalone, A. V. et al., Search-and-replace genome editing without double-strand breaks or donor DNA. Nature 576, 149-157 (2019), which is incorporated herein by reference.

Prime editing represents a platform for genome editing that is a versatile and precise method to directly write new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand (or is homologous to it) immediately downstream of the nick site of the target site to be edited (with the exception that it includes the desired edit). Through DNA repair and/or replication machinery, the endogenous strand downstream of the nick site is replaced by the newly synthesized replacement strand containing the desired edit. In some cases, prime editing may be thought of as a “search-and-replace” genome editing technology since the prime editors not only search and locate the desired target site to be edited, but at the same time, encode a replacement strand containing a desired edit that is installed in place of the corresponding target site endogenous DNA strand. The prime editors of the present disclosure relate, in part, to the discovery that the mechanism of prime editing can be leveraged for conducting precision CRISPR/Cas-based genome editing with high efficiency and genetic flexibility. TPRT is naturally used by mobile DNA elements, such as mammalian non-LTR retrotransposons and bacterial Group II introns. Cas protein-reverse transcriptase fusions or related systems are used to target a specific DNA sequence with a guide RNA, generate a single strand nick at the target site, and use the nicked DNA as a primer for reverse transcription of an engineered reverse transcriptase template that is integrated with the guide RNA. However, while the concept begins with prime editors that use reverse transcriptase as the DNA polymerase component, the prime editors described herein are not limited to reverse transcriptases but may include the use of virtually any DNA polymerase. Indeed, while the application throughout may refer to prime editors with “reverse transcriptases,” it is set forth here that reverse transcriptases are only one type of DNA polymerase that may work with prime editing. Thus, wherever the specification mentions a “reverse transcriptase,” the person having ordinary skill in the art should appreciate that any suitable DNA polymerase may be used in place of the reverse transcriptase. Thus, in one aspect, the prime editors may comprise any Cas14a1 variant disclosed herein, which is programmed to target a DNA sequence by associating it with a specialized guide RNA (i.e., PEgRNA) containing a spacer sequence that anneals to a complementary protospacer in the target DNA. The specialized guide RNA also contains new genetic information in the form of an extension that encodes a replacement strand of DNA containing a desired genetic alteration that is used to replace a corresponding endogenous DNA strand at the target site. To transfer information from the PEgRNA to the target DNA, the mechanism of prime editing involves nicking the target site in one strand of the DNA to expose a 3′-hydroxyl group. The exposed 3′-hydroxyl group can then be used to prime the DNA polymerization of the edit-encoding extension on PEgRNA directly into the target site. In various embodiments, the extension—which provides the template for polymerization of the replacement strand containing the edit—can be formed from RNA or DNA. In the case of an RNA extension, the polymerase of the prime editor can be an RNA-dependent DNA polymerase (such as a reverse transcriptase). In the case of a DNA extension, the polymerase of the prime editor may be a DNA-dependent DNA polymerase. The newly synthesized strand (i.e., the replacement DNA strand containing the desired edit) that is formed by the prime editors would be homologous to the genomic target sequence (i.e., have the same sequence as) except for the inclusion of a desired nucleotide change (e.g., a single nucleotide change, a deletion, or an insertion, or a combination thereof). The newly synthesized (or replacement) strand of DNA may also be referred to as a single strand DNA flap, which would compete for hybridization with the complementary homologous endogenous DNA strand, thereby displacing the corresponding endogenous strand. In certain embodiments, the system can be combined with the use of an error-prone reverse transcriptase enzyme (e.g., provided as a fusion protein with the Cas14a1 variant domain, or provided in trans to the Cas14a1 variant domain). The error-prone reverse transcriptase enzyme can introduce alterations during synthesis of the single strand DNA flap. Thus, in certain embodiments, error-prone reverse transcriptase can be utilized to introduce nucleotide changes to the target DNA. Depending on the error-prone reverse transcriptase that is used with the system, the changes can be random or non-random. Resolution of the hybridized intermediate (comprising the single strand DNA flap synthesized by the reverse transcriptase hybridized to the endogenous DNA strand) can include removal of the resulting displaced flap of endogenous DNA (e.g., with a 5′ end DNA flap endonuclease, FEN1), ligation of the synthesized single strand DNA flap to the target DNA, and assimilation of the desired nucleotide change as a result of cellular DNA repair and/or replication processes. Because templated DNA synthesis offers single nucleotide precision for the modification of any nucleotide, including insertions and deletions, the scope of this approach is very broad and could foreseeably be used for myriad applications in basic science and therapeutics.

In various embodiments, prime editing operates by contacting a target DNA molecule (for which a change in the nucleotide sequence is desired to be introduced) with a nucleic acid programmable DNA binding protein (napDNAbp) complexed with a prime editing guide RNA (PEgRNA). In various embodiments, the prime editing guide RNA (PEgRNA) comprises an extension at the 3′ or 5′ end of the guide RNA, or at an intramolecular location in the guide RNA and encodes the desired nucleotide change (e.g., single nucleotide change, insertion, or deletion). In step (a), the napDNAbp/extended gRNA complex contacts the DNA molecule, and the extended gRNA guides the napDNAbp to bind to a target locus. In step (b), a nick in one of the strands of DNA of the target locus is introduced (e.g., by a nuclease or chemical agent), thereby creating an available 3′ end in one of the strands of the target locus. In certain embodiments, the nick is created in the strand of DNA that corresponds to the R-loop strand, i.e., the strand that is not hybridized to the guide RNA sequence, i.e., the “non-target strand.” The nick, however, could be introduced in either of the strands. That is, the nick could be introduced into the R-loop “target strand” (i.e., the strand hybridized to the protospacer of the extended gRNA) or the “non-target strand” (i.e., the strand forming the single-stranded portion of the R-loop, which is complementary to the target strand). In step (c), the 3′ end of the DNA strand (formed by the nick) interacts with the extended portion of the guide RNA in order to prime reverse transcription (i.e., “target-primed RT”). In certain embodiments, the 3′ end DNA strand hybridizes to a specific RT priming sequence on the extended portion of the guide RNA, i.e., the “reverse transcriptase priming sequence” or “primer binding site” on the PEgRNA. In step (d), a reverse transcriptase (or other suitable DNA polymerase) is introduced that synthesizes a single strand of DNA from the 3′ end of the primed site towards the 5′ end of the prime editing guide RNA. The DNA polymerase (e.g., reverse transcriptase) can be fused to the napDNAbp or alternatively can be provided in trans to the napDNAbp. This forms a single-strand DNA flap comprising the desired nucleotide change (e.g., the single base change, insertion, or deletion, or a combination thereof) and that is otherwise homologous to the endogenous DNA at or adjacent to the nick site. In step (e), the napDNAbp and guide RNA are released. Steps (f) and (g) relate to the resolution of the single strand DNA flap such that the desired nucleotide change becomes incorporated into the target locus. This process can be driven towards the desired product formation by removing the corresponding 5′ endogenous DNA flap that forms once the 3′ single strand DNA flap invades and hybridizes to the endogenous DNA sequence. Without being bound by theory, the cell's endogenous DNA repair and replication processes resolve the mismatched DNA to incorporate the nucleotide change(s) to form the desired altered product. The process can also be driven towards product formation with “second strand nicking.” This process may introduce at least one or more of the following genetic changes: transversions, transitions, deletions, and insertions.

The term “prime editor (PE) system” or “prime editor (PE)” or “PE system” or “PE editing system” refers the compositions involved in the method of genome editing using target-primed reverse transcription (TPRT) describe herein, including, but not limited to, the napDNAbps (e.g., Cas14a1 variants), reverse transcriptases, fusion proteins (e.g., comprising a napDNAbps such as a Cas14a1 variant, and a reverse transcriptase), prime editing guide RNAs, and complexes comprising fusion proteins and prime editing guide RNAs, as well as accessory elements, such as second strand nicking components (e.g., second strand sgRNAs) and 5′ endogenous DNA flap removal endonucleases (e.g., FEN1) for helping to drive the prime editing process towards the edited product formation.

Although in the embodiments described thus far the PEgRNA constitutes a single molecule comprising a guide RNA (which itself comprises a spacer sequence and a gRNA core or scaffold) and a 5′ or 3′ extension arm comprising the primer binding site and a DNA synthesis template, the PEgRNA may also take the form of two individual molecules comprised of a guide RNA and a trans prime editor RNA template (tPERT), which essentially houses the extension arm (including, in particular, the primer binding site and the DNA synthesis domain) and an RNA-protein recruitment domain (e.g., MS2 aptamer or hairpin) in the same molecule that becomes co-localized or recruited to a modified prime editor complex that comprises a tPERT recruiting protein (e.g., MS2cp protein, which binds to the MS2 aptamer).

### Prime Editor

The term “prime editor” refers to fusion constructs comprising a napDNAbp (e.g., any of the Cas14a1 variant provided herein) and a reverse transcriptase and is capable of carrying out prime editing on a target nucleotide sequence in the presence of a PEgRNA (or “extended guide RNA”). The term “prime editor” may refer to the fusion protein or to the fusion protein complexed with a PEgRNA, and/or further complexed with a second-strand nicking sgRNA. In some embodiments, the prime editor may also refer to the complex comprising a fusion protein (reverse transcriptase fused to a napDNAbp), a PEgRNA, and a regular guide RNA capable of directing the second-site nicking step of the non-edited strand as described herein.

### Primer Binding Site

The term “primer binding site” or “the PBS” refers to the nucleotide sequence located on a PEgRNA as a component of the extension arm (typically at the 3′ end of the extension arm) and serves to bind to the primer sequence that is formed after napDNAbp nicking of the target sequence by the prime editor. As detailed elsewhere, when the napDNAbp component of a prime editor nicks one strand of the target DNA sequence, a 3′-ended ssDNA flap is formed, which serves a primer sequence that anneals to the primer binding site on the PEgRNA to prime reverse transcription.

### Protein, Peptide, and Polypeptide

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein, or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the contents of which are incorporated herein by reference.

### Protospacer

As used herein, the term “protospacer” refers to the sequence (˜20 bp) in DNA adjacent to the PAM (protospacer adjacent motif) sequence. The protospacer shares the same sequence as the spacer sequence of the guide RNA. The guide RNA anneals to the complement of the protospacer sequence on the target DNA (specifically, one strand thereof, i.e., the “target strand” versus the “non-target strand” of the target DNA sequence). The skilled person will appreciate that the literature in the state of the art sometimes refers to the “protospacer” as the ˜20-nt target-specific guide sequence on the guide RNA itself, rather than referring to it as a “spacer.” Thus, in some cases, the term “protospacer” as used herein may be used interchangeably with the term “spacer.” The context of the description surrounding the appearance of either “protospacer” or “spacer” will help inform the reader as to whether the term is in reference to the gRNA or the DNA target.

### Reverse Transcriptase

The term “reverse transcriptase” describes a class of polymerases characterized as RNA-dependent DNA polymerases. All known reverse transcriptases require a primer to synthesize a DNA transcript from an RNA template. Historically, reverse transcriptase has been used primarily to transcribe mRNA into cDNA, which can then be cloned into a vector for further manipulation. Avian myoblastosis virus (AMV) reverse transcriptase was the first widely used RNA-dependent DNA polymerase (Verma, Biochim. Biophys. Acta 473:1 (1977)). The enzyme has 5′-3′ RNA-directed DNA polymerase activity, 5′-3′ DNA-directed DNA polymerase activity, and RNase H activity. RNase H is a processive 5′ and 3′ ribonuclease specific for the RNA strand for RNA-DNA hybrids (Perbal, A Practical Guide to Molecular Cloning, New York: Wiley & Sons (1984)). Errors in transcription cannot be corrected by reverse transcriptase because known viral reverse transcriptases lack the 3′-5′ exonuclease activity necessary for proofreading (Saunders and Saunders, Microbial Genetics Applied to Biotechnology, London: Croom Helm (1987)). A detailed study of the activity of AMV reverse transcriptase and its associated RNaseH activity has been presented by Berger et al., Biochemistry 22:2365-2372 (1983). Another reverse transcriptase that is used extensively in molecular biology is reverse transcriptase originating from Moloney murine leukemia virus (M-MLV or “MMLV”). See, e.g., Gerard, G. R., DNA 5:271-279 (1986) and Kotewicz, M. L., et al., Gene 35:249-258 (1985). M-MLV reverse transcriptase substantially lacking in RNase H activity has also been described. See, e.g., U.S. Pat. No. 5,244,797. The invention contemplates the use of any such reverse transcriptases, or variants or mutants thereof.

In addition, the invention contemplates the use of reverse transcriptases that are error-prone, i.e., that may be referred to as error-prone reverse transcriptases or reverse transcriptases that do not support high fidelity incorporation of nucleotides during polymerization. During synthesis of the single-strand DNA flap based on the RT template integrated with the guide RNA, the error-prone reverse transcriptase can introduce one or more nucleotides that are mismatched with the RT template sequence, thereby introducing changes to the nucleotide sequence through erroneous polymerization of the single-strand DNA flap. These errors introduced during synthesis of the single strand DNA flap then become integrated into the double stranded molecule through hybridization to the corresponding endogenous target strand, removal of the endogenous displaced strand, ligation, and then through one more round of endogenous DNA repair and/or sequencing processes. The present disclosure provides, in some embodiments, prime editor fusion proteins comprising MMLV RT (e.g., fused to any of the Cas14a1 variants disclosed herein).

### Reverse Transcription

As used herein, the term “reverse transcription” indicates the capability of an enzyme to synthesize a DNA strand (that is, complementary DNA or cDNA) using RNA as a template. In some embodiments, the reverse transcription can be “error-prone reverse transcription,” which refers to the properties of certain reverse transcriptase enzymes that are error-prone in their DNA polymerization activity.

### Spacer Sequence

As used herein, the term “spacer sequence” in connection with a guide RNA or a PEgRNA refers to the portion of the guide RNA or PEgRNA of about 20 nucleotides that contains a nucleotide sequence that shares the same sequence as the protospacer sequence in the target DNA sequence. The spacer sequence anneals to the complement of the protospacer sequence to form a ssRNA/ssDNA hybrid structure at the target site and a corresponding R loop ssDNA structure of the endogenous DNA strand.

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex, and at any stage of development.

### Substitution

The term “substitution,” as used herein, refers to replacement of a residue within a sequence, e.g., a nucleic acid or amino acid sequence, with another residue, or a deletion or insertion of one or more residues within a sequence. The term “mutation” may also be used throughout the present disclosure to refer to a substitution. Substitutions are typically described herein by identifying the original residue followed by the position of the residue within the sequence and the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)).

### Target Site

The term “target site” refers to a sequence within a nucleic acid molecule that is modified (e.g., edited) by a fusion protein disclosed herein (e.g., a base editor, prime editor, or other fusion protein as described herein). The target site further refers to the sequence within a nucleic acid molecule to which a complex of, for example, a Cas protein-containing fusion protein and a gRNA binds.

### Treatment

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### Variant

As used herein, the term “variant” should be taken to mean the exhibition of qualities that have a pattern that deviates from what occurs in nature, e.g., a variant Cas14a1 is a Cas14a1 comprising one or more changes in amino acid residues (i.e., “substitutions”) as compared to a wild type Cas14a1 amino acid sequence. The term “variant” encompasses homologous proteins having at least 75%, at least 80%, at least 85%, at least 90%, at least 95%, or at least 99% identity with a reference sequence and having the same or substantially the same functional activity or activities as the reference sequence. The term also encompasses mutants, truncations, or domains of a reference sequence that display the same or substantially the same functional activity or activities as the reference sequence.

### Vector

The term “vector,” as used herein, refers to a nucleic acid that can be modified to encode a gene of interest and that is able to enter a host cell, mutate, and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

### Wild Type

As used herein the term “wild type” is a term of the art understood by skilled persons and means the typical form of an organism, strain, gene or characteristic as it occurs in nature, as distinguished from mutant or variant forms.

### DETAILED DESCRIPTION

Streptococcus pyogenes Cas9 (SpCas9) is a widely utilized genome-editing tool, but due to its large size, alternative, smaller-sized nucleic acid-programmable DNA-binding proteins are needed for use in genome editing agents, such as base editors and prime editors. The present disclosure is based on the evolution and engineering of variants of Cas14a1 with improved activity (e.g., improved editing efficiency when used, for example, in the context of a base editor). Multiple rounds of PACE and PANCE of Cas14a1 were performed to yield several variants with improved activity when used in base editors in bacteria and human cells. Rational engineering of the Cas14a1 guide RNA was also performed (specifically, to remove a poly-U tract in the gRNA backbone sequence), further enabling robust activity of the improved Cas14a1 variants provided herein in human cells. Because Cas14a1 is only 529 amino acids long, and therefore small enough to enable single-AAV delivery of various CRISPR-based genome editing agents into cells, including base editors and prime editors, the evolved Cas variants described herein are useful in various genome editing agents and systems.

Thus, the present disclosure provides Cas protein variants comprising one or more amino acid substitutions relative to wild-type Cas14a1. Fusion proteins comprising the Cas protein variants described herein are also provided by the present disclosure. Further provided herein are methods of modifying a target nucleic acid using the Cas proteins and fusion proteins provided herein. The present disclosure also provides guide RNAs, complexes, systems (e.g., comprising a Cas protein variant, gRNA, and/or effector protein in trans), polynucleotides, vectors, cells, kits, and pharmaceutical compositions. Uses of the Cas protein variants provided herein (e.g., in medicine) are also provided by the present disclosure.

napDNAbps

Some aspects of the present disclosure provide nucleic acid-programmable DNA binding proteins (napDNAbps) that exhibit improved activity (e.g., when used for base editing in the context of a fusion protein). In some embodiments, a napDNAbp is a Cas protein (e.g., Cas14a1). In some aspects, the present disclosure provides Cas14a1 variants that exhibit improved activity (e.g., increased editing efficiency when used, for example, in the context of a base editor fusion protein). The Cas proteins described herein comprise various amino acid substitutions relative to the amino acid sequence of wild-type Cas14a1, which is provided below:

Wild-type Cas14a1:
(SEQ ID NO: 2)
MAKNTITKTLKLRIVRPYNSAEVEKIVADEKNNREKIALEKNKDKVKEA
CSKHLKVAAYCTTQVERNACLFCKARKLDDKFYQKLRGQFPDAVFWQEI
SEIFRQLQKQAAEIYNQSLIELYYEIFIKGKGIANASSVEHYLSDVCYT
RAAELFKNAAIASGLRSKIKSNFRLKELKNMKSGLPTTKSDNFPIPLVK
QKGGQYTGFEISNHNSDFIIKIPFGRWQVKKEIDKYRPWEKFDFEQVQK
SPKPISLLLSTQRRKRNKGWSKDEGTEAEIKKVMNGDYQTSYIEVKRGS
KIGEKSAWMLNLSIDVPKIDKGVDPSIIGGIDVGVKSPLVCAINNAFSR
YSISDNDLFHENKKMFARRRILLKKNRHKRAGHGAKNKLKPITILTEKS
ERFRKKLIERWACEIADFFIKNKVGTVQMENLESMKRKEDSYFNIRLRG
FWPYAEMQNKIEFKLKQYGIEIRKVAPNNTSKTCSKCGHLNNYFNFEYR
KKNKFPHFKCEKCNFKENADYNAALNISNPKLKSTKEEP

Cas14a1 was first discovered from an archea of the DPANN super-phylum as described in Harrington, L. B. et al., Programmed DNA destruction by miniature CRISPR-Cas14 enzymes. Science 2018, 362(6416), 839-842, which is incorporated herein by reference.

It should be appreciated that any of the amino acid mutations described herein, (e.g., A58T) from a first amino acid residue (e.g., A) to a second amino acid residue (e.g., T) may also include mutations from the first amino acid residue to an amino acid residue that is similar to (e.g., conserved) the second amino acid residue. For example, mutation of an amino acid with a hydrophobic side chain (e.g., alanine, valine, isoleucine, leucine, methionine, phenylalanine, tyrosine, or tryptophan) may be a mutation to a second amino acid with a different hydrophobic side chain (e.g., alanine, valine, isoleucine, leucine, methionine, phenylalanine, tyrosine, or tryptophan). For example, a mutation of an alanine to a threonine (e.g., an A58T mutation) may also be a mutation from an alanine to an amino acid that is similar in size and chemical properties to a threonine, for example, serine. As another example, mutation of an amino acid with a positively charged side chain (e.g., arginine, histidine, or lysine) may be a mutation to a second amino acid with a different positively charged side chain (e.g., arginine, histidine, or lysine). As another example, mutation of an amino acid with a polar side chain (e.g., serine, threonine, asparagine, or glutamine) may be a mutation to a second amino acid with a different polar side chain (e.g., serine, threonine, asparagine, or glutamine). Additional similar amino acid pairs include, but are not limited to, the following: phenylalanine and tyrosine; asparagine and glutamine; methionine and cysteine; aspartic acid and glutamic acid; and arginine and lysine.

The skilled artisan would recognize that such conservative amino acid substitutions will likely have minor effects on protein structure and are likely to be well tolerated without compromising function. In some embodiments, any of the amino acid mutations provided herein from one amino acid to a threonine may be an amino acid mutation to a serine. In some embodiments, any of the amino acid mutations provided herein from one amino acid to an arginine may be an amino acid mutation to a lysine. In some embodiments, any of the amino acid mutations provided herein from one amino acid to an isoleucine may be an amino acid mutation to an alanine, valine, methionine, or leucine. In some embodiments, any of the amino acid mutations provided herein from one amino acid to a lysine may be an amino acid mutation to an arginine. In some embodiments, any of the amino acid mutations provided herein from one amino acid to an aspartic acid may be an amino acid mutation to a glutamic acid or asparagine. In some embodiments, any of the amino acid mutations provided herein from one amino acid to a valine may be an amino acid mutation to an alanine, isoleucine, methionine, or leucine. In some embodiments, any of the amino acid mutations provided herein from one amino acid to a glycine may be an amino acid mutation to an alanine. It should be appreciated, however, that additional conserved amino acid residues would be recognized by the skilled artisan, and any of the amino acid mutations to other conserved amino acid residues are also within the scope of this disclosure.

In some aspects, the present disclosure provides Cas proteins comprising an amino acid sequence that is at least 70%, at least 75%, at least 80%, at least 85%, at least 90%, at least 95%, or at least 99% identical to the amino acid sequence of a Cas protein of SEQ ID NO: 2, wherein the amino acid sequence of the Cas protein comprises at least 1, at least 2, at least 3, at least 4, at least 5, at least 6, at least 7, at least 8, at least 9, at least 10, at least 11, or at least 12 substitutions at positions selected from the group consisting of amino acid residues 1, 2, 11, 25, 32, 37, 41, 43, 44, 46, 58, 66, 76, 87, 118, 131, 134, 137, 138, 148, 157, 179, 201, 203, 206, 209, 210, 228, 260, 266, 268, 274, 282, 284, 296, 297, 298, 301, 303, 305, 309, 313, 320, 330, 341, 349, 352, 353, 366, 367, 372, 378, 392, 423, 425, 430, 461, 471, 477, 483, 486, 507, 508, 510, 513, 519, 528, and 529 of the amino acid sequence provided in SEQ ID NO: 2. In some embodiments, the Cas protein comprises an amino acid sequence that is not identical to the amino acid sequence of wild-type Cas14a1. In some embodiments, the amino acid sequence of the Cas protein comprises at least 1, at least 2, at least 3, at least 4, at least 5, at least 6, at least 7, at least 8, at least 9, at least 10, at least 11, or at least 12 substitutions selected from the group consisting of M1X, A2X, K11X, K25X, N32X, I37X, K41X, K43X, D44X, V46X, A58X, R66X, K76X, G87X, I118X, I131X, A134X, V137X, E138X, R148X, A157X, K179X, Q201X, T203X, E206X, N209X, H210X, E228X, K260X, S266X, D268X, E274X, D282X, Q284X, I296X, C297X, E298X, A301X, M303X, N305X, D309X, I313X, S320X, K330X, F341X, N349X, F352X, H353X, L366X, K367X, K372X, A378X, S392X, N423X, E425X, K430X, I461X, T471X, K477X, N483X, N486X, E507X, N508X, A510X, A513X, N519X, E528X, and P529X, relative to the amino acid sequence provided in SEQ ID NO: 2, wherein X represents any amino acid other than the wild type amino acid. In certain embodiments, the amino acid sequence of the Cas protein comprises at least 1, at least 2, at least 3, at least 4, at least 5, at least 6, at least 7, at least 8, at least 9, at least 10, at least 11, or at least 12 substitutions selected from the group consisting of M1R, A2S, K11T, K25R, N32D, I37V, K41E, K43R, D44G, V46G, A58T, R66S, K76E, K76T, G87E, I118F, I131T, A134T, V137A, E138A, R148K, A157T, K179T, Q201R, T203R, E206K, N209K, H210Y, E228D, K260R, S266I, D268A, E274D, D282E, Q284R, I296N, I296F, C297G, E298G, A301T, M303V, N305H, D309A, I313V, S320N, K330T, F341S, F341C, N349S, F352Y, H353Y, L366M, K367E, K372M, A378V, S392I, N423T, N423S, N423D, E425K, K430R, I461V, T471I, K477E, N483D, N486D, E507D, N508D, A510D, A513S, N519I, E528K, and P529S, relative to the amino acid sequence of SEQ ID NO: 2.

In some embodiments, the amino acid sequence of the Cas protein comprises a substitution at amino acid position 1 of SEQ ID NO: 2, or a corresponding mutation in another Cas14 protein. In some embodiments, the substitution is an M1X substitution, wherein X is any amino acid other than M. In certain embodiments, the substitution is an M1R substitution.

In some embodiments, the amino acid sequence of the Cas protein comprises a substitution at amino acid position 2 of SEQ ID NO: 2, or a corresponding mutation in another Cas14 protein. In some embodiments, the substitution is an A2X substitution, wherein X is any amino acid other than A. In certain embodiments, the substitution is an A2S substitution.

In some embodiments, the amino acid sequence of the Cas protein comprises a substitution at amino acid position 11 of SEQ ID NO: 2, or a corresponding mutation in another Cas14 protein. In some embodiments, the substitution is a K11X substitution, wherein X is any amino acid other than K. In certain embodiments, the substitution is a K11T substitution.

In some embodiments, the amino acid sequence of the Cas protein comprises a substitution at amino acid position 25 of SEQ ID NO: 2, or a corresponding mutation in another Cas14 protein. In some embodiments, the substitution is a K25X substitution, wherein X is any amino acid other than K. In certain embodiments, the substitution is a K25R substitution.

In some embodiments, the amino acid sequence of the Cas protein comprises a substitution at amino acid position 32 of SEQ ID NO: 2, or a corresponding mutation in another Cas14 protein. In some embodiments, the substitution is an N32X substitution, wherein X is any amino acid other than N. In certain embodiments, the substitution is an N32D substitution.

In some embodiments, the amino acid sequence of the Cas protein comprises a substitution at amino acid position 37 of SEQ ID NO: 2, or a corresponding mutation in another Cas14 protein. In some embodiments, the substitution is an I37X substitution, wherein X is any amino acid other than I. In certain embodiments, the substitution is an I37V substitution.

In some embodiments, the amino acid sequence of the Cas protein comprises a substitution at amino acid position 41 of SEQ ID NO: 2, or a corresponding mutation in another Cas14 protein. In some embodiments, the substitution is a K41X substitution, wherein X is any amino acid other than K. In certain embodiments, the substitution is a K41E substitution.

In some embodiments, the amino acid sequence of the Cas protein comprises a substitution at amino acid position 43 of SEQ ID NO: 2, or a corresponding mutation in another Cas14 protein. In some embodiments, the substitution is a K43X substitution, wherein X is any amino acid other than K. In certain embodiments, the substitution is a K43R substitution.

In some embodiments, the amino acid sequence of the Cas protein comprises a substitution at amino acid position 44 of SEQ ID NO: 2, or a corresponding mutation in another Cas14 protein. In some embodiments, the substitution is a D44X substitution, wherein X is any amino acid other than D. In certain embodiments, the substitution is a D44G substitution.

In some embodiments, the amino acid sequence of the Cas protein comprises a substitution at amino acid position 46 of SEQ ID NO: 2, or a corresponding mutation in another Cas14 protein. In some embodiments, the substitution is a V46X substitution, wherein X is any amino acid other than V. In certain embodiments, the substitution is a V46G substitution.

In some embodiments, the amino acid sequence of the Cas protein comprises a substitution at amino acid position 58 of SEQ ID NO: 2, or a corresponding mutation in another Cas14 protein. In some embodiments, the substitution is an A58X substitution, wherein X is any amino acid other than A. In certain embodiments, the substitution is an A58T substitution.

In some embodiments, the amino acid sequence of the Cas protein comprises a substitution at amino acid position 66 of SEQ ID NO: 2, or a corresponding mutation in another Cas14 protein. In some embodiments, the substitution is an R66X substitution, wherein X is any amino acid other than R. In certain embodiments, the substitution is an R66S substitution.

In some embodiments, the amino acid sequence of the Cas protein comprises a substitution at amino acid position 76 of SEQ ID NO: 2, or a corresponding mutation in another Cas14 protein. In some embodiments, the substitution is a K76X substitution, wherein X is any amino acid other than K. In certain embodiments, the substitution is a K76E substitution. In certain embodiments, the substitution is a K76T substitution.

In some embodiments, the amino acid sequence of the Cas protein comprises a substitution at amino acid position 87 of SEQ ID NO: 2, or a corresponding mutation in another Cas14 protein. In some embodiments, the substitution is a G87X substitution, wherein X is any amino acid other than G. In certain embodiments, the substitution is a G87E substitution.

In some embodiments, the amino acid sequence of the Cas protein comprises a substitution at amino acid position 118 of SEQ ID NO: 2, or a corresponding mutation in another Cas14 protein. In some embodiments, the substitution is an I118X substitution, wherein X is any amino acid other than I. In certain embodiments, the substitution is an I118F substitution.

In some embodiments, the amino acid sequence of the Cas protein comprises a substitution at amino acid position 131 of SEQ ID NO: 2, or a corresponding mutation in another Cas14 protein. In some embodiments, the substitution is an I131X substitution, wherein X is any amino acid other than I. In certain embodiments, the substitution is an I131T substitution.

In some embodiments, the amino acid sequence of the Cas protein comprises a substitution at amino acid position 134 of SEQ ID NO: 2, or a corresponding mutation in another Cas14 protein. In some embodiments, the substitution is an A134X substitution, wherein X is any amino acid other than A. In certain embodiments, the substitution is an A134T substitution.

In some embodiments, the amino acid sequence of the Cas protein comprises a substitution at amino acid position 137 of SEQ ID NO: 2, or a corresponding mutation in another Cas14 protein. In some embodiments, the substitution is a V137X substitution, wherein X is any amino acid other than V. In certain embodiments, the substitution is an V137A substitution.

In some embodiments, the amino acid sequence of the Cas protein comprises a substitution at amino acid position 138 of SEQ ID NO: 2, or a corresponding mutation in another Cas14 protein. In some embodiments, the substitution is an E138X substitution, wherein X is any amino acid other than E. In certain embodiments, the substitution is an E138A substitution.

In some embodiments, the amino acid sequence of the Cas protein comprises a substitution at amino acid position 148 of SEQ ID NO: 2, or a corresponding mutation in another Cas14 protein. In some embodiments, the substitution is an R148X substitution, wherein X is any amino acid other than R. In certain embodiments, the substitution is an R148K substitution.

In some embodiments, the amino acid sequence of the Cas protein comprises a substitution at amino acid position 157 of SEQ ID NO: 2, or a corresponding mutation in another Cas14 protein. In some embodiments, the substitution is an A157X substitution, wherein X is any amino acid other than A. In certain embodiments, the substitution is an A157T substitution.

In some embodiments, the amino acid sequence of the Cas protein comprises a substitution at amino acid position 179 of SEQ ID NO: 2, or a corresponding mutation in another Cas14 protein. In some embodiments, the substitution is a K179X substitution, wherein X is any amino acid other than K. In certain embodiments, the substitution is a K179T substitution.

In some embodiments, the amino acid sequence of the Cas protein comprises a substitution at amino acid position 201 of SEQ ID NO: 2, or a corresponding mutation in another Cas14 protein. In some embodiments, the substitution is a Q201X substitution, wherein X is any amino acid other than Q. In certain embodiments, the substitution is a Q201R substitution.

In some embodiments, the amino acid sequence of the Cas protein comprises a substitution at amino acid position 203 of SEQ ID NO: 2, or a corresponding mutation in another Cas14 protein. In some embodiments, the substitution is a T203X substitution, wherein X is any amino acid other than T. In certain embodiments, the substitution is a T203R substitution.

In some embodiments, the amino acid sequence of the Cas protein comprises a substitution at amino acid position 206 of SEQ ID NO: 2, or a corresponding mutation in another Cas14 protein. In some embodiments, the substitution is an E206X substitution, wherein X is any amino acid other than E. In certain embodiments, the substitution is an E206K substitution.

In some embodiments, the amino acid sequence of the Cas protein comprises a substitution at amino acid position 209 of SEQ ID NO: 2, or a corresponding mutation in another Cas14 protein. In some embodiments, the substitution is an N209X substitution, wherein X is any amino acid other than N. In certain embodiments, the substitution is an N209K substitution.

In some embodiments, the amino acid sequence of the Cas protein comprises a substitution at amino acid position 210 of SEQ ID NO: 2, or a corresponding mutation in another Cas14 protein. In some embodiments, the substitution is an H210X substitution, wherein X is any amino acid other than H. In certain embodiments, the substitution is an H210Y substitution.

In some embodiments, the amino acid sequence of the Cas protein comprises a substitution at amino acid position 228 of SEQ ID NO: 2, or a corresponding mutation in another Cas14 protein. In some embodiments, the substitution is an E228X substitution, wherein X is any amino acid other than E. In certain embodiments, the substitution is an E228D substitution.

In some embodiments, the amino acid sequence of the Cas protein comprises a substitution at amino acid position 260 of SEQ ID NO: 2, or a corresponding mutation in another Cas14 protein. In some embodiments, the substitution is a K260X substitution, wherein X is any amino acid other than K. In certain embodiments, the substitution is a K260R substitution.

In some embodiments, the amino acid sequence of the Cas protein comprises a substitution at amino acid position 266 of SEQ ID NO: 2, or a corresponding mutation in another Cas14 protein. In some embodiments, the substitution is an S266X substitution, wherein X is any amino acid other than S. In certain embodiments, the substitution is an S266I substitution.

In some embodiments, the amino acid sequence of the Cas protein comprises a substitution at amino acid position 268 of SEQ ID NO: 2, or a corresponding mutation in another Cas14 protein. In some embodiments, the substitution is a D268X substitution, wherein X is any amino acid other than D. In certain embodiments, the substitution is a D268A substitution.

In some embodiments, the amino acid sequence of the Cas protein comprises a substitution at amino acid position 274 of SEQ ID NO: 2, or a corresponding mutation in another Cas14 protein. In some embodiments, the substitution is an E274X substitution, wherein X is any amino acid other than E. In certain embodiments, the substitution is an E274D substitution.

In some embodiments, the amino acid sequence of the Cas protein comprises a substitution at amino acid position 282 of SEQ ID NO: 2, or a corresponding mutation in another Cas14 protein. In some embodiments, the substitution is a D282X substitution, wherein X is any amino acid other than D. In certain embodiments, the substitution is a D282E substitution.

In some embodiments, the amino acid sequence of the Cas protein comprises a substitution at amino acid position 284 of SEQ ID NO: 2, or a corresponding mutation in another Cas14 protein. In some embodiments, the substitution is a Q284X substitution, wherein X is any amino acid other than Q. In certain embodiments, the substitution is a Q284R substitution.

In some embodiments, the amino acid sequence of the Cas protein comprises a substitution at amino acid position 296 of SEQ ID NO: 2, or a corresponding mutation in another Cas14 protein. In some embodiments, the substitution is an I296X substitution, wherein X is any amino acid other than I. In certain embodiments, the substitution is an I296N substitution. In certain embodiments, the substitution in an I296F substitution.

In some embodiments, the amino acid sequence of the Cas protein comprises a substitution at amino acid position 297 of SEQ ID NO: 2, or a corresponding mutation in another Cas14 protein. In some embodiments, the substitution is a C297X substitution, wherein X is any amino acid other than C. In certain embodiments, the substitution is an C297G substitution.

In some embodiments, the amino acid sequence of the Cas protein comprises a substitution at amino acid position 298 of SEQ ID NO: 2, or a corresponding mutation in another Cas14 protein. In some embodiments, the substitution is an E298X substitution, wherein X is any amino acid other than E. In certain embodiments, the substitution is an E298G substitution.

In some embodiments, the amino acid sequence of the Cas protein comprises a substitution at amino acid position 301 of SEQ ID NO: 2, or a corresponding mutation in another Cas14 protein. In some embodiments, the substitution is an A301X substitution, wherein X is any amino acid other than A. In certain embodiments, the substitution is an A301T substitution.

In some embodiments, the amino acid sequence of the Cas protein comprises a substitution at amino acid position 303 of SEQ ID NO: 2, or a corresponding mutation in another Cas14 protein. In some embodiments, the substitution is an M303X substitution, wherein X is any amino acid other than M. In certain embodiments, the substitution is an M303V substitution.

In some embodiments, the amino acid sequence of the Cas protein comprises a substitution at amino acid position 305 of SEQ ID NO: 2, or a corresponding mutation in another Cas14 protein. In some embodiments, the substitution is an N305X substitution, wherein X is any amino acid other than N. In certain embodiments, the substitution is an N305H substitution.

In some embodiments, the amino acid sequence of the Cas protein comprises a substitution at amino acid position 309 of SEQ ID NO: 2, or a corresponding mutation in another Cas14 protein. In some embodiments, the substitution is a D309X substitution, wherein X is any amino acid other than D. In certain embodiments, the substitution is a D309A substitution.

In some embodiments, the amino acid sequence of the Cas protein comprises a substitution at amino acid position 313 of SEQ ID NO: 2, or a corresponding mutation in another Cas14 protein. In some embodiments, the substitution is an I313X substitution, wherein X is any amino acid other than I. In certain embodiments, the substitution is an I313V substitution.

In some embodiments, the amino acid sequence of the Cas protein comprises a substitution at amino acid position 320 of SEQ ID NO: 2, or a corresponding mutation in another Cas14 protein. In some embodiments, the substitution is an S320X substitution, wherein X is any amino acid other than S. In certain embodiments, the substitution is an S320N substitution.

In some embodiments, the amino acid sequence of the Cas protein comprises a substitution at amino acid position 330 of SEQ ID NO: 2, or a corresponding mutation in another Cas14 protein. In some embodiments, the substitution is a K330X substitution, wherein X is any amino acid other than K. In certain embodiments, the substitution is a K330T substitution.

In some embodiments, the amino acid sequence of the Cas protein comprises a substitution at amino acid position 341 of SEQ ID NO: 2, or a corresponding mutation in another Cas14 protein. In some embodiments, the substitution is an F341X substitution, wherein X is any amino acid other than F. In certain embodiments, the substitution is an F341S substitution. In certain embodiments, the substitution is an F341C substitution.

In some embodiments, the amino acid sequence of the Cas protein comprises a substitution at amino acid position 349 of SEQ ID NO: 2, or a corresponding mutation in another Cas14 protein. In some embodiments, the substitution is an N349X substitution, wherein X is any amino acid other than N. In certain embodiments, the substitution is an N349S substitution.

In some embodiments, the amino acid sequence of the Cas protein comprises a substitution at amino acid position 352 of SEQ ID NO: 2, or a corresponding mutation in another Cas14 protein. In some embodiments, the substitution is an F352X substitution, wherein X is any amino acid other than F. In certain embodiments, the substitution is an F352Y substitution.

In some embodiments, the amino acid sequence of the Cas protein comprises a substitution at amino acid position 353 of SEQ ID NO: 2, or a corresponding mutation in another Cas14 protein. In some embodiments, the substitution is an H353X substitution, wherein X is any amino acid other than H. In certain embodiments, the substitution is an H353Y substitution.

In some embodiments, the amino acid sequence of the Cas protein comprises a substitution at amino acid position 366 of SEQ ID NO: 2, or a corresponding mutation in another Cas14 protein. In some embodiments, the substitution is an L366X substitution, wherein X is any amino acid other than L. In certain embodiments, the substitution is an L366M substitution.

In some embodiments, the amino acid sequence of the Cas protein comprises a substitution at amino acid position 367 of SEQ ID NO: 2, or a corresponding mutation in another Cas14 protein. In some embodiments, the substitution is a K367X substitution, wherein X is any amino acid other than K. In certain embodiments, the substitution is a K367E substitution.

In some embodiments, the amino acid sequence of the Cas protein comprises a substitution at amino acid position 372 of SEQ ID NO: 2, or a corresponding mutation in another Cas14 protein. In some embodiments, the substitution is a K372X substitution, wherein X is any amino acid other than K. In certain embodiments, the substitution is a K372M substitution.

In some embodiments, the amino acid sequence of the Cas protein comprises a substitution at amino acid position 378 of SEQ ID NO: 2, or a corresponding mutation in another Cas14 protein. In some embodiments, the substitution is an A378X substitution, wherein X is any amino acid other than A. In certain embodiments, the substitution is an A378V substitution.

In some embodiments, the amino acid sequence of the Cas protein comprises a substitution at amino acid position 392 of SEQ ID NO: 2, or a corresponding mutation in another Cas14 protein. In some embodiments, the substitution is an S392X substitution, wherein X is any amino acid other than S. In certain embodiments, the substitution is an S392I substitution.

In some embodiments, the amino acid sequence of the Cas protein comprises a substitution at amino acid position 423 of SEQ ID NO: 2, or a corresponding mutation in another Cas14 protein. In some embodiments, the substitution is an N423X substitution, wherein X is any amino acid other than N. In certain embodiments, the substitution is an N423T substitution. In certain embodiments, the substitution is an N423S substitution. In certain embodiments, the substitution is an N423D substitution.

In some embodiments, the amino acid sequence of the Cas protein comprises a substitution at amino acid position 425 of SEQ ID NO: 2, or a corresponding mutation in another Cas14 protein. In some embodiments, the substitution is an E425X substitution, wherein X is any amino acid other than E. In certain embodiments, the substitution is an E425K substitution.

In some embodiments, the amino acid sequence of the Cas protein comprises a substitution at amino acid position 430 of SEQ ID NO: 2, or a corresponding mutation in another Cas14 protein. In some embodiments, the substitution is a K430X substitution, wherein X is any amino acid other than K. In certain embodiments, the substitution is a K430R substitution.

In some embodiments, the amino acid sequence of the Cas protein comprises a substitution at amino acid position 461 of SEQ ID NO: 2, or a corresponding mutation in another Cas14 protein. In some embodiments, the substitution is an I461X substitution, wherein X is any amino acid other than I. In certain embodiments, the substitution is an I461V substitution.

In some embodiments, the amino acid sequence of the Cas protein comprises a substitution at amino acid position 471 of SEQ ID NO: 2, or a corresponding mutation in another Cas14 protein. In some embodiments, the substitution is a T471X substitution, wherein X is any amino acid other than T. In certain embodiments, the substitution is a T471I substitution.

In some embodiments, the amino acid sequence of the Cas protein comprises a substitution at amino acid position 477 of SEQ ID NO: 2, or a corresponding mutation in another Cas14 protein. In some embodiments, the substitution is a K477X substitution, wherein X is any amino acid other than K. In certain embodiments, the substitution is a K477E substitution.

In some embodiments, the amino acid sequence of the Cas protein comprises a substitution at amino acid position 483 of SEQ ID NO: 2, or a corresponding mutation in another Cas14 protein. In some embodiments, the substitution is an N483X substitution, wherein X is any amino acid other than N. In certain embodiments, the substitution is an N483D substitution.

In some embodiments, the amino acid sequence of the Cas protein comprises a substitution at amino acid position 486 of SEQ ID NO: 2, or a corresponding mutation in another Cas14 protein. In some embodiments, the substitution is an N486X substitution, wherein X is any amino acid other than N. In certain embodiments, the substitution is an N486D substitution.

In some embodiments, the amino acid sequence of the Cas protein comprises a substitution at amino acid position 507 of SEQ ID NO: 2, or a corresponding mutation in another Cas14 protein. In some embodiments, the substitution is an E507X substitution, wherein X is any amino acid other than E. In certain embodiments, the substitution is an E507D substitution.

In some embodiments, the amino acid sequence of the Cas protein comprises a substitution at amino acid position 508 of SEQ ID NO: 2, or a corresponding mutation in another Cas14 protein. In some embodiments, the substitution is an N508X substitution, wherein X is any amino acid other than N. In certain embodiments, the substitution is an N508D substitution.

In some embodiments, the amino acid sequence of the Cas protein comprises a substitution at amino acid position 510 of SEQ ID NO: 2, or a corresponding mutation in another Cas14 protein. In some embodiments, the substitution is an A510X substitution, wherein X is any amino acid other than A. In certain embodiments, the substitution is an A510D substitution.

In some embodiments, the amino acid sequence of the Cas protein comprises a substitution at amino acid position 513 of SEQ ID NO: 2, or a corresponding mutation in another Cas14 protein. In some embodiments, the substitution is an A513X substitution, wherein X is any amino acid other than A. In certain embodiments, the substitution is an A513S substitution.

In some embodiments, the amino acid sequence of the Cas protein comprises a substitution at amino acid position 519 of SEQ ID NO: 2, or a corresponding mutation in another Cas14 protein. In some embodiments, the substitution is an N519X substitution, wherein X is any amino acid other than N. In certain embodiments, the substitution is an N519I substitution.

In some embodiments, the amino acid sequence of the Cas protein comprises a substitution at amino acid position 528 of SEQ ID NO: 2, or a corresponding mutation in another Cas14 protein. In some embodiments, the substitution is an E528X substitution, wherein X is any amino acid other than E. In certain embodiments, the substitution is an E528K substitution.

In some embodiments, the amino acid sequence of the Cas protein comprises a substitution at amino acid position 529 of SEQ ID NO: 2, or a corresponding mutation in another Cas14 protein. In some embodiments, the substitution is a P529X substitution, wherein X is any amino acid other than P. In certain embodiments, the substitution is a P529S substitution.

In some embodiments, the Cas protein comprises a combination of substitutions of any one of the Cas clones listed in Table 1 below:

TABLE 1
Cas Protein Variants - First Round of Evolution

| Cas14a1 Variant Clone | Amino Acid Substitutions |
|---|---|
| P21-L1.7-1 | R148K, E206K, S266I, N305H, F341S, and K372M |
| P21-L1.7-2 | Q201R, H210Y, E274D, D282E, A301T, F341C, N423T, |
| | E425K, and N483D |
| P21-L1.7-3 | I37V, K43R, R66S, I118F, R148K, E206K, N209K, S266I, |
| | A301T, N305H, F341S, and K372M |
| P21-L1.7-4 | R66S, I118F, R148K, E206K, N209K, S266I, A301T, N305H, |
| | F341S, K372M, A378V, and K430R |
| P21-L1.7-5 | K76E, Q201R, H210Y, E274D, A301T, F341C, E425K, and |
| | N486D |
| P21-L1.7-6 | K76E, Q201R, H210Y, E274D, A301T, F341C, E425K, and |
| | N486D |
| P21-L1.7-7 | M1R, R66S, I118F, A134T, R148K, A157T, E206K, N209K, |
| | S266I, A301T, N305H, F341S, and K372M |
| P21-L1.7-8 | R66S, I118F, R148K, E206K, N209K, S266I, A301T, N305H, |
| | F341S, and K372M |
| P21-L2.7-1 | K41E, D44G, V46G, E206K, N209K, S266I, E298G, F352Y, |
| | L366M, S392I, N423S, I461V, E507D, and N508D |
| P21-L2.7-2 | K41E, D44G, E206K, N209K, K260R, S266I, F352Y, L366M, |
| | S392I, N423S, I461V, E507D, and N508D |
| P21-L2.7-3 | N32D, D44G, E206K, N209K, D309A, F352Y, H353Y, S392I, |
| | I461V, E507D, and A510D |
| P21-L2.7-4 | D44G, E138A, E206K, N209K, S266I, I296N, F352Y, L366M, |
| | S392I, I461V, E507D, and N508D |
| P21-L2.7-5 | E206K, N209K, S266I, Q284R, S320N, F352Y, S392I, E507D, |
| | and N508D |
| P21-L2.7-6 | A58T, K76T, E206K, N209K, S266I, F352Y, S392I, N483D, and |
| | E507D |
| P21-L2.7-7 | K41E, D44G, E206K, N209K, S266I, E298G, K330T, F352Y, |
| | L366M, S392I, N423S, I461V, E507D, and N508D |
| P21-L2.7-8 | D44G, I118F, E206K, N209K, S266I, M303V, F352Y, S392I, |
| | I461V, E507D, and A510D |
| P21-L3.7-1 | E206K, N209K, D268A, E298G, I313V, F341S, and P529S |
| P21-L3.7-2 | I131T, E206K, N209K, D268A, E298G, S392I, N423D, and |
| | P529S |
| P21-L3.7-3 | E206K, N209K, D268A, E298G, F341S, and P529S |
| P21-L3.7-4 | K25R, K179T, E206K, N209K, D268A, E298G, F341S, and |
| | P529S |
| P21-L3.7-5 | T203R, N209K, D268A, and C297G |
| P21-L3.7-6 | E206K, N209K, D268A, E298G, F341S, N349S, and P529S |
| P21-L3.7-7 | E206K, N209K, D268A, E298G, F341S, and P529S |
| P21-L3.7-8 | E206K, N209K, D268A, E298G, and P529S |
| P21-L4.7-1 | A2S, G87E, E228D, M303V, F341S, K367E, N423D, T471I, |
| | K477E, N508D, N519I, and E528K |
| P21-L4.7-2 | A2S, G87E, E228D, M303V, F341S, K367E, N423D, T471I, |
| | K477E, N508D, N519I, and E528K |
| P21-L4.7-3 | A2S, G87E, E228D, M303V, F341S, K367E, N423D, T471I, |
| | K477E, N508D, A513S, N519I, and E528K |
| P21-L4.7-4 | K11T, G87E, E228D, E298G, F341S, K367E, N423D, T471I, |
| | K477E, N508D, N519I, and E528K |
| P21-L4.7-5 | A2S, G87E, E228D, M303V, F341S, K367E, N423D, T471I, |
| | K477E, N508D, N519I, and E528K |
| P21-L4.7-6 | A2S, G87E, V137A, E228D, M303V, F341S, K367E, N423D, |
| | T471I, K477E, N508D, N519I, and E528K |
| P21-L4.7-7 | A2S, G87E, E228D, M303V, F341S, K367E, N423D, T471I, |
| | K477E, N508D, N519I, and E528K |
| P21-L4.7-8 | G87E, E228D, I296F, M303V, N423D, T471I, K477E, A510D, |
| | N519I, and E528K |
| P24-L4.7-2 | K76E, Q201R, H210Y, A301T, I313V, F341C, E425K, N486D, |
| | and S524A |
| P24-L4.7-4 | A58T, K76E, Q201R, H210Y, E274D, A301T, F341C, E425K, |
| | N486D, and S524P |
| P24-L4.7-5 | Q201R, H210Y, S246F, E274D, A301T, F341C, N369S, N423T, |
| | E425K, N486D, and S524P |
| P24-L4.7-6 | K76E, Q201R, H210Y, E274D, A301T, F341C, E425K, N486D, |
| | K506E, and N508D |

In some embodiments, the Cas protein comprises a combination of substitutions of any one of the clones selected from the group consisting of P21-L1.7-1, P21-L1.7-2, P21-L1.7-3, P21-L1.7-4, P21-L1.7-5, P21-L1.7-6, P21-L1.7-7, P21-L1.7-8, P21-L2.7-1, P21-L2.7-2, P21-L2.7-3, P21-L2.7-4, P21-L2.7-5, P21-L2.7-6, P21-L2.7-7, P21-L2.7-8, P21-L3.7-1, P21-L3.7-2, P21-L3.7-3, P21-L3.7-4, P21-L3.7-5, P21-L3.7-6, P21-L3.7-7, P21-L3.7-8, P21-L4.7-1, P21-L4.7-2, P21-L4.7-3, P21-L4.7-4, P21-L4.7-5, P21-L4.7-6, P21-L4.7-7, P21-L4.7-8, P24-L4.7-2, P24-L4.7-4, P24-L4.7-5, and P24-L4.7-6. In certain embodiments, the Cas protein comprises the combination of substitutions of clone P24-L4.7-4. In some embodiments, the Cas protein comprises an amino acid sequence that is at least 70%, at least 75%, at least 80%, at least 85%, at least 90%, at least 95%, or at least 99% identical to the amino acid sequence of any of the Cas proteins provided in Table 1. In some embodiments, the Cas protein comprises an amino acid sequence that is 100% identical to the amino acid sequence of any of the Cas proteins provided in Table 1. In some embodiments, the present disclosure provides fragments or truncated variants of any of the Cas proteins provided herein.

In some embodiments, the amino acid sequence of the Cas protein comprises at least 1, at least 2, at least 3, at least 4, at least 5, at least 6, at least 7, at least 8, at least 9, at least 10, at least 11, or at least 12 amino acid substitutions relative to a wild-type Cas14a1 protein of SEQ ID NO: 2. In some embodiments, the amino acid sequence of the Cas protein comprises more than 12 amino acid substitutions relative to a wild-type Cas14a1 protein of SEQ ID NO: 2. In some embodiments, the Cas protein comprises at least 13, at least 14, at least 15, at least 16, at least 17, at least 18, at least 19, at least 20, or more than 20 amino acid substitutions relative to a wild-type Cas protein of SEQ ID NO: 2.

In some embodiments, the Cas protein comprises substitutions at any of the following groups of positions: K76, Q201, H210, E274, A301, F341, E425, and N486; A58, K76, E206, N209, S266, F352, S392, N483, and E507; E206, N209, D268, E298, I313, F341, and P529; I131, E206, N209, D268, E298, S392, N423, and P529; and T203, N209, D268, and C297. In certain embodiments, the Cas protein comprises any of the following groups of substitutions: K76E, Q201R, H210Y, E274D, A301T, F341C, E425K, and N486D; A58T, K76T, E206K, N209K, S266I, F352Y, S392I, N483D, and E507D; E206K, N209K, D268A, E298G, I313V, F341S, and P529S; I131T, E206K, N209K, D268A, E298G, S392I, N423D, and P529S; and T203R, N209K, D268A, and C297G.

In some embodiments, the Cas protein comprises substitutions at any of the following groups of positions: K76, Q201, H210, E274, A301, I313, F341, E425, N486, and S524; A58, K76, Q201, H210, E274, A301, F341, E425, N486, and S524; Q201, H210, S246, E274, A301, F341, N369, N423, E425, N486, and S524; and K76, Q201, H210, E274, A301, F341, E425, N486, K506, and N508. In certain embodiments, the Cas protein comprises any of the following groups of substitutions: K76E, Q201R, H210Y, E274D, A301T, I313V, F341C, E425K, N486D, and S524A; A58T, K76E, Q201R, H210Y, E274D, A301T, F341C, E425K, N486D, and S524P; Q201R, H210Y, S246F, E274D, A301T, F341C, N369S, N423T, E425K, N486D, and S524P; and K76E, Q201R, H210Y, E274D, A301T, F341C, E425K, N486D, K506E, and N508D.

In some aspects, the present disclosure provides Cas proteins comprising additional mutations in combination with any of those described above. In some embodiments, the Cas protein comprises at least 2, at least 3, at least 4, at least 5, at least 6, at least 7, at least 8, at least 9, at least 10, at least 11, or at least 12 substitutions at positions selected from the group consisting of amino acid residues 1, 79, 111, 121, 133, 135, 151, 179, 202, 213, 228, 232, 236, 244, 260, 261, 280, 285, 313, 344, 369, 374, 388, 392, 393, 423, 425, 429, 430, 448, 459, 460, 464, 497, 513, 516, 525, and 526 of the amino acid sequence provided in SEQ ID NO: 2. In some embodiments, the Cas protein comprises at least 2, at least 3, at least 4, at least 5, at least 6, at least 7, at least 8, at least 9, at least 10, at least 11, or at least 12 substitutions selected from the group consisting of M1X, D79X, E111X, Y121X, N133X, S135X, E151X, K179X, Y202X, D213X, E228X, Y232X, E236X, Q244X, K260X, R261X, N280X, T285X, I313X, Y344X, N369X, A374X, L388X, S392X, E393X, N423X, K425X, R429X, K430X, M448X, Y459X, G460X, R464X, H497X, A513X, N516X, T525X, and K526X, relative to the amino acid sequence provided in SEQ ID NO: 2, wherein X represents any amino acid other than the wild type amino acid. In certain embodiments, the Cas protein comprises at least 2, at least 3, at least 4, at least 5, at least 6, at least 7, at least 8, at least 9, at least 10, at least 11, or at least 12 substitutions selected from the group consisting of M1K, M1I, D79Y, E111K, Y121H, N133T, N133K, S135R, E151K, E151A, K179E, Y202D, Y202C, D213A, D213N, E228G, Y232C, Y232F, E236D, Q244K, Q244R, K260R, R261K, N280S, T285I, I313V, I313T, Y344C, N369D, A374V, L388R, S392I, E393K, N423T, N423D, K425E, R429L, K430R, M448I, Y459S, G460A, R464I, H497P, A513V, N516S, T525A, and K526R, relative to the amino acid sequence provided in SEQ ID NO: 2.

In some embodiments, the amino acid sequence of the Cas protein comprises a substitution at amino acid position 1 of SEQ ID NO: 2, or a corresponding mutation in another Cas14 protein. In some embodiments, the substitution is an M1X substitution, wherein X is any amino acid other than M. In certain embodiments, the substitution is an M1K substitution. In certain embodiments, the substitution is an M1I substitution.

In some embodiments, the amino acid sequence of the Cas protein comprises a substitution at amino acid position 79 of SEQ ID NO: 2, or a corresponding mutation in another Cas14 protein. In some embodiments, the substitution is an D79X substitution, wherein X is any amino acid other than D. In certain embodiments, the substitution is an D79Y substitution.

In some embodiments, the amino acid sequence of the Cas protein comprises a substitution at amino acid position 111 of SEQ ID NO: 2, or a corresponding mutation in another Cas14 protein. In some embodiments, the substitution is an E111X substitution, wherein X is any amino acid other than E. In certain embodiments, the substitution is an E111K substitution.

In some embodiments, the amino acid sequence of the Cas protein comprises a substitution at amino acid position 121 of SEQ ID NO: 2, or a corresponding mutation in another Cas14 protein. In some embodiments, the substitution is a Y121X substitution, wherein X is any amino acid other than Y. In certain embodiments, the substitution is a Y121H substitution.

In some embodiments, the amino acid sequence of the Cas protein comprises a substitution at amino acid position 133 of SEQ ID NO: 2, or a corresponding mutation in another Cas14 protein. In some embodiments, the substitution is an N133X substitution, wherein X is any amino acid other than N. In certain embodiments, the substitution is an N133T substitution.

In some embodiments, the amino acid sequence of the Cas protein comprises a substitution at amino acid position 135 of SEQ ID NO: 2, or a corresponding mutation in another Cas14 protein. In some embodiments, the substitution is an S135X substitution, wherein X is any amino acid other than S. In certain embodiments, the substitution is an S135R substitution.

In some embodiments, the amino acid sequence of the Cas protein comprises a substitution at amino acid position 151 of SEQ ID NO: 2, or a corresponding mutation in another Cas14 protein. In some embodiments, the substitution is an E151X substitution, wherein X is any amino acid other than E. In certain embodiments, the substitution is an E151K substitution. In certain embodiments, the substitution is an E151A substitution.

In some embodiments, the amino acid sequence of the Cas protein comprises a substitution at amino acid position 179 of SEQ ID NO: 2, or a corresponding mutation in another Cas14 protein. In some embodiments, the substitution is a K179X substitution, wherein X is any amino acid other than K. In certain embodiments, the substitution is a K179E substitution.

In some embodiments, the amino acid sequence of the Cas protein comprises a substitution at amino acid position 202 of SEQ ID NO: 2, or a corresponding mutation in another Cas14 protein. In some embodiments, the substitution is a Y202X substitution, wherein X is any amino acid other than Y. In certain embodiments, the substitution is a Y202D substitution.

In some embodiments, the amino acid sequence of the Cas protein comprises a substitution at amino acid position 213 of SEQ ID NO: 2, or a corresponding mutation in another Cas14 protein. In some embodiments, the substitution is an D213X substitution, wherein X is any amino acid other than D. In certain embodiments, the substitution is an D213A substitution.

In certain embodiments, the substitution is a D213N substitution.

In some embodiments, the amino acid sequence of the Cas protein comprises a substitution at amino acid position 228 of SEQ ID NO: 2, or a corresponding mutation in another Cas14 protein. In some embodiments, the substitution is an E228X substitution, wherein X is any amino acid other than E. In certain embodiments, the substitution is an E228G substitution.

In some embodiments, the amino acid sequence of the Cas protein comprises a substitution at amino acid position 232 of SEQ ID NO: 2, or a corresponding mutation in another Cas14 protein. In some embodiments, the substitution is a Y232X substitution, wherein X is any amino acid other than Y. In certain embodiments, the substitution is a Y232C substitution. In certain embodiments, the substitution is a Y232F substitution.

In some embodiments, the amino acid sequence of the Cas protein comprises a substitution at amino acid position 236 of SEQ ID NO: 2, or a corresponding mutation in another Cas14 protein. In some embodiments, the substitution is an E236X substitution, wherein X is any amino acid other than E. In certain embodiments, the substitution is an E236D substitution.

In some embodiments, the amino acid sequence of the Cas protein comprises a substitution at amino acid position 244 of SEQ ID NO: 2, or a corresponding mutation in another Cas14 protein. In some embodiments, the substitution is a Q244X substitution, wherein X is any amino acid other than Q. In certain embodiments, the substitution is a Q244K substitution. In certain embodiments, the substitution is a Q244R substitution.

In some embodiments, the amino acid sequence of the Cas protein comprises a substitution at amino acid position 260 of SEQ ID NO: 2, or a corresponding mutation in another Cas14 protein. In some embodiments, the substitution is a K260X substitution, wherein X is any amino acid other than K. In certain embodiments, the substitution is a K260R substitution.

In some embodiments, the amino acid sequence of the Cas protein comprises a substitution at amino acid position 261 of SEQ ID NO: 2, or a corresponding mutation in another Cas14 protein. In some embodiments, the substitution is an R261X substitution, wherein X is any amino acid other than R. In certain embodiments, the substitution is an R261K substitution.

In some embodiments, the amino acid sequence of the Cas protein comprises a substitution at amino acid position 280 of SEQ ID NO: 2, or a corresponding mutation in another Cas14 protein. In some embodiments, the substitution is an N280X substitution, wherein X is any amino acid other than N. In certain embodiments, the substitution is an N280S substitution.

In some embodiments, the amino acid sequence of the Cas protein comprises a substitution at amino acid position 285 of SEQ ID NO: 2, or a corresponding mutation in another Cas14 protein. In some embodiments, the substitution is a T285X substitution, wherein X is any amino acid other than T. In certain embodiments, the substitution is a T285I substitution.

In some embodiments, the amino acid sequence of the Cas protein comprises a substitution at amino acid position 313 of SEQ ID NO: 2, or a corresponding mutation in another Cas14 protein. In some embodiments, the substitution is an I313X substitution, wherein X is any amino acid other than I. In certain embodiments, the substitution is an I313V substitution. In certain embodiments, the substitution is an I313T substitution.

In some embodiments, the amino acid sequence of the Cas protein comprises a substitution at amino acid position 344 of SEQ ID NO: 2, or a corresponding mutation in another Cas14 protein. In some embodiments, the substitution is a Y344X substitution, wherein X is any amino acid other than Y. In certain embodiments, the substitution is a Y344C substitution.

In some embodiments, the amino acid sequence of the Cas protein comprises a substitution at amino acid position 369 of SEQ ID NO: 2, or a corresponding mutation in another Cas14 protein. In some embodiments, the substitution is an N369X substitution, wherein X is any amino acid other than N. In certain embodiments, the substitution is an N369D substitution.

In some embodiments, the amino acid sequence of the Cas protein comprises a substitution at amino acid position 374 of SEQ ID NO: 2, or a corresponding mutation in another Cas14 protein. In some embodiments, the substitution is an A374X substitution, wherein X is any amino acid other than A. In certain embodiments, the substitution is an A374V substitution.

In some embodiments, the amino acid sequence of the Cas protein comprises a substitution at amino acid position 388 of SEQ ID NO: 2, or a corresponding mutation in another Cas14 protein. In some embodiments, the substitution is an L388X substitution, wherein X is any amino acid other than L. In certain embodiments, the substitution is an L388R substitution.

In some embodiments, the amino acid sequence of the Cas protein comprises a substitution at amino acid position 392 of SEQ ID NO: 2, or a corresponding mutation in another Cas14 protein. In some embodiments, the substitution is an S392X substitution, wherein X is any amino acid other than S. In certain embodiments, the substitution is an S392I substitution.

In some embodiments, the amino acid sequence of the Cas protein comprises a substitution at amino acid position 393 of SEQ ID NO: 2, or a corresponding mutation in another Cas14 protein. In some embodiments, the substitution is an E393X substitution, wherein X is any amino acid other than E. In certain embodiments, the substitution is an E393K substitution.

In some embodiments, the amino acid sequence of the Cas protein comprises a substitution at amino acid position 423 of SEQ ID NO: 2, or a corresponding mutation in another Cas14 protein. In some embodiments, the substitution is an N423X substitution, wherein X is any amino acid other than N. In certain embodiments, the substitution is an N423T substitution.

In certain embodiments, the substitution is an N423D substitution.

In some embodiments, the amino acid sequence of the Cas protein comprises a substitution at amino acid position 425 of SEQ ID NO: 2, or a corresponding mutation in another Cas14 protein. In some embodiments, the substitution is a K425X substitution, wherein X is any amino acid other than K. In certain embodiments, the substitution is a K425E substitution.

In some embodiments, the amino acid sequence of the Cas protein comprises a substitution at amino acid position 429 of SEQ ID NO: 2, or a corresponding mutation in another Cas14 protein. In some embodiments, the substitution is an R429X substitution, wherein X is any amino acid other than R. In certain embodiments, the substitution is an R429L substitution.

In some embodiments, the amino acid sequence of the Cas protein comprises a substitution at amino acid position 430 of SEQ ID NO: 2, or a corresponding mutation in another Cas14 protein. In some embodiments, the substitution is a K430X substitution, wherein X is any amino acid other than K. In certain embodiments, the substitution is a K430R substitution.

In some embodiments, the amino acid sequence of the Cas protein comprises a substitution at amino acid position 448 of SEQ ID NO: 2, or a corresponding mutation in another Cas14 protein. In some embodiments, the substitution is an M448X substitution, wherein X is any amino acid other than M. In certain embodiments, the substitution is an M448I substitution.

In some embodiments, the amino acid sequence of the Cas protein comprises a substitution at amino acid position 459 of SEQ ID NO: 2, or a corresponding mutation in another Cas14 protein. In some embodiments, the substitution is a Y459X substitution, wherein X is any amino acid other than Y. In certain embodiments, the substitution is a Y459S substitution.

In some embodiments, the amino acid sequence of the Cas protein comprises a substitution at amino acid position 460 of SEQ ID NO: 2, or a corresponding mutation in another Cas14 protein. In some embodiments, the substitution is a G460X substitution, wherein X is any amino acid other than G. In certain embodiments, the substitution is a G460A substitution.

In some embodiments, the amino acid sequence of the Cas protein comprises a substitution at amino acid position 464 of SEQ ID NO: 2, or a corresponding mutation in another Cas14 protein. In some embodiments, the substitution is an R464X substitution, wherein X is any amino acid other than R. In certain embodiments, the substitution is an R464I substitution.

In some embodiments, the amino acid sequence of the Cas protein comprises a substitution at amino acid position 497 of SEQ ID NO: 2, or a corresponding mutation in another Cas14 protein. In some embodiments, the substitution is an H497X substitution, wherein X is any amino acid other than H. In certain embodiments, the substitution is an H497P substitution.

In some embodiments, the amino acid sequence of the Cas protein comprises a substitution at amino acid position 513 of SEQ ID NO: 2, or a corresponding mutation in another Cas14 protein. In some embodiments, the substitution is an A513X substitution, wherein X is any amino acid other than A. In certain embodiments, the substitution is an A513V substitution.

In some embodiments, the amino acid sequence of the Cas protein comprises a substitution at amino acid position 516 of SEQ ID NO: 2, or a corresponding mutation in another Cas14 protein. In some embodiments, the substitution is an N516X substitution, wherein X is any amino acid other than N. In certain embodiments, the substitution is an N516S substitution.

In some embodiments, the amino acid sequence of the Cas protein comprises a substitution at amino acid position 525 of SEQ ID NO: 2, or a corresponding mutation in another Cas14 protein. In some embodiments, the substitution is a T525X substitution, wherein X is any amino acid other than T. In certain embodiments, the substitution is a T525A substitution.

In some embodiments, the amino acid sequence of the Cas protein comprises a substitution at amino acid position 526 of SEQ ID NO: 2, or a corresponding mutation in another Cas14 protein. In some embodiments, the substitution is an K526X substitution, wherein X is any amino acid other than K. In certain embodiments, the substitution is an K526R substitution.

In some embodiments, the Cas protein comprises a combination of substitutions of any one of the Cas clones listed in Table 2 below, relative to a wild-type Cas14a1 protein, or relative to one of the Cas clones provided in Table 1, for example, P24-L4.7-4:

TABLE 2
Cas Protein Variants - Second Round of Evolution

| Cas14a1 Variant Clone | Amino Acid Substitutions |
|---|---|
| P28L1.5-1 | E111K, K179E, E228G, E236D, R261K, T285I, Y344C, N369D, |
| | L388R, E393K, and K430R |
| P28L1.5-2 | E111K, N133T, K179E, E228G, Y344C, N369D, L388R, |
| | E393K, K430R, and N516S |
| P28L1.5-3A | E111K, K179E, E228G, T285I, Y344C, N369D, L388R, E393K, |
| | and K430R |
| P28L1.5-4 | E111K, K179E, E228G, R261K, Y344C, N369D, E393K, |
| | K430R, Y459S, and G460A |
| P28L1.5-4A | E111K, E151K, E228G, R261K, Y344C, N369D, E393K, |
| | K430R, Y459S, and G460A |
| P28L1.5-5 | E111K, K179E, E228G, E236D, R261K, Y344C, N369D, |
| | L388R, E393K, and K430R |
| P28L1.5-5A | E111K, K179E, Y202D, E228G, E236D, R261K, Y344C, |
| | N369D, L388R, E393K, N423T, and K430R |
| P28L1.5-6 | Y121H, K179E, E228G, E236D, R261K, T285I, Y344C, N369D, |
| | L388R, E393K, and K430R |
| P28L1.5-6A | E111K, Y121H, E151K, E228G, E236D, R261K, Y344C, |
| | N369D, E393K, and K430R |
| P28L1.5-7 | E111K, K179E, Y202C, Y344C, N369D, L388R, E393K, |
| | K430R, and N516S |
| P28L2.5-1A | N133K, E228G, E236D, Q244K, K260R, T285I, A374V, and |
| | K425E |
| P28L2.5-2 | E228G, E236D, and K425E |
| P28L2.5-2A | N133K, E228G, E236D, Q244K, K260R, T285I, A374V, and |
| | K425E |
| P28L2.5-3 | N133K, E228G, E236D, Q244K, K260R, T285I, I313V, A374V, |
| | and K425E |
| P28L2.5-3A | N133K, E228G, E236D, Q244K, K260R, T285I, I313V, A374V, |
| | and K425E |
| P28L2.5-4A | N133K, E228G, E236D, Q244K, K260R, T285I, A374V, and |
| | K425E |
| P28L2.5-5A | N133K, E228G, E236D, Q244K, K260R, T285I, A374V, and |
| | K425E |
| P28L2.5-6 | E228G, E236D, T285I, and K425E |
| P28L2.5-6A | N133K, E228G, E236D, Q244K, K260R, T285I, A374V, and |
| | K425E |
| P28L2.5-7 | N133K, E228G, E236D, Q244K, K260R, T285I, A374V, and |
| | K425E |
| P28L3.5-1 | M1K, K179E, E228G, E236D, T285I, N423D, M448I, and |
| | T525A |
| P28L3.5-2 | M1K, S135R, K179E, E228G, E236D, T285I, N423D, M448I, |
| | H497P, T525A, and K526R |
| P28L3.5-3 | M1I, E228G, Y232C, T285I, and S392I |
| P28L3.5-4 | M1K, S135R, K179E, E228G, E236D, T285I, N423D, M448I, |
| | and T525A |
| P28L3.5-5 | E228G, Y232C, R261K, T285I, S392I, and N423D |
| P28L3.5-6 | E228G, Y232C, T285I, S392I, and R429L |
| P28L3.5-7 | E228G, Y232C, T285I, S392I, and N423D |
| P28L3.5-8 | M1K, S135R, K179E, E228G, E236D, T285I, N423D, M448I, |
| | H497P, and T525A |
| P28L4.5-2 | N133K, K179E, D213A, E228G, T285I, and S392I |
| P28L4.5-3 | D79Y, K179E, D213N, Q244R, N280S, T285I, E393K, R464I, |
| | and A513V |
| P28L4.5-4 | D79Y, K179E, D213N, Q244R, N280S, T285I, E393K, R464I, |
| | and A513V |
| P28L4.5-5 | K179E, D213A, E228G, Y232F, T285I, and S392I |
| P28L4.5-6 | E151A, K179E, D213A, E228G, E236D, I313T, and S392I |

In some embodiments, the Cas protein comprises a combination of substitutions of any one of the clones selected from the group consisting of P28L1.5-1, P28L1.5-2, P28L1.5-3A, P28L1.5-4, P28L1.5-4A, P28L1.5-5, P28L1.5-5A, P28L1.5-6, P28L1.5-6A, P28L1.5-7, P28L2.5-1A, P28L2.5-2, P28L2.5-2A, P28L2.5-3, P28L2.5-3A, P28L2.5-4A, P28L2.5-5A, P28L2.5-6, P28L2.5-6A, P28L2.5-7, P28L3.5-1, P28L3.5-2, P28L3.5-3, P28L3.5-4, P28L3.5-5, P28L3.5-6, P28L3.5-7, P28L3.5-8, P28L4.5-2, P28L4.5-3, P28L4.5-4, P28L4.5-5, and P28L4.5-6. In certain embodiments, the Cas protein comprises the substitutions of the clone P28-L2.5-2A. In certain embodiments, the Cas protein comprises the substitutions of the clones P24-L4.7-4 and P28-L2.5-2A. In some embodiments, the Cas protein comprises an amino acid sequence that is at least 70%, at least 75%, at least 80%, at least 85%, at least 90%, at least 95%, or at least 99% identical to the amino acid sequence of any of the Cas proteins provided in Table 2. In some embodiments, the Cas protein comprises an amino acid sequence that is 100% identical to the amino acid sequence of any of the Cas proteins provided in Table 2.

In some embodiments, the Cas protein comprises substitutions at any of the following groups of positions: A58, K76, Q201, H210, E274, A301, F341, E425, N486, and S524; A58, K76, N133, Q201, H210, E228, E236, Q244, K260, E274, T285, A301, F341, A374, N486, and S524; A58, K76, N133, K179, Q201, H210, D213, E228, E274, T285, A301, F341, S392, E425, N486, and S524; and A58, K76, D79, D91, K179, Q201, H210, D213, Q244, E274, N280, T285, E298, A301, F341, E393, E425, N486, A510, A513, and S524. In certain embodiments, the Cas protein comprises any of the following groups of substitutions: A58T, K76E, Q201R, H210Y, E274D, A301T, F341C, E425K, N486D, and S524P; A58T, K76E, N133K, Q201R, H210Y, E228G, E236D, Q244K, K260R, E274D, T285I, A301T, F341C, A374V, N486D, and S524P; A58T, K76E, N133K, K179E, Q201R, H210Y, D213A, E228G, E274D, T285I, A301T, F341C, S392I, E425K, N486D, and S524P; and A58T, K76E, D79Y, D91A, K179E, Q201R, H210Y, D213N, Q244R, E274D, N280S, T285I, E298D, A301T, F341C, E393K, E425K, N486D, A510D, A513V, and S524P.

In certain embodiments, the Cas protein comprises the substitutions: A58T, K76E, Q201R, H210Y, E274D, A301T, F341C, E425K, N486D, and S524P. In certain embodiments, the Cas protein comprises the substitutions A58T, K76E, N133K, Q201R, H210Y, E228G, E236D, Q244K, K260R, E274D, T285I, A301T, F341C, A374V, N486D, and S524P.

In some aspects, the present disclosure provides Cas proteins comprising substitutions corresponding to any of the substitutions disclosed herein, or any combination thereof, in another Cas14 protein. Exemplary amino acid sequences of additional Cas14 proteins include, but are not limited to, the following:

| Cas Protein | Amino Acid Sequence |
|---|---|
| Cas14a2 | MEEAKTVSKTLSLRILRPLYSAEIEKEIKEEKERRKQGGKSGELDSGFY |
| | KKLEKKHTQMFGWDKLNLMLSQLQRQIARVFNQSISELYIETVIQGK |
| | KSNKHYTSKIVYNRAYSVFYNAYLALGITSKVEANFRSTELLMQKSS |
| | LPTAKSDNFPILLHKQKGVEGEEGGFKISADGNDLIFEIPIPFYEYDSAN |
| | KKEPFKWIKKGGQKPTIKLILSTFRRQRNKGWAKDEGTDAEIRKVIEG |
| | KYQVSHIEINRGKKLGDHQKWFVNFTIEQPIYERKLDKNIIGGIDVGIK |
| | SPLVCAVNNSFARYSVDSNDVLKFSKQAFAFRRRLLSKNSLKRSGHG |
| | SKNKLDPITRMTEKNDRFRKKIIERWAKEVTNFFIKNQVGTVQIEDLS |
| | TMKDRQDNFFNQYLRGFWPYYQMQNLIENKLKEYGIETKRIKARYTS |
| | QLCSNPSCRHWNSYFSFDHRKTNNFPKFKCEKCALEISADYNAARNIS |
| | TPDIEKFVAKATKGINLPDKNENVILE (SEQ ID NO: 3) |
| Cas14a3 | MEVQKTVMKTLSLRILRPLYSQEIEKEIKEEKERRKQAGGTGELDGGF |
| | YKKLEKKHSEMFSFDRLNLLLNQLQREIAKVYNHAISELYIATIAQGN |
| | KSNKHYISSIVYNRAYGYFYNAYIALGICSKVEANFRSNELLTQQSAL |
| | PTAKSDNFPIVLHKQKGAEGEDGGFRISTEGSDLIFEIPIPFYEYNGENR |
| | KEPYKWVKKGGQKPVLKLILSTFRRQRNKGWAKDEGTDAEIRKVTE |
| | GKYQVSQIEINRGKKLGEHQKWFANFSIEQPIYERKPNRSIVGGLDVGI |
| | RSPLVCAINNSFSRYSVDSNDVFKFSKQVFAFRRRLLSKNSLKRKGHG |
| | AAHKLEPITEMTEKNDKFRKKIIERWAKEVTNFFVKNQVGIVQIEDLS |
| | TMKDREDHFFNQYLRGFWPYYQMQTLIENKLKEYGIEVKRVQAKYT |
| | SQLCSNPNCRYWNNYFNFEYRKVNKFPKFKCEKCNLEISADYNAAR |
| | NLSTPDIEKFVAKATKGINLPEK (SEQ ID NO: 4) |
| Cas14a4 | MERQKVPQIRKIVRVVPLRILRPKYSDVIENALKKFKEKGDDTNTNDF |
| | WRAIRDRDTEFFRKELNFSEDEINQLERDTLFRVGLDNRVLFSYFDFL |
| | QEKLMKDYNKIISKLFINRQSKSSFENDLTDEEVEELIEKDVTPFYGAY |
| | IGKGIKSVIKSNLGGKFIKSVKIDRETKKVTKLTAINIGLMGLPVAKSD |
| | TFPIKIIKTNPDYITFQKSTKENLQKIEDYETGIEYGDLLVQITIPWFKNE |
| | NKDFSLIKTKEAIEYYKLNGVGKKDLLNINLVLTTYHIRKKKSWQIDG |
| | SSQSLVREMANGELEEKWKSFFDTFIKKYGDEGKSALVKRRVNKKSR |
| | AKGEKGRELNLDERIKRLYDSIKAKSFPSEINLIPENYKWKLHFSIEIPP |
| | MVNDIDSNLYGGIDFGEQNIATLCVKNIEKDDYDFLTIYGNDLLKHA |
| | QASYARRRIMRVQDEYKARGHGKSRKTKAQEDYSERMQKLRQKITE |
| | RLVKQISDFFLWRNKFHMAVCSLRYEDLNTLYKGESVKAKRMRQFI |
| | NKQQLFNGIERKLKDYNSEIYVNSRYPHYTSRLCSKCGKLNLYFDFLK |
| | FRTKNIIIRKNPDGSEIKYMPFFICEFCGWKQAGDKNASANIADKDYQ |
| | DKLNKEKEFCNIRKPKSKKEDIGEENEEERDYSRRFNRNSFIYNSLKK |
| | DNKLNQEKLFDEWKNQLKRKIDGRNKFEPKEYKDRFSYLFAYYQEII |
| | KNESES (SEQ ID NO: 5) |
| Cas14a5 | MVPTELITKTLQLRVIRPLYFEEIEKELAELKEQKEKEFEETNSLLLESK |
| | KIDAKSLKKLKRKARSSAAVEFWKIAKEKYPDILTKPEMEFIFSEMQK |
| | MMARFYNKSMTNIFIEMNNDEKVNPLSLISKASTEANQVIKCSSISSGL |
| | NRKIAGSINKTKFKQVRDGLISLPTARTETFPISFYKSTANKDEIPISKIN |
| | LPSEEEADLTITLPFPFFEIKKEKKGQKAYSYFNIIEKSGRSNNKIDLLLS |
| | THRRQRRKGWKEEGGTSAEIRRLMEGEFDKEWEIYLGEAEKSEKAK |
| | NDLIKNMTRGKLSKDIKEQLEDIQVKYFSDNNVESWNDLSKEQKQEL |
| | SKLRKKKVEELKDWKHVKEILKTRAKIGWVELKRGKRQRDRNKWF |
| | VNITITRPPFINKELDDTKFGGIDLGVKVPFVCAVHGSPARLIIKENEIL |
| | QFNKMVSARNRQITKDSEQRKGRGKKNKFIKKEIFNERNELFRKKIIE |
| | RWANQIVKFFEDQKCATVQIENLESFDRTSYK (SEQ ID NO: 6) |
| Cas14b1 | MKLSEQENITTGVKFKLKLDKETSEGLNDYFDEYGKAINFAIKVIQKE |
| | LAEDRFAGKVRLDENKKPLLNEDGKKIWDFPNEFCSCGKQVNRYVN |
| | GKSLCQECYKNKFTEYGIRKRMYSAKGRKAEQDINIKNSTNKISKTHF |
| | NYAIREAFILDKSIKKQRKERFRRLREMKKKLQEFIEIRDGNKILCPKIE |
| | KQRVERYIHPSWINKEKKLEDFRGYSMSNVLGKIKILDRNIKREEKSL |
| | KEKGQINFKARRLMLDKSVKFLNDNKISFTISKNLPKEYELDLPEKEK |
| | RLNWLKEKIKIIKNQKPKYAYLLRKDDNFYLQYTLETEFNLKEDYSGI |
| | VGIDRGVSHIAVYTFVHNNGKNERPLFLNSSEILRLKNLQKERDRFLR |
| | RKHNKKRKKSNMRNIEKKIQLILHNYSKQIVDFAKNKNAFIVFEKLEK |
| | PKKNRSKMSKKSQYKLSQFTFKKLSDLVDYKAKREGIKVLYISPEYTS |
| | KECSHCGEKVNTQRPFNGNSSLFKCNKCGVELNADYNASINIAKKGL |
| | NILNSTN (SEQ ID NO: 7) |
| Cas14b2 | MEESIITGVKFKLRIDKETTKKLNEYFDEYGKAINFAVKIIQKELADDR |
| | FAGKAKLDQNKNPILDENGKKIYEFPDEFCSCGKQVNKYVNNKPFCQ |
| | ECYKIRFTENGIRKRMYSAKGRKAEHKINILNSTNKISKTHFNYAIREA |
| | FILDKSIKKQRKKRNERLRESKKRLQQFIDMRDGKREICPTIKGQKVD |
| | RFIHPSWITKDKKLEDFRGYTLSIINSKIKILDRNIKREEKSLKEKGQIIF |
| | KAKRLMLDKSIRFVGDRKVLFTISKTLPKEYELDLPSKEKRLNWLKE |
| | KIEIIKNQKPKYAYLLRKNIESEKKPNYEYYLQYTLEIKPELKDFYDGA |
| | IGIDRGINHIAVCTFISNDGKVTPPKFFSSGEILRLKNLQKERDRFLLRK |
| | HNKNRKKGNMRVIENKINLILHRYSKQIVDMAKKLNASIVFEELGRIG |
| | KSRTKMKKSQRYKLSLFIFKKLSDLVDYKSRREGIRVTYVPPEYTSKE |
| | CSHCGEKVNTQRPFNGNYSLFKCNKCGIQLNSDYNASINIAKKGLKIP |
| | NST (SEQ ID NO: 8) |
| Cas14b3 | MWTIVIGDFIEMPKQDLVTTGIKFKLDVDKETRKKLDDYFDEYGKAI |
| | NFAVKIIQKNLKEDRFAGKIALGEDKKPLLDKDGKKIYNYPNESCSCG |
| | NQVRRYVNAKPFCVDCYKLKFTENGIRKRMYSARGRKADSDINIKNS |
| | TNKISKTHFNYAIREGFILDKSLKKQRSKRIKKLLELKRKLQEFIDIRQG |
| | QMVLCPKIKNQRVDKFIHPSWLKRDKKLEEFRGYSLSVVEGKIKIFNR |
| | NILREEDSLRQRGHVNFKANRIMLDKSVRFLDGGKVNFNLNKGLPKE |
| | YLLDLPKKENKLSWLNEKISLIKLQKPKYAYLLRREGSFFIQYTIENVP |
| | KTFSDYLGAIGIDRGISHIAVCTFVSKNGVNKAPVFFSSGEILKLKSLQ |
| | KQRDLFLRGKHNKIRKKSNMRNIDNKINLILHKYSRNIVNLAKSEKAF |
| | IVFEKLEKIKKSRFKMSKSLQYKLSQFTFKKLSDLVEYKAKIEGIKVD |
| | YVPPEYTSKECSHCGEKVDTQRPFNGNSSLFKCNKCRVQLNADYNAS |
| | INIAKKSLNISNN (SEQ ID NO: 9) |
| Cas14b4 | MNMSKTTISVKLKIIDLSSEKKEFLDNYFNEYAKATTFCQLRIRRLLRN |
| | THWLGKKEKSSKKWIFESGICDLCGENKELVNEDRNSGEPAKICKRC |
| | YNGRYGNQMIRKLFVSTKKREVQENMDIRRVAKLNNTHYHRIPEEAF |
| | DMIKAADTAEKRRKKNVEYDKKRQMEFIEMFNDEKKRAARPKKPNE |
| | RETRYVHISKLESPSKGYTLNGIKRKIDGMGKKIERAEKGLSRKKIFG |
| | YQGNRIKLDSNWVRFDLAESEITIPSLFKEMKLRITGPTNVHSKSGQIY |
| | FAEWFERINKQPNNYCYLIRKTSSNGKYEYYLQYTYEAEVEANKEYA |
| | GCLGVDIGCSKLAAAVYYDSKNKKAQKPIEIFTNPIKKIKMRREKLIK |
| | LLSRVKVRHRRRKLMQLSKTEPIIDYTCHKTARKIVEMANTAKAFISM |
| | ENLETGIKQKQQARETKKQKFYRNMFLFRKLSKLIEYKALLKGIKIVY |
| | VKPDYTSQTCSSCGADKEKTERPSQAIFRCLNPTCRYYQRDINADFNA |
| | AVNIAKKALNNTEVVTTLL (SEQ ID NO: 10) |
| Cas14b5 | MARAKNQPYQKLTTTTGIKFKLDLSEEEGKRFDEYFSEYAKAVNFCA |
| | KVIYQLRKNLKFAGKKELAAKEWKFEISNCDFCNKQKEIYYKNIANG |
| | QKVCKGCHRTNFSDNAIRKKMIPVKGRKVESKFNIHNTTKKISGTHR |
| | HWAFEDAADIIESMDKQRKEKQKRLRREKRKLSYFFELFGDPAKRYE |
| | LPKVGKQRVPRYLHKIIDKDSLTKKRGYSLSYIKNKIKISERNIERDEK |
| | SLRKASPIAFGARKIKMSKLDPKRAFDLENNVFKIPGKVIKGQYKFFG |
| | TNVANEHGKKFYKDRISKILAGKPKYFYLLRKKVAESDGNPIFEYYV |
| | QWSIDTETPAITSYDNILGIDAGITNLATTVLIPKNLSAEHCSHCGNNH |
| | VKPIFTKFFSGKELKAIKIKSRKQKYFLRGKHNKLVKIKRIRPIEQKVD |
| | GYCHVVSKQIVEMAKERNSCIALEKLEKPKKSKFRQRRREKYAVSMF |
| | VFKKLATFIKYKAAREGIEIIPVEPEGTSYTCSHCKNAQNNQRPYFKPN |
| | SKKSWTSMFKCGKCGIELNSDYNAAFNIAQKALNMTSA (SEQ ID NO: |
| | 11) |
| Cas14b6 | MDEKHFFCSYCNKELKISKNLINKISKGSIREDEAVSKAISIHNKKEHS |
| | LILGIKFKLFIENKLDKKKLNEYFDNYSKAVTFAARIFDKIRSPYKFIGL |
| | KDKNTKKWTFPKAKCVFCLEEKEVAYANEKDNSKICTECYLKEFGE |
| | NGIRKKIYSTRGRKVEPKYNIFNSTKELSSTHYNYAIRDAFQLLDALK |
| | KQRQKKLKSIFNQKLRLKEFEDIFSDPQKRIELSLKPHQREKRYIHLSK |
| | SGQESINRGYTLRFVRGKIKSLTRNIEREEKSLRKKTPIHFKGNRLMIFP |
| | AGIKFDFASNKVKISISKNLPNEFNFSGTNVKNEHGKSFFKSRIELIKTQ |
| | KPKYAYVLRKIKREYSKLRNYEIEKIRLENPNADLCDFYLQYTIETESR |
| | NNEEINGIIGIDRGITNLACLVLLKKGDKKPSGVKFYKGNKILGMKIA |
| | YRKHLYLLKGKRNKLRKQRQIRAIEPKINLILHQISKDIVKIAKEKNFA |
| | IALEQLEKPKKARFAQRKKEKYKLALFTFKNLSTLIEYKSKREGIPVIY |
| | VPPEKTSQMCSHCAINGDEHVDTQRPYKKPNAQKPSYSLFKCNKCGI |
| | ELNADYNAAFNIAQKGLKTLMLNHSH (SEQ ID NO: 12) |
| Cas14b10 | MISLKLKLLPDEEQKKLLDEMFWKWASICTRVGFGRADKEDLKPPKD |
| | AEGVWFSLTQLNQANTDINDLREAMKHQKHRLEYEKNRLEAQRDDT |
| | QDALKNPDRREISTKRKDLFRPKASVEKGFLKLKYHQERYWVRRLKE |
| | INKLIERKTKTLIKIEKGRIKFKATRITLHQGSFKIRFGDKPAFLIKALSG |
| | KNQIDAPFVVVPEQPICGSVVNSKKYLDEITTNFLAYSVNAMLFGLSR |
| | SEEMLLKAKRPEKIKKKEEKLAKKQSAFENKKKELQKLLGRELTQQE |
| | EAIIEETRNQFFQDFEVKITKQYSELLSKIANELKQKNDFLKVNKYPIL |
| | LRKPLKKAKSKKINNLSPSEWKYYLQFGVKPLLKQKSRRKSRNVLGI |
| | DRGLKHLLAVTVLEPDKKTFVWNKLYPNPITGWKWRRRKLLRSLKR |
| | LKRRIKSQKHETIHENQTRKKLKSLQGRIDDLLHNISRKIVETAKEYD |
| | AVIVVEDLQSMRQHGRSKGNRLKTLNYALSLFDYANVMQLIKYKAG |
| | IEGIQIYDVKPAGTSQNCAYCLLAQRDSHEYKRSQENSKIGVCLNPNC |
| | QNHKKQIDADLNAARVIASCYALKINDSQPFGTRKRFKKRTTN (SEQ |
| | ID NO: 13) |
| Cas14b11 | METLSLKLKLNPSKEQLLVLDKMFWKWASICTRLGLKKAEMSDLEPP |
| | KDAEGVWFSKTQLNQANTDVNDLRKAMQHQGKRIEYELDKVENRR |
| | NEIQEMLEKPDRRDISPNRKDLFRPKAAVEKGYLKLKYHKLGYWSKE |
| | LKTANKLIERKRKTLAKIDAGKMKFKPTRISLHTNSFRIKFGEEPKIAL |
| | STTSKHEKIELPLITSLQRPLKTSCAKKSKTYLDAAILNFLAYSTNAAL |
| | FGLSRSEEMLLKAKKPEKIEKRDRKLATKRESFDKKLKTLEKLLERKL |
| | SEKEKSVFKRKQTEFFDKFCITLDETYVEALHRIAEELVSKNKYLEIKK |
| | YPVLLRKPESRLRSKKLKNLKPEDWTYYIQFGFQPLLDTPKPIKTKTV |
| | LGIDRGVRHLLAVSIFDPRTKTFTFNRLYSNPIVDWKWRRRKLLRSIK |
| | RLKRRLKSEKHVHLHENQFKAKLRSLEGRIEDHFHNLSKEIVDLAKE |
| | NNSVIVVENLGGMRQHGRGRGKWLKALNYALSHFDYAKVMQLIKY |
| | KAELAGVFVYDVAPAGTSINCAYCLLNDKDASNYTRGKVINGKKNT |
| | KIGECKTCKKEFDADLNAARVIALCYEKRLNDPQPFGTRKQFKPKKP |
| | (SEQ ID NO: 14) |
| Cas14b12 | MKALKLQLIPTRKQYKILDEMFWKWASLANRVSQKGESKETLAPKK |
| | DIQKIQFNATQLNQIEKDIKDLRGAMKEQQKQKERLLLQIQERRSTISE |
| | MLNDDNNKERDPHRPLNFRPKGWRKFHTSKHWVGELSKILRQEDRV |
| | KKTIERIVAGKISFKPKRIGIWSSNYKINFFKRKISINPLNSKGFELTLMT |
| | EPTQDLIGKNGGKSVLNNKRYLDDSIKSLLMFALHSRFFGLNNTDTYL |
| | LGGKINPSLVKYYKKNQDMGEFGREIVEKFERKLKQEINEQQKKIIMS |
| | QIKEQYSNRDSAFNKDYLGLINEFSEVFNQRKSERAEYLLDSFEDKIK |
| | QIKQEIGESLNISDWDFLIDEAKKAYGYEEGFTEYVYSKRYLEILNKIV |
| | KAVLITDIYFDLRKYPILLRKPLDKIKKISNLKPDEWSYYIQFGYDSINP |
| | VQLMSTDKFLGIDRGLTHLLAYSVFDKEKKEFIINQLEPNPIMGWKW |
| | KLRKVKRSLQHLERRIRAQKMVKLPENQMKKKLKSIEPKIEVHYHNI |
| | SRKIVNLAKDYNASIVVESLEGGGLKQHGRKKNARNRSLNYALSLFD |
| | YGKIASLIKYKADLEGVPMYEVLPAYTSQQCAKCVLEKGSFVDPEIIG |
| | YVEDIGIKGSLLDSLFEGTELSSIQVLKKIKNKIELSARDNHNKEINLIL |
| | KYNFKGLVIVRGQDKEEIAEHPIKEINGKFAILDFVYKRGKEKVGKKG |
| | NQKVRYTGNKKVGYCSKHGQVDADLNASRVIALCKYLDINDPILFGE |
| | QRKSFK (SEQ ID NO: 15) |
| Cas14b13 | MVTRAIKLKLDPTKNQYKLLNEMFWKWASLANRFSQKGASKETLAP |
| | KDGTQKIQFNATQLNQIKKDVDDLRGAMEKQGKQKERLLIQIQERLL |
| | TISEILRDDSKKEKDPHRPQNFRPFGWRRFHTSAYWSSEASKLTRQVD |
| | RVRRTIERIKAGKINFKPKRIGLWSSTYKINFLKKKINISPLKSKSFELD |
| | LITEPQQKIIGKEGGKSVANSKKYLDDSIKSLLIFAIKSRLFGLNNKDKP |
| | LFENIITPNLVRYHKKGQEQENFKKEVIKKFENKLKKEISQKQKEIIFS |
| | QIERQYENRDATFSEDYLRAISEFSEIFNQRKKERAKELLNSFNEKIRQ |
| | LKKEVNGNISEEDLKILEVEAEKAYNYENGFIEWEYSEQFLGVLEKIA |
| | RAVLISDNYFDLKKYPILIRKPTNKSKKITNLKPEEWDYYIQFGYGLIN |
| | SPMKIETKNFMGIDRGLTHLLAYSIFDRDSEKFTINQLELNPIKGWKW |
| | KLRKVKRSLQHLERRMRAQKGVKLPENQMKKRLKSIEPKIESYYHNL |
| | SRKIVNLAKANNASIVVESLEGGGLKQHGRKKNSRHRALNYALSLFD |
| | YGKIASLIKYKSDLEGVPMYEVLPAYTSQQCAKCVLKKGSFVEPEIIG |
| | YIEEIGFKENLLTLLFEDTGLSSVQVLKKSKNKMTLSARDKEGKMVD |
| | LVLKYNFKGLVISQEKKKEEIVEFPIKEIDGKFAVLDSAYKRGKERISK |
| | KGNQKLVYTGNKKVGYCSVHGQVDADLNASRVIALCKYLGINEPIVF |
| | GEQRKSFK (SEQ ID NO: 16) |
| Cas14b14 | MDLITEPIQPHKSSSLRSKEFLEYQISDFLNFSLHSLFFGLASNEGPLVD |
| | FKIYDKIVIPKPEERFPKKESEEGKKLDSFDKRVEEYYSDKLEKKIERK |
| | LNTEEKNVIDREKTRIWGEVNKLEEIRSIIDEINEIKKQKHISEKSKLLG |
| | EKWKKVNNIQETLLSQEYVSLISNLSDELTNKKKELLAKKYSKFDDKI |
| | KKIKEDYGLEFDENTIKKEGEKAFLNPDKFSKYQFSSSYLKLIGEIARS |
| | LITYKGFLDLNKYPIIFRKPINKVKKIHNLEPDEWKYYIQFGYEQINNP |
| | KLETENILGIDRGLTHILAYSVFEPRSSKFILNKLEPNPIEGWKWKLRK |
| | LRRSIQNLERRWRAQDNVKLPENQMKKNLRSIEDKVENLYHNLSRKI |
| | VDLAKEKNACIVFEKLEGQGMKQHGRKKSDRLRGLNYKLSLFDYGK |
| | IAKLIKYKAEIEGIPIYRIDSAYTSQNCAKCVLESRRFAQPEEISCLDDF |
| | KEGDNLDKRILEGTGLVEAKIYKKLLKEKKEDFEIEEDIAMFDTKKVI |
| | KENKEKTVILDYVYTRRKEIIGTNHKKNIKGIAKYTGNTKIGYCMKHG |
| | QVDADLNASRTIALCKNFDINNPEIWK (SEQ ID NO: 17) |
| Cas14c1 | MVINLFGYKFALYPNKTQEELLNKHLGECGWLYNKAIEQNEYYKAD |
| | SNIEEAQKKFELLPDKNSDEAKVLRGNISKDNYVYRTLVKKKKSEINV |
| | QIRKAVVLRPAETIRNLAKVKKKGLSVGRLKFIPIREWDVLPFKQSDQ |
| | IRLEENYLILEPYGRLKFKMHRPLLGKPKTFCIKRTATDRWTISFSTEY |
| | DDSNMRKNDGGQVGIDVGLKTHLRLSNENPDEDPRYPNPKIWKRYD |
| | RRLTILQRRISKSKKLGKNRTRLRLRLSRLWEKIRNSRADLIQNETYEI |
| | LSENKLIAIEDLNVKGMQEKKDKKGRKGRTRAQEKGLHRSISDAAFS |
| | EFRRVLEYKAKRFGSEVKPVSAIDSSKECHNCGNKKGMPLESRIYECP |
| | KCGLKIDRDLNSAKVILARATGVRPGSNARADTKISATAGASVQTEG |
| | TVSEDFRQQMETSDQKPMQGEGSKEPPMNPEHKSSGRGSKHVNIGCK |
| | NKVGLYNEDENSRSTEKQIMDENRSTTEDMVEIGALHSPVLTT (SEQ |
| | ID NO: 18) |
| Cas14d1 | MSQSLLKWHDMAGRDKDASRSLQKSAVEGVLLHLTASHRVALEML |
| | EKSVSQTVAVTMEAAQQRLVIVLEDDPTKATSRKRVISADLQFTREEF |
| | GSLPNWAQKLASTCPEIATKYADKHINSIRIAWGVAKESTNGDAVEQ |
| | KLQWQIRLLDVTMFLQQLVLQLADKALLEQIPSSIRGGIGQEVAQQVT |
| | SHIQLLDSGTVLKAELPTISDRNSELARKQWEDAIQTVCTYALPFSRER |
| | ARILDPGKYAAEDPRGDRLINIDPMWARVLKGPTVKSLPLLFVSGSSI |
| | RIVKLTLPRKHAAGHKHTFTATYLVLPVSREWINSLPGTVQEKVQW |
| | WKKPDVLATQELLVGKGALKKSANTLVIPISAGKKRFFNHILPALQR |
| | GFPLQWQRIVGRSYRRPATHRKWFAQLTIGYTNPSSLPEMALGIHFG |
| | MKDILWWALADKQGNILKDGSIPGNSILDFSLQEKGKIERQQKAGKN |
| | VAGKKYGKSLLNATYRVVNGVLEFSKGISAEHASQPIGLGLETIRFVD |
| | KASGSSPVNARHSNWNYGQLSGIFANKAGPAGFSVTEITLKKAQRDL |
| | SDAEQARVLAIEATKRFASRIKRLATKRKDDTLFV (SEQ ID NO: 19) |
| Cas14d2 | MEPVEKERFYYRTYTFRLDGQPRTQNLTTQSGWGLLTKAVLDNTKH |
| | YWEIVHHARIANQPIVFENPVIDEQGNPKLNKLGQPRFWKRPISDIVN |
| | QLRALFENQNPYQLGSSLIQGTYWDVAENLASWYALNKEYLAGTAT |
| | WGEPSFPEPHPLTEINQWMPLTFSSGKVVRLLKNASGRYFIGLPILGEN |
| | NPCYRMRTIEKLIPCDGKGRVTSGSLILFPLVGIYAQQHRRMTDICESI |
| | RTEKGKLAWAQVSIDYVREVDKRRRMRRTRKSQGWIQGPWQEVFIL |
| | RLVLAHKAPKLYKPRCFAGISLGPKTLASCVILDQDERVVEKQQWSG |
| | SELLSLIHQGEERLRSLREQSKPTWNAAYRKQLKSLINTQVFTIVTFLR |
| | ERGAAVRLESIARVRKSTPAPPVNFLLSHWAYRQITERLKDLAIRNGM |
| | PLTHSNGSYGVRFTCSQCGATNQGIKDPTKYKVDIESETFLCSICSHRE |
| | IAAVNTATNLAKQLLDE (SEQ ID NO: 20) |
| Cas14d3 | MNDTETSETLTSHRTVCAHLHVVGETGSLPRLVEAALAELITLNGRA |
| | TQALLSLAKNGLVLRRDKEENLIAAELTLPCRKNKYADVAAKAGEPI |
| | LATRINNKGKLVTKKWYGEGNSYHIVRFTPETGMFTVRVFDRYAFDE |
| | ELLHLHSEVVFGSDLPKGIKAKTDSLPANFLQAVFTSFLELPFQGFPDI |
| | VVKPAMKQAAEQLLSYVQLEAGENQQAEYPDTNERDPELRLVEWQ |
| | KSLHELSVRTEPFEFVRARDIDYYAETDRRGNRFVNITPEWTKFAESP |
| | FARRLPLKIPPEFCILLRRKTEGHAKIPNRIYLGLQIFDGVTPDSTLGVL |
| | ATAEDGKLFWWHDHLDEFSNLEGKPEPKLKNKPQLLMVSLEYDREQ |
| | RFEESVGGDRKICLVTLKETRNFRRGRHGHTRTDRLPAGNTLWRADF |
| | ATSAEVAAPKWNGRILGIHFQHNPVITWALMDHDAEVLEKGFIEGNA |
| | FLGKALDKQALNEYLQKGGKWVGDRSFGNKLKGITHTLASLIVRLA |
| | REKDAWIALEEISWVQKQSADSVANRRFSMWNYSRLATLIEWLGTDI |
| | ATRDCGTAAPLAHKVSDYLTHFTCPECGACRKAGQKKEIADTVRAG |
| | DILTCRKCGFSGPIPDNFIAEFVAKKALERMLKKKPV (SEQ ID NO: 21) |
| Cas14e1 | MAKRNFGEKSEALYRAVRFEVRPSKEELSILLAVSEVLRMLFNSALAE |
| | RQQVFTEFIASLYAELKSASVPEEISEIRKKLREAYKEHSISLFDQINAL |
| | TARRVEDEAFASVTRNWQEETLDALDGAYKSFLSLRRKGDYDAHSP |
| | RSRDSGFFQKIPGRSGFKIGEGRIALSCGAGRKLSFPIPDYQQGRLAET |
| | TKLKKFELYRDQPNLAKSGRFWISVVYELPKPEATTCQSEQVAFVAL |
| | GASSIGVVSQRGEEVIALWRSDKHWVPKIEAVEERMKRRVKGSRGW |
| | LRLLNSGKRRMHMISSRQHVQDEREIVDYLVRNHGSHFVVTELVVRS |
| | KEGKLADSSKPERGGSLGLNWAAQNTGSLSRLVRQLEEKVKEHGGS |
| | VRKHKLTLTEAPPARGAENKLWMARKLRESFLKEV (SEQ ID NO: 22) |
| Cas14e3 | MAKGKKKEGKPLYRAVRFEIFPTSDQITLFLRVSKNLQQVWNEAWQ |
| | ERQSCYEQFFGSIYERIGQAKKRAQEAGFSEVWENEAKKGLNKKLRQ |
| | QEISMQLVSEKESLLQELSIAFQEHGVTLYDQINGLTARRIIGEFALIPR |
| | NWQEETLDSLDGSFKSFLALRKNGDPDAKPPRQRVSENSFYKIPGRSG |
| | FKVSNGQIYLSFGKIGQTLTSVIPEFQLKRLETAIKLKKFELCRDERDM |
| | AKPGRFWISVAYEIPKPEKVPVVSKQITYLAIGASRLGVVSPKGEFCLN |
| | LPRSDYHWKPQINALQERLEGVVKGSRKWKKRMAACTRMFAKLGH |
| | QQKQHGQYEVVKKLLRHGVHFVVTELKVRSKPGALADASKSDRKGS |
| | PTGPNWSAQNTGNIARLIQKLTDKASEHGGTVIKRNPPLLSLEERQLP |
| | DAQRKIFIAKKLREEFLADQK (SEQ ID NO: 23) |
| Cas14f1 | MAKREKKDDVVLRGTKMRIYPTDRQVTLMDMWRRRCISLWNLLLN |
| | LETAAYGAKNTRSKLGWRSIWARVVEENHAKALIVYQHGKCKKDGS |
| | FVLKRDGTVKHPPRERFPGDRKILLGLFDALRHTLDKGAKCKCNVNQ |
| | PYALTRAWLDETGHGARTADIIAWLKDFKGECDCTAISTAAKYCPAP |
| | PTAELLTKIKRAAPADDLPVDQAILLDLFGALRGGLKQKECDHTHAR |
| | TVAYFEKHELAGRAEDILAWLIAHGGTCDCKIVEEAANHCPGPRLFI |
| | WEHELAMIMARLKAEPRTEWIGDLPSHAAQTVVKDLVKALQTMLKE |
| | RAKAAAGDESARKTGFPKFKKQAYAAGSVYFPNTTMFFDVAAGRVQ |
| | LPNGCGSMRCEIPRQLVAELLERNLKPGLVIGAQLGLLGGRIWRQGD |
| | RWYLSCQWERPQPTLLPKTGRTAGVKIAASIVFTTYDNRGQTKEYPM |
| | PPADKKLTAVHLVAGKQNSRALEAQKEKEKKLKARKERLRLGKLEK |
| | GHDPNALKPLKRPRVRRSKLFYKSAARLAACEAIERDRRDGFLHRVT |
| | NEIVHKFDAVSVQKMSVAPMMRRQKQKEKQIESKKNEAKKEDNGA |
| | AKKPRNLKPVRKLLRHVAMARGRQFLEYKYNDLRGPGSVLIADRLE |
| | PEVQECSRCGTKNPQMKDGRRLLRCIGVLPDGTDCDAVLPRNRNAA |
| | RNAEKRLRKHREAHNA (SEQ ID NO: 24) |
| Cas14g1 | MSVRSFQARVECDKQTMEHLWRTHKVFNERLPEIIKILFKMKRGECG |
| | QNDKQKSLYKSISQSILEANAQNADYLLNSVSIKGWKPGTAKKYRNA |
| | SFTWADDAAKLSSQGIHVYDKKQVLGDLPGMMSQMVCRQSVEAISG |
| | HIELTKKWEKEHNEWLKEKEKWESEDEHKKYLDLREKFEQFEQSIGG |
| | KITKRRGRWHLYLKWLSDNPDFAAWRGNKAVINPLSEKAQIRINKAK |
| | PNKKNSVERDEFFKANPEMKALDNLHGYYERNFVRRRKTKKNPDGF |
| | DHKPTFTLPHPTIHPRWFVFNKPKTNPEGYRKLILPKKAGDLGSLEMR |
| | LLTGEKNKGNYPDDWISVKFKADPRLSLIRPVKGRRVVRKGKEQGQT |
| | KETDSYEFFDKHLKKWRPAKLSGVKLIFPDKTPKAAYLYFTCDIPDEP |
| | LTETAKKIQWLETGDVTKKGKKRKKKVLPHGLVSCAVDLSMRRGTT |
| | GFATLCRYENGKIHILRSRNLWVGYKEGKGCHPYRWTEGPDLGHIAK |
| | HKREIRILRSKRGKPVKGEESHIDLQKHIDYMGEDRFKKAARTIVNFA |
| | LNTENAASKNGFYPRADVLLLENLEGLIPDAEKERGINRALAGWNRR |
| | HLVERVIEMAKDAGFKRRVFEIPPYGTSQVCSKCGALGRRYSIIRENN |
| | RREIRFGYVEKLFACPNCGYCANADHNASVNLNRRFLIEDSFKSYYD |
| | WKRLSEKKQKEEIETIESKLMDKLCAMHKISRGSISK (SEQ ID NO: 25) |
| Cas14u3 | MAEENTLHLTLAMSLPLNDLPENRTRSELWRRQWLPQKKLSLLLGV |
| | NQSVRKAAADCLRWFEPYQELLWWEPTDPDGKKLLDKEGRPIKRTA |
| | GHMRVLRKLEEIAPFRGYQLGSAVKNGLRHKVADLLLSYAKRKLDP |
| | QFTDKTSYPSIGDQFPIVWTGAFVCYEQSITGQLYLYLPLFPRGSHQED |
| | ITNNYDPDRGPALQVFGEKEIARLSRSTSGLLLPLQFDKWGEATFIRGE |
| | NNPPTWKATHRRSDKKWLSEVLLREKDFQPKRVELLVRNGRIFVNV |
| | ACEIPTKPLLEVENFMGVSFGLEHLVTVVVINRDGNVVHQRQEPARR |
| | YEKTYFARLERLRRRGGPFSQELETFHYRQVAQIVEEALRFKSVPAVE |
| | QVGNIPKGRYNPRLNLRLSYWPFGKLADLTSYKAVKEGLPKPYSVYS |
| | ATAKMLCSTCGAANKEGDQPISLKGPTVYCGNCGTRHNTGENTALN |
| | LARRAQELFVKGVVAR (SEQ ID NO: 26) |
| Cas14u4 | MTTQKTYNFCFYDQRFFELSKEAGEVYSRSLEEFWKIYDETGVWLSK |
| | FDLQKHMRNKLERKLLHSDSFLGAMQQVHANLASWKQAKKVVPDA |
| | CPPRKPKFLQAILFKKSQIKYKNGFLRLTLGTEKEFLYLKWDINIPLPIY |
| | GSVTYSKTRGWKINLCLETEVEQKNLSENKYLSIDLGVKRVATIFDGE |
| | NTITLSGKKFMGLMHYRNKLNGKTQSRLSHKKKGSNNYKKIQRAKR |
| | KTTDRLLNIQKEMLHKYSSFIVNYAIRNDIGNIIIGDNSSTHDSPNMRG |
| | KTNQKISQNPEQKLKNYIKYKFESISGRVDIVPEPYTSRKCPHCKNIKK |
| | SSPKGRTYKCKKCGFIFDRDGVGAINIYNENVSFGQIISPGRIRSLTEPI |
| | GMKFHNEIYFKSYVAA (SEQ ID NO: 27) |

In some aspects, the present disclosure provides napDNAbp proteins comprising substitutions corresponding to any of the substitutions disclosed herein, or any combination thereof, in another Cas protein homolog. The amino acid substitutions disclosed herein are compatible with a variety of Cas homologs known in the art. The amino acid substitutions disclosed herein are broadly compatible with and may be made at corresponding positions in a variety of napDNAbps that include, but are not limited to, Cas9 proteins and Cas12 proteins. Additional Cas variants and homologs include Cas9 (e.g., dCas9 and nCas9), Cpf1, CasX, CasY, C2c1, C2c2, C2c3, GeoCas9, CjCas9, Cas12a, Cas12b, Cas12g, Cas12h, Cas12i, Cas13b, Cas13c, Cas13d, Cas14, Csn2, xCas9, SpCas9-NG, Nme2Cas9, circularly permuted Cas9, Argonaute (Ago), Cas9-KKH, SmacCas9, Spy-macCas9, SpCas9-VRQR, SpCas9-NRRH, SpaCas9-NRTH, SpCas9-NRCH, LbCas12a, AsCas12a, CeCas12a, MbCas12a, Cas3, CasΦ, and circularly permuted Cas9 domains such as CP1012, CP1028, CP1041, CP1249, and CP1300, and variants and homologs thereof. The amino acid substitutions disclosed herein may be made at corresponding positions in any Cas protein or other napDNAbp, homolog thereof, or variant thereof known in the art, and the present disclosure is not limited in this respect.

### Fusion Proteins

In some aspects, the present disclosure provides fusion proteins comprising any of the Cas14a1 variants provided herein.

In some embodiments, the fusion proteins comprise (i) any of the Cas14a1 variants provided herein, and (ii) an effector domain. In some embodiments, the effector domain comprises nuclease activity, nickase activity, recombinase activity, deaminase activity, methyltransferase activity, methylase activity, acetylase activity, acetyltransferase activity, transcriptional activation activity, transcriptional repression activity, or polymerase activity. In certain embodiments, the effector domain is a nucleic acid editing domain (e.g., a deaminase domain). A fusion protein comprising a Cas protein and a deaminase domain may be referred to herein as a “base editor.” In some embodiments, the deaminase domain is an adenosine deaminase domain (e.g., an E. coli Tad A (ecTadA) deaminase domain) or a cytosine deaminase domain (e.g., an APOBEC family deaminase domain). In some embodiments, a base editor fusion protein comprising any of the Cas proteins provided herein exhibits increased base editing activity on a target sequence as compared to a fusion protein comprising a wild-type Cas14a1 protein as provided by SEQ ID NO: 2. In certain embodiments, the activity is increased by at least 2-fold, at least 3-fold, at least 4-fold, at least 5-fold, at least 6-fold, at least 7-fold, at least 8-fold, at least 9-fold, or at least 10-fold as compared to a wild-type Cas14a1 protein as provided by SEQ ID NO: 2.

In some embodiments, the fusion proteins comprise (i) any of the Cas14a1 variants provided herein, and (ii) a domain comprising an RNA-dependent DNA polymerase activity. In certain embodiments, the domain comprising an RNA-dependent DNA polymerase activity is a reverse transcriptase. A fusion protein comprising a Cas protein and a reverse transcriptase domain may be referred to herein as a “prime editor.”

Additional exemplary deaminase domains and reverse transcriptase domains are provided below. The present disclosure contemplates the use of any deaminase domain or reverse transcriptase domain described herein or known in the art in the fusion proteins provided herein.

### Base Editing and Deaminase Domains

In some embodiments, the fusion proteins described herein comprise a deaminase domain (e.g., when the Cas proteins provided herein are being used in the context of a base editor). A deaminase domain may be a cytosine deaminase domain or an adenosine deaminase domain.

Base editor fusion proteins that convert a C to T, in some embodiments, comprise a cytosine deaminase. A “cytosine deaminase” refers to an enzyme that catalyzes the chemical reaction “cytosine+H₂O→uracil+NH₃” or “5-methyl-cytosine+H₂O→thymine+NH₃.” As it may be apparent from the reaction formula, such chemical reactions result in a C to U/T nucleobase change. In the context of a gene, such a nucleotide change, or mutation, may in turn lead to an amino acid change in the protein, which may affect the protein's function, e.g., loss-of-function or gain-of-function. In some embodiments, the C to T base editor comprises a Cas14a1 variant provided herein fused to a cytosine deaminase. In some embodiments, the cytosine deaminase domain is fused to the N-terminus of the Cas14a1 variant.

Non-limiting examples of suitable cytosine deaminase domains are provided below, as SEQ ID NOs: 33-56, 177-186.

### Human AID

(SEQ ID NO: 33)
MDSLLMNRRKFLYQFKNVRWAKGRRETYLCYVVKRRDSATSFSLDFGYL
RNKNGCHVELLFLRYISDWDLDPGRCYRVTWFTSWSPCYDCARHVADFL
RGNPNLSLRIFTARLYFCEDRKAEPEGLRRLHRAGVQIAIMTFKDYFYC
WNTFVENHERTFKAWEGLHENSVRLSRQLRRILLPLYEVDDLRDAFRTL
GL

### Mouse AID

(SEQ ID NO: 34)
MDSLLMKQKKFLYHFKNVRWAKGRHETYLCYVVKRRDSATSCSLDFGHL
RNKSGCHVELLFLRYISDWDLDPGRCYRVTWFTSWSPCYDCARHVAEFL
RWNPNLSLRIFTARLYFCEDRKAEPEGLRRLHRAGVQIGIMTFKDYFYC
WNTFVENRERTFKAWEGLHENSVRLTRQLRRILLPLYEVDDLRDAFRML
GF

### Dog AID

(SEQ ID NO: 35)
MDSLLMKQRKFLYHFKNVRWAKGRHETYLCYVVKRRDSATSFSLDFGHL
RNKSGCHVELLFLRYISDWDLDPGRCYRVTWFTSWSPCYDCARHVADFL
RGYPNLSLRIFAARLYFCEDRKAEPEGLRRLHRAGVQIAIMTFKDYFYC
WNTFVENREKTFKAWEGLHENSVRLSRQLRRILLPLYEVDDLRDAFRTL
GL

### Bovine AID

(SEQ ID NO: 36)
MDSLLKKQRQFLYQFKNVRWAKGRHETYLCYVVKRRDSPTSFSLDFGHL
RNKAGCHVELLFLRYISDWDLDPGRCYRVTWFTSWSPCYDCARHVADFL
RGYPNLSLRIFTARLYFCDKERKAEPEGLRRLHRAGVQIAIMTFKDYFY
CWNTFVENHERTFKAWEGLHENSVRLSRQLRRILLPLYEVDDLRDAFRT
LGL

### Mouse APOBEC-3

(SEQ ID NO: 37)
MGPFCLGCSHRKCYSPIRNLISQETFKFHFKNLGYAKGRKDTFLCYEVT
RKDCDSPVSLHHGVFKNKDNIHAEICFLYWFHDKVLKVLSPREEFKITW
YMSWSPCFECAEQIVRFLATHHNLSLDIFSSRLYNVQDPETQQNLCRLV
QEGAQVAAMDLYEFKKCWKKFVDNGGRRFRPWKRLLTNFRYQDSKLQEI
LRPCYIPVPSSSSSTLSNICLTKGLPETRFCVEGRRMDPLSEEEFYSQF
YNQRVKHLCYYHRMKPYLCYQLEQFNGQAPLKGCLLSEKGKQHAEILFL
DKIRSMELSQVTITCYLTWSPCPNCAWQLAAFKRDRPDLILHIYTSRLY
FHWKRPFQKGLCSLWQSGILVDVMDLPQFTDCWTNFVNPKRPFWPWKGL
EIISRRTQRRLRRIKESWGLQDLVNDFGNLQLGPPMS

### Rat APOBEC-3

(SEQ ID NO: 38)
MGPFCLGCSHRKCYSPIRNLISQETFKFHFKNLRYAIDRKDTFLCYEVT
RKDCDSPVSLHHGVFKNKDNIHAEICFLYWFHDKVLKVLSPREEFKITW
YMSWSPCFECAEQVLRFLATHHNLSLDIFSSRLYNIRDPENQQNLCRLV
QEGAQVAAMDLYEFKKCWKKFVDNGGRRFRPWKKLLTNFRYQDSKLQEI
LRPCYIPVPSSSSSTLSNICLTKGLPETRFCVERRRVHLLSEEEFYSQF
YNQRVKHLCYYHGVKPYLCYQLEQFNGQAPLKGCLLSEKGKQHAEILFL
DKIRSMELSQVIITCYLTWSPCPNCAWQLAAFKRDRPDLILHIYTSRLY
FHWKRPFQKGLCSLWQSGILVDVMDLPQFTDCWTNFVNPKRPFWPWKGL
EIISRRTQRRLHRIKESWGLQDLVNDFGNLQLGPPMS

### Rhesus Macaque APOBEC-3G

(SEQ ID NO: 39)
MVEPMDPRTFVSNFNNRPILSGLNTVWLCCEVKTKDPSGPPLDAKIFQG
KVYSKAKYHPEMRFLRWFHKWRQLHHDQEYKVTWYVSWSPCTRCANSVA
TFLAKDPKVTLTIFVARLYYFWKPDYQQALRILCQKRGGPHATMKIMNY
NEFQDCWNKFVDGRGKPFKPRNNLPKHYTLLQATLGELLRHLMDPGTFT
SNFNNKPWVSGQHETYLCYKVERLHNDTWVPLNQHRGFLRNQAPNIHGF
PKGRHAELCFLDLIPFWKLDGQQYRVTCFTSWSPCFSCAQEMAKFISNN
EHVSLCIFAARIYDDQGRYQEGLRALHRDGAKIAMMNYSEFEYCWDTFV
DRQGRPFQPWDGLDEHSQALSGRLRAI

### Chimpanzee APOBEC-3G

(SEQ ID NO: 40)
MKPHFRNPVERMYQDTFSDNFYNRPILSHRNTVWLCYEVKTKGPSRPPL
DAKIFRGQVYSKLKYHPEMRFFHWFSKWRKLHRDQEYEVTWYISWSPCT
KCTRDVATFLAEDPKVTLTIFVARLYYFWDPDYQEALRSLCQKRDGPRA
TMKIMNYDEFQHCWSKFVYSQRELFEPWNNLPKYYILLHIMLGEILRHS
MDPPTFTSNFNNELWVRGRHETYLCYEVERLHNDTWVLLNQRRGFLCNQ
APHKHGFLEGRHAELCFLDVIPFWKLDLHQDYRVTCFTSWSPCFSCAQE
MAKFISNNKHVSLCIFAARIYDDQGRCQEGLRTLAKAGAKISIMTYSEF
KHCWDTFVDHQGCPFQPWDGLEEHSQALSGRLRAILQNQGN

### Green Monkey APOBEC-3G

(SEQ ID NO: 41)
MNPQIRNMVEQMEPDIFVYYFNNRPILSGRNTVWLCYEVKTKDPSGPPL
DANIFQGKLYPEAKDHPEMKFLHWFRKWRQLHRDQEYEVTWYVSWSPCT
RCANSVATFLAEDPKVTLTIFVARLYYFWKPDYQQALRILCQERGGPHA
TMKIMNYNEFQHCWNEFVDGQGKPFKPRKNLPKHYTLLHATLGELLRHV
MDPGTFTSNFNNKPWVSGQRETYLCYKVERSHNDTWVLLNQHRGFLRNQ
APDRHGFPKGRHAELCFLDLIPFWKLDDQQYRVTCFTSWSPCFSCAQKM
AKFISNNKHVSLCIFAARIYDDQGRCQEGLRTLHRDGAKIAVMNYSEFE
YCWDTFVDRQGRPFQPWDGLDEHSQALSGRLRAI

### Human APOBEC-3G

(SEQ ID NO: 42)
MKPHFRNTVERMYRDTFSYNFYNRPILSRRNTVWLCYEVKTKGPSRPPL
DAKIFRGQVYSELKYHPEMRFFHWFSKWRKLHRDQEYEVTWYISWSPCT
KCTRDMATFLAEDPKVTLTIFVARLYYFWDPDYQEALRSLCQKRDGPRA
TMKIMNYDEFQHCWSKFVYSQRELFEPWNNLPKYYILLHIMLGEILRHS
MDPPTFTFNFNNEPWVRGRHETYLCYEVERMHNDTWVLLNQRRGFLCNQ
APHKHGFLEGRHAELCFLDVIPFWKLDLDQDYRVTCFTSWSPCFSCAQE
MAKFISKNKHVSLCIFTARIYDDQGRCQEGLRTLAEAGAKISIMTYSEF
KHCWDTFVDHQGCPFQPWDGLDEHSQDLSGRLRAILQNQEN

### Human APOBEC-3F

(SEQ ID NO: 43)
MKPHFRNTVERMYRDTFSYNFYNRPILSRRNTVWLCYEVKTKGPSRPRL
DAKIFRGQVYSQPEHHAEMCFLSWFCGNQLPAYKCFQITWFVSWTPCPD
CVAKLAEFLAEHPNVTLTISAARLYYYWERDYRRALCRLSQAGARVKIM
DDEEFAYCWENFVYSEGQPFMPWYKFDDNYAFLHRTLKEILRNPMEAMY
PHIFYFHFKNLRKAYGRNESWLCFTMEVVKHHSPVSWKRGVFRNQVDPE
THCHAERCFLSWFCDDILSPNTNYEVTWYTSWSPCPECAGEVAEFLARH
SNVNLTIFTARLYYFWDTDYQEGLRSLSQEGASVEIMGYKDFKYCWENF
VYNDDEPFKPWKGLKYNFLFLDSKLQEILE

### Human APOBEC-3B

(SEQ ID NO: 44)
MNPQIRNPMERMYRDTFYDNFENEPILYGRSYTWLCYEVKIKRGRSNLL
WDTGVFRGQVYFKPQYHAEMCFLSWFCGNQLPAYKCFQITWFVSWTPCP
DCVAKLAEFLSEHPNVTLTISAARLYYYWERDYRRALCRLSQAGARVTI
MDYEEFAYCWENFVYNEGQQFMPWYKFDENYAFLHRTLKEILRYLMDPD
TFTFNFNNDPLVLRRRQTYLCYEVERLDNGTWVLMDQHMGFLCNEAKNL
LCGFYGRHAELRFLDLVPSLQLDPAQIYRVTWFISWSPCFSWGCAGEVR
AFLQENTHVRLRIFAARIYDYDPLYKEALQMLRDAGAQVSIMTYDEFEY
CWDTFVYRQGCPFQPWDGLEEHSQALSGRLRAILQNQGN

### Human APOBEC-3C

(SEQ ID NO: 45)
MNPQIRNPMKAMYPGTFYFQFKNLWEANDRNETWLCFTVEGIKRRSVVS
WKTGVFRNQVDSETHCHAERCFLSWFCDDILSPNTKYQVTWYTSWSPCP
DCAGEVAEFLARHSNVNLTIFTARLYYFQYPCYQEGLRSLSQEGVAVEI
MDYEDFKYCWENFVYNDNEPFKPWKGLKTNFRLLKRRLRESLQ

### Human APOBEC-3A

(SEQ ID NO: 46)
MEASPASGPRHLMDPHIFTSNFNNGIGRHKTYLCYEVERLDNGTSVKMD
QHRGFLHNQAKNLLCGFYGRHAELRFLDLVPSLQLDPAQIYRVTWFISW
SPCFSWGCAGEVRAFLQENTHVRLRIFAARIYDYDPLYKEALQMLRDAG
AQVSIMTYDEFKHCWDTFVDHQGCPFQPWDGLDEHSQALSGRLRAILQN
QGN

### Human APOBEC-3H

(SEQ ID NO: 47)
MALLTAETFRLQFNNKRRLRRPYYPRKALLCYQLTPQNGSTPTRGYFEN
KKKCHAEICFINEIKSMGLDETQCYQVTCYLTWSPCSSCAWELVDFIKA
HDHLNLGIFASRLYYHWCKPQQKGLRLLCGSQVPVEVMGFPKFADCWEN
FVDHEKPLSFNPYKMLEELDKNSRAIKRRLERIKIPGVRAQGRYMDILC
DAEV

### Human APOBEC-3D

(SEQ ID NO: 48)
MNPQIRNPMERMYRDTFYDNFENEPILYGRSYTWLCYEVKIKRGRSNLL
WDTGVFRGPVLPKRQSNHRQEVYFRFENHAEMCFLSWFCGNRLPANRRF
QITWFVSWNPCLPCVVKVTKFLAEHPNVTLTISAARLYYYRDRDWRWVL
LRLHKAGARVKIMDYEDFAYCWENFVCNEGQPFMPWYKFDDNYASLHRT
LKEILRNPMEAMYPHIFYFHFKNLLKACGRNESWLCFTMEVTKHHSAVF
RKRGVFRNQVDPETHCHAERCFLSWFCDDILSPNTNYEVTWYTSWSPCP
ECAGEVAEFLARHSNVNLTIFTARLCYFWDTDYQEGLCSLSQEGASVKI
MGYKDFVSCWKNFVYSDDEPFKPWKGLQTNFRLLKRRLREILQ

### Human APOBEC-1

(SEQ ID NO: 49)
MTSEKGPSTGDPTLRRRIEPWEFDVFYDPRELRKEACLLYEIKWGMSRK
IWRSSGKNTTNHVEVNFIKKFTSERDFHPSMSCSITWFLSWSPCWECSQ
AIREFLSRHPGVTLVIYVARLFWHMDQQNRQGLRDLVNSGVTIQIMRAS
EYYHCWRNFVNYPPGDEAHWPQYPPLWMMLYALELHCIILSLPPCLKIS
RRWQNHLTFFRLHLQNCHYQTIPPHILLATGLIHPSVAWR

### Mouse APOBEC-1

(SEQ ID NO: 50)
MSSETGPVAVDPTLRRRIEPHEFEVFFDPRELRKETCLLYEINWGGRHS
VWRHTSQNTSNHVEVNFLEKFTTERYFRPNTRCSITWFLSWSPCGECSR
AITEFLSRHPYVTLFIYIARLYHHTDQRNRQGLRDLISSGVTIQIMTEQ
EYCYCWRNFVNYPPSNEAYWPRYPHLWVKLYVLELYCIILGLPPCLKIL
RRKQPQLTFFTITLQTCHYQRIPPHLLWATGLK

### Rat APOBEC-1

(SEQ ID NO: 51)
MSSETGPVAVDPTLRRRIEPHEFEVFFDPRELRKETCLLYEINWGGRHS
IWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQ
ESGYCWRNFVNYSPSNEAHWPRYPHLWVRLYVLELYCIILGLPPCLNIL
RRKQPQLTFFTIALQSCHYQRLPPHILWATGLK

Petromyzon marinus CDA1 (pmCDA1)

(SEQ ID NO: 52)
MTDAEYVRIHEKLDIYTFKKQFFNNKKSVSHRCYVLFELKRRGERRACF
WGYAVNKPQSGTERGIHAEIFSIRKVEEYLRDNPGQFTINWYSSWSPCA
DCAEKILEWYNQELRGNGHTLKIWACKLYYEKNARNQIGLWNLRDNGVG
LNVMVSEHYQCCRKIFIQSSHNQLNENRWLEKTLKRAEKRRSELSIMIQ
VKILHTTKSPAV

Evolved pmCDA1 (evoCDA1)

(SEQ ID NO: 53)
MTDAEYVRIHEKLDIYTFKKQFSNNKKSVSHRCYVLFELKRRGERRACF
WGYAVNKPQSGTERGIHAEIFSIRKVEEYLRDNPGQFTINWYSSWSPCA
DCAEKILEWYNQELRGNGHTLKIWVCKLYYEKNARNQIGLWNLRDNGVG
LNVMVSEHYQCCRKIFIQSSHNQLNENRWLEKTLKRAEKRRSELSIMFQ
VKILHTTKSPAV

### Human APOBEC3G D316R_D317R

(SEQ ID NO: 54)
MKPHFRNTVERMYRDTFSYNFYNRPILSRRNTVWLCYEVKTKGPSRPPL
DAKIFRGQVYSELKYHPEMRFFHWFSKWRKLHRDQEYEVTWYISWSPCT
KCTRDMATFLAEDPKVTLTIFVARLYYFWDPDYQEALRSLCQKRDGPRA
TMKIMNYDEFQHCWSKFVYSQRELFEPWNNLPKYYILLHIMLGEILRHS
MDPPTFTFNFNNEPWVRGRHETYLCYEVERMHNDTWVLLNQRRGFLCNQ
APHKHGFLEGRHAELCFLDVIPFWKLDLDQDYRVTCFTSWSPCFSCAQE
MAKFISKNKHVSLCIFTARIYRRQGRCQEGLRTLAEAGAKISIMTYSEF
KHCWDTFVDHQGCPFQPWDGLDEHSQDLSGRLRAILQNQEN

### Human APOBEC3G Chain A

(SEQ ID NO: 55)
MDPPTFTFNFNNEPWVRGRHETYLCYEVERMHNDTWVLLNQRRGFLCNQ
APHKHGFLEGRHAELCFLDVIPFWKLDLDQDYRVTCFTSWSPCFSCAQE
MAKFISKNKHVSLCIFTARIYDDQGRCQEGLRTLAEAGAKISIMTYSEF
KHCWDTFVDHQGCPFQPWDGLDEHSQDLSGRLRAILQ

### Human APOBEC3G Chain A D120R_D121R

(SEQ ID NO: 56)
MDPPTFTFNFNNEPWVRGRHETYLCYEVERMHNDTWVLLNQRRGFLCNQ
APHKHGFLEGRHAELCFLDVIPFWKLDLDQDYRVTCFTSWSPCFSCAQE
MAKFISKNKHVSLCIFTARIYRRQGRCQEGLRTLAEAGAKISIMTYSEF
KHCWDTFVDHQGCPFQPWDGLDEHSQDLSGRLRAILQ

### evo APOBEC1

(SEQ ID NO: 177)
MSSKTGPVAVDPTLRRRIEPHEFEVFFDPRELRKETCLLYEINWGGRHS
IWRHTSQNTNKHVEVNFIEKFTTERYFCPNTRCSITWFLSWSPCGECSR
AITEFLSRYPNVTLFIYIARLYHLANPRNRQGLRDLISSGVTIQIMTEQ
ESGYCWHNFVNYSPSNESHWPRYPHLWVRLYVLELYCIILGLPPCLNIL
RRKQSQLTSFTIALQSCHYQRLPPHILWATGLK

### YE1

(SEQ ID NO: 178)
MSSETGPVAVDPTLRRRIEPHEFEVFFDPRELRKETCLLYEINWGGRHS
IWRHTSQNTNKHVEVNFIEKFTTERYFCPNTRCSITWFLSYSPCGECSR
AITEFLSRYPHVTLFIYIARLYHHADPENRQGLRDLISSGVTIQIMTEQ
ESGYCWRNFVNYSPSNEAHWPRYPHLWVRLYVLELYCIILGLPPCLNIL
RRKQPQLTFFTIALQSCHYQRLPPHILWATGLK

### YE2

(SEQ ID NO: 179)
MSSETGPVAVDPTLRRRIEPHEFEVFFDPRELRKETCLLYEINWGGRHS
IWRHTSQNTNKHVEVNFIEKFTTERYFCPNTRCSITWFLSYSPCGECSR
AITEFLSRYPHVTLFIYIARLYHHADPRNRQGLEDLISSGVTIQIMTEQ
ESGYCWRNFVNYSPSNEAHWPRYPHLWVRLYVLELYCIILGLPPCLNIL
RRKQPQLTFFTIALQSCHYQRLPPHILWATGLK

### YEE

(SEQ ID NO: 180)
MSSETGPVAVDPTLRRRIEPHEFEVFFDPRELRKETCLLYEINWGGRHS
IWRHTSQNTNKHVEVNFIEKFTTERYFCPNTRCSITWFLSYSPCGECSR
AITEFLSRYPHVTLFIYIARLYHHADPENRQGLEDLISSGVTIQIMTEQ
ESGYCWRNFVNYSPSNEAHWPRYPHLWVRLYVLELYCIILGLPPCLNIL
RRKQPQLTFFTIALQSCHYQRLPPHILWATGLK

### EE

(SEQ ID NO: 181)
MSSETGPVAVDPTLRRRIEPHEFEVFFDPRELRKETCLLYEINWGGRHS
IWRHTSQNTNKHVEVNFIEKFTTERYFCPNTRCSITWFLSWSPCGECSR
AITEFLSRYPHVTLFIYIARLYHHADPENRQGLEDLISSGVTIQIMTEQ
ESGYCWRNFVNYSPSNEAHWPRYPHLWVRLYVLELYCIILGLPPCLNIL
RRKQPQLTFFTIALQSCHYQRLPPHILWATGLK

### R33A

(SEQ ID NO: 182)
MSSETGPVAVDPTLRRRIEPHEFEVFFDPRELAKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLK

### R33A+K34A

(SEQ ID NO: 183)
MSSETGPVAVDPTLRRRIEPHEFEVFFDPRELAAETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLK

### AALN

(SEQ ID NO: 184)
MSSETGPVAVDPTLRRRIEPHEFEVFFDPRELAAETCLLYEINWGGRHSI
WRHTSQNTNKHVEVNFIEKFTTERYFCPNTRCSITWFLSWSPCGECSRAI
TEFLSRYPHVTLFIYIARLYHLANPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLK

### FERNY

(SEQ ID NO: 185)
MFERNYDPRELRKETYLLYEIKWGKSGKLWRHWCQNNRTQHAEVYFLENI
FNARRFNPSTHCSITWYLSWSPCAECSQKIVDFLKEHPNVNLEIYVARLY
YHEDERNRQGLRDLVNSGVTIRIMDLPDYNYCWKTFVSDQGGDEDYWPGH
FAPWIKQYSLKL

### evo FERNY

(SEQ ID NO: 186)
MFERNYDPRELRKETYLLYEIKWGKSGKLWRHWCQNNRTQHAEVYFLENI
FNARRFNPSTHCSITWYLSWSPCAECSQKIVDFLKEHPNVNLEIYVARLY
YPENERNRQGLRDLVNSGVTIRIMDLPDYNYCWKTFVSDQGGDEDYWPGH
FAPWIKQYSLKL

In some embodiments, a base editor fusion protein converts an A to G. In some embodiments, the base editor comprises an adenosine deaminase. An “adenosine deaminase” is an enzyme involved in purine metabolism. It is needed for the breakdown of adenosine from food and for the turnover of nucleic acids in tissues. Its primary function in humans is the development and maintenance of the immune system. An adenosine deaminase catalyzes hydrolytic deamination of adenosine (forming inosine, which base pairs as G) in the context of DNA. There are no known adenosine deaminases that act on DNA. Instead, known adenosine deaminase enzymes only act on RNA (tRNA or mRNA). Evolved deoxyadenosine deaminase enzymes that accept DNA substrates and deaminate dA to deoxyinosine for use in adenosine nucleobase editors have been described, e.g., in PCT Application PCT/US2017/045381, filed Aug. 3, 2017, which published as WO 2018/027078, PCT Application No. PCT/US2019/033848, which published as WO 2019/226953, PCT Application No PCT/US2019/033848, filed May 23, 2019, and PCT Application No. PCT/US2020/028568, filed Apr. 17, 2020; each of which is herein incorporated by reference. Non-limiting examples of evolved adenosine deaminases that accept DNA as substrates are provided below. In some embodiments, an adenosine deaminase comprises any of the following amino acid sequences, or an amino acid sequence at least 70%, at least 75%, at least 80%, at least 85%, at least 90%, at least 95%, at least 99%, or at least 99.9% identical to any of the following amino acid sequences (SEQ ID NOs: 29, 57-123):

ecTadA

(SEQ ID NO: 57)
SEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRI
GRVVFGARDAKTGAAGSLMDVLHHPGMNHRVEITEGILADECAALLSDF
FRMRRQEIKAQKKAQSSTD

ecTadA (D108N)

(SEQ ID NO: 58)
SEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRI
GRVVFGARNAKTGAAGSLMDVLHHPGMNHRVEITEGILADECAALLSDF
FRMRRQEIKAQKKAQSSTD

ecTadA (D108G)

(SEQ ID NO: 59)
SEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRI
GRVVFGARGAKTGAAGSLMDVLHHPGMNHRVEITEGILADECAALLSDF
FRMRRQEIKAQKKAQSSTD

ecTadA (D108V)

(SEQ ID NO: 60)
SEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRI
GRVVFGARVAKTGAAGSLMDVLHHPGMNHRVEITEGILADECAALLSDF
FRMRRQEIKAQKKAQSSTD

ecTadA (H8Y, D108N, N127S)

(SEQ ID NO: 61)
SEVEFSYEYWMRHALTLAKRAWDEREVPVGAVLVHNNRVIGEGWNRPIG
RHDPTAHAEIMALRQGGLVMQNYRLIDATLYVTLEPCVMCAGAMIHSRI
GRVVFGARNAKTGAAGSLMDVLHHPGMSHRVEITEGILADECAALLSDF
FRMRRQEIKAQKKAQSSTD

ecTadA (H8Y, D108N, N127S, E155D)

(SEQ ID NO: 62)
SEVEFSYEYWMRHALTLAKRAWDEREVPVGAVLVHNNRVIGEGWNRPIG
RHDPTAHAEIMALRQGGLVMQNYRLIDATLYVTLEPCVMCAGAMIHSRI
GRVVFGARNAKTGAAGSLMDVLHHPGMSHRVEITEGILADECAALLSDF
FRMRRQDIKAQKKAQSSTD

ecTadA (H8Y, D108N, N127S, E155G)

(SEQ ID NO: 63)
SEVEFSYEYWMRHALTLAKRAWDEREVPVGAVLVHNNRVIGEGWNRPIG
RHDPTAHAEIMALRQGGLVMQNYRLIDATLYVTLEPCVMCAGAMIHSRI
GRVVFGARNAKTGAAGSLMDVLHHPGMSHRVEITEGILADECAALLSDF
FRMRRQGIKAQKKAQSSTD

ecTadA (H8Y, D108N, N127S, E155V)

(SEQ ID NO: 64)
SEVEFSYEYWMRHALTLAKRAWDEREVPVGAVLVHNNRVIGEGWNRPIG
RHDPTAHAEIMALRQGGLVMQNYRLIDATLYVTLEPCVMCAGAMIHSRI
GRVVFGARNAKTGAAGSLMDVLHHPGMSHRVEITEGILADECAALLSDF
FRMRRQVIKAQKKAQSSTD

ecTadA (A106V, D108N, D147Y, and E155V)

(SEQ ID NO: 65)
SEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRI
GRVVFGVRNAKTGAAGSLMDVLHHPGMNHRVEITEGILADECAALLSYF
FRMRRQVIKAQKKAQSSTD

ecTadA (S2A, I49F, A106V, D108N, D147Y, E155V)

(SEQ ID NO: 66)
AEVEFSHEYWMRHALTLAKRAWDEREVPVGAVLVHNNRVIGEGWNRPFG
RHDPTAHAEIMALRQGGLVMQNYRLIDATLYVTLEPCVMCAGAMIHSRI
GRVVFGVRNAKTGAAGSLMDVLHHPGMNHRVEITEGILADECAALLSYF
FRMRRQVIKAQKKAQSSTD

ecTadA (H8Y, A106T, D108N, N127S, K160S)

(SEQ ID NO: 67)
SEVEFSYEYWMRHALTLAKRAWDEREVPVGAVLVHNNRVIGEGWNRPIG
RHDPTAHAEIMALRQGGLVMQNYRLIDATLYVTLEPCVMCAGAMIHSRI
GRVVFGTRNAKTGAAGSLMDVLHHPGMSHRVEITEGILADECAALLSDF
FRMRRQEIKAQSKAQSSTD

ecTadA (R26G, L84F, A106V, R107H, D108N, H123Y, A142N, A143D, D147Y, E155V, I156F)

(SEQ ID NO: 68)
SEVEFSHEYWMRHALTLAKRAWDEGEVPVGAVLVHNNRVIGEGWNRPIG
RHDPTAHAEIMALRQGGLVMQNYRLIDATLYVTFEPCVMCAGAMIHSRI
GRVVFGVHNAKTGAAGSLMDVLHYPGMNHRVEITEGILADECNDLLSYF
FRMRRQVFKAQKKAQSSTD

ecTadA (E25G, R26G, L84F, A106V, R107H, D108N, H123Y, A142N, A143D, D147Y, E155V, I156F)

(SEQ ID NO: 69)
SEVEFSHEYWMRHALTLAKRAWDGGEVPVGAVLVHNNRVIGEGWNRPIG
RHDPTAHAEIMALRQGGLVMQNYRLIDATLYVTFEPCVMCAGAMIHSRI
GRVVFGVHNAKTGAAGSLMDVLHYPGMNHRVEITEGILADECNDLLSYF
FRMRRQVFKAQKKAQSSTD

ecTadA (E25D, R26G, L84F, A106V, R107K, D108N, H123Y, A142N, A143G, D147Y, E155V, I156F

(SEQ ID NO: 70)
SEVEFSHEYWMRHALTLAKRAWDDGEVPVGAVLVHNNRVIGEGWNRPIG
RHDPTAHAEIMALRQGGLVMQNYRLIDATLYVTFEPCVMCAGAMIHSRI
GRVVFGVKNAKTGAAGSLMDVLHYPGMNHRVEITEGILADECNGLLSYF
FRMRRQVFKAQKKAQSSTD

ecTadA (R26Q, L84F, A106V, D108N, H123Y, A142N, D147Y, E155V, I156F

(SEQ ID NO: 71)
SEVEFSHEYWMRHALTLAKRAWDEQEVPVGAVLVHNNRVIGEGWNRPIG
RHDPTAHAEIMALRQGGLVMQNYRLIDATLYVTFEPCVMCAGAMIHSRI
GRVVFGVRNAKTGAAGSLMDVLHYPGMNHRVEITEGILADECNALLSYF
FRMRRQVFKAQKKAQSSTD

ecTadA (E25M, R26G, L84F, A106V, R107P, D108N, H123Y, A142N, A143D, D147Y, E155V, I156F

(SEQ ID NO: 72)
SEVEFSHEYWMRHALTLAKRAWDMGEVPVGAVLVHNNRVIGEGWNRPIG
RHDPTAHAEIMALRQGGLVMQNYRLIDATLYVTFEPCVMCAGAMIHSRI
GRVVFGVPNAKTGAAGSLMDVLHYPGMNHRVEITEGILADECNDLLSYF
FRMRRQVFKAQKKAQSSTD

ecTadA (R26C, L84F, A106V, R107H, D108N, H123Y, A142N, D147Y, E155V, I156F)

(SEQ ID NO: 73)
SEVEFSHEYWMRHALTLAKRAWDECEVPVGAVLVHNNRVIGEGWNRPIG
RHDPTAHAEIMALRQGGLVMQNYRLIDATLYVTFEPCVMCAGAMIHSRI
GRVVFGVHNAKTGAAGSLMDVLHYPGMNHRVEITEGILADECNALLSYF
FRMRRQVFKAQKKAQSSTD

ecTadA (L84F, A106V, D108N, H123Y, A142N, A143L, D147Y, E155V, I156F)

(SEQ ID NO: 74)
SEVEFSHEYWMRHALTLAKRAWDEREVPVGAVLVHNNRVIGEGWNRPIG
RHDPTAHAEIMALRQGGLVMQNYRLIDATLYVTFEPCVMCAGAMIHSRI
GRVVFGVRNAKTGAAGSLMDVLHYPGMNHRVEITEGILADECNLLLSYF
FRMRRQVFKAQKKAQSSTD

ecTadA (R26G, L84F, A106V, D108N, H123Y, A142N, D147Y, E155V, I156F)

(SEQ ID NO: 75)
SEVEFSHEYWMRHALTLAKRAWDEGEVPVGAVLVHNNRVIGEGWNRPIG
RHDPTAHAEIMALRQGGLVMQNYRLIDATLYVTFEPCVMCAGAMIHSRI
GRVVFGVRNAKTGAAGSLMDVLHYPGMNHRVEITEGILADECNALLSYF
FRMRRQVFKAQKKAQSSTD

ecTadA (R51H, L84F, A106V, D108N, H123Y, D147Y, E155V, I156F, K157N)

(SEQ ID NO: 76)
SEVEFSHEYWMRHALTLAKRAWDEREVPVGAVLVHNNRVIGEGWNRPIG
HHDPTAHAEIMALRQGGLVMQNYRLIDATLYVTFEPCVMCAGAMIHSRI
GRVVFGVRNAKTGAAGSLMDVLHYPGMNHRVEITEGILADECAALLSYF
FRMRRQVFNAQKKAQSSTD

ecTadA (E25A, R26G, L84F, A106V, R107N, D108N, H123Y, A142N, A143E, D147Y, E155V, I156F)

(SEQ ID NO: 77)
SEVEFSHEYWMRHALTLAKRAWDAGEVPVGAVLVHNNRVIGEGWNRPIG
RHDPTAHAEIMALRQGGLVMQNYRLIDATLYVTFEPCVMCAGAMIHSRI
GRVVFGVNNAKTGAAGSLMDVLHYPGMNHRVEITEGILADECNELLSYF
FRMRRQVFKAQKKAQSSTD

ecTadA (N37T, P48T, L84F, A106V, D108N, H123Y, D147Y, E155V, I156F)

(SEQ ID NO: 78)
SEVEFSHEYWMRHALTLAKRAWDEREVPVGAVLVHTNRVIGEGWNRTIG
RHDPTAHAEIMALRQGGLVMQNYRLIDATLYVTFEPCVMCAGAMIHSRI
GRVVFGVRNAKTGAAGSLMDVLHYPGMNHRVEITEGILADECAALLSYF
FRMRRQVFKAQKKAQSSTD

ecTadA (N37S, L84F, A106V, D108N, H123Y, D147Y, E155V, I156F)

(SEQ ID NO: 79)
SEVEFSHEYWMRHALTLAKRAWDEREVPVGAVLVHSNRVIGEGWNRPIG
RHDPTAHAEIMALRQGGLVMQNYRLIDATLYVTFEPCVMCAGAMIHSRI
GRVVFGVRNAKTGAAGSLMDVLHYPGMNHRVEITEGILADECAALLSYF
FRMRRQVFKAQKKAQSSTD

ecTadA (H36L, L84F, A106V, D108N, H123Y, D147Y, E155V, I156F)

(SEQ ID NO: 80)
SEVEFSHEYWMRHALTLAKRAWDEREVPVGAVLVLNNRVIGEGWNRPIG
RHDPTAHAEIMALRQGGLVMQNYRLIDATLYVTFEPCVMCAGAMIHSRI
GRVVFGVRNAKTGAAGSLMDVLHYPGMNHRVEITEGILADECAALLSY
FFRMRRQVFKAQKKAQSSTD

ecTadA (H36L, P48L, L84F, A106V, D108N, H123Y, D147Y, E155V, I156F)

(SEQ ID NO: 81)
SEVEFSHEYWMRHALTLAKRAWDEREVPVGAVLVLNNRVIGEGWNRLIG
RHDPTAHAEIMALRQGGLVMQNYRLIDATLYVTFEPCVMCAGAMIHSRI
GRVVFGVRNAKTGAAGSLMDVLHYPGMNHRVEITEGILADECAALLSYF
FRMRRQVFKAQKKAQSSTD

ecTadA (H36L, L84F, A106V, D108N, H123Y, D147Y, E155V, K57N, I156F)

(SEQ ID NO: 82)
SEVEFSHEYWMRHALTLAKRAWDEREVPVGAVLVLNNRVIGEGWNRPIG
RHDPTAHAEIMALRQGGLVMQNYRLIDATLYVTFEPCVMCAGAMIHSRI
GRVVFGVRNAKTGAAGSLMDVLHYPGMNHRVEITEGILADECAALLSYF
FRMRRQVFNAQKKAQSSTD

ecTadA (H36L, L84F, A106V, D108N, H123Y, S146C, D147Y, E155V, I156F)

(SEQ ID NO: 83)
SEVEFSHEYWMRHALTLAKRAWDEREVPVGAVLVLNNRVIGEGWNRPIG
RHDPTAHAEIMALRQGGLVMQNYRLIDATLYVTFEPCVMCAGAMIHSRI
GRVVFGVRNAKTGAAGSLMDVLHYPGMNHRVEITEGILADECAALLCYF
FRMRRQVFKAQKKAQSSTD

ecTadA (L84F, A106V, D108N, H123Y, S146R, D147Y, E155V, I156F)

(SEQ ID NO: 84)
SEVEFSHEYWMRHALTLAKRAWDEREVPVGAVLVHNNRVIGEGWNRPIG
RHDPTAHAEIMALRQGGLVMQNYRLIDATLYVTFEPCVMCAGAMIHSRI
GRVVFGVRNAKTGAAGSLMDVLHYPGMNHRVEITEGILADECAALLRYF
FRMRRQVFKAQKKAQSSTD

ecTadA (N37S, R51H, L84F, A106V, D108N, H123Y, D147Y, E155V, I156F

(SEQ ID NO: 85)
SEVEFSHEYWMRHALTLAKRAWDEREVPVGAVLVHSNRVIGEGWNRPIG
HHDPTAHAEIMALRQGGLVMQNYRLIDATLYVTFEPCVMCAGAMIHSRI
GRVVFGVRNAKTGAAGSLMDVLHYPGMNHRVEITEGILADECAALLSYF
FRMRRQVFKAQKKAQSSTD

ecTadA (R51L, L84F, A106V, D108N, H123Y, D147Y, E155V, I156F, K157N

(SEQ ID NO: 86)
SEVEFSHEYWMRHALTLAKRAWDEREVPVGAVLVHNNRVIGEGWNRPIG
LHDPTAHAEIMALRQGGLVMQNYRLIDATLYVTFEPCVMCAGAMIHSRI
GRVVFGVRNAKTGAAGSLMDVLHYPGMNHRVEITEGILADECAALLSYF
FRMRRQVFNAQKKAQSSTD

saTadA (D108N)

(SEQ ID NO: 87)
GSHMTNDIYFMTLAIEEAKKAAQLGEVPIGAIITKDDEVIARAHNLRETL
QQPTAHAEHIAIERAAKVLGSWRLEGCTLYVTLEPCVMCAGTIVMSRIPR
VVYGADNPKGGCSGSLMNLLQQSNFNHRAIVDKGVLKEACSTLLTTFFKN
LRANKKSTN

saTadA (D107A_D108N)

(SEQ ID NO: 88)
GSHMTNDIYFMTLAIEEAKKAAQLGEVPIGAIITKDDEVIARAHNLRETL
QQPTAHAEHIAIERAAKVLGSWRLEGCTLYVTLEPCVMCAGTIVMSRIPR
VVYGAANPKGGCSGSLMNLLQQSNFNHRAIVDKGVLKEACSTLLTTFFKN
LRANKKSTN

saTadA (G26P_D107A_D108N)

(SEQ ID NO: 89)
GSHMTNDIYFMTLAIEEAKKAAQLPEVPIGAIITKDDEVIARAHNLRETL
QQPTAHAEHIAIERAAKVLGSWRLEGCTLYVTLEPCVMCAGTIVMSRIPR
VVYGAANPKGGCSGSLMNLLQQSNFNHRAIVDKGVLKEACSTLLTTFFKN
LRANKKSTN

saTadA (G26P_D107A_D108N_S142A)

(SEQ ID NO: 90)
GSHMTNDIYFMTLAIEEAKKAAQLPEVPIGAIITKDDEVIARAHNLRETL
QQPTAHAEHIAIERAAKVLGSWRLEGCTLYVTLEPCVMCAGTIVMSRIPR
VVYGAANPKGGCSGSLMNLLQQSNFNHRAIVDKGVLKEACATLLTTFFKN
LRANKKSTN

saTadA (D107A_D108N_S142A)

(SEQ ID NO: 91)
GSHMTNDIYFMTLAIEEAKKAAQLGEVPIGAIITKDDEVIARAHNLRETL
QQPTAHAEHIAIERAAKVLGSWRLEGCTLYVTLEPCVMCAGTIVMSRIPR
VVYGAANPKGGCSGSLMNLLQQSNFNHRAIVDKGVLKEACATLLTTFFKN
LRANKKSTN

ecTadA (P48S)

(SEQ ID NO: 92)
SEVEFSHEYWMRHALTLAKRAWDEREVPVGAVLVHNNRVIGEGWNRSIGR
HDPTAHAEIMALRQGGLVMQNYRLIDATLYVTLEPCVMCAGAMIHSRIGR
VVFGARDAKTGAAGSLMDVLHHPGMNHRVEITEGILADECAALLSDFFRM
RRQEIKAQKKAQSSTD

ecTadA (P48T)

(SEQ ID NO: 93)
SEVEFSHEYWMRHALTLAKRAWDEREVPVGAVLVHNNRVIGEGWNRTIGR
HDPTAHAEIMALRQGGLVMQNYRLIDATLYVTLEPCVMCAGAMIHSRIGR
VVFGARDAKTGAAGSLMDVLHHPGMNHRVEITEGILADECAALLSDFFRM
RRQEIKAQKKAQSSTD

ecTadA (P48A)

(SEQ ID NO: 94)
SEVEFSHEYWMRHALTLAKRAWDEREVPVGAVLVHNNRVIGEGWNRAIGR
HDPTAHAEIMALRQGGLVMQNYRLIDATLYVTLEPCVMCAGAMIHSRIGR
VVFGARDAKTGAAGSLMDVLHHPGMNHRVEITEGILADECAALLSDFFRM
RRQEIKAQKKAQSSTD

ecTadA (A142N)

(SEQ ID NO: 95)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGARDAKTGAAGSLMDVLHHPGMNHRVEITEGILADECNALLSDFFRM
RRQEIKAQKKAQSSTD

ecTadA (W23R)

(SEQ ID NO: 96)
SEVEFSHEYWMRHALTLAKRARDEREVPVGAVLVHNNRVIGEGWNRPIGR
HDPTAHAEIMALRQGGLVMQNYRLIDATLYVTLEPCVMCAGAMIHSRIGR
VVFGARDAKTGAAGSLMDVLHHPGMNHRVEITEGILADECAALLSDFFRM
RRQEIKAQKKAQSSTD

ecTadA (W23L)

(SEQ ID NO: 97)
SEVEFSHEYWMRHALTLAKRALDEREVPVGAVLVHNNRVIGEGWNRPIGR
HDPTAHAEIMALRQGGLVMQNYRLIDATLYVTLEPCVMCAGAMIHSRIGR
VVFGARDAKTGAAGSLMDVLHHPGMNHRVEITEGILADECAALLSDFFRM
RRQEIKAQKKAQSSTD

ecTadA (R152P)

(SEQ ID NO: 98)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGARDAKTGAAGSLMDVLHHPGMNHRVEITEGILADECAALLSDFFRM
PRQEIKAQKKAQSSTD

ecTadA (R152H)

(SEQ ID NO: 99)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGR
VVFGARDAKTGAAGSLMDVLHHPGMNHRVEITEGILADECAALLSDFFRM
HRQEIKAQKKAQSSTD

ecTadA (L84F, A106V, D108N, H123Y, D147Y, E155V, I156F)

(SEQ ID NO: 100)
SEVEFSHEYWMRHALTLAKRAWDEREVPVGAVLVHNNRVIGEGWNRPIGR
HDPTAHAEIMALRQGGLVMQNYRLIDATLYVTFEPCVMCAGAMIHSRIGR
VVFGVRNAKTGAAGSLMDVLHYPGMNHRVEITEGILADECAALLSYFFRM
RRQVFKAQKKAQSSTD

ecTadA (H36L, R51L, L84F, A106V, D108N, H123Y, S146C, D147Y, E155V, I156F, K157N)

(SEQ ID NO: 101)
SEVEFSHEYWMRHALTLAKRAWDEREVPVGAVLVLNNRVIGEGWNRPIGL
HDPTAHAEIMALRQGGLVMQNYRLIDATLYVTFEPCVMCAGAMIHSRIGR
VVFGVRNAKTGAAGSLMDVLHYPGMNHRVEITEGILADECAALLCYFFRM
RRQVFNAQKKAQSSTD

ecTadA (H36L, P48S, R51L, L84F, A106V, D108N, H123Y, S146C, D147Y, E155V, I156F, K157N)

(SEQ ID NO: 102)
SEVEFSHEYWMRHALTLAKRAWDEREVPVGAVLVLNNRVIGEGWNRSIG
LHDPTAHAEIMALRQGGLVMQNYRLIDATLYVTFEPCVMCAGAMIHSRI
GRVVFGVRNAKTGAAGSLMDVLHYPGMNHRVEITEGILADECAALLCYF
FRMRRQVFNAQKKAQSSTD

ecTadA (H36L, P48A, R51L, L84F, A106V, D108N, H123Y, S146C, D147Y, E155V, I156F, K157N)

(SEQ ID NO: 103)
SEVEFSHEYWMRHALTLAKRAWDEREVPVGAVLVLNNRVIGEGWNRAIG
LHDPTAHAEIMALRQGGLVMQNYRLIDATLYVTFEPCVMCAGAMIHSRI
GRVVFGVRNAKTGAAGSLMDVLHYPGMNHRVEITEGILADECAALLCYF
FRMRRQVFNAQKKAQSSTD

ecTadA (W23L, H36L, P48A, R51L, L84F, A106V, D108N, H123Y, S146C, D147Y, R152P, E155V, I156F, K157N)

(SEQ ID NO: 104)
SEVEFSHEYWMRHALTLAKRALDEREVPVGAVLVLNNRVIGEGWNRAIG
LHDPTAHAEIMALRQGGLVMQNYRLIDATLYVTFEPCVMCAGAMIHSRI
GRVVFGVRNAKTGAAGSLMDVLHYPGMNHRVEITEGILADECAALLCYF
FRMPRQVFNAQKKAQSSTD

ecTadA (W23R, H36L, P48A, R51L, L84F, A106V, D108N, H123Y, S146C, D147Y, R152P, E155V, I156F, K157N)

(SEQ ID NO: 29)
SEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRI
GRVVFGVRNAKTGAAGSLMDVLHYPGMNHRVEITEGILADECAALLCYF
FRMPRQVFNAQKKAQSSTD

Staphylococcus aureus TadA:

(SEQ ID NO: 105)
MGSHMTNDIYFMTLAIEEAKKAAQLGEVPIGAIITKDDEVIARAHNLRE
TLQQPTAHAEHIAIERAAKVLGSWRLEGCTLYVTLEPCVMCAGTIVMSR
IPRVVYGADDPKGGCSGSLMNLLQQSNFNHRAIVDKGVLKEACSTLLTT
FFKNLRANKKSTN

Bacillus subtilis TadA:

(SEQ ID NO: 106)
MTQDELYMKEAIKEAKKAEEKGEVPIGAVLVINGEIIARAHNLRETEQR
SIAHAEMLVIDEACKALGTWRLEGATLYVTLEPCPMCAGAVVLSRVEKV
VFGAFDPKGGCSGTLMNLLQEERFNHQAEVVSGVLEEECGGMLSAFFRE
LRKKKKAARKNLSE

Salmonella typhimurium (S. typhimurium) TadA:

(SEQ ID NO: 107)
MPPAFITGVTSLSDVELDHEYWMRHALTLAKRAWDEREVPVGAVLVHNH
RVIGEGWNRPIGRHDPTAHAEIMALRQGGLVLQNYRLLDTTLYVTLEPC
VMCAGAMVHSRIGRVVFGARDAKTGAAGSLIDVLHHPGMNHRVEIIEGV
LRDECATLLSDFFRMRRQEIKALKKADRAEGAGPAV

Shewanella putrefaciens (S. putrefaciens) TadA:

(SEQ ID NO: 108)
MDEYWMQVAMQMAEKAEAAGEVPVGAVLVKDGQQIATGYNLSISQHDPT
AHAEILCLRSAGKKLENYRLLDATLYITLEPCAMCAGAMVHSRIARVVY
GARDEKTGAAGTVVNLLQHPAFNHQVEVTSGVLAEACSAQLSRFFKRRR
DEKKALKLAQRAQQGIE

Haemophilus influenzae F3031 (H. influenzae) TadA:

(SEQ ID NO: 109)
MDAAKVRSEFDEKMMRYALELADKAEALGEIPVGAVLVDDARNIIGEGW
NLSIVQSDPTAHAEIIALRNGAKNIQNYRLLNSTLYVTLEPCTMCAGAI
LHSRIKRLVFGASDYKTGAIGSRFHFFDDYKMNHTLEITSGVLAEECSQ
KLSTFFQKRREEKKIEKALLKSLSDK

Caulobacter crescentus (C. crescentus) TadA:

(SEQ ID NO: 110)
MRTDESEDQDHRMMRLALDAARAAAEAGETPVGAVILDPSTGEVIATAG
NGPIAAHDPTAHAEIAAMRAAAAKLGNYRLTDLTLVVTLEPCAMCAGAI
SHARIGRVVFGADDPKGGAVVHGPKFFAQPTCHWRPEVTGGVLADESAD
LLRGFFRARRKAKI

Geobacter sulfurreducens (G. sulfurreducens) TadA:

(SEQ ID NO: 111)
MSSLKKTPIRDDAYWMGKAIREAAKAAARDEVPIGAVIVRDGAVIGRGH
NLREGSNDPSAHAEMIAIRQAARRSANWRLTGATLYVTLEPCLMCMGAI
ILARLERVVFGCYDPKGGAAGSLYDLSADPRLNHQVRLSPGVCQEECGT
MLSDFFRDLRRRKKAKATPALFIDERKVPPEP

Streptococcus pyogenes (S. pyogenes) TadA

(SEQ ID NO: 112)
MPYSLEEQTYFMQEALKEAEKSLQKAEIPIGCVIVKDGEIIGRGHNARE
ESNQAIMHAEIMAINEANAHEGNWRLLDTTLFVTIEPCVMCSGAIGLAR
IPHVIYGASNQKFGGADSLYQILTDERLNHRVQVERGLLAADCANIMQT
FFRQGRERKKIAKHLIKEQSDPFD

### TadA 7.10:

(SEQ ID NO: 113)
SEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRI
GRVVFGVRNAKTGAAGSLMDVLHYPGMNHRVEITEGILADECAALLCYF
FRMPRQVFNAQKKAQSSTD

TadA 7.10 (V106W) (E. coli)

(SEQ ID NO: 114)
SEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRI
GRVVFGWRNAKTGAAGSLMDVLHYPGMNHRVEITEGILADECAALLCYF
FRMPRQVFNAQKKAQSSTD

TadA-8e (E. coli)

(SEQ ID NO: 115)
SEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRI
GRVVFGVRNSKRGAAGSLMNVLNYPGMNHRVEITEGILADECAALLCDF
YRMPRQVFNAQKKAQSSIN

TadA-8e(V106W) (E. coli)

(SEQ ID NO: 116)
SEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRI
GRVVFGWRNSKRGAAGSLMNVLNYPGMNHRVEITEGILADECAALLCDF
YRMPRQVFNAQKKAQSSIN

Aquifex aeolicus (A. aeolicus) TadA

(SEQ ID NO: 117)
MGKEYFLKVALREAKRAFEKGEVPVGAIIVKEGEIISKAHNSVEELKDP
TAHAEMLAIKEACRRLNTKYLEGCELYVTLEPCIMCSYALVLSRIEKVI
FSALDKKHGGVVSVFNILDEPTLNHRVKWEYYPLEEASELLSEFFKKLR
NNII

### Tad1

(SEQ ID NO: 118)
SEVEFSHEYWMRHALTLAKRARDEGEVPVGAVLVLNNRVIGEGWNRAIG
LYDPTAHAEIMALRQGGLVMQNYRLIDATLYVTFEPCVMCAGAMIHSRI
GRVVFGVRNSKRGAAGSLMNVLNYPGMDHRVEITEGILADECAALLCDF
YRMPRQVFNAQKKAQSSIN

### Tad2

(SEQ ID NO: 119)
SEVEFSHEYWMRHALTLAKRARDEGEVPVGAVLVLNNRVIGEGWNRAIG
LHDPTAHAEIMALRQGGLVMQNYRLIDATLYVTFEPCVMCAGAMIHSRI
GRVVFGVRNSKRGAAGSLMNVLNYPGMDHRVEITEGILADECAALLCDF
YRMPRQVFNAQKKAQSSIN

### Tad3

(SEQ ID NO: 120)
SEVEFSHEYWMRHALTLAKRARDEREVPVGAVLVLNNRVIGEGWNRAIG
LHDPTAHAEIMALRQGGLVMQNYGLIDATLYVTFEPCVMCAGAIIHSRI
GRVVFGVRNSKRGAAGSLMNVLNYPGMNHRVEITEGILADECAALLCDF
YRMPRQVFNAQKKAQSSIN

### Tad4

(SEQ ID NO: 121)
SEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRI
GRVVFGVRNSKRGAAGSLMNVLNYPGMDHRVEITEGILADECAALLCDF
YRMPRQVFNAQKKAQSSIN

### Tad6

(SEQ ID NO: 122)
SEVEFSHEYWMRHALTLAKRARDEGEVPVGAVLVLNNRVIGEGWNRAIG
LYDPTAHAEIMALRQGGLVMQNYGLIDATLYVTFEPCVMCAGAMIHSRI
GRVVFGVRNSKRGAAGSLMNVLNYPGMDHRVEITEGILADECAALLCDF
YRMPRQVFNAQKKAQSSIN

### Tad6-SR

(SEQ ID NO: 123)
SEVEFSHEYWMRHALTLAKRARDEGEVPVGAVLVLNNRVIGEGWNRAIG
LYDPTAHAEIMALRQGGLVMQNYGLIDATLYSTFEPCVMCAGAMIHSRI
GRVVFGVRNSKRGAAGSLMNVLNYPGMDHRVEITEGILADECAALLCDF
YRMPRRVFNAQKKAQSSIN

In some aspects, the fusion proteins of the present disclosure comprise cytidine base editors (CBEs) comprising a napDNAbp domain (e.g., any of the Cas14a1 variants provided herein) and a cytosine deaminase domain that enzymatically deaminates a cytosine nucleobase of a C:G nucleobase pair to a uracil. The uracil may be subsequently converted to a thymine (T) by the cell's DNA repair and replication machinery. The mismatched guanine (G) on the opposite strand may subsequently be converted to an adenine (A) by the cell's DNA repair and replication machinery. In this manner, a target C:G nucleobase pair is ultimately converted to a T:A nucleobase pair. Other cytosine deaminase domains besides those provided herein are known in the art, and a person of ordinary skill in the art would recognize which cytosine deaminase domains could be used in the fusion proteins of the present disclosure.

The CBE fusion proteins described herein may further comprise one or more nuclear localization signals (NLSs) and/or one or more uracil glycosylase inhibitor (UGI) domains. Thus, the base editor fusion proteins may comprise the structure: NH₂-[first nuclear localization sequence]-[cytosine deaminase domain]-[napDNAbp domain]-[first UGI domain]-[second UGI domain]-[second nuclear localization sequence]-COOH, wherein each instance of “]-[” indicates the presence of an optional linker sequence. The CBE fusion proteins of the present disclosure may comprise modified (or evolved) cytosine deaminase domains, such as deaminase domains that recognize an expanded PAM sequence, have improved efficiency of deaminating 5′-GC targets, and/or make edits in a narrower target window.

In some aspects, the fusion proteins of the disclosure comprise an adenine base editor. Some aspects of the disclosure provide fusion proteins that comprise a nucleic acid programmable DNA binding protein (napDNAbp), such as any of the Cas14a1 variants provided herein, and at least two adenosine deaminase domains. Without wishing to be bound by any particular theory, dimerization of adenosine deaminases (e.g., in cis or in trans) may improve the ability (e.g., efficiency) of the fusion protein to modify a nucleic acid base (for example, to deaminate adenine). In some embodiments, any of the fusion proteins may comprise 2, 3, 4, or 5 adenosine deaminase domains. In some embodiments, any of the fusion proteins provided herein comprises two adenosine deaminases. In some embodiments, any of the fusion proteins provided herein contain only two adenosine deaminases. In some embodiments, the adenosine deaminases are the same. In some embodiments, the adenosine deaminases are any of the adenosine deaminases provided herein. In some embodiments, the adenosine deaminases are different. Other adenosine deaminase domains besides those provided herein are known in the art, and a person of ordinary skill in the art would recognize which adenosine deaminase domains could be used in the fusion proteins of the present disclosure.

In some embodiments, the general architecture of exemplary fusion proteins with a first adenosine deaminase, a second adenosine deaminase, and a napDNAbp (e.g., any of the Cas14a1 variants provided herein) comprises any one of the following structures, where NLS is a nuclear localization sequence (e.g., any NLS provided herein), NH₂ is the N-terminus of the fusion protein, and COOH is the C-terminus of the fusion protein: NH₂-[first adenosine deaminase]-[second adenosine deaminase]-[napDNAbp]-COOH; NH₂-[first adenosine deaminase]-[napDNAbp]-[second adenosine deaminase]-COOH; NH₂-[napDNAbp]-[first adenosine deaminase]-[second adenosine deaminase]-COOH; NH₂-[second adenosine deaminase]-[first adenosine deaminase]-[napDNAbp]-COOH; NH₂-[second adenosine deaminase]-[napDNAbp]-[first adenosine deaminase]-COOH; NH₂-[napDNAbp]-[second adenosine deaminase]-[first adenosine deaminase]-COOH.

In some embodiments, the fusion proteins provided herein do not comprise a linker. In some embodiments, a linker is present between one or more of the domains or proteins (e.g., first adenosine deaminase, second adenosine deaminase, and/or napDNAbp). In some embodiments, the “]-[” used in the general architecture above indicates the presence of an optional linker. Exemplary fusion proteins comprising a first adenosine deaminase, a second adenosine deaminase, a napDNAbp, and an NLS are provided: NH₂-[NLS]-[first adenosine deaminase]-[second adenosine deaminase]-[napDNAbp]-COOH; NH₂-[first adenosine deaminase]-[NLS]-[second adenosine deaminase]-[napDNAbp]-COOH; NH₂-[first adenosine deaminase]-[second adenosine deaminase]-[NLS]-[napDNAbp]-COOH; NH₂-[first adenosine deaminase]-[second adenosine deaminase]-[napDNAbp]-[NLS]-COOH; NH₂-[NLS]-[first adenosine deaminase]-[napDNAbp]-[second adenosine deaminase]-COOH; NH₂-[first adenosine deaminase]-[NLS]-[napDNAbp]-[second adenosine deaminase]-COOH; NH₂-[first adenosine deaminase]-[napDNAbp]-[NLS]-[second adenosine deaminase]-COOH; NH₂-[first adenosine deaminase]-[napDNAbp]-[second adenosine deaminase]-[NLS]-COOH; NH₂-[NLS]-[napDNAbp]-[first adenosine deaminase]-[second adenosine deaminase]-COOH; NH₂-[napDNAbp]-[NLS]-[first adenosine deaminase]-[second adenosine deaminase]-COOH; NH₂-[napDNAbp]-[first adenosine deaminase]-[NLS]-[second adenosine deaminase]-COOH; NH₂-[napDNAbp]-[first adenosine deaminase]-[second adenosine deaminase]-[NLS]-COOH; NH₂-[NLS]-[second adenosine deaminase]-[first adenosine deaminase]-[napDNAbp]-COOH; NH₂-[second adenosine deaminase]-[NLS]-[first adenosine deaminase]-[napDNAbp]-COOH; NH₂-[second adenosine deaminase]-[first adenosine deaminase]-[NLS]-[napDNAbp]-COOH; NH₂-[second adenosine deaminase]-[first adenosine deaminase]-[napDNAbp]-[NLS]-COOH; NH₂-[NLS]-[second adenosine deaminase]-[napDNAbp]-[first adenosine deaminase]-COOH; NH₂-[second adenosine deaminase]-[NLS]-[napDNAbp]-[first adenosine deaminase]-COOH; NH₂-[second adenosine deaminase]-[napDNAbp]-[NLS]-[first adenosine deaminase]-COOH; NH₂-[second adenosine deaminase]-[napDNAbp]-[first adenosine deaminase]-[NLS]-COOH; NH₂-[NLS]-[napDNAbp]-[second adenosine deaminase]-[first adenosine deaminase]-COOH; NH₂-[napDNAbp]-[NLS]-[second adenosine deaminase]-[first adenosine deaminase]-COOH; NH₂-[napDNAbp]-[second adenosine deaminase]-[NLS]-[first adenosine deaminase]-COOH; NH₂-[napDNAbp]-[second adenosine deaminase]-[first adenosine deaminase]-[NLS]-COOH.

### Prime Editing and Reverse Transcriptase Domains

In various embodiments, the fusion proteins described herein comprise a Cas protein and a reverse transcriptase domain (i.e., the fusion protein is a prime editor or otherwise useful for performing prime editing). Prime editing is a versatile and precise genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors not only search and locate the desired target site to be edited, but at the same time, encode a replacement strand containing a desired edit that is installed in place of the corresponding target site endogenous DNA strand.

Prime editing relates, in part, to the discovery that the mechanism of target-primed reverse transcription (TPRT) or “prime editing” can be leveraged or adapted for conducting precision CRISPR/Cas-based genome editing with high efficiency and genetic flexibility. TPRT is naturally used by mobile DNA elements, such as mammalian non-LTR retrotransposons and bacterial Group II introns. Cas protein-reverse transcriptase fusions or related systems can be used to target a specific DNA sequence with a guide RNA, generate a single strand nick at the target site, and use the nicked DNA as a primer for reverse transcription of an engineered reverse transcriptase template that is integrated with the guide RNA. However, while the concept begins with prime editors that use reverse transcriptases as the DNA polymerase component, the prime editors described herein (e.g., prime editors comprising any of the Cas14a1 variants disclosed herein) are not limited to reverse transcriptases but may include the use of virtually any DNA polymerase. Indeed, while the application throughout may refer to prime editors with “reverse transcriptases,” it is set forth here that reverse transcriptases are only one type of DNA polymerase that may work with prime editing. Thus, wherever the specification mentions “reverse transcriptases,” the person having ordinary skill in the art should appreciate that any suitable DNA polymerase may be used in place of the reverse transcriptase. Thus, in one aspect, the prime editors may comprise a Cas14a1 variant described herein that is programmed to target a DNA sequence by associating it with a specialized guide RNA (i.e., PEgRNA) containing a spacer sequence that anneals to a complementary protospacer in the target DNA. The specialized guide RNA also contains new genetic information in the form of an extension that encodes a replacement strand of DNA containing a desired genetic alteration that is used to replace a corresponding endogenous DNA strand at the target site. To transfer information from the PEgRNA to the target DNA, the mechanism of prime editing involves nicking the target site in one strand of the DNA to expose a 3′-hydroxyl group. The exposed 3′-hydroxyl group can then be used to prime the DNA polymerization of the edit-encoding extension on PEgRNA directly into the target site. In various embodiments, the extension—which provides the template for polymerization of the replacement strand containing the edit—can be formed from RNA or DNA. In the case of an RNA extension, the polymerase of the prime editor can be an RNA-dependent DNA polymerase (such as, a reverse transcriptase). In the case of a DNA extension, the polymerase of the prime editor may be a DNA-dependent DNA polymerase.

The newly synthesized strand (i.e., the replacement DNA strand containing the desired edit) that is formed by the prime editors would be homologous to the genomic target sequence (i.e., have the same sequence as) except for the inclusion of a desired nucleotide change (e.g., a single nucleotide change, a deletion, an insertion, or a combination thereof). The newly synthesized (or replacement) strand of DNA may also be referred to as a single strand DNA flap, which would compete for hybridization with the complementary homologous endogenous DNA strand, thereby displacing the corresponding endogenous strand. In certain embodiments, the system can be combined with the use of an error-prone reverse transcriptase enzyme (e.g., provided as a fusion protein with the Cas14a1 domain, or provided in trans to the Cas14a1 domain). The error-prone reverse transcriptase enzyme can introduce alterations during synthesis of the single strand DNA flap. Thus, in certain embodiments, error-prone reverse transcriptase can be utilized to introduce nucleotide changes to the target DNA. Depending on the error-prone reverse transcriptase that is used with the system, the changes can be random or non-random.

Resolution of the hybridized intermediate (comprising the single strand DNA flap synthesized by the reverse transcriptase hybridized to the endogenous DNA strand) can include removal of the resulting displaced flap of endogenous DNA (e.g., with a 5′ end DNA flap endonuclease, FEN1), ligation of the synthesized single strand DNA flap to the target DNA, and assimilation of the desired nucleotide change as a result of cellular DNA repair and/or replication processes. Because templated DNA synthesis offers single nucleotide precision for the modification of any nucleotide, including insertions and deletions, the scope of this approach is very broad and could foreseeably be used for myriad applications in basic science and therapeutics.

The prime editor (PE) system described herein contemplate fusion proteins comprising a napDNAbp and a polymerase (e.g., DNA-dependent DNA polymerase or RNA-dependent DNA polymerase, such as, reverse transcriptase), and optionally joined by a linker. The application contemplates any suitable napDNAbp and polymerase (e.g., DNA-dependent DNA polymerase or RNA-dependent DNA polymerase, such as, reverse transcriptase) to be combined in a single fusion protein. Examples of napDNAbps and polymerases (e.g., DNA-dependent DNA polymerase or RNA-dependent DNA polymerase, such as, reverse transcriptase) are each defined herein. Since polymerases are well-known in the art, and the amino acid sequences are readily available, this disclosure is not meant in any way to be limited to those specific polymerases identified herein.

In various embodiments, the prime editor fusion proteins may comprise any suitable structural configuration. For example, the fusion protein may comprise from the N-terminus to the C-terminus direction, a napDNAbp (e.g., Cas14a1 variant) fused to a polymerase (e.g., DNA-dependent DNA polymerase or RNA-dependent DNA polymerase, such as, reverse transcriptase). In other embodiments, the fusion protein may comprise from the N-terminus to the C-terminus direction, a polymerase (e.g., a reverse transcriptase) fused to a napDNAbp. The fused domain may optionally be joined by a linker, e.g., an amino acid sequence. In other embodiments, the fusion proteins may comprise the structure NH₂-[napDNAbp]-[polymerase]-COOH; or NH₂-[polymerase]-[napDNAbp]-COOH, wherein each instance of “]-[” indicates the presence of an optional linker sequence. In embodiments wherein the polymerase is a reverse transcriptase, the fusion proteins may comprise the structure NH₂-[napDNAbp]-[RT]-COOH; or NH₂-[RT]-[napDNAbp]-COOH, wherein each instance of “]-[” indicates the presence of an optional linker sequence.

In some embodiments, the reverse transcriptase domain is a wild type MMLV reverse transcriptase. In some embodiments, the reverse transcriptase domain is a variant of wild type MMLV reverse transcriptase having the amino acid sequence of SEQ ID NO: 141.

For example, the present disclosure provides fusion proteins comprising any of the Cas14a1 variants described herein, and a variant reverse transcriptase domain of SEQ ID NO: 141, which is based on the wild type MMLV reverse transcriptase domain of SEQ ID NO: 124 (and, in particular, a Genscript codon optimized MMLV reverse transcriptase having the nucleotide sequence of SEQ ID NO: 124), and which comprises amino acid substitutions D200N, T306K, W313F, T330P, and L603W relative to the wild type MMLV RT of SEQ ID NO: 141.

The prime editor fusion proteins provided herein may also comprise other variant RTs as well. In various embodiments, the fusion proteins described herein (with RT provided as either a fusion partner or in trans) can include a variant RT comprising one or more of the following mutations: P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, or D653N in the wild type M-MLV RT of SEQ ID NO: 124, or at a corresponding amino acid position in another wild type RT polypeptide sequence.

Some exemplary reverse transcriptases that can be fused to napDNAbp proteins (e.g., any of the Cas14a1 variants described herein) or provided as individual proteins according to various embodiments of this disclosure are provided below. Exemplary reverse transcriptases include variants with at least 80%, at least 85%, at least 90%, at least 95%, or at least 99% sequence identity to the following wild-type enzymes or partial enzymes (SEQ ID NOs: 124-141):

| Description | Sequence (variant substitutions relative to wild type) |
|---|---|
| Reverse | TLNIEDEYRLHETSKEPDVSLGSTWLSDFPQAWAETGGMGLAVRQAP |
| transcriptase | LIIPLKATSTPVSIKQYPMSQEARLGIKPHIQRLLDQGILVPCQSPWNTPL |
| (M-MLV RT) | LPVKKPGTNDYRPVQDLREVNKRVEDIHPTVPNPYNLLSGLPPSHQWY |
| wild type | TVLDLKDAFFCLRLHPTSQPLFAFEWRDPEMGISGQLTWTRLPQGFKN |
| Moloney | SPTLFDEALHRDLADFRIQHPDLILLQYVDDLLLAATSELDCQQGTRAL |
| murine | LQTLGNLGYRASAKKAQICQKQVKYLGYLLKEGQRWLTEARKETVM |
| leukemia | GQPTPKTPRQLREFLGTAGFCRLWIPGFAEMAAPLYPLTKTGTLFNWG |
| virus | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGLLTSEGKEIKNKDEILALLK |
| | ALFLPKRLSIIHCPGHQKGHSAEARGNRMADQAARKAAITETPDTSTLL |
| | IENSSP (SEQ ID NO: 124) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| | LPVKKPGTNDYRPVQDLREVNKRVEDIHPTVPNPYNLLSGLPPSHQWY |
| | TVLDLKDAFFCLRLHPTSQPLFAFEWRDPEMGISGQLTWTRLPQGFKN |
| | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKTGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGLLTSEGKEIKNKDEILALLK |
| | ALFLPKRLSIIHCPGHQKGHSAEARGNRMADQAARKAAITETPDTSTLL |
| | IENSSP (SEQ ID NO: 125) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| | TVLDLKDAFFCLRLHPTSQPLFAFEWRDPEMGISGQLTWTRLPQGFKN |
| | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGLLTSEGKEIKNKDEILALLK |
| | ALFLPKRLSIIHCPGHQKGHSAEARGNRMADQAARKAAITETPDTSTLL |
| | IENSSP (SEQ ID NO: 126) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 127) |
| M-MLV RT | TLNIEDEYRLHETSKEPDVSLGSTWLSDFPQAWAETGGMGLAVRQAP |
| D200N | LIIPLKATSTPVSIKQYPMSQKARLGIKPHIQRLLDQGILVPCQSPWNTP |
| T330P | LLPVKKPGTNDYRPVQDLREVNKRVEDIHPTVPNPYNLLSGLPPSHQW |
| L603W | YTVLDLKDAFFCLRLHPTSQPLFAFEWRDPEMGISGQLTWTRLPQGFK |
| E69K | NSPTLFNEALHRDLADFRIQHPDLILLQYVDDLLLAATSELDCQQGTRA |
| | LLQTLGNLGYRASAKKAQICQKQVKYLGYLLKEGQRWLTEARKETV |
| | MGQPTPKTPRQLREFLGTAGFCRLWIPGFAEMAAPLYPLTKPGTLFNW |
| | GPDQQKAYQEIKQALLTAPALGLPDLTKPFELFVDEKQGYAKGVLTQ |
| | KLGPWRRPVAYLSKKLDPVAAGWPPCLRMVAAIAVLTKDAGKLTMG |
| | QPLVILAPHAVEALVKQPPDRWLSNARMTHYQALLLDTDRVQFGPVV |
| | ALNPATLLPLPEEGLQHNCLDILAEAHGTRPDLTDQPLPDADHTWYTD |
| | GSSLLQEGQRKAGAAVTTETEVIWAKALPAGTSAQRAELIALTQALK |
| | MAEGKKLNVYTDSRYAFATAHIHGEIYRRRGWLTSEGKEIKNKDEILA |
| | LLKALFLPKRLSIIHCPGHQKGHSAEARGNRMADQAARKAAITETPDT |
| | STLLIENSSP (SEQ ID NO: 128) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| E302R | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLRRFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP(SEQ ID NO: 129) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| E607K | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSKGKEIKNKDEILALL |
| | KALFLPKRLSIIHCPGHQKGHSAEARGNRMADQAARKAAITETPDTST |
| | LLIENSSP (SEQ ID NO: 130) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGPPPSHQWY |
| L603W | TVLDLKDAFFCLRLHPTSQPLFAFEWRDPEMGISGQLTWTRLPQGFKN |
| L139P | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 131) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| L435G | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVIGAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 132) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| N454K | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSKARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 133) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| T306K | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGKAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 134) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| W313F | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLFIPGFAEMAAPLYPLTKPGTLFNWGP |
| | DQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 135) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| D524G | SPTLFNEALHRDLADFRIQHPDLILLQYVDDLLLAATSELDCQQGTRAL |
| E562Q | LQTLGNLGYRASAKKAQICQKQVKYLGYLLKEGQRWLTEARKETVM |
| D583N | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTGGS |
| | SLLQEGQRKAGAAVTTETEVIWAKALPAGTSAQRAQLIALTQALKMA |
| | EGKKLNVYTNSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 136) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| E302R | SPTLFNEALHRDLADFRIQHPDLILLQYVDDLLLAATSELDCQQGTRAL |
| W313F | LQTLGNLGYRASAKKAQICQKQVKYLGYLLKEGQRWLTEARKETVM |
| | GQPTPKTPRQLRRFLGTAGFCRLFIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 137) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGPPPSHQWY |
| L603W | TVLDLKDAFFCLRLHPTSQPLFAFEWRDPEMGISGQLTWTRLPQGFKN |
| E607K | SPTLFNEALHRDLADFRIQHPDLILLQYVDDLLLAATSELDCQQGTRAL |
| L139P | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSKGKEIKNKDEILALL |
| | KALFLPKRLSIIHCPGHQKGHSAEARGNRMADQAARKAAITETPDTST |
| | LLIENSSP (SEQ ID NO: 138) |
| M-MLV RT | TLNIEDEYRLHETSKEPDVSLGSTWLSDFPQAWAETGGMGLAVRQAP |
| P51L S67K | LIILLKATSTPVSIKQYPMKQEARLGIKPHIQRLLDQGILVPCQSPWNTP |
| T197A | LLPVKKPGTNDYRPVQDLREVNKRVEDIHPTVPNPYNLLSGLPPSHQW |
| H204R | YTVLDLKDAFFCLRLHPTSQPLFAFEWRDPEMGISGQLTWTRLPQGFK |
| E302K | NSPALFDEALRRDLADFRIQHPDLILLQYVDDLLLAATSELDCQQGTR |
| F309N | ALLQTLGNLGYRASAKKAQICQKQVKYLGYLLKEGQRWLTEARKET |
| W313F | VMGQPTPKTPRQLRKFLGTAGNCRLFIPGFAEMAAPLYPLTKPGTLFN |
| T330P | WGPDQQKAYQEIKQALLTAPALGLPDLTKPFELFVDEKQGYAKGVLT |
| L435G | QKLGPWRRPVAYLSKKLDPVAAGWPPCLRMVAAIAVLTKDAGKLTM |
| N454K | GQPLVIGAPHAVEALVKQPPDRWLSKARMTHYQALLLDTDRVQFGPV |
| D524G | VALNPATLLPLPEEGLQHNCLDILAEAHGTRPDLTDQPLPDADHTWYT |
| D583N | GGSSLLQEGQRKAGAAVTTETEVIWAKALPAGTSAQRAELIALTQALK |
| H594Q | MAEGKKLNVYTNSRYAFATAHIQGEIYRRRGLLTSEGKEIKNKDEILA |
| D653N | LLKALFLPKRLSIIHCPGHQKGHSAEARGNRMANQAARKAAITETPDT |
| | STLLIENSSP (SEQ ID NO: 139) |
| M-MLV RT | TLNIEDEYRLHETSKEPDVSLGSTWLSDFPQAWAETGGMGLAVRQAP |
| D200N P51L | LIILLKATSTPVSIKQYPMKQEARLGIKPHIQRLLDQGILVPCQSPWNTP |
| S67K T197A | LLPVKKPGTNDYRPVQDLREVNKRVEDIHPTVPNPYNLLSGLPPSHQW |
| H204R | YTVLDLKDAFFCLRLHPTSQPLFAFEWRDPEMGISGQLTWTRLPQGFK |
| E302K | NSPALFNEALRRDLADFRIQHPDLILLQYVDDLLLAATSELDCQQGTR |
| F309N | ALLQTLGNLGYRASAKKAQICQKQVKYLGYLLKEGQRWLTEARKET |
| W313F | VMGQPTPKTPRQLRKFLGTAGNCRLFIPGFAEMAAPLYPLTKPGTLFN |
| T330P L345G | WGPDQQKAYQEIKQALLTAPALGLPDLTKPFELFVDEKQGYAKGVLT |
| N454K | QKLGPWRRPVAYLSKKLDPVAAGWPPCLRMVAAIAVLTKDAGKLTM |
| D524G | GQPLVIGAPHAVEALVKQPPDRWLSKARMTHYQALLLDTDRVQFGPV |
| D583N | VALNPATLLPLPEEGLQHNCLDILAEAHGTRPDLTDQPLPDADHTWYT |
| H594Q | GGSSLLQEGQRKAGAAVTTETEVIWAKALPAGTSAQRAELIALTQALK |
| D653N | MAEGKKLNVYTNSRYAFATAHIQGEIYRRRGLLTSEGKEIKNKDEILA |
| | LLKALFLPKRLSIIHCPGHQKGHSAEARGNRMANQAARKAAITETPDT |
| | STLLIENSSP (SEQ ID NO: 140) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| T306K | SPTLFNEALHRDLADFRIQHPDLILLQYVDDLLLAATSELDCQQGTRAL |
| W313F | LQTLGNLGYRASAKKAQICQKQVKYLGYLLKEGQRWLTEARKETVM |
| | GQPTPKTPRQLREFLGKAGFCRLFIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 141) |

In various other embodiments, the prime editor fusion proteins described herein (with RT provided as either a fusion partner or in trans) can include a variant RT comprising one or more of the following mutations: P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X in the wild type M-MLV RT of SEQ ID NO: 124, or at a corresponding amino acid position in another wild type RT polypeptide sequence, wherein “X” can be any amino acid.

In various other embodiments, the prime editor fusion proteins described herein (with RT provided as either a fusion partner or in trans) can include a variant RT comprising a P51X mutation in the wild type M-MLV RT of SEQ ID NO: 124, or at a corresponding amino acid position in another wild type RT polypeptide sequence, wherein “X” can be any amino acid. In certain embodiments, X is L.

In various other embodiments, the prime editor fusion proteins described herein (with RT provided as either a fusion partner or in trans) can include a variant RT comprising an S67X mutation in the wild type M-MLV RT of SEQ ID NO: 124, or at a corresponding amino acid position in another wild type RT polypeptide sequence, wherein “X” can be any amino acid. In certain embodiments, X is K.

In various other embodiments, the prime editor fusion proteins described herein (with RT provided as either a fusion partner or in trans) can include a variant RT comprising an E69X mutation in the wild type M-MLV RT of SEQ ID NO: 124, or at a corresponding amino acid position in another wild type RT polypeptide sequence, wherein “X” can be any amino acid. In certain embodiments, X is K.

In various other embodiments, the prime editor fusion proteins described herein (with RT provided as either a fusion partner or in trans) can include a variant RT comprising an L139X mutation in the wild type M-MLV RT of SEQ ID NO: 124, or at a corresponding amino acid position in another wild type RT polypeptide sequence, wherein “X” can be any amino acid. In certain embodiments, X is P.

In various other embodiments, the prime editor fusion proteins described herein (with RT provided as either a fusion partner or in trans) can include a variant RT comprising a T197X mutation in the wild type M-MLV RT of SEQ ID NO: 124, or at a corresponding amino acid position in another wild type RT polypeptide sequence, wherein “X” can be any amino acid. In certain embodiments, X is A.

In various other embodiments, the prime editor fusion proteins described herein (with RT provided as either a fusion partner or in trans) can include a variant RT comprising a D200X mutation in the wild type M-MLV RT of SEQ ID NO: 124, or at a corresponding amino acid position in another wild type RT polypeptide sequence, wherein “X” can be any amino acid. In certain embodiments, X is N.

In various other embodiments, the prime editor fusion proteins described herein (with RT provided as either a fusion partner or in trans) can include a variant RT comprising an H204X mutation in the wild type M-MLV RT of SEQ ID NO: 124, or at a corresponding amino acid position in another wild type RT polypeptide sequence, wherein “X” can be any amino acid. In certain embodiments, X is R.

In various other embodiments, the prime editor fusion proteins described herein (with RT provided as either a fusion partner or in trans) can include a variant RT comprising an F209X mutation in the wild type M-MLV RT of SEQ ID NO: 124, or at a corresponding amino acid position in another wild type RT polypeptide sequence, wherein “X” can be any amino acid. In certain embodiments, X is N.

In various other embodiments, the prime editor fusion proteins described herein (with RT provided as either a fusion partner or in trans) can include a variant RT comprising an E302X mutation in the wild type M-MLV RT of SEQ ID NO: 124, or at a corresponding amino acid position in another wild type RT polypeptide sequence, wherein “X” can be any amino acid. In certain embodiments, X is K.

In various other embodiments, the prime editor fusion proteins described herein (with RT provided as either a fusion partner or in trans) can include a variant RT comprising an E302X mutation in the wild type M-MLV RT of SEQ ID NO: 124, or at a corresponding amino acid position in another wild type RT polypeptide sequence, wherein “X” can be any amino acid. In certain embodiments, X is R.

In various other embodiments, the prime editor fusion proteins described herein (with RT provided as either a fusion partner or in trans) can include a variant RT comprising a T306X mutation in the wild type M-MLV RT of SEQ ID NO: 124, or at a corresponding amino acid position in another wild type RT polypeptide sequence, wherein “X” can be any amino acid. In certain embodiments, X is K.

In various other embodiments, the prime editor fusion proteins described herein (with RT provided as either a fusion partner or in trans) can include a variant RT comprising an F309X mutation in the wild type M-MLV RT of SEQ ID NO: 124, or at a corresponding amino acid position in another wild type RT polypeptide sequence, wherein “X” can be any amino acid. In certain embodiments, X is N.

In various other embodiments, the prime editor fusion proteins described herein (with RT provided as either a fusion partner or in trans) can include a variant RT comprising a W313X mutation in the wild type M-MLV RT of SEQ ID NO: 124, or at a corresponding amino acid position in another wild type RT polypeptide sequence, wherein “X” can be any amino acid. In certain embodiments, X is F.

In various other embodiments, the prime editor fusion proteins described herein (with RT provided as either a fusion partner or in trans) can include a variant RT comprising a T330X mutation in the wild type M-MLV RT of SEQ ID NO: 124, or at a corresponding amino acid position in another wild type RT polypeptide sequence, wherein “X” can be any amino acid. In certain embodiments, X is P.

In various other embodiments, the prime editor fusion proteins described herein (with RT provided as either a fusion partner or in trans) can include a variant RT comprising an L345X mutation in the wild type M-MLV RT of SEQ ID NO: 124, or at a corresponding amino acid position in another wild type RT polypeptide sequence, wherein “X” can be any amino acid. In certain embodiments, X is G.

In various other embodiments, the prime editor fusion proteins described herein (with RT provided as either a fusion partner or in trans) can include a variant RT comprising an L435X mutation in the wild type M-MLV RT of SEQ ID NO: 124, or at a corresponding amino acid position in another wild type RT polypeptide sequence, wherein “X” can be any amino acid. In certain embodiments, X is G.

In various other embodiments, the prime editor fusion proteins described herein (with RT provided as either a fusion partner or in trans) can include a variant RT comprising an N454X mutation in the wild type M-MLV RT of SEQ ID NO: 124, or at a corresponding amino acid position in another wild type RT polypeptide sequence, wherein “X” can be any amino acid. In certain embodiments, X is K.

In various other embodiments, the prime editor fusion proteins described herein (with RT provided as either a fusion partner or in trans) can include a variant RT comprising a D524X mutation in the wild type M-MLV RT of SEQ ID NO: 124, or at a corresponding amino acid position in another wild type RT polypeptide sequence, wherein “X” can be any amino acid. In certain embodiments, X is G.

In various other embodiments, the prime editor fusion proteins described herein (with RT provided as either a fusion partner or in trans) can include a variant RT comprising an E562X mutation in the wild type M-MLV RT of SEQ ID NO: 124, or at a corresponding amino acid position in another wild type RT polypeptide sequence, wherein “X” can be any amino acid. In certain embodiments, X is Q.

In various other embodiments, the prime editors fusion proteins described herein (with RT provided as either a fusion partner or in trans) can include a variant RT comprising a D583X mutation in the wild type M-MLV RT of SEQ ID NO: 124, or at a corresponding amino acid position in another wild type RT polypeptide sequence, wherein “X” can be any amino acid. In certain embodiments, X is N.

In various other embodiments, the prime editor fusion proteins described herein (with RT provided as either a fusion partner or in trans) can include a variant RT comprising an H594X mutation in the wild type M-MLV RT of SEQ ID NO: 124, or at a corresponding amino acid position in another wild type RT polypeptide sequence, wherein “X” can be any amino acid. In certain embodiments, X is Q.

In various other embodiments, the prime editor fusion proteins described herein (with RT provided as either a fusion partner or in trans) can include a variant RT comprising an L603X mutation in the wild type M-MLV RT of SEQ ID NO: 124, or at a corresponding amino acid position in another wild type RT polypeptide sequence, wherein “X” can be any amino acid. In certain embodiments, X is W.

In various other embodiments, the prime editor fusion proteins described herein (with RT provided as either a fusion partner or in trans) can include a variant RT comprising an E607X mutation in the wild type M-MLV RT of SEQ ID NO: 124, or at a corresponding amino acid position in another wild type RT polypeptide sequence, wherein “X” can be any amino acid. In certain embodiments, X is K.

In various other embodiments, the prime editor fusion proteins described herein (with RT provided as either a fusion partner or in trans) can include a variant RT comprising a D653X mutation in the wild type M-MLV RT of SEQ ID NO: 124, or at a corresponding amino acid position in another wild type RT polypeptide sequence, wherein “X” can be any amino acid. In certain embodiments, X is N.

Some exemplary reverse transcriptases that can be fused to napDNAbp proteins (e.g., any of the Cas14a1 variants described herein) or provided as individual proteins according to various embodiments of this disclosure are provided below. Exemplary reverse transcriptases include variants with at least 80%, at least 85%, at least 90%, at least 95%, or at least 99% sequence identity to the wild-type enzymes or partial enzymes described in SEQ ID NOs: 124-141.

The present disclosure contemplates the use of any publicly-available reverse transcriptase described or disclosed in any of the following U.S. patents (each of which are incorporated by reference in their entireties) in the fusion proteins provided herein: U.S. Pat. Nos. 10,202,658; 10,189,831; 10,150,955; 9,932,567; 9,783,791; 9,580,698; 9,534,201; and 9,458,484, and any variant thereof that can be made using known methods for installing mutations, or known methods for evolving proteins. The following references also describe reverse transcriptases known in the art, the disclosures of each of which are incorporated herein by reference.
Herzig, E., Voronin, N., Kucherenko, N. & Hizi, A. A Novel Leu92 Mutant of HIV-1 Reverse Transcriptase with a Selective Deficiency in Strand Transfer Causes a Loss of Viral Replication. J. Virol. 89, 8119-8129 (2015).
Mohr, G. et al. A Reverse Transcriptase-Cas1 Fusion Protein Contains a Cas6 Domain Required for Both CRISPR RNA Biogenesis and RNA Spacer Acquisition. Mol. Cell 72, 700-714.e8 (2018).
Zhao, C., Liu, F. & Pyle, A. M. An ultraprocessive, accurate reverse transcriptase encoded by a metazoan group II intron. RNA 24, 183-195 (2018).
Zimmerly, S. & Wu, L. An Unexplored Diversity of Reverse Transcriptases in Bacteria. Microbiol Spectr 3, MDNA3-0058-2014 (2015).
Ostertag, E. M. & Kazazian Jr, H. H. Biology of Mammalian L1 Retrotransposons. Annual Review of Genetics 35, 501-538 (2001).
Perach, M. & Hizi, A. Catalytic Features of the Recombinant Reverse Transcriptase of Bovine Leukemia Virus Expressed in Bacteria. Virology 259, 176-189 (1999).
Lim, D. et al. Crystal structure of the moloney murine leukemia virus RNase H domain. J. Virol. 80, 8379-8389 (2006).
Zhao, C. & Pyle, A. M. Crystal structures of a group II intron maturase reveal a missing link in spliceosome evolution. Nature Structural & Molecular Biology 23, 558-565 (2016).
Griffiths, D. J. Endogenous retroviruses in the human genome sequence. Genome Biol. 2, REVIEWS1017 (2001).
Baranauskas, A. et al. Generation and characterization of new highly thermostable and processive M-MuLV reverse transcriptase variants. Protein Eng Des Sel 25, 657-668 (2012).
Zimmerly, S., Guo, H., Perlman, P. S. & Lambowltz, A. M. Group II intron mobility occurs by target DNA-primed reverse transcription. Cell 82, 545-554 (1995).
Feng, Q., Moran, J. V., Kazazian, H. H. & Boeke, J. D. Human L1 retrotransposon encodes a conserved endonuclease required for retrotransposition. Cell 87, 905-916 (1996).
Berkhout, B., Jebbink, M. & Zsíros, J. Identification of an Active Reverse Transcriptase Enzyme Encoded by a Human Endogenous HERV-K Retrovirus. Journal of Virology 73, 2365-2375 (1999).
Kotewicz, M. L., Sampson, C. M., D'Alessio, J. M. & Gerard, G. F. Isolation of cloned Moloney murine leukemia virus reverse transcriptase lacking ribonuclease H activity. Nucleic Acids Res 16, 265-277 (1988).
Arezi, B. & Hogrefe, H. Novel mutations in Moloney Murine Leukemia Virus reverse transcriptase increase thermostability through tighter binding to template-primer. Nucleic Acids Res 37, 473-481 (2009).
Blain, S. W. & Goff, S. P. Nuclease activities of Moloney murine leukemia virus reverse transcriptase. Mutants with altered substrate specificities. J. Biol. Chem. 268, 23585-23592 (1993).
Xiong, Y. & Eickbush, T. H. Origin and evolution of retroelements based upon their reverse transcriptase sequences. EMBO J 9, 3353-3362 (1990).
Herschhorn, A. & Hizi, A. Retroviral reverse transcriptases. Cell. Mol. Life Sci. 67, 2717-2747 (2010).
Taube, R., Loya, S., Avidan, O., Perach, M. & Hizi, A. Reverse transcriptase of mouse mammary tumour virus: expression in bacteria, purification and biochemical characterization. Biochem. J. 329 (Pt 3), 579-587 (1998).
Liu, M. et al. Reverse Transcriptase-Mediated Tropism Switching in Bordetella Bacteriophage. Science 295, 2091-2094 (2002).
Luan, D. D., Korman, M. H., Jakubczak, J. L. & Eickbush, T. H. Reverse transcription of R2Bm RNA is primed by a nick at the chromosomal target site: a mechanism for non-LTR retrotransposition. Cell 72, 595-605 (1993).
Nottingham, R. M. et al. RNA-seq of human reference RNA samples using a thermostable group II intron reverse transcriptase. RNA 22, 597-613 (2016).
Telesnitsky, A. & Goff, S. P. RNase H domain mutations affect the interaction between Moloney murine leukemia virus reverse transcriptase and its primer-template. Proc. Natl. Acad. Sci. U.S.A. 90, 1276-1280 (1993).
Halvas, E. K., Svarovskaia, E. S. & Pathak, V. K. Role of Murine Leukemia Virus Reverse Transcriptase Deoxyribonucleoside Triphosphate-Binding Site in Retroviral Replication and In Vivo Fidelity. Journal of Virology 74, 10349-10358 (2000).
Nowak, E. et al. Structural analysis of monomeric retroviral reverse transcriptase in complex with an RNA/DNA hybrid. Nucleic Acids Res 41, 3874-3887 (2013).
Stamos, J. L., Lentzsch, A. M. & Lambowitz, A. M. Structure of a Thermostable Group II Intron Reverse Transcriptase with Template-Primer and Its Functional and Evolutionary Implications. Molecular Cell 68, 926-939.e4 (2017).
Das, D. & Georgiadis, M. M. The Crystal Structure of the Monomeric Reverse Transcriptase from Moloney Murine Leukemia Virus. Structure 12, 819-829 (2004).
Avidan, O., Meer, M. E., Oz, I. & Hizi, A. The processivity and fidelity of DNA synthesis exhibited by the reverse transcriptase of bovine leukemia virus. European Journal of Biochemistry 269, 859-867 (2002).
Gerard, G. F. et al. The role of template-primer in protection of reverse transcriptase from thermal inactivation. Nucleic Acids Res 30, 3118-3129 (2002).
Monot, C. et al. The Specificity and Flexibility of L1 Reverse Transcription Priming at Imperfect T-Tracts. PLOS Genetics 9, e1003499 (2013).
Mohr, S. et al. Thermostable group II intron reverse transcriptase fusion proteins and their use in cDNA synthesis and next-generation RNA sequencing. RNA 19, 958-970 (2013).

### Nuclear Localization Sequences (NLS)

In various embodiments, the Cas proteins described herein may be fused to one or more nuclear localization sequences (NLS), which help promote translocation of a protein into the cell nucleus. In some embodiments, the fusion proteins described herein may comprise one or more NLS. Such sequences are well-known in the art and can include the following examples:

| Description | Sequence | SEQ ID NO: |
|---|---|---|
| NLS of SV40 large T-Ag | PKKKRKV | 142 |
| NLS | MKRTADGSEFESPKKKRKV | 143 |
| NLS | MDSLLMNRRKFLYQFKNVRWAKG | 144 |
| | RRETYLC | |
| NLS of nucleoplasmin | AVKRPAATKKAGQAKKKKLD | 145 |
| NLS of EGL-13 | MSRRRKANPTKLSENAKKLAKEV | 146 |
| | EN | |
| NLS of c-MYC | PAAKRVKLD | 147 |
| NLS of TUS-protein | KLKIKRPVK | 148 |
| NLS of polyoma large T- | VSRKRPRP | 149 |
| Ag | | |
| NLS of Hepatitis D virus | EGAPPAKRAR | 150 |
| antigen | | |
| NLS of murine p53 | PPQPKKKPLDGE | 151 |
| NLS of PE1 and PE2 | SGGSKRTADGSEFEPKKKRKV | 152 |
| Bipartite sv40 nls | KRTADGSEFESPKKKRKV | 153 |

The NLS examples above are non-limiting. The fusion proteins provided herein may comprise any known NLS sequence, including any of those described in Cokol et al., “Finding nuclear localization signals,” EMBO Rep., 2000, 1(5): 411-415; and Freitas et al., “Mechanisms and Signals for the Nuclear Import of Proteins,” Current Genomics, 2009, 10(8): 550-7, each of which are incorporated herein by reference.

In various embodiments, the fusion proteins and constructs encoding the fusion proteins disclosed herein further comprise one or more, preferably at least two, nuclear localization sequences. In certain embodiments, the fusion proteins comprise at least two NLSs. In embodiments with at least two NLSs, the NLSs can be the same NLSs, or they can be different NLSs. In some embodiments, one or more of the NLSs are bipartite NLSs (“bpNLS”). In certain embodiments, the disclosed fusion proteins comprise two bipartite NLSs. In some embodiments, the disclosed fusion proteins comprise more than two bipartite NLSs.

The location of the NLS fusion can be at the N-terminus, the C-terminus, or within a sequence of a fusion protein (e.g., inserted between the encoded napDNAbp component (e.g., any of the Cas14a1 variants disclosed herein) and a deaminase domain (e.g., an adenosine or cytosine deaminase) or a reverse transcriptase domain).

The NLSs may be any known NLS sequence in the art. The NLSs may also be any future-discovered NLSs for nuclear localization. The NLSs also may be any naturally-occurring NLS, or any non-naturally occurring NLS (e.g., an NLS with one or more desired mutations).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., International PCT application PCT/EP2000/011690, filed Nov. 23, 2000, published as WO/2001/038547 on May 31, 2001, the contents of which are incorporated herein by reference. In some embodiments, an NLS comprises the amino acid sequence PKKKRKV (SEQ ID NO: 142), MDSLLMNRRKFLYQFKNVRWAKGRRETYLC (SEQ ID NO: 144), KRTADGSEFESPKKKRKV (SEQ ID NO: 153), or KRTADGSEFEPKKKRKV (SEQ ID NO: 155). In other embodiments, NLS comprises the amino acid sequences NLSKRPAAIKKAGQAKKKK (SEQ ID NO: 204), PAAKRVKLD (SEQ ID NO: 147), RQRRNELKRSF (SEQ ID NO: 205), or NQSSNFGPMKGGNFGGRSSGPYGGGGQYFAKPRNQGGY (SEQ ID NO: 206).

In one aspect of the disclosure, a base editor, prime editor, or other fusion protein may be modified with one or more nuclear localization sequences (NLS), preferably at least two NLSs. In certain embodiments, the fusion proteins are modified with two or more NLSs. The disclosure contemplates the use of any nuclear localization sequence known in the art at the time of the disclosure, or any nuclear localization sequence that is identified or otherwise made available in the state of the art after the time of the instant filing. A representative nuclear localization sequence is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273: 14731-37, incorporated herein by reference) to eight amino acids, and is typically rich in lysine and arginine residues (Magin et al., (2000) Virology 274: 11-16, incorporated herein by reference). Nuclear localization sequences often comprise proline residues. A variety of nuclear localization sequences have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett. 461:229-34, which is incorporated herein by reference. Translocation is currently thought to involve nuclear pore proteins.

Most NLSs can be classified in three general groups: (i) a monopartite NLS exemplified by the SV40 large T antigen NLS (PKKKRKV (SEQ ID NO: 142)); (ii) a bipartite motif consisting of two basic domains separated by a variable number of spacer amino acids and exemplified by the Xenopus nucleoplasmin NLS (KRXXXXXXXXXXKKKL (SEQ ID NO: 154)); and (iii) noncanonical sequences such as M9 of the hnRNP A1 protein, the influenza virus nucleoprotein NLS, and the yeast Gal4 protein NLS (Dingwall and Laskey 1991).

Nuclear localization sequences appear at various points in the amino acid sequences of proteins. NLS have been identified at the N-terminus, the C-terminus, and in the central region of proteins. Thus, the disclosure provides fusion proteins that may be modified with one or more NLSs at the C-terminus and/or the N-terminus, as well as at internal regions of the fusion protein. The residues of a longer sequence that do not function as component NLS residues should be selected so as not to interfere, for example, tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a fusion protein to include one or more NLSs. In one aspect, the fusion proteins may be engineered to express a fusion protein that is translationally fused at its N-terminus or its C-terminus (or both) to one or more NLSs, i.e., to form a Cas protein-NLS fusion construct, base editor-NLS fusion construct, or prime editor-NLS fusion construct. In other embodiments, a fusion protein-encoding nucleotide sequence may be genetically modified to incorporate a reading frame that encodes one or more NLSs in an internal region of the encoded base editor. In addition, the NLSs may include various amino acid linkers or spacer regions encoded between the fusion protein and the N-terminally, C-terminally, or internally-attached NLS amino acid sequence, e.g., and in the central region of proteins. Thus, the present disclosure also provides for nucleotide constructs, vectors, and host cells for expressing fusion proteins that comprise a base editor or prime editor and one or more NLSs, among other components.

The fusion proteins described herein may also comprise nuclear localization sequences that are linked to the fusion protein through one or more linkers, e.g., a polymeric, amino acid, nucleic acid, polysaccharide, chemical, or nucleic acid linker element. The linkers within the contemplated scope of the disclosure are not intended to have any limitations and can be any suitable type of molecule (e.g., polymer, amino acid, polysaccharide, nucleic acid, lipid, or any synthetic chemical linker domain) and can be joined to the fusion protein by any suitable strategy that effectuates forming a bond (e.g., covalent linkage, hydrogen bonding) between the fusion protein and the one or more NLSs.

### UGI Domains and Other Fusion Protein Components

In some aspects, the fusion proteins (e.g., base editors) described herein may comprise one or more uracil glycosylase inhibitor (UGI) domains. In some embodiments, the fusion proteins comprise two UGI domains. The UGI domain refers to a protein that is capable of inhibiting a uracil-DNA glycosylase base-excision repair enzyme.

In some embodiments, a UGI domain comprises a wild-type UGI or a UGI as set forth in SEQ ID NO: 28, or a variant thereof. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 28. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 28. In some embodiments, a UGI comprises an amino acid sequence homologous to the amino acid sequence set forth in SEQ ID NO: 28, or an amino acid sequence homologous to a fragment of the amino acid sequence set forth in SEQ ID NO: 28. In some embodiments, proteins comprising UGI, fragments of UGI, or homologs of UGI are referred to as “UGI variants.” A UGI variant shares homology to UGI, or a fragment thereof. For example, a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 28. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 28. In some embodiments, the UGI comprises the following amino acid sequence:

>sp|P14739|UNGI_BPPB2 Uracil-DNA glycosylase inhibitor MTNLSDIIEKETGKQLVIQESILMLPEEVEEVIGNKPESDILVHTAYDESTDENVMLLTSD APEYKPWALVIQDSNGENKIKML (SEQ ID NO: 28).

The fusion proteins (e.g., base editors) described herein also may include one or more additional elements. In certain embodiments, an additional element may comprise an effector of base repair, such as an inhibitor of base repair.

In some embodiments, the base editors described herein may comprise one or more heterologous protein domains (e.g., about, or more than about, 1, 2, 3, 4, 5, 6, 7, 8, 9, 10, or more domains in addition to the base editor components). A base editor may comprise any additional protein sequence, and optionally a linker sequence between any two domains. Other exemplary features that may be present are localization sequences, such as cytoplasmic localization sequences, export sequences, such as nuclear export sequences, or other localization sequences, as well as sequence tags.

Examples of protein domains that may be fused to a base editor or component thereof (e.g., the napDNAbp domain, the cytidine deaminase domain, or the NLS domain) include, without limitation, epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A base editor may be fused to a gene sequence encoding a protein or a fragment of a protein that binds DNA molecules or binds other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that may form part of a base editor are described in U.S. Patent Publication No. 2011/0059502, published Mar. 10, 2011, and incorporated herein by reference in its entirety.

The reporter gene sequences that may be used with the base editors, methods and systems disclosed herein include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), HSV thymidine kinase, rpoB, may be introduced into a cell to encode a gene into which a mutation may be introduced that will confer resistance to a particular medium in a growth selection assay for the described system.

Other exemplary features that may be present are tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, bgh-PolyA tags, polyhistidine tags, and also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein may comprise one or more His tags.

### Linkers

The fusion proteins described herein may include one or more linkers. As defined above, the term “linker,” as used herein, refers to a chemical group or a molecule linking two molecules or moieties, e.g., a binding domain and a cleavage domain of a nuclease. In some embodiments, a linker joins a gRNA binding domain of an RNA-programmable nuclease and the catalytic domain of a deaminase (e.g., a cytosine deaminase or an adenosine deaminase). In some embodiments, a linker joins a Cas14a1 variant provided herein and a deaminase. In some embodiments, a linker joins a Cas14a1 protein provided herein and a reverse transcriptase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The linker may be as simple as a covalent bond, or it may be a polymeric linker many atoms in length. In certain embodiments, the linker is a polypeptide, or amino acid-based. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS)ₙ (SEQ ID NO: 156), (G)ₙ(SEQ ID NO: 157), (EAAAK)ₙ (SEQ ID NO: 158), (GGS)ₙ (SEQ ID NO: 159), (SGGS)ₙ(SEQ ID NO: 160), (XP)ₙ (SEQ ID NO: 161), or any combination thereof, wherein n is independently an integer between 1 and 30, and wherein X is any amino acid. In some embodiments, the linker comprises the amino acid sequence (GGS)n(SEQ ID NO: 159), wherein n is 1, 3, or 7. In some embodiments, the linker comprises the amino acid sequence SGSETPGTSESATPES (SEQ ID NO: 162). In some embodiments, the linker comprises the amino acid sequence SGSETPGTSESA (SEQ ID NO: 163). In some embodiments, the linker comprises the amino acid sequence SGSETPGTSESATPEGGSGGS (SEQ ID NO: 164). In some embodiments, the linker comprises the amino acid sequence SGGSSGGSSGSETPGTSESATPESSGGSSGGS (SEQ ID NO: 165). In some embodiments, the linker comprises the amino acid sequence SGGSGGSGGS (SEQ ID NO: 166). In some embodiments, the linker comprises the amino acid sequence SGGS (SEQ ID NO: 1). In other embodiments, the linker comprises the amino acid sequence SGGSSGGSSGSETPGTSESATPESAGSYPYDVPDYAGSAAPAAKKKKLDGSGSGGSSGG S (SEQ ID NO: 167, 60AA). In some embodiments, the linker comprises the amino acid sequence GGS, GGSGGS (SEQ ID NO: 168), GGSGGSGGS (SEQ ID NO: 169), SGGSSGGSSGSETPGTSESATPESSGGSSGGSS (SEQ ID NO: 170), SGSETPGTSESATPES (SEQ ID NO: 162), or SGGSSGGSSGSETPGTSESATPESAGSYPYDVPDYAGSAAPAAKKKKLDGSGSGGSSGG S (SEQ ID NO: 171).

In certain embodiments, linkers may be used to link any of the peptides or peptide domains or moieties of the invention (e.g., a napDNAbp linked or fused to a deaminase domain or a reverse transcriptase). Any of the domains of the fusion proteins described herein may also be connected to one another through any of the presently described linkers.

Guide RNAs (gRNAs)

In various embodiments, the Cas proteins and fusion proteins provided herein may be complexed, bound, or otherwise associated with (e.g., via any type of covalent or non-covalent bond) one or more guide sequences, i.e., the guide sequence becomes associated or bound to the Cas protein or fusion protein and directs its localization to a specific target sequence having complementarity to the guide sequence or a portion thereof. The design of a guide sequence will depend upon the nucleotide sequence of a genomic target site of interest (i.e., the desired site to be edited) and the type of napDNAbp (e.g., type of Cas protein), among other factors, such as PAM sequence locations, percent G/C content in the target sequence, the degree of microhomology regions, secondary structures, etc.

In some aspects, the present disclosure provides engineered Cas14a1 gRNAs. As described herein, the inventors have found that rational engineering of the Cas14a1 guide RNA significantly increased robust activity of Cas14a1 and the variants disclosed herein in human cells. In particular, it was found that Cas14a1 gRNAs comprising mutations in a particular poly-U region of the wild-type Cas14a1 gRNA backbone sequence are compatible with Cas14a1 and result in increased activity of the Cas14a1 variants disclosed herein. In some embodiments, the UUUUU region of the Cas14a1 gRNA backbone sequence is mutated to UUUCC. In some embodiments, the UUUUU region of the Cas14a1 gRNA backbone sequence is mutated to UUCUU. In some embodiments, the UUUUU region of the Cas14a1 gRNA backbone sequence is mutated to UAUUU. In some embodiments, the UUUUU region of the Cas14a1 gRNA backbone sequence is mutated to UUUCA.

The wild-type Cas14a1 gRNA comprises the following sequence, with the poly-U sequence discussed above underlined:

(SEQ ID NO: 172)
CUUCACUGAUAAAGUGGAGAACCGCUUCACCAAAAGCUGUCCCUUAGGG
GAUUAGAACUUGAGUGAAGGUGGGCUGCUUGCAUCAGCCUAAUGUCGAG
AAGUGCUUUCUUCGGAAAGUAACCCUCGAAACAAAUUCAUUUUUCCUCU
CCAAUUCUGCACAAGAAAGUUGCAGAACCCGAAUAGACGAAUGAAGGAA
UGCAAC

Thus, in some aspects, the present disclosure provides gRNAs comprising a nucleic acid sequence of any one of the following nucleotide sequences:

Engineered Cas14a1 sgRNA 1:

(SEQ ID NO: 173)
CUUCACUGAUAAAGUGGAGAACCGCUUCACCAAAAGCUGUCCCUUAGGG
GAUUAGAACUUGAGUGAAGGUGGGCUGCUUGCAUCAGCCUAAUGUCGAG
AAGUGCUUUCUUCGGAAAGUAACCCUCGAAACAAAUUCAUUUCCUCUCC
AAUUCUGCACAAGAAAGUUGCAGAACCCGAAUAGAAAUGAAGGAAUGCA
AC

Engineered Cas14a1 sgRNA 2:

(SEQ ID NO: 174)
CUUCACUGAUAAAGUGGAGAACCGCUUCACCAAAAGCUGUCCCUUAGGG
GAUUAGAACUUGAGUGAAGGUGGGCUGCUUGCAUCAGCCUAAUGUCGAG
AAGUGCUUUCUUCGGAAAGUAACCCUCGAAACAAAUUCAUUCUUCCUCU
CCAAUUCUGCACAAGAAAGUUGCAGAACCCGAAUAGACGAAUGAAGGAA
UGCAAC

Engineered Cas14a1 sgRNA 3:

(SEQ ID NO: 175)
CUUCACUGAUAAAGUGGAGAACCGCUUCACCAAAAGCUGUCCCUUAGGG
GAUUAGAACUUGAGUGAAGGUGGGCUGCUUGCAUCAGCCUAAUGUCGAG
AAGUGCUUUCUUCGGAAAGUAACCCUCGAAACAAAUUCAUAUUUCCUCU
CCAAUUCUGCACAAGAAAGUUGCAGAACCCGAAUAGACGUAUGAAGGAA
UGCAAC

Engineered Cas14a1 sgRNA 4:

(SEQ ID NO: 176)
CUUCACUGAUAAAGUGGAGAACCGCUUCACCAAAAGCUGUCCCUUAGGG
GAUUAGAACUUGAGUGAAGGUGGGCUGCUUGCAUCAGCCUAAUGUCGAG
AAGUGCUUUCUUCGGAAAGUAACCCUCGAAACAAAUUCAUUUCAAGAAA
GUGAAUGAAGGAAUGCAAC

In some embodiments, the gRNA comprises a nucleic acid sequence of any one of SEQ ID NOs: 173-176, or a nucleic acid sequence that is at least 70%, at least 75%, at least 80%, at least 85%, at least 90%, at least 95%, or at least 99% identical to the nucleic acid sequence of any one of SEQ ID NOs: 173-176. In some embodiments, the gRNA comprises a nucleic acid sequence that is 100% identical to the nucleic acid sequence of any one of SEQ ID NOs: 173-176. In certain embodiments, the gRNA comprises the nucleic acid sequence of engineered Cas14a1 sgRNA 4 provided above (SEQ ID NO: 176).

In some embodiments, the gRNA exhibits increased expression from a U6 promoter compared to a wild-type Cas14a1 gRNA. In certain embodiments, the backbone sequence of the gRNA comprises one or more substitutions relative to a wild-type Cas14a1 gRNA. In certain embodiments, the portions of the gRNA besides the backbone sequence do not comprise any substitutions relative to a wild-type Cas14a1 gRNA.

The sequences of suitable guide RNAs for targeting the Cas proteins and fusion proteins described herein to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein. Additional guide sequences are well known in the art and can be used with the fusion proteins described herein.

In general, a guide sequence is any polynucleotide sequence having sufficient complementarity with a target polynucleotide sequence to hybridize with the target sequence and direct sequence-specific binding of a napDNAbp (e.g., Cas14a1, or a Cas14a1 variant disclosed herein) to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

In some embodiments, a guide sequence is less than about 75, 50, 45, 40, 35, 30, 25, 20, 15, 12, or fewer nucleotides in length. The ability of a guide sequence to direct sequence-specific binding of a fusion protein to a target sequence may be assessed by any suitable assay. For example, the components of a fusion protein, including the guide sequence to be tested, may be provided to a host cell having the corresponding target sequence, such as by transfection with vectors encoding the components of a fusion protein disclosed herein, followed by an assessment of preferential cleavage within the target sequence, such as by Surveyor assay. Similarly, cleavage of a target polynucleotide sequence may be evaluated in a test tube by providing the target sequence, components of a fusion protein, including the guide sequence to be tested and a control guide sequence different from the test guide sequence, and comparing binding or rate of cleavage at the target sequence between the test and control guide sequence reactions. Other assays are possible, and will be apparent to those skilled in the art.

In various embodiments, a guide RNA may comprise additional components for use with a fusion protein comprising a Cas protein and a reverse transcriptase (i.e., a prime editor). Such guide RNAs may be referred to herein as prime editing guide RNAs (PEgRNAs) or extended guide RNAs.

In some embodiments, an extended guide RNA is used in the prime editor fusion proteins disclosed herein (e.g., comprising any of the Cas14a1 variants provided herein and a reverse transcriptase). A traditional guide RNA includes a ˜20 nt protospacer sequence and a gRNA core region, which binds with the napDNAbp. In some embodiments, the guide RNA includes an extended RNA segment at the 5′ end, i.e., a 5′ extension. In some embodiments, the 5′ extension includes a reverse transcription template sequence, a reverse transcription primer binding site, and an optional 5-20 nucleotide linker sequence. The RT primer binding site hybridizes to the free 3′ end that is formed after a nick is formed in the non-target strand of the R-loop, thereby priming reverse transcriptase for DNA polymerization in the 5′-3′ direction.

In some embodiments, the guide RNA includes an extended RNA segment at the 3′ end, i.e., a 3′ extension. In some embodiments, the 3′ extension includes a reverse transcription template sequence, and a reverse transcription primer binding site. The RT primer binding site hybridizes to the free 3′ end that is formed after a nick is formed in the non-target strand of the R-loop, thereby priming reverse transcriptase for DNA polymerization in the 5′-3′ direction.

In some embodiments, the guide RNA includes an extended RNA segment at an intermolecular position within the gRNA core, i.e., an intramolecular extension. In some embodiments, the intramolecular extension includes a reverse transcription template sequence, and a reverse transcription primer binding site. The RT primer binding site hybridizes to the free 3′ end that is formed after a nick is formed in the non-target strand of the R-loop, thereby priming reverse transcriptase for DNA polymerization in the 5′-3′ direction.

In one embodiment, the position of the intermolecular RNA extension is not in the protospacer sequence of the guide RNA. In another embodiment, the position of the intermolecular RNA extension in the gRNA core. In still another embodiment, the position of the intermolecular RNA extension is anywhere within the guide RNA molecule except within the protospacer sequence, or at a position which disrupts the protospacer sequence. In one embodiment, the intermolecular RNA extension is inserted downstream from the 3′ end of the protospacer sequence. In another embodiment, the intermolecular RNA extension is inserted at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, or at least 25 nucleotides downstream of the 3′ end of the protospacer sequence.

The length of the RNA extension (which includes at least the RT template and primer binding site) can be any useful length. In various embodiments, the RNA extension is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The RT template sequence can also be any suitable length. For example, the RT template sequence can be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In still other embodiments, the reverse transcription primer binding site sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In other embodiments, the optional linker or spacer sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The RT template sequence, in certain embodiments, encodes a single-stranded DNA molecule that is homologous to the non-target strand (and thus, complementary to the corresponding site of the target strand) but includes one or more nucleotide changes. The one or more nucleotide changes may include one or more single-base nucleotide changes, one or more deletions, and/or one or more insertions.

The synthesized single-stranded DNA product of the RT template sequence is homologous to the non-target strand and contains one or more nucleotide changes. The single-stranded DNA product of the RT template sequence hybridizes in equilibrium with the complementary target strand sequence, thereby displacing the homologous endogenous target strand sequence. The displaced endogenous strand may be referred to in some embodiments as a 5′ endogenous DNA flap species. This 5′ endogenous DNA flap species can be removed by a 5′ flap endonuclease (e.g., FEN1), and the single-stranded DNA product, now hybridized to the endogenous target strand, may be ligated, thereby creating a mismatch between the endogenous sequence and the newly synthesized strand. The mismatch may be resolved by the cell's innate DNA repair and/or replication processes.

In various embodiments, the nucleotide sequence of the RT template sequence corresponds to the nucleotide sequence of the non-target strand that becomes displaced as the 5′ flap species and that overlaps with the site to be edited.

In various embodiments of the extended guide RNAs, the reverse transcription template sequence may encode a single-strand DNA flap that is complementary to an endogenous DNA sequence adjacent to a nick site, wherein the single-strand DNA flap comprises a desired nucleotide change. The single-stranded DNA flap may displace an endogenous single-strand DNA at the nick site. The displaced endogenous single-strand DNA at the nick site can have a 5′ end and form an endogenous flap, which can be excised by the cell. In various embodiments, excision of the 5′ end endogenous flap can help drive product formation, since removing the 5′ end endogenous flap encourages hybridization of the single-strand 3′ DNA flap to the corresponding complementary DNA strand, and the incorporation or assimilation of the desired nucleotide change carried by the single-strand 3′ DNA flap into the target DNA.

In various embodiments of the extended guide RNAs, the cellular repair of the single-strand DNA flap results in installation of the desired nucleotide change, thereby forming a desired product.

In still other embodiments, the desired nucleotide change is installed in an editing window that is between about −5 to +5 of the nick site, or between about −10 to +10 of the nick site, or between about −20 to +20 of the nick site, or between about −30 to +30 of the nick site, or between about −40 to +40 of the nick site, or between about −50 to +50 of the nick site, or between about −60 to +60 of the nick site, or between about −70 to +70 of the nick site, or between about −80 to +80 of the nick site, or between about −90 to +90 of the nick site, or between about −100 to +100 of the nick site, or between about −200 to +200 of the nick site.

In other embodiments, the desired nucleotide change is installed in an editing window that is between about +1 to +2 from the nick site, or about +1 to +3, +1 to +4, +1 to +5, +1 to +6, +1 to +7, +1 to +8, +1 to +9, +1 to +10, +1 to +11, +1 to +12, +1 to +13, +1 to +14, +1 to +15, +1 to +16, +1 to +17, +1 to +18, +1 to +19, +1 to +20, +1 to +21, +1 to +22, +1 to +23, +1 to +24, +1 to +25, +1 to +26, +1 to +27, +1 to +28, +1 to +29, +1 to +30, +1 to +31, +1 to +32, +1 to +33, +1 to +34, +1 to +35, +1 to +36, +1 to +37, +1 to +38, +1 to +39, +1 to +40, +1 to +41, +1 to +42, +1 to +43, +1 to +44, +1 to +45, +1 to +46, +1 to +47, +1 to +48, +1 to +49, +1 to +50, +1 to +51, +1 to +52, +1 to +53, +1 to +54, +1 to +55, +1 to +56, +1 to +57, +1 to +58, +1 to +59, +1 to +60, +1 to +61, +1 to +62, +1 to +63, +1 to +64, +1 to +65, +1 to +66, +1 to +67, +1 to +68, +1 to +69, +1 to +70, +1 to +71, +1 to +72, +1 to +73, +1 to +74, +1 to +75, +1 to +76, +1 to +77, +1 to +78, +1 to +79, +1 to +80, +1 to +81, +1 to +82, +1 to +83, +1 to +84, +1 to +85, +1 to +86, +1 to +87, +1 to +88, +1 to +89, +1 to +90, +1 to +90, +1 to +91, +1 to +92, +1 to +93, +1 to +94, +1 to +95, +1 to +96, +1 to +97, +1 to +98, +1 to +99, +1 to +100, +1 to +101, +1 to +102, +1 to +103, +1 to +104, +1 to +105, +1 to +106, +1 to +107, +1 to +108, +1 to +109, +1 to +110, +1 to +111, +1 to +112, +1 to +113, +1 to +114, +1 to +115, +1 to +116, +1 to +117, +1 to +118, +1 to +119, +1 to +120, +1 to +121, +1 to +122, +1 to +123, +1 to +124, or +1 to +125 from the nick site.

In still other embodiments, the desired nucleotide change is installed in an editing window that is between about +1 to +2 from the nick site, or about +1 to +5, +1 to +10, +1 to +15, +1 to +20, +1 to +25, +1 to +30, +1 to +35, +1 to +40, +1 to +45, +1 to +50, +1 to +55, +1 to +100, +1 to +105, +1 to +110, +1 to +115, +1 to +120, +1 to +125, +1 to +130, +1 to +135, +1 to +140, +1 to +145, +1 to +150, +1 to +155, +1 to +160, +1 to +165, +1 to +170, +1 to +175, +1 to +180, +1 to +185, +1 to +190, +1 to +195, or +1 to +200, from the nick site.

In various aspects, the extended guide RNAs are modified versions of a guide RNA. Guide RNAs maybe naturally occurring, expressed from an encoding nucleic acid, or synthesized chemically. Methods are well known in the art for obtaining or otherwise synthesizing guide RNAs, and for determining the appropriate sequence of the guide RNA, including the protospacer sequence that interacts and hybridizes with the target strand of a genomic target site of interest.

### Methods for Nucleic Acid Modification

Some aspects of the present disclosure provide methods of using the Cas proteins (e.g., any of the disclosed Cas14a1 variants), fusion proteins, and complexes provided herein.

In one aspect, the present disclosure provides methods for modifying (e.g., editing, cutting, nicking, recombining, or making epigenetic changes such as methylation or acetylation) a target nucleic acid molecule comprising contacting the target nucleic acid molecule with any of the fusion proteins provided herein and a gRNA (e.g., any of the gRNAs disclosed herein, including those of SEQ ID NOs: 172-176, or gRNAs comprising a nucleic acid sequence that is at least at least 70%, at least 75%, at least 80%, at least 85%, at least 90%, at least 95%, or at least 99% identical to the nucleic acid sequence of any one of SEQ ID NOs: 172-176).

In another aspect, the present disclosure provides methods for modifying (e.g., editing, cutting, nicking, recombining, or making epigenetic changes such as methylation or acetylation) a target nucleic acid molecule comprising contacting the target nucleic acid molecule with any of the complexes provided herein.

In some embodiments, the contacting step of any of the methods described herein is performed in vitro. In some embodiments, the contacting is performed in vivo. In certain embodiments, the contacting is performed in a subject. A subject may have been diagnosed with a disease or disorder, or be at risk for having a disease or disorder.

In some embodiments, the target sequence comprises a sequence associated with a disease or disorder. In some embodiments, the target sequence comprises a point mutation associated with a disease or disorder. In certain embodiments, the point mutation comprises aT→C point mutation associated with a disease or disorder. In certain embodiments, the point mutation comprises an A→G point mutation associated with a disease or disorder. In some embodiments, the step of editing the target nucleic acid results in correction of the point mutation. In some embodiments, the target sequence comprises a T→C point mutation associated with a disease or disorder, and deamination of the mutant C base results in a sequence that is not associated with a disease or disorder. In some embodiments, the target sequence comprises an A→G point mutation associated with a disease or disorder, and deamination of the C that is base-paired to the mutant G base results in a sequence that is not associated with a disease or disorder. In some embodiments, the target sequence encodes a protein, and the point mutation is in a codon that results in a change in the amino acid encoded by the mutant codon as compared to the wild-type codon. In some embodiments, deamination of the mutant C results in a change in the amino acid encoded by the mutant codon. In some embodiments, deamination of the mutant C results in a codon encoding the wild-type amino acid. In some embodiments, the target DNA sequence comprises a G→A point mutation associated with a disease or disorder, and the deamination of the mutant A base results in a sequence that is not associated with a disease or disorder. In some embodiments, the target DNA sequence comprises a C→T point mutation associated with a disease or disorder, and deamination of the A that is base-paired with the mutant T results in a sequence that is not associated with a disease or disorder. In some embodiments, the target DNA sequence encodes a protein, and the point mutation is in a codon that results in a change in the amino acid encoded by the mutant codon as compared to the wild-type codon. In some embodiments, deamination of the mutant A results in a change in the amino acid encoded by the mutant codon. In some embodiments, deamination of the mutant A results in a codon encoding the wild-type amino acid.

In some embodiments, the fusion protein is used to replace a sequence associated with a disease or disorder with a sequence that is not associated with a disease or disorder (e.g., when the fusion protein comprises a reverse transcriptase and is a prime editor).

In some embodiments, the disease or disorder is a proliferative disease or disorder. In some embodiments, the disease or disorder is a genetic disease or disorder. In some embodiments, the disease or disorder is a neoplastic disease or disorder. In some embodiments, the disease or disorder is a metabolic disease or disorder. In some embodiments, the disease or disorder is a lysosomal storage disease or disorder. In some embodiments, the disease or disorder is cystic fibrosis, phenylketonuria, epidermolytic hyperkeratosis (EHK), Charcot-Marie-Toot disease type 4J, neuroblastoma (NB), von Willebrand disease (vWD), myotonia congenital, hereditary renal amyloidosis, dilated cardiomyopathy (DCM), hereditary lymphedema, familial Alzheimer's disease, HIV, Prion disease, chronic infantile neurologic cutaneous articular syndrome (CINCA), desmin-related myopathy (DRM), or a neoplastic disease associated with a mutant PI3KCA protein, a mutant CTNNB1 protein, a mutant HRAS protein, or a mutant p53 protein. Other diseases that can be treated by correcting a point mutation or introducing a deactivating mutation into a disease-associated gene will be known to those of skill in the art, and the disclosure is not limited in this respect.

In some aspects, the present disclosure contemplates use of any of the Cas proteins, fusion proteins, gRNAs complexes, systems, polynucleotides, vectors, and/or pharmaceutical compositions disclosed herein in the manufacture of a medicament for the treatment of a disease or disorder. In some aspects, any of the Cas proteins, fusion proteins, gRNAs, complexes, systems, polynucleotides, vectors, and/or pharmaceutical compositions disclosed herein are for use in medicine.

### Methods of Treatment

The present disclosure provides methods for the treatment of a subject diagnosed with a disease associated with or caused by a point mutation that can be corrected by a fusion protein provided herein (e.g., a base editor fusion protein comprising any of the Cas14a1 variants described herein, and a deaminase). For example, in some embodiments, a method is provided that comprises administering to a subject having such a disease, e.g., a disease such as cancer associated with a point mutation, an effective amount of a base editor, and a gRNA that forms a complex with the base editor, that corrects the point mutation or introduces a deactivating mutation into a disease-associated gene. In some embodiments, a method is provided that comprises administering to a subject having such a disease, e.g., a cancer associated with a point mutation, an effective amount of a base editor-gRNA complex that corrects the point mutation or introduces a deactivating mutation into a disease-associated gene. Further provided herein are methods comprising administering to a subject one or more vectors that contains a nucleotide sequence that expresses the base editor and gRNA that forms a complex with the base editor.

In some embodiments, the disease is a proliferative disease. In some embodiments, the disease is a genetic disease. In some embodiments, the disease is a neoplastic disease. In some embodiments, the disease is a metabolic disease. Other diseases that can be treated by correcting a point mutation or introducing a deactivating mutation into a disease-associated gene will be known to those of skill in the art, and the disclosure is not limited in this respect.

The present disclosure provides methods for the treatment of additional diseases or disorders, e.g., diseases or disorders that are associated or caused by a point mutation that can be corrected by base editing. Some such diseases are described herein, and additional suitable diseases that can be treated with the strategies and fusion proteins (e.g., base editors) provided herein will be apparent to those of skill in the art based on the present disclosure. Exemplary suitable diseases and disorders are listed below. It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues. Exemplary suitable diseases and disorders include, without limitation: Non-Bruton type Agammaglobulinemia, Hypomyelinating Leukodystrophy, 21-hydroxylase deficiency, familial Breast-ovarian cancer, Immunodeficiency with basal ganglia calcification, Congenital myasthenic syndrome, Shprintzen-Goldberg syndrome, Peroxisome biogenesis disorder, Nephronophthisis, autosomal recessive early-onset, digenic, PINK1/DJ1 Parkinson disease, Cerebral visual impairment and intellectual disability, Neurodevelopmental disorder with or without anomalies of the brain, eye, or heart, Immunodeficiency, Leber congenital amaurosis, Amyotrophic lateral sclerosis type 10, Motor neuron disease, Malignant melanoma of skin, Focal cortical dysplasia type II, papillary Renal cell carcinoma, Glioblastoma, Colorectal Neoplasms, Uterine cervical neoplasms, sporadic Papillary renal cell carcinoma, Malignant neoplasm of body of uterus, Kidney Carcinoma, Neoplasm of the breast, Glioblastoma, Smith-Kingsmore syndrome, Homocysteinemia due to MTHFR deficiency, type 2A2A Charcot-Marie-Tooth disease, Bartter syndrome type 3, Cataract, multiple types, Gastrointestinal stroma tumor, Paragangliomas, Pheochromocytoma, Hereditary cancer-predisposing syndrome, Paragangliomas, Hereditary cancer-predisposing syndrome, Gastrointestinal stroma tumor, Paragangliomas, Pheochromocytoma, Hereditary Paraganglioma-Pheochromocytoma Syndromes, Hereditary cancer-predisposing syndrome, Gastrointestinal stroma tumor, Paraganglioma and gastric stromal sarcoma, Uncombable hair syndrome, Parkinson's disease, autosomal recessive early-onset, Childhood hypophosphatasia, Odontohypophosphatasia, Takenouchi-Kosaki syndrome, C1q deficiency, Prostate cancer/brain cancer susceptibility, UDPglucose-4-epimerase deficiency, Deficiency of hydroxymethylglutaryl-CoA lyase, Fucosidosis, nonsyndromic cleft palate, Van der Woude syndrome, autosomal recessive Hypercholesterolemia, Eichsfeld type congenital muscular dystrophy, autosomal dominant Mental retardation, Hyperphosphatasia with mental retardation syndrome, Hyperphosphatasia-intellectual disability syndrome, Obesity, mild, early-onset, Ectodermal dysplasia, hypohidrotic/hair/tooth/nail type, Dystonia, torsion, autosomal recessive, Reticular dysgenesis, Erythrokeratodermia variabilis et progressiva, Corneal dystrophy, Fuchs endothelial, Corneal dystrophy, posterior polymorphous, Hereditary neutrophilia, Ceroid lipofuscinosis neuronal, Neuronal ceroid lipofuscinosis, Lethal tight skin contracture syndrome, DFNA 2 Nonsyndromic Hearing Loss, Osteogenesis imperfecta type 8, GLUT1 deficiency syndrome, autosomal recessive, Glucose transporter type 1 deficiency syndrome, Congenital amegakaryocytic thrombocytopenia, Myelofibrosis with myeloid metaplasia, somatic, Myelofibrosis with myeloid metaplasia, Thrombocythemia, somatic, Hematologic neoplasm, Early infantile epileptic encephalopathy, Mental retardation, autosomal recessive, Familial porphyria cutanea tarda, MYH-associated polyposis, Hereditary cancer-predisposing syndrome, MUTYH-associated polyposis, Hereditary cancer-predisposing syndrome, Methylmalonic acidemia with homocystinuria, Methylmalonic aciduria and homocystinuria, cblC type, digenic, Muscle eye brain disease, Congenital Muscular Dystrophy, alpha-dystroglycan related, Limb-Girdle Muscular Dystrophy, Recessive, Muscle eye brain disease, Congenital muscular dystrophy-dystroglycanopathy with brain and eye anomalies, type A3, Adenocarcinoma of the colon, Congenital primary aphakia, Hepatic failure, early-onset, and neurologic disorder due to cytochrome C oxidase deficiency, Carnitine palmitoyltransferase II deficiency, infantile, Carnitine palmitoyltransferase II deficiency, myopathic, stress-induced, Carnitine palmitoyltransferase II deficiency, Carnitine palmitoyltransferase II deficiency, myopathic, stress-induced, Sensorineural deafness with mild renal dysfunction, Bartter syndrome type 4, Hypercholesterolemia, autosomal dominant, Low density lipoprotein cholesterol level quantitative trait locus, Familial hypercholesterolemia, Hypocholesterolemia, Hypercholesterolemia, autosomal dominant, Familial hypercholesterolemia, Low density lipoprotein cholesterol level quantitative trait locus, Hypocholesterolemia, Lattice corneal dystrophy Type III, Epileptic encephalopathy, early infantile, Hypobetalipoproteinemia, familial, Congenital disorder of glycosylation type 1t, Leber congenital amaurosis, Retinitis pigmentosa, Medium-chain acyl-coenzyme A dehydrogenase deficiency, Dilated cardiomyopathy 1CC, Venous malformation, Aase syndrome, Stargardt disease, Cone-rod dystrophy, Retinitis pigmentosa, Stargardt disease, Congenital stationary night blindness, Retinal dystrophy, Nonsyndromic cleft lip with or without cleft palate, Glycogen storage disease type III, Glycogen storage disease IIIa, Intermediate maple syrup urine disease type 2, Maple syrup urine disease, Chorea, childhood-onset, with psychomotor retardation, Marshall syndrome, Stickler syndrome, type 2, Marshall/Stickler syndrome, Chudley-McCullough syndrome, Auriculocondylar syndrome, Pontocerebellar hypoplasia, type 9, Epileptic encephalopathy, early infantile, Spinocerebellar ataxia, Muscle AMP deaminase deficiency, Congenital giant melanocytic nevus, Liver cancer, Chronic lymphocytic leukemia, Neurocutaneous melanosis, Malignant melanoma of skin, Multiple myeloma, Neuroblastoma, Lung adenocarcinoma, Non-small cell lung cancer, Acute myeloid leukemia, Renal cell carcinoma, papillary, Neoplasm of brain, Cutaneous melanoma, Glioblastoma, Hepatocellular carcinoma, Transitional cell carcinoma of the bladder, Colorectal Neoplasms, Nasopharyngeal Neoplasms, Adrenocortical carcinoma, Adenocarcinoma of stomach, Ovarian Serous Cystadenocarcinoma, Malignant neoplasm of body of uterus, RAS Inhibitor response, Malignant lymphoma, non-Hodgkin, Medulloblastoma, Malignant melanoma of skin, Multiple myeloma, Acute myeloid leukemia, Myelodysplastic syndrome, Cutaneous melanoma, Transitional cell carcinoma of the bladder, Neoplasm, Colorectal Neoplasms, Adenocarcinoma of stomach, Cutaneous melanoma, Malignant melanoma of skin, Multiple myeloma, Acute myeloid leukemia, Noonan syndrome, Myelodysplastic syndrome, Cutaneous melanoma, Colorectal Neoplasms, Adenocarcinoma of stomach, Malignant neoplasm of body of uterus, Malignant melanoma of skin, Multiple myeloma, Non-small cell lung cancer, Acute myeloid leukemia, Myelodysplastic syndrome, Cutaneous melanoma, Colorectal Neoplasms, Adenocarcinoma of stomach, Malignant neoplasm of body of uterus, Secondary hypothyroidism, Cardiovascular phenotype, Neurodevelopmental disorder, mitochondrial, with abnormal movements and lactic acidosis, with or without seizures, beta-Hydroxysteroid dehydrogenase deficiency, Hajdu-Cheney syndrome, Hemochromatosis type 2A, Hemochromatosis type 1, Atrial fibrillation, familial, Nager syndrome, Severe congenital neutropenia, autosomal recessive, Retinitis pigmentosa, Neurodevelopmental disorder with microcephaly, hypotonia, and variable brain anomalies, Abnormality of brain morphology, Neurodevelopmental disorder with microcephaly, hypotonia, and variable brain anomalies, Paget disease of bone, White-sutton syndrome, Ichthyosis vulgaris, Dermatitis, atopic, Ichthyosis vulgaris, Mental retardation, autosomal dominant, Nemaline myopathy, Congenital myopathy with fiber type disproportion, Epilepsy, nocturnal frontal lobe, type 3, Aicardi-Goutieres syndrome, Gaucher disease, perinatal lethal, Gaucher's disease, type 1, Subacute neuronopathic Gaucher's disease, Pyruvate kinase deficiency of red cells, Mental retardation, autosomal dominant, myopathy, mitochondrial, and ataxia, Grange syndrome, Charcot-Marie-Tooth disease, type 2, Familial partial lipodystrophy, Hutchinson-Gilford progeria syndrome, childhood-onset, Charcot-Marie-Tooth disease, Charcot-Marie-Tooth disease, type 2, Familial partial lipodystrophy, Benign scapuloperoneal muscular dystrophy with cardiomyopathy, Mandibuloacral dysostosis, Dilated cardiomyopathy, Encephalopathy, progressive, early-onset, with brain edema and/or leukoencephalopathy, Infantile encephalopathy, Hereditary insensitivity to pain with anhidrosis, Familial medullary thyroid carcinoma, Hereditary insensitivity to pain with anhidrosis Spherocytosis, type 3, autosomal recessive, Spherocytosis, Recessive, Elliptocytosis, Hereditary pyropoikilocytosis, Elliptocytosis, Spherocytosis, Recessive, Enlarged vestibular aqueduct, Alternating hemiplegia of childhood, Autoimmune interstitial lung, joint, and kidney disease, Mitochondrial complex I deficiency, Charcot-Marie-Tooth disease, demyelinating, type 1b, Charcot-Marie-Tooth disease, type I, Roussy-Lévy syndrome, Neuropathy, congenital hypomyelinating, autosomal dominant, Charcot-Marie-Tooth disease, demyelinating, type 1b, Charcot-Marie-Tooth disease type 2J, Charcot-Marie-Tooth disease dominant intermediate, Charcot-Marie-Tooth disease, type I, Gastrointestinal stroma tumor, Paragangliomas, Hereditary cancer-predisposing syndrome, Achromatopsia, Thrombophilia due to activated protein C resistance, Geroderma osteodysplastica, Trimethylaminuria, FMO3 activity, decreased, Trimethylaminuria, Primary open angle glaucoma juvenile onset, Glaucoma, open angle, digenic, Glaucoma, primary congenital, digenic, MYOC-Related Disorders, Leukoencephalopathy with Brainstem and Spinal Cord Involvement and Lactate Elevation, Antithrombin III deficiency, Antithrombin deficiency, Antithrombin III deficiency, Hereditary nephrotic syndrome, Nephrotic syndrome, idiopathic, steroid-resistant, Pituitary hormone deficiency, combined, Glutamine deficiency, congenital, Prostate cancer, hereditary, Junctional epidermolysis bullosa gravis of Herlitz, Hyperparathyroidism, Factor H deficiency, Basal laminar drusen, CFHR5 deficiency, Factor XIII subunit B deficiency, Primary autosomal recessive microcephaly 5, Macular dystrophy, Leber congenital amaurosis, Retinitis pigmentosa, Leber congenital amaurosis, Macular dystrophy, Acute myeloid leukemia with maturation, Microcephaly, primary, autosomal recessive, Hypokalemic periodic paralysis, Left ventricular noncompaction, Familial hypertrophic cardiomyopathy, Left ventricular noncompaction, Familial restrictive cardiomyopathy, Cardiovascular phenotype, Renal dysplasia, Amelogenesis imperfecta, type IA, Popliteal pterygium syndrome, Van der Woude syndrome, Zimmermann-Laband syndrome, Leber congenital amaurosis, Stromme syndrome, Ciliary dyskinesia, primary, Usher syndrome, type 2A, Retinitis pigmentosa, Usher syndrome, Usher syndrome, type 2A, Retinal dystrophy, USH2A-Related Disorders, Usher syndrome, Blindness, Rod-cone dystrophy, Pigmentary retinopathy, Abnormal macular morphology, Retinal pigment epithelial atrophy, Loeys-Dietz syndrome, Holt-Oram syndrome, Cardiovascular phenotype, Martsolf syndrome, Warburg micro syndrome, Skraban-Deardorff syndrome, Coenzyme Q10 deficiency, primary, Multiple mitochondrial dysfunctions syndrome, Nemaline myopathy, Myopathy, scapulohumeroperoneal, Nemaline myopathy, autosomal dominant or recessive, Myopathy, actin, congenital, with cores, Cardioencephalomyopathy, fatal infantile, due to cytochrome c oxidase deficiency, Ch6diak-Higashi syndrome, Familial hypertrophic cardiomyopathy, Methylcobalamin deficiency, cblG type, Catecholaminergic polymorphic ventricular tachycardia, Catecholaminergic polymorphic ventricular tachycardia type 1, Catecholaminergic polymorphic ventricular tachycardia, Tooth agenesis, selective, Multiple cutaneous leiomyomas, Fumarase deficiency, Hereditary cancer-predisposing syndrome, Mental retardation, autosomal dominant, Diamond-Blackfan anemia, Maturity-onset diabetes of the young, type 7, Myoglobinuria, acute recurrent, autosomal recessive, Feingold syndrome, Cranioectodermal dysplasia, Short rib polydactyly syndrome, Jeune thoracic dystrophy, Short-rib thoracic dysplasia without polydactyly, Short-rib thoracic dysplasia with polydactyly, digenic, Multiple epiphyseal dysplasia, Familial hypobetalipoproteinemia, Familial hypercholesterolemia, Hypercholesterolemia, autosomal dominant, type B, Hypobetalipoproteinemia, familial, Hypobetalipoproteinemia, familial, Proopiomelanocortin deficiency, Acute myeloid leukemia, Shashi-Pena syndrome, Primary pulmonary hypertension 4, Navajo neurohepatopathy, Retinitis pigmentosa, Retinitis pigmentosa, Neuroblastoma, Neuroblastoma, Lung adenocarcinoma, Neuroblastoma, Non-small cell lung cancer, Benign Soft Tissue Neoplasm of Uncertain Differentiation, 3-Oxo-5 alpha-steroid delta 4-dehydrogenase deficiency, Spastic paraplegia, autosomal dominant, Glaucoma, primary congenital, Gingival fibromatosis, Noonan syndrome, Noonan syndrome, Rasopathy, Noonan syndrome, Short-rib thoracic dysplasia with polydactyly, Sitosterolemia, Cystinuria, Holoprosencephaly, Single median maxillary incisor, Schizencephaly, Erythrocytosis, familial, Factor v and factor viii, combined deficiency of, Multiple gastrointestinal atresias, Lynch syndrome, Lynch syndrome, Hereditary cancer-predisposing syndrome, Lynch syndrome, Hereditary nonpolyposis colon cancer, Lynch syndrome, Hereditary cancer-predisposing syndrome, Hereditary nonpolyposis colon cancer, Lynch syndrome, Hereditary cancer-predisposing syndrome, Hereditary nonpolyposis colorectal cancer type 5, Lynch syndrome, Hereditary cancer-predisposing syndrome, Colorectal cancer, non-polyposis, Hereditary nonpolyposis colon cancer, Leydig hypoplasia, type I, Leydig cell agenesis, Ovarian dysgenesis 1, Ovarian hyperstimulation syndrome, Combined oxidative phosphorylation deficiency, Intellectual developmental disorder with persistence of fetal hemoglobin, Bardet-Biedl syndrome, Multiple mitochondrial dysfunctions syndrome, Miyoshi muscular dystrophy, Limb-girdle muscular dystrophy, type 2B, Limb-girdle muscular dystrophy, type 2B, Miyoshi muscular dystrophy, Limb-girdle muscular dystrophy, type 2B, Dysferlinopathy, Limb-girdle muscular dystrophy, type 2B, Miyoshi muscular dystrophy, Limb-girdle muscular dystrophy, type 2B, Radiohumeral fusions with other skeletal and craniofacial anomalies, Sepiapterin reductase deficiency, Alstrom syndrome, Microcephaly-capillary malformation syndrome, Visceral myopathy, Chronic intestinal pseudoobstruction, Progressive external ophthalmoplegia with mitochondrial DNA deletions, autosomal recessive, Mitochondrial DNA-depletion syndrome, hepatocerebral, Congenital disorder of glycosylation type 2B, Vitamin k-dependent clotting factors, combined deficiency of, Surfactant metabolism dysfunction, pulmonary, Wolcott-Rallison dysplasia, Pheochromocytoma, Hereditary cancer-predisposing syndrome, Retinitis pigmentosa, Cone-rod dystrophy amelogenesis imperfecta, Cd8 deficiency, familial, Severe combined immunodeficiency, atypical, Achromatopsia, Monochromacy, Ectodermal dysplasia, hypohidrotic/hair/tooth type, autosomal dominant, Autosomal recessive hypohidrotic ectodermal dysplasia syndrome, Autosomal dominant hypohidrotic ectodermal dysplasia, Colorectal cancer with chromosomal instability, Retinitis pigmentosa, Osteomyelitis, sterile multifocal, with periostitis and pustulosis, Hypochromic microcytic anemia with iron overload, Culler-Jones syndrome, Autosomal recessive centronuclear myopathy, Thrombophilia, hereditary, due to protein C deficiency, autosomal dominant, Congenital disorders of glycosylation type II, Congenital disorder of glycosylation, type IIo, Warburg micro syndrome, Hypomyelination with brainstem and spinal cord involvement and leg spasticity, Warts, hypogammaglobulinemia, infections, and myelokathexis, Congenital NAD deficiency disorder, Vertebral, cardiac, renal, and limb defects syndrome, Mowat-Wilson syndrome, Homocystinuria, cb1D type, variant 1, Nemaline myopathy, Nemaline myopathy, Idiopathic generalized epilepsy, Epilepsy, idiopathic generalized, Juvenile myoclonic epilepsy, Episodic ataxia, type 5, Progressive myositis ossificans, Amelogenesis imperfecta, type IH, Benign familial neonatal-infantile seizures, Early infantile epileptic encephalopathy, Episodic ataxia, Early infantile epileptic encephalopathy, Seizures, Vertigo, Benign familial neonatal-infantile seizures, Mental retardation, autosomal dominant, Tumoral calcinosis, familial, hyperphosphatemic, Short rib-polydactyly syndrome, Majewski type, Severe myoclonic epilepsy in infancy, Generalized epilepsy with febrile seizures plus, type 2, Severe myoclonic epilepsy in infancy, Seizures, Delayed speech and language development, Early infantile epileptic encephalopathy, Severe myoclonic epilepsy in infancy, Familial hemiplegic migraine type 3, Paroxysmal extreme pain disorder, Hereditary sensory and autonomic neuropathy type IIA, Generalized epilepsy with febrile seizures plus, type 7, Rolandic epilepsy, Small fiber neuropathy, Primary erythromelalgia, Hereditary sensory and autonomic neuropathy type IIA, Generalized epilepsy with febrile seizures plus, type 7, Inherited Erythromelalgia, Primary erythromelalgia, Indifference to pain, congenital, autosomal recessive, Febrile seizures, familial, 3b, Benign recurrent intrahepatic cholestasis, Progressive familial intrahepatic cholestasis, Myasthenic syndrome, slow-channel congenital, Lethal multiple pterygium syndrome, Duane syndrome type 2, Synpolydactyly, Brachydactyly-syndactyly-oligodactyly syndrome, Brachydactyl-syndactyly-oligodactyly syndrome (1 patient), immunodeficiency, developmental delay, and hypohomocysteinemia, Hereditary myopathy with early respiratory failure, Familial dilated cardiomyopathy, Dilated cardiomyopathy, Primary dilated cardiomyopathy, Limb-girdle muscular dystrophy, type 2J, Primary dilated cardiomyopathy, Familial dilated cardiomyopathy, Familial hypertrophic cardiomyopathy, Diabetes mellitus type 2, Ehlers-Danlos syndrome, type 4, Cardiovascular phenotype, Ehlers-Danlos syndrome, type 2, Ehlers-Danlos syndrome, classic type, Hemochromatosis type 4, Immunodeficiency, Mycobacterial and viral infections, susceptibility to, autosomal recessive, Immunodeficiency, Mental retardation, autosomal recessive, Acute myeloid leukemia, Myelodysplastic syndrome, Myelodysplastic syndrome progressed to acute myeloid leukemia, Mitochondrial complex I deficiency, Joubert syndrome, Infantile-onset ascending hereditary spastic paralysis, ALS2-Related Disorders, Amyotrophic lateral sclerosis type 2, Pulmonary venoocclusive disease, Primary pulmonary hypertension, Autoimmune lymphoproliferatiVe syndrome, type V, Aculeiform cataract, Congenital cataract, Cataract, coppock-like, Liver cancer, Medulloblastoma, Malignant melanoma of skin, Multiple myeloma, Lung adenocarcinoma, Acute myeloid leukemia, Myelodysplastic syndrome, Neoplasm of brain, Neoplasm of the breast, Glioblastoma, Hepatocellular carcinoma, Transitional cell carcinoma of the bladder, Brainstem glioma, Colorectal Neoplasms, Adenoid cystic carcinoma, Adenocarcinoma of prostate, Hypotonia, infantile, with psychomotor retardation and characteristic facies, Congenital hyperammonemia, type I, Hereditary cancer-predisposing syndrome, Familial cancer of breast, Hereditary cancer-predisposing syndrome, Hereditary cancer-predisposing syndrome, Spondylometaphyseal dysplasia—Sutcliffe type, Spondylometaphyseal dysplasia, Short stature, Focal segmental glomerulosclerosis, Microcephaly, Small for gestational age, Disproportionate short-trunk short stature, Decreased body weight, Atrioventricular canal defect, Congenital microcephaly, Steroid-resistant nephrotic syndrome, Schimke immunoosseous dysplasia, Short stature, Focal segmental glomerulosclerosis, Microcephaly, Small for gestational age, Disproportionate short-trunk short stature, Decreased body weight, Atrioventricular canal defect, Congenital microcephaly, Steroid-resistant nephrotic syndrome, Gracile syndrome, Cholestanol storage disease, Odontoonychodermal dysplasia, Schopf-Schulz-Passarge syndrome, Tooth agenesis, selective, Type A1 brachydactyly, Dyschromatosis universalis hereditaria, Charcot-Marie-Tooth disease, axonal, type 2T, Myopathy, centronuclear, Three M syndrome, Waardenburg syndrome type 1, Alport syndrome, autosomal recessive, Benign familial hematuria, Basal ganglia disease, biotin-responsive, ARMC9-related Joubert syndrome, ARMC9-related Joubert syndrome, Jourbert syndrome, Arthrogryposis, distal, type 5d, Microphthalmia, isolated, Myasthenic syndrome, congenital, fast-channel, Congenital myasthenic syndrome, fast-channel, Oguchi's disease, Crigler Najjar syndrome, type 1, Crigler-Najjar syndrome, type II, Crigler-Najjar syndrome, Crigler-Najjar syndrome, type II, Gilbert's syndrome, Crigler Najjar syndrome, type 1, Hyperbilirubinemia, Ullrich congenital muscular dystrophy, Bethlem myopathy, Ullrich congenital muscular dystrophy, Bethlem myopathy, Primary hyperoxaluria, type I, D-2-hydroxyglutaric aciduria, Sideroblastic anemia with B-cell immunodeficiency, periodic fevers, and developmental delay, Multiple sulfatase deficiency, Gillespie syndrome, Limb-girdle muscular dystrophy, type 1C, Rippling muscle disease, Familial partial lipodystrophy, Severe congenital neutropenia, autosomal recessive, Severe congenital neutropenia, Von Hippel-Lindau syndrome, Hereditary cancer-predisposing syndrome, Erythrocytosis, familial, Von Hippel-Lindau syndrome, Erythrocytosis, familial, Von Hippel-Lindau syndrome, Renal cell carcinoma, papillary, Metabolic syndrome, susceptibility to, Obesity, age at onset of, Morbid obesity, Noonan syndrome, Rasopathy, Xeroderma pigmentosum, group C, Endplate acetylcholinesterase deficiency, Biotinidase deficiency, Thyroid hormone resistance, generalized, autosomal dominant, Thyroid hormone resistance, selective pituitary, Microphthalmia, syndromic, Congenital disorder of deglycosylation, Cardiovascular phenotype, Loeys-Dietz syndrome, Thoracic aortic aneurysm and aortic dissection, Congenital disorder of glycosylation type 1x, Mucopolysaccharidosis, MPS-IV-B, Osteogenesis imperfecta type 7, Lynch syndrome I, Hereditary cancer-predisposing syndrome, Turcot syndrome, Hereditary nonpolyposis colon cancer, Atrial fibrillation, Atrial fibrillation, familial, Atrial fibrillation, Brugada syndrome, Congenital long QT syndrome, Cardiac arrhythmia, Sudden infant death syndrome, Long qt syndrome, acquired, susceptibility to, Long QT syndrome, Romano-Ward syndrome, Brugada syndrome, Sick sinus syndrome, Progressive familial heart block, Cardiovascular phenotype, Paroxysmal familial ventricular fibrillation, Dilated Cardiomyopathy, Dominant, Long QT syndrome, Congenital long QT syndrome, Cardiac conduction defect, nonprogressive, Cardiac conduction defect, nonspecific, Brugada syndrome, Asplenia, isolated congenital, Liver cancer, Medulloblastoma, Malignant melanoma of skin, Pilomatrixoma, Hepatoblastoma, Hepatocellular carcinoma, Transitional cell carcinoma of the bladder, Uterine cervical neoplasms, Craniopharyngioma, Adenocarcinoma of stomach, Malignant neoplasm of body of uterus, Adenocarcinoma of prostate, Liver cancer, Medulloblastoma, Malignant melanoma of skin, Pilomatrixoma, Lung adenocarcinoma, Carcinoma of colon, Endometrial neoplasm, Hepatocellular carcinoma, Pancreatic adenocarcinoma, Transitional cell carcinoma of the bladder, Carcinoma of esophagus, Colorectal Neoplasms, Adenocarcinoma of stomach, Malignant neoplasm of body of uterus, Adenocarcinoma of prostate, Liver cancer, Medulloblastoma, Malignant melanoma of skin, Squamous cell carcinoma of the head and neck, Malignant tumor of prostate, Lung adenocarcinoma, Hepatoblastoma, Cutaneous melanoma, Hepatocellular carcinoma, Craniopharyngioma, Adrenocortical carcinoma, Adenocarcinoma of stomach, Malignant neoplasm of body of uterus, Liver cancer, Medulloblastoma, Lung adenocarcinoma, Neoplasm of stomach, Cutaneous melanoma, Hepatocellular carcinoma, Transitional cell carcinoma of the bladder, Carcinoma of esophagus, Uterine cervical neoplasms, Adenocarcinoma of stomach, Malignant neoplasm of body of uterus, Adenocarcinoma of prostate, Liver cancer, Malignant melanoma of skin, Lung adenocarcinoma, Cutaneous melanoma, Hepatocellular carcinoma, Transitional cell carcinoma of the bladder, Colorectal Neoplasms, Adrenocortical carcinoma, Malignant neoplasm of body of uterus, Adenocarcinoma of prostate, Nemaline myopathy, Spinocerebellar ataxia, autosomal recessive, Perrault syndrome, Hydrops, lactic acidosis, and sideroblastic anemia, Perrault syndrome, Bardet-Biedl syndrome, Bardet-Biedl syndrome, Failure of tooth eruption, primary, Gray platelet syndrome, Pretibial epidermolysis bullosa, Epidermolysis bullosa pruriginosa, autosomal dominant, Recessive dystrophic epidermolysis bullosa, Microcephaly, progressive, with seizures and cerebral and cerebellar atrophy, Epileptic encephalopathy, Nephrotic syndrome, type 5, with or without ocular abnormalities, Muscular dystrophy-dystroglycanopathy (congenital with brain and eye anomalies), type a, Tumor susceptibility linked to germline BAP1 mutations, Dilated cardiomyopathy 1Z, Dilated cardiomyopathy 1S, Familial hypertrophic cardiomyopathy, Cardiovascular phenotype, Familial hypertrophic cardiomyopathy, Hypogonadotropic hypogonadism with anosmia, Atelosteogenesis type 1, Atelosteogenesis type 3, Spondylocarpotarsal synostosis syndrome, Nemaline myopathy, Mental retardation with language impairment and with or without autistic features, Glycogen storage disease, type IV, Glycogen storage disease IV, congenital neuromuscular, Glycogen storage disease, type IV, Frontotemporal Dementia, Chromosome 3-Linked, Amyotrophic lateral sclerosis, Pituitary hormone deficiency, combined 1, Joubert syndrome, Neuropathy, hereditary motor and sensory, Okinawa type, Macular dystrophy, vitelliform, Retinitis pigmentosa, Combined oxidative phosphorylation deficiency, Spermatogenic failure, epileptic encephalopathy, infantile or early childhood, Alkaptonuria, Senior-Loken syndrome, Leber congenital amaurosis, Nephronophthisis, congenital deafness, Hypocalciuric hypercalcemia, familial, type 1, Neonatal severe hyperparathyroidism, Hypocalcemia, autosomal dominant, Hypocalciuric hypercalcemia, familial, type 1, Neonatal severe hyperparathyroidism, Hypocalciuric hypercalcemia, familial, type 1, Hypocalcemia, autosomal dominant, Hypocalcemia, autosomal dominant, with bartter syndrome, Dyskinesia, familial, with facial myokymia, Visceral myopathy, Lymphedema, primary, with myelodysplasia, Dendritic cell, monocyte, B lymphocyte, and natural killer lymphocyte deficiency, Acyl-CoA dehydrogenase family, member, deficiency of, Retinitis pigmentosa, Retinitis pigmentosa, autosomal recessive, Congenital stationary night blindness, autosomal dominant, Familial benign pemphigus, Epileptic encephalopathy, early infantile, Adolescent nephronophthisis, Primary hypertrophic osteoarthropathy, autosomal recessive, Myopathy, myofibrillar, Propionyl-CoA carboxylase deficiency, Blepharophimosis, ptosis, and epicanthus inversus, Seckel syndrome, Bruck syndrome, Craniosynostosis, Deficiency of ferroxidase, Usher syndrome, type 3A, Usher syndrome, type 3A, Retinitis pigmentosam, Deficiency of butyrylcholine esterase, BCHE, fluoride, Retinitis pigmentosa, Fanconi-Bickel syndrome, Short stature, idiopathic, autosomal, Liver cancer, Malignant melanoma of skin, Squamous cell carcinoma of the head and neck, Small cell lung cancer, Lung adenocarcinoma, Squamous cell lung carcinoma, Renal cell carcinoma, papillary, Neoplasm of brain, Neoplasm of the breast, Glioblastoma, Hepatocellular carcinoma, Pancreatic adenocarcinoma, Transitional cell carcinoma of the bladder, Brainstem glioma, Carcinoma of esophagus, PIK3CA related overgrowth spectrum, Colorectal Neoplasms, Uterine cervical neoplasms, Papillary renal cell carcinoma, sporadic, Nasopharyngeal Neoplasms, Adenocarcinoma of stomach, Ovarian Serous Cystadenocarcinoma, Malignant neoplasm of body of uterus, Adenocarcinoma of prostate, Uterine Carcinosarcoma, Carcinoma of gallbladder, Lung cancer, Medulloblastoma, Malignant melanoma of skin, Squamous cell carcinoma of the head and neck, Malignant tumor of prostate, Ovarian epithelial cancer, Carcinoma of colon, Neoplasm of brain, Neoplasm of the breast, Glioblastoma, Transitional cell carcinoma of the bladder, PIK3CA related overgrowth spectrum, Ovarian Neoplasms, Colorectal Neoplasms, Uterine cervical neoplasms, Adenocarcinoma of stomach, Malignant neoplasm of body of uterus, Adenocarcinoma of prostate, Uterine Carcinosarcoma, Cowden syndrome, PIK3CA related overgrowth spectrum, Colorectal Neoplasms, Ciliary dyskinesia, Ciliary dyskinesia, primary, Microphthalmia syndromic, Methylcrotonyl-CoA carboxylase deficiency, Epidermolysis bullosa simplex, Koebner type, Epidermolysis bullosa simplex, generalized, with scarring and hair loss, Leukoencephalopathy with vanishing white matter, Congenital disorder of glycosylation type 1D, Woolly hair, autosomal recessive, with or without hypotrichosis, Membranous cataract, Myopia, high, with cataract and vitreoretinal degeneration, Primary hypomagnesemia, Dominant hereditary optic atrophy, Autosomal dominant optic atrophy plus syndrome, Dominant hereditary optic atrophy, Abortive cerebellar ataxia, Dominant hereditary optic atrophy, Retinitis pigmentosa, Retinal dystrophy, Congenital stationary night blindness, autosomal dominant, Retinitis pigmentosa, Retinitis pigmentosa, Epileptic encephalopathy, early infantile, Abnormality of brain morphology, Dysostosis multiplex, Mucopolysaccharidosis type I, Hypochondroplasia, Thanatophoric dysplasia type 1, Epidermal nevus, Bladder carcinoma, Achondroplasia, Crouzon syndrome with acanthosis nigricans, Craniosynostosis, Carcinoma, Thanatophoric dysplasia type 1, Achondroplasia, Hypochondroplasia, Craniosynostosis, Camptodactyly, tall stature, and hearing loss syndrome, Hypochondroplasia, Thanatophoric dysplasia type 1, Craniosynostosis, Fibrous dysplasia of jaw, Myasthenia, limb-girdle, familial, Selective tooth agenesis, Orofacial cleft, Hypoplastic enamel-onycholysis-hypohidrosis syndrome, Jeune thoracic dystrophy, Ellis-van Creveld Syndrome, Short rib-polydactyly syndrome, Majewski type, Chondroectodermal dysplasia, Short rib-polydactyly syndrome, Majewski type, Diabetes mellitus type 2, Diabetes mellitus and insipidus with optic atrophy and deafness, Wolfram syndrome, Joubert syndrome, Coach syndrome, Retinitis pigmentosa, Cone-rod dystrophy, Retinal dystrophy, Spastic paraplegia, autosomal recessive, Epileptic encephalopathy, early infantile, Retinitis pigmentosa, Limb-girdle muscular dystrophy, type 2E, Gastrointestinal stroma tumor, Gastrointestinal stromal tumor, familial, Cutaneous mastocytosis, Gastrointestinal stroma tumor, Cutaneous melanoma, Gastrointestinal stroma tumor, Acute myeloid leukemia, Hematologic neoplasm, Cutaneous melanoma, Congenital disorder of glycosylation type 1Q, Hypogonadotropic hypogonadism with or without anosmia, Epilepsy, progressive myoclonic, with or without renal failure, Cryptophthalmos syndrome, Hyaline fibromatosis syndrome, Deafness, autosomal dominant nonsyndromic sensorineural, with dentinogenesis imperfecta, Dentinogenesis imperfecta-Shield's type II, Dentinogenesis imperfecta-Shield's type III, Deafness, autosomal dominant nonsyndromic sensorineural, with dentinogenesis imperfecta, Basan syndrome, Adermatoglyphia, Type A2 brachydactyly, Acromesomelic dysplasia, Demirhan type, Fibular hypoplasia and complex brachydactyly, Brachydactyly, type a1, Abetalipoproteinaemia, SLC39A8 deficiency, congenital disorder of glycosylation, type IIn, Beta-D-mannosidosis, Sudden cardiac failure, infantile, Deficiency of 3-hydroxyacyl-CoA dehydrogenase, Hyperinsulinemic hypoglycemia, familial, Fibrosis of extraocular muscles, congenital, Cardiac arrhythmia, Cardiac arrhythmia, ankyrin B-related, Long QT syndrome, Cardiovascular phenotype, Cardiac arrhythmia, Cardiac arrhythmia, ankyrin B-related, Long QT syndrome, Arrhythmia, Cardiovascular phenotype, Bardet-Biedl syndrome, Van Maldergem syndrome, short-rib thoracic dysplasia with polydactyly, Ceroid lipofuscinosis neuronal, Macular dystrophy with central cone involvement, Ceroid lipofuscinosis neuronal, Methylmalonic aciduria cblA type, Pseudohypoaldosteronism type 1 autosomal dominant, Pseudohypoaldosteronism, Common variable immunodeficiency, with autoimmunity, Afibrinogenemia, congenital, Familial visceral amyloidosis, Ostertag type, Hypodysfibrinogenemia, congenital, Afibrinogenemia, congenital, Glutaric acidemia IIC, Glutaric aciduria, type 2, Short rib-polydactyly syndrome, Majewski type, Dilated cardiomyopathy 1A, Limb-girdle muscular dystrophy, type 2S, Mitochondrial myopathy, Myopia, Mitochondrial DNA depletion syndrome (cardiomyopathic type), autosomal recessive, Progressive sensorineural hearing impairment, Hypertrophic cardiomyopathy, Left ventricular hypertrophy, Vertigo, Abnormality of mitochondrial metabolism, Mitochondrial respiratory chain defects, Bietti crystalline corneoretinal dystrophy, Corneal Dystrophy, Recessive, Bietti crystalline corneoretinal dystrophy, Hereditary factor XI deficiency disease, Mitochondrial complex II deficiency, Paragangliomas, Hereditary cancer-predisposing syndrome, Mitochondrial complex II deficiency, Dyskeratosis congenita autosomal dominant, Ciliary dyskinesia, Mental retardation, autosomal dominant, Chondrocalcinosis, Oculocutaneous albinism type 4, Inherited bone marrow failure syndrome, Bone marrow failure syndrome, Cornelia de Lange syndrome, Joubert syndrome, Orofaciodigital syndrome, Complement component deficiency, C7 and C6 deficiency, combined subtotal, Succinyl-CoA acetoacetate transferase deficiency, Laron syndrome with undetectable serum GH-binding protein, Laron-type isolated somatotropin defect, Levy-Hollister syndrome, Molybdenum cofactor deficiency, complementation group B, Distal hereditary motor neuronopathy type 2C, Kartagener syndrome, Acrodysostosis, with or without hormone resistance, UV-sensitive syndrome, Cockayne syndrome type A, Retinitis pigmentosa with or without skeletal anomalies, Immunodeficiency, Kugelberg-Welander disease, Werdnig-Hoffmann disease, 3-methylcrotonyl CoA carboxylase deficiency, Striatal degeneration, autosomal dominant, Hermansky Pudlak syndrome, Mucopolysaccharidosis, type vi, intermediate, Short stature, microcephaly, and endocrine dysfunction, Wagner syndrome, Basal cell carcinoma, somatic, Capillary malformation-arteriovenous malformation, Usher syndrome, type 2C, Febrile seizures, familial, Bosch-Boonstra-Schaaf optic atrophy syndrome, Proprotein convertase deficiency, Familial adenomatous polyposis, Familial colorectal cancer, Familial adenomatous polyposis, Familial adenomatous polyposis, Hereditary cancer-predisposing syndrome, Familial adenomatous polyposis, Colorectal cancer, susceptibility to, Familial adenomatous polyposis, Hereditary cancer-predisposing syndrome, Familial adenomatous polyposis, Hereditary cancer-predisposing syndrome, Familial adenomatous polyposis, Hereditary cancer-predisposing syndrome, Familial adenomatous polyposis, Anencephalus, Aortic aneurysm, familial thoracic, Pyridoxine-dependent epilepsy, Seizures, Ventriculomegaly, Pyridoxine-dependent epilepsy, Myopathy, areflexia, respiratory distress, and dysphagia, early-onset, Congenital contractural arachnodactyly, Neuromyotonia and axonal neuropathy, autosomal recessive, Renal camitine transport defect, Hereditary cancer-predisposing syndrome, Chylomicron retention disease, Groenouw corneal dystrophy type I, Reis-Bucklers' corneal dystrophy, Lattice corneal dystrophy type 3A, Lattice corneal dystrophy Type I, Pseudohypoaldosteronism, type 2, Pseudohypoaldosteronism type 2D, Myotilinopathy, Charcot-Marie-Tooth disease, axonal, type 2w, Leber congenital amaurosis, Retinitis pigmentosa, Diastrophic dysplasia, de la Chapelle dysplasia, Achondrogenesis, type IB, Multiple epiphyseal dysplasia, Diastrophic dysplasia, Hereditary diffuse leukoencephalopathy with spheroids, Infantile myofibromatosis, Mental retardation, autosomal recessive, Tay-Sachs disease, variant AB, Hyperglycinuria, Iminoglycinuria, digenic, Hyperekplexia hereditary, epileptic encephalopathy, early infantile, Autosomal recessive congenital ichthyosis, Congenital ichthyosiform erythroderma, Epilepsy, childhood absence, Familial febrile seizures, Leukodystrophy, hypomyelinating, Atrial septal defect with or without atrioventricular conduction defects, Ventricular septal defect, Primary dilated cardiomyopathy, Atrial septal defect, Ventricular fibrillation, Noncompaction cardiomyopathy, Abnormality of cardiovascular system morphology, Malformation of the heart and great vessels, Cardiovascular phenotype, Congenital heart disease, Atrial septal defect with or without atrioventricular conduction defects, Hypothyroidism, congenital, nongoitrous, Cardiovascular phenotype, Congenital heart disease, Craniosynostosis, Lewy body dementia, Sotos syndrome, Hypercalcemia, infantile, Hereditary angioneurotic edema with normal C1 esterase inhibitor activity, Hereditary angioneurotic edema, Acute myeloid leukemia, Myelodysplasia, Ehlers-Danlos syndrome progeroid type, Axenfeld-Rieger syndrome type 3, Polymicrogyria, asymmetric, Combined oxidative phosphorylation deficiency, Combined oxidative phosphorylation deficiency, Factor XIII subunit A deficiency, Cardiovascular phenotype, Bicuspid aortic valve, Arrhythmia, Sudden cardiac death, Ventricular fibrillation, Aortic dilatation, Bicuspid aortic valve, Branchiooculofacial syndrome, Hypoparathyroidism familial isolated, Auriculocondylar syndrome, Lafora disease, Hemochromatosis type 1, Transient neonatal diabetes mellitus, Michelin-tire baby, Combined oxidative phosphorylation deficiency, Peeling skin syndrome, Thrombocytopenia, anemia, and myelofibrosis, Premature ovarian failure, Sialidosis type I, 21-hydroxylase deficiency, Adenoma, cortisol-producing, Carcinoma, adrenocortical, androgen-secreting, Nakajo syndrome, Otospondylomegaepiphyseal dysplasia, Nonsyndromic Deafness, Mental retardation, autosomal dominant, Leber congenital amaurosis, Polycystic lipomembranous osteodysplasia with sclerosing leukoencephalopathy, Macular dystrophy, vitelliform, adult-onset, Retinitis pigmentosa, Choroidal dystrophy, central areolar, Glycine N-methyltransferase deficiency, Heimler syndrome, Three M syndrome, Xeroderma pigmentosum, variant type, Jaberi-Elahi syndrome, Ciliary dyskinesia, Platelet-activating factor acetylhydrolase deficiency, Methylmalonic aciduria due to methylmalonyl-CoA mutase deficiency, methylmalonic aciduria, mut(−) type, Methylmalonic aciduria due to methylmalonyl-CoA mutase deficiency, methylmalonic aciduria, mut(0) type, Methylmalonic acidemia, Methylmalonic aciduria due to methylmalonyl-CoA mutase deficiency, methylmalonic aciduria, mut(−) type Rh-null, regulator type, Rh-mod syndrome, Char syndrome, Autosomal recessive polycystic kidney disease, Polycystic kidney dysplasia, Autosomal recessive polycystic kidney disease, Spinocerebellar ataxia, Retinitis pigmentosa, Retinitis pigmentosa, mental retardation, autosomal dominant, Hydatidiform mole, recurrent, Deafness, autosomal dominant, Macular dystrophy, vitelliform, developmental delay, intellectual disability, obesity, and dysmorphic features, Leber congenital amaurosis, Maple syrup urine disease, Immunodeficiency, Hyper-IgE syndrome, Calcification of joints and arteries, Spinocerebellar ataxia, autosomal recessive, Forney Robinson Pascoe syndrome, Mitochondrial DNA depletion syndrome (encephalomyopathic type), North Carolina macular dystrophy, Spastic paraplegia and psychomotor retardation with or without seizures, Osteopetrosis, autosomal recessive, Amyotrophic lateral sclerosis type, Progressive pseudorheumatoid dysplasia, Metaphyseal chondrodysplasia, Schmid type, Ovarian dysgenesis, Alopecia congenita keratosis palmoplantaris, Oculodentodigital dysplasia, Merosin deficient congenital muscular dystrophy, Laminin alpha 2-related dystrophy, Merosin deficient congenital muscular dystrophy, Arginase deficiency, Arterial calcification of infancy, Hypophosphatemic rickets, autosomal recessive, Arterial calcification of infancy, Hypophosphatemic Rickets, Recessive, Arterial calcification of infancy, Joubert syndrome, Leber congenital amaurosis, Disseminated atypical mycobacterial infection, neurodegeneration with brain iron accumulation, Mental retardation, autosomal dominant, Congenital heart defects, multiple types, Mitochondrial diseases, Combined oxidative phosphorylation deficiency, Mitochondrial diseases, Estrogen resistance, Neoplasm of the breast, Spinocerebellar ataxia, autosomal recessive, Liver cancer, Hepatocellular carcinoma, Plasminogen deficiency, type I, Dysplasminogenemia, Plasminogen deficiency, type I, Parkinson disease, Dentin dysplasia, type I, with extreme microdontia and misshapen teeth, Ciliary dyskinesia, Spondylocostal dysostosis, Baraitser-Winter syndrome, Hereditary cancer-predisposing syndrome, Hereditary nonpolyposis colon cancer, Lynch syndrome, Neurodevelopmental abnormality, leukodystrophy, hypomyelinating, Congenital muscular dystrophy-dystroglycanopathy with brain and eye anomalies, type A7, Congenital muscular dystrophy-dystroglycanopathy with brain and eye anomalies, type A7, Muscular dystrophy-dystroglycanopathy (limb-girdle), type c, Congenital muscular dystrophy-dystroglycanopathy with brain and eye anomalies, type A7, Saethre-Chotzen syndrome, Ciliary dyskinesia, primary, Hypomyelination and Congenital Cataract, microtia without hearing impairment, Microtia, hearing impairment, and cleft palate, Isolated growth hormone deficiency type 1B, Uridine 5-prime monophosphate hydrolase deficiency, hemolytic anemia due to, Bardet-Biedl syndrome, Focal segmental glomerulosclerosis, Wilms tumor and radial bilateral aplasia, Pallister-Hall syndrome, Greig cephalopolysyndactyly syndrome, Pallister-Hall syndrome, Pallister-Hall syndrome, Hyperbiliverdinemia, Ehlers-Danlos syndrome, classic-like, Permanent neonatal diabetes mellitus, Maturity-onset diabetes of the young, type 2, Immunodeficiency, common variable, Cowden syndrome, Lung adenocarcinoma, Non-small cell lung cancer, Nonsmall cell lung cancer, response to tyrosine kinase inhibitor in, somatic, Glioblastoma, Non-small cell lung cancer, Squamous cell lung carcinoma, Carcinoma of esophagus, Non-small cell lung cancer, Mucopolysaccharidosis type VII, Argininosuccinate lyase deficiency, Epilepsy, progressive myoclonic, Shwachman syndrome, Disordered steroidogenesis due to cytochrome p450 oxidoreductase deficiency, Charcot-Marie-Tooth disease, Charcot-Marie-Tooth disease type 2F, Cholestasis, intrahepatic, of pregnancy, Progressive familial intrahepatic cholestasis, Progressive familial intrahepatic cholestasis, Intrahepatic cholestasis, Colchicine resistance, Cerebral cavernous malformation, Cerebral cavernous malformation, Cerebral cavernous malformations, Zellweger syndrome, Deafness enamel hypoplasia nail defects, Myelocerebellar disorder, COL1A2-Related Disorder, Ehlers-Danlos syndrome, classic type, Osteogenesis imperfecta type I, Osteogenesis imperfecta type III, Osteogenesis imperfecta type I, Osteogenesis imperfecta, recessive perinatal lethal, Ehlers-Danlos syndrome, classic type, Osteogenesis imperfecta type I, Osteogenesis imperfecta with normal sclerae, dominant form, Osteogenesis imperfecta type III, Osteogenesis imperfecta with normal sclerae, dominant form, Osteogenesis imperfecta type III, Ehlers-Danlos syndrome, autosomal recessive, cardiac valvular form, Neonatal intrahepatic cholestasis caused by citrin deficiency, Citrullinemia type II, Split-hand/foot malformation, Asparagine synthetase deficiency, Epilepsy, familial temporal lobe, Lissencephaly, Epilepsy, familial temporal lobe, Rolandic epilepsy, Epilepsy, familial temporal lobe, Enlarged vestibular aqueduct, Pendred's syndrome, Pendred's syndrome, Enlarged vestibular aqueduct, Pendred's syndrome, SLC26A4-Related Disorders, Enlarged vestibular aqueduct, Pendred's syndrome, Enlarged vestibular aqueduct, Congenital secretory diarrhea, chloride type, Maple syrup urine disease, type 3, DLD-Related Disorders, Lissencephaly, Lipodystrophy, congenital generalized, type 3, Renal cell carcinoma, papillary, Cystic fibrosis, Hereditary pancreatitis, Cystic fibrosis, Hereditary pancreatitis, ataluren response—Efficacy, Persistent hyperplastic primary vitreous, autosomal recessive, Atrophia bulborum hereditaria, Exudative vitreoretinopathy, Leptin dysfunction, Myofibrillar myopathy, filamin C-related, Myopathy, distal, Cardiomyopathy, familial hypertrophic, Dilated Cardiomyopathy, Dominant, Dilated Cardiomyopathy, Dominant, Basal cell carcinoma, somatic, Ghosal hematodiaphyseal syndrome, Multiple myeloma, Lung adenocarcinoma, Rasopathy, Glioblastoma, Transitional cell carcinoma of the bladder, Cardio-facio-cutaneous syndrome, Malignant melanoma of skin, Multiple myeloma, Lung adenocarcinoma, Non-small cell lung cancer, Squamous cell lung carcinoma, Squamous cell carcinoma of the skin, Transitional cell carcinoma of the bladder, Neoplasm, Colorectal Neoplasms, Adenocarcinoma of prostate, Lung cancer, Malignant melanoma of skin, Multiple myeloma, Squamous cell carcinoma of the head and neck, Lung adenocarcinoma, Non-small cell lung cancer, Squamous cell lung carcinoma, Colorectal Neoplasms, Non-small cell lung cancer, Rasopathy, Neoplasm of the breast, Neoplasm, Carcinoma of colon, Noonan syndrome, Cataract and cardiomyopathy, Myotonia congenital, Congenital myotonia, autosomal recessive form, Premature ovarian failure, Cortical dysplasia-focal epilepsy syndrome, Rolandic epilepsy, Pitt-Hopkins-like syndrome, Rolandic epilepsy, Long QT syndrome, Congenital long QT syndrome, Short QT syndrome, Cardiovascular phenotype, Long QT syndrome, Glaucoma, open angle, F, Glycogen storage disease of heart, lethal congenital, Familial hypertrophic cardiomyopathy, Primary familial hypertrophic cardiomyopathy, Holoprosencephaly, Currarino triad, Limb-girdle muscular dystrophy, type 1E, Neuronal ceroid lipofuscinosis, Maturity-onset diabetes of the young, type 11, Congenital heart disease, Atrial septal defect, Congenital heart disease, Atrial septal defect, Tetralogy of Fallot, Ventricular septal defect, Atrioventricular septal defect, Idiopathic transverse myelitis, Jankovic Rivera syndrome, Farber disease, Hyperlipoproteinemia, type I, Hyperlipoproteinemia, type I, lipoprotein lipase (Olbia), Surfactant metabolism dysfunction, pulmonary, Osteogenesis imperfecta, type xiii, Hypermanganesemia with dystonia, Charcot-Marie-Tooth disease, demyelinating, type 1f, Charcot-Marie-Tooth disease type 2E, Charcot-Marie-Tooth disease, demyelinating, type 1f, Trichothiodystrophy 6, nonphotosensitive, Cholesterol monooxygenase (side-chain cleaving) deficiency, Kallmann syndrome, Hartsfield syndrome, Medulloblastoma, Neuroblastoma, Encephalocraniocutaneous lipomatosis, Astrocytoma, Brainstem glioma, Adenocarcinoma of stomach, Rosette-forming glioneuronal tumor, Hypogonadotropic hypogonadism with anosmia, Spherocytosis type 1, Mental retardation, autosomal dominant, Idiopathic basal ganglia calcification, Basal ganglia calcification, idiopathic, Dystonia, torsion, Mucopolysaccharidosis, MPS-III-C, Retinitis pigmentosa, Mucopolysaccharidosis, MPS-III-C, Vesicoureteral reflux, CHARGE association, Ataxia with vitamin E deficiency, nocturnal frontal lobe epilepsy, Joubert syndrome, Melnick-Fraser syndrome, Osteopetrosis with renal tubular acidosis, carbonic anhydrase II variant, Achromatopsia, Hereditary cancer-predisposing syndrome, Microcephaly, normal intelligence and immunodeficiency, Microcephaly, normal intelligence and immunodeficiency, Joubert syndrome, Meckel syndrome type 3, Nephronophthisis, Meckel-Gruber syndrome, coach syndrome, Pyruvate dehydrogenase phosphatase deficiency, Carcinoma of colon, Leigh syndrome, multiple synostoses syndrome, Microphthalmia, isolated, Klippel-Feil syndrome, autosomal dominant, Leber congenital amaurosis, Klippel-Feil syndrome, autosomal dominant, Anauxetic dysplasia, Cohen syndrome, Cohen syndrome, Abnormality of the eye, Ciliary dyskinesia, primary, 28, Epilepsy, nocturnal frontal lobe, Corneal dystrophy, corneal dystrophy, posterior polymorphous, RRM2B-related mitochondrial disease, Mitochondrial DNA depletion syndrome, encephalomyopathic form, with renal tubulopathy, RRM2B-related mitochondrial disease, Nail disorder, nonsyndromic congenital, Nail disease, Dihydropyrimidinase deficiency, Tetraamelia syntrome, Trichorhinophalangeal dysplasia type I, Multiple congenital exostosis, Dandy-Walker like malformation with atrioventricular septal defect, Benign familial neonatal seizures, Ciliary dyskinesia, primary, Iodotyrosyl coupling defect, Mental retardation, autosomal recessive, Deficiency of steroid 11-beta-monooxygenase, Corticosterone methyloxidase type 1 deficiency, Hyperlipoproteinemia, type ID, Amelogenesis imperfecta, hypocalcification type, 5-Oxoprolinase deficiency, Mitochondrial complex III deficiency, nuclear type 6, Brown-Vialetto-Van Laere syndrome, Hereditary acrodermatitis enteropathica, Rothmund-Thomson syndrome, Baller-Gerold syndrome, Hyperimmunoglobulin E recurrent infection syndrome, autosomal recessive, Nicolaides-Baraitser syndrome, Cerebellar ataxia, mental retardation, and dysequilibrium syndrome, Retinal cone dystrophy, Familial erythrocytosis, Chronic myelogenous leukemia, Polycythemia vera, Budd-Chiari syndrome, Myelofibrosis, Budd-Chiari syndrome, susceptibility to, somatic, Acute myeloid leukemia, Thrombocythemia, Myeloproliferative disorder, Subacute lymphoid leukemia, Non-ketotic hyperglycinemia, Hydrocephalus, Melanoma-pancreatic cancer syndrome, Hereditary cutaneous melanoma, Hereditary cancer-predisposing syndrome, Cutaneous malignant melanoma, Hereditary cutaneous melanoma, Hereditary cancer-predisposing syndrome, Hereditary cutaneous melanoma, Melanoma-pancreatic cancer syndrome, Hereditary cutaneous melanoma, Hereditary cancer-predisposing syndrome, neurodevelopmental disorder with progressive microcephaly, spasticity, and brain anomalies, Bardet-Biedl syndrome, Glaucoma, primary congenital, Singleton-Merten syndrome, Ciliary dyskinesia, Distal spinal muscular atrophy, autosomal recessive, Deficiency of UDPglucose-hexose-1-phosphate uridylyltransferase, Deficiency of UDPglucose-hexose-1-phosphate uridylyltransferase, Galactosemia, Inclusion body myopathy with early-onset paget disease and frontotemporal dementia, Fanconi anemia, complementation group G, Metaphyseal chondrodysplasia, McKusick type, Acromesomelic dysplasia Maroteaux type, Inclusion body myopathy, Nonaka myopathy, Sialuria, GNE myopathy, Sialuria, Inclusion body myopathy, Nonaka myopathy, Sialuria, Primary hyperoxaluria, type II, Pontocerebellar hypoplasia, type 1b, Friedreich's ataxia, Progressive familial intrahepatic cholestasis, Hypomagnesemia, intestinal, Cone-rod dystrophy and hearing loss, Obesity, hyperphagia, and developmental delay, AGTPBP1-related condition, Type B brachydactyly, Fructose-biphosphatase deficiency, Fanconi anemia, Fanconi anemia, complementation group C, Hereditary cancer-predisposing syndrome, Gorlin syndrome, Gorlin syndrome, Hereditary cancer-predisposing syndrome, Xeroderma pigmentosum, type 1, Spondyloepimetaphyseal dysplasia Genevieve type, Early infantile epileptic encephalopathy 59, Loeys-Dietz syndrome, Thoracic aortic aneurysm and aortic dissection, Loeys-Dietz syndrome, Congenital disorder of glycosylation type 1, Hereditary fructosuria, Familial hypoalphalipoproteinemia, Tangier disease, Limb-girdle muscular dystrophy-dystroglycanopathy, type C4, Congenital muscular dystrophy-dystroglycanopathy with brain and eye anomalies, type A4, Primary autosomal recessive microcephaly, Meretoja syndrome, adrenal insufficiency, NR5A1-related, 46,XY sex reversal, type 3, Nail-patella syndrome, Early infantile epileptic encephalopathy 4, Epileptic encephalopathy, Primary pulmonary hypertension, Osler hemorrhagic telangiectasia syndrome, Coenzyme Q10 deficiency, primary, Ichthyosis prematurity syndrome, Congenital disorder of glycosylation type 1M, Citrullinemia type I, Citrullinemia type I, Citrullinemia, mild, Neuropathy, hereditary sensory and autonomic, type VIII, short stature, hearing loss, retinis pigmentosa, and distinctive facies, Cortical malformations, occipital, Limb-girdle muscular dystrophy-dystroglycanopathy, type C1, Congenital muscular dystrophy-dystroglycanopathy with mental retardation, type B1, Walker-Warburg congenital muscular dystrophy, Spinocerebellar ataxia autosomal recessive, Tuberous sclerosis syndrome, Tuberous sclerosis, Lymphangiomyomatosis, Congenital nonprogressive myopathy with Moebius and Robin sequences, Dopamine beta hydroxylase deficiency, Ehlers-Danlos syndrome, type 2, Ehlers-Danlos syndrome, classic type, Early infantile epileptic encephalopathy, Epilepsy, nocturnal frontal lobe, Joubert syndrome, Adams-Oliver syndrome, Aortic valve disorder, Adams-Oliver syndrome, Congenital generalized lipodystrophy type 1, Neurodevelopmental disorder with or without hyperkinetic movements and seizures, autosomal dominant, Autosomal recessive hypophosphatemic bone disease, Chromosome 9q deletion syndrome, Neoplasm of stomach, Prostate cancer, somatic, Refsum disease, adult, Severe combined immunodeficiency, athabascan-type, Renal adysplasia, Megaloblastic anemia due to inborn errors of metabolism, Primary ciliary dyskinesia, Kartagener syndrome, Desanto-shinawi syndrome, Neural tube defect, Familial medullary thyroid carcinoma, Multiple endocrine neoplasia, type 2, MEN2A and FMTC, Multiple endocrine neoplasia, type 2, MEN2A and Unclassified, MEN2A and FMTC, Multiple endocrine neoplasia, type 2, MEN2A and FMTC, Hereditary cancer-predisposing syndrome, Multiple endocrine neoplasia, type 2b, Familial medullary thyroid carcinoma, Multiple endocrine neoplasia, type 2a, Multiple endocrine neoplasia, type 2, MEN2A and FMTC, FMTC and Unclassified, Multiple endocrine neoplasia, type 2a, Hereditary cancer-predisposing syndrome, Pheochromocytoma, Familial medullary thyroid carcinoma, Multiple endocrine neoplasia, type 2a, Multiple endocrine neoplasia, type 2, MEN2A and FMTC, Medullary thyroid carcinoma, Multiple endocrine neoplasia, type 2a, Multiple endocrine neoplasia, type 2, MEN2 phenotype: Unknown, Hereditary cancer-predisposing syndrome, Multiple endocrine neoplasia, type 2b, Multiple endocrine neoplasia, type 2a, MEN2 phenotype: Unclassified, Multiple endocrine neoplasia, type 2, MEN2A and FMTC, Hereditary cancer-predisposing syndrome, Familial medullary thyroid carcinoma, Multiple endocrine neoplasia, type 2a, MEN2A and FMTC, Hereditary cancer-predisposing syndrome, Multiple endocrine neoplasia, Telangiectasia, hereditary hemorrhagic, type 5, Cockayne syndrome B, Premature ovarian failure, Familial infantile myasthenia, Charcot-Marie-Tooth disease, demyelinating, type 1d, Congenital hypomyelinating neuropathy, Neuropathy, congenital hypomyelinating, autosomal dominant, Shprintzen-Goldberg syndrome, Goldberg-Shprintzen megacolon syndrome, Shprintzen-Goldberg syndrome, Diarrhea, malabsorptive, congenital, Aplastic anemia, Hemophagocytic lymphohistiocytosis, familial, nephrotic syndrome, Hyperphenylalaninemia, BH4-deficient, D, Histiocytosis-lymphadenopathy plus syndrome, Usher syndrome, type 1D, pituitary adenoma, multiple types, Usher syndrome, type 1D, Usher syndrome, type 1D, Gaucher disease, atypical, due to saposin C deficiency, Krabbe disease atypical due to Saposin A deficiency, Combined saposin deficiency, Sphingolipid activator protein deficiency, Gaucher disease, atypical, due to saposin C deficiency, Spondyloepiphyseal dysplasia with congenital joint dislocations, Dilated cardiomyopathy 1W, Familial hypertrophic cardiomyopathy, Cardiovascular phenotype, Hypermethioninemia due to adenosine kinase deficiency, Genitopatellar syndrome, Young Simpson syndrome, Hypomyelinating leukodystrophy, Idiopathic fibrosing alveolitis, chronic form, Hepatic methionine adenosyltransferase deficiency, Hereditary cancer-predisposing syndrome, Juvenile polyposis syndrome, Juvenile polyposis syndrome, Hereditary cancer-predisposing syndrome, Hyperinsulinism-hyperammonemia syndrome, Spondyloepimetaphyseal dysplasia, pakistani type, hyperekplexia, Cowden syndrome, PTEN hamartoma tumor syndrome, Hereditary cancer-predisposing syndrome, Hereditary cancer-predisposing syndrome, Neoplasm of the breast, PTEN hamartoma tumor syndrome, Malignant melanoma of skin, Squamous cell carcinoma of the head and neck, Small cell lung cancer, Squamous cell lung carcinoma, Renal cell carcinoma, papillary, Neoplasm of the breast, Glioblastoma, Hereditary cancer-predisposing syndrome, Colorectal Neoplasms, Uterine cervical neoplasms, Adenocarcinoma of stomach, Malignant neoplasm of body of uterus, Adenocarcinoma of prostate, Uterine Carcinosarcoma, PTEN hamartoma tumor syndrome, Cowden syndrome, Hereditary cancer-predisposing syndrome, Hereditary cancer-predisposing syndrome, Lhermitte-Duclos disease, Neoplasm of the breast, Colorectal Neoplasms, Hereditary cancer-predisposing syndrome, Macrocephaly/autism syndrome, Hereditary cancer-predisposing syndrome, PTEN hamartoma tumor syndrome, Cutaneous melanoma, Hereditary cancer-predisposing syndrome, PTEN hamartoma tumor syndrome, Hereditary cancer-predisposing syndrome, Autoimmune lymphoproliferative syndrome, type 1a, Lysosomal acid lipase deficiency, Microcephaly with or without chorioretinopathy, lymphedema, or mental retardation, Hydranencephaly with renal aplasia-dysplasia, Spastic paraplegia, Cutis laxa, autosomal dominant, Primary hyperoxaluria, type III, Spastic tetraparesis, Hermansky-Pudlak syndrome, Dubin-Johnson syndrome, Renal coloboma syndrome, Autosomal dominant progressive external ophthalmoplegia with mitochondrial DNA deletions, Mitochondrial diseases, Autosomal dominant progressive external ophthalmoplegia with mitochondrial DNA deletions, Mitochondrial diseases, Kallmann syndrome, Combined partial 17-alpha-hydroxylase/17,20-lyase deficiency, Complete combined 17-alpha-hydroxylase/17,20-lyase deficiency, Cerebroretinal microangiopathy with calcifications and cysts, Adult junctional epidermolysis bullosa, Epidermolysis bullosa, junctional, spermatogenic failure, Primary dilated cardiomyopathy, Dilated cardiomyopathy, Microphthalmia, syndromic, Myofibrillar myopathy, BAG3-related, Myofibrillar myopathy, BAG3-related, Dilated cardiomyopathy, Jackson-Weiss syndrome, Craniosynostosis, nonsyndromic unicoronal, Pfeiffer syndrome, Craniofacial-skeletal-dermatologic dysplasia, FGFR2 related craniosynostosis, Pfeiffer syndrome, FGFR2 related craniosynostosis, Cerebral arteriopathy, autosomal dominant, with subcortical infarcts and leukoencephalopathy, type 2, Ornithine aminotransferase deficiency, Congenital erythropoietic porphyria, Muscular hypotonia, Muscular hypotonia, Intellectual disability (severe), Hypotonia, ataxia, and delayed development syndrome, Global developmental delay, Expressive language delay, Intellectual disability, Ataxia, Muscular hypotonia, Hypotonia, ataxia, and delayed development syndrome, Mitochondrial short-chain enoyl-coa hydratase deficiency, Noonan syndrome, Follicular thyroid carcinoma, Spermatocytic seminoma, somatic, Spermatocytic seminoma, Neoplasm of the breast, Costello syndrome, Myopathy, congenital, with excess of muscle spindles, Liver cancer, Chronic lymphocytic leukemia, Malignant melanoma of skin, Multiple myeloma, Squamous cell carcinoma of the head and neck, Costello syndrome, Lung adenocarcinoma, Squamous cell lung carcinoma, Acute myeloid leukemia, Neoplasm of the breast, Hepatocellular carcinoma, Pancreatic adenocarcinoma, Squamous cell carcinoma of the skin, Transitional cell carcinoma of the bladder, Colorectal Neoplasms, Uterine cervical neoplasms, Thymoma, Adenocarcinoma of stomach, Malignant neoplasm of body of uterus, Liver cancer, Chronic lymphocytic leukemia, Malignant melanoma of skin, Multiple myeloma, Squamous cell carcinoma of the head and neck, Costello syndrome, Lung adenocarcinoma, Squamous cell lung carcinoma, Acute myeloid leukemia, Rasopathy, Neoplasm of the breast, Hepatocellular carcinoma, Pancreatic adenocarcinoma, Squamous cell carcinoma of the skin, Transitional cell carcinoma of the bladder, Neoplasm, Colorectal Neoplasms, Uterine cervical neoplasms, Neoplasm of the thyroid gland, Adenocarcinoma of stomach, Malignant neoplasm of body of uterus, Malignant tumor of urinary bladder, Costello syndrome, Epidermal nevus, Myopathy, congenital, with excess of muscle spindles, Cutaneous melanoma, Neoplasm of the thyroid gland, Liver cancer, Malignant melanoma of skin, Multiple myeloma, Squamous cell carcinoma of the head and neck, Costello syndrome, Epidermal nevus, Lung adenocarcinoma, Acute myeloid leukemia, Myelodysplastic syndrome, Nevus sebaceous, Nevus sebaceous, somatic, Rasopathy, Neoplasm of the breast, Glioblastoma, Bladder carcinoma, Hepatocellular carcinoma, Pancreatic adenocarcinoma, Squamous cell carcinoma of the skin, Transitional cell carcinoma of the bladder, Carcinoma of esophagus, Colorectal Neoplasms, Uterine cervical neoplasms, Neoplasm of the thyroid gland, Papillary renal cell carcinoma, sporadic, Adenoid cystic carcinoma, Nasopharyngeal Neoplasms, Adenocarcinoma of stomach, Ovarian Serous Cystadenocarcinoma, Malignant neoplasm of body of uterus, Adenocarcinoma of prostate, Uterine Carcinosarcoma, Early myoclonic encephalopathy, Neutral lipid storage disease with myopathy, Ceroid lipofuscinosis neuronal, Growth restriction, severe, with distinctive facies, Hyperproinsulinemia, Permanent neonatal diabetes mellitus, Hyperproinsulinemia, Segawa syndrome, autosomal recessive, Dystonia, Segawa syndrome, autosomal recessive, Jervell and Lange-Nielsen syndrome, Long QT syndrome, Cardiovascular phenotype, Congenital long QT syndrome, Long QT syndrome, Congenital long QT syndrome, Long QT syndrome, Long QT syndrome 1/2, digenic, Long QT syndrome, Congenital long QT syndrome, Cardiovascular phenotype, Long QT syndrome, Congenital long QT syndrome, Long QT syndrome, Cardiovascular phenotype, Beckwith-Wiedemann syndrome, Intrauterine growth retardation, metaphyseal dysplasia, adrenal hypoplasia congenita, and genital anomalies, Intrauterine growth retardation, metaphyseal dysplasia, adrenal hypoplasia congenita, and genital anomalies, Russell-Silver syndrome, Beckwith-Wiedemann syndrome, Myopathy with tubular aggregates, hemoglobin Ohio, erythrocytosis, hemoglobin TY gard, erythrocytosis, Beta-thalassemia, dominant inclusion body type, Hemoglobinopathy, Beta-plus-thalassemia, Beta thalassemia intermedia, hemoglobin Ypsilanti, erythrocytosis, beta\hat{ }O\hat{ } Thalassemia, Heinz body anemia, Beta-plus-thalassemia, Beta thalassemia major, beta\hat{ }O\hat{ } Thalassemia, beta Thalassemia, Beta-plus-thalassemia, Hemoglobin Knossos, Beta-knossos-thalassemia, beta Thalassemia, Hemoglobin Palmerston north, erythrocytosis, Hb niigata, Beta-plus-thalassemia, beta Thalassemia, Beta thalassemia intermedia, beta Thalassemia, delta Thalassemia, hemoglobin A(2) Yialousa, Fetal hemoglobin quantitative trait locus 1, Sphingomyelin/cholesterol lipidosis, Niemann-Pick disease, type B, Niemann-Pick disease, type A, Niemann-pick disease, intermediate, protracted neurovisceral, Sphingomyelin/cholesterol lipidosis, Niemann-Pick disease, type B, Niemann-Pick disease, type A, Niemann-Pick disease, type B, Niemann-Pick disease, type A, Sphingomyelin/cholesterol lipidosis, Ceroid lipofuscinosis neuronal, Neuronal ceroid lipofuscinosis, Van Maldergem syndrome, Permanent neonatal diabetes mellitus, Permanent neonatal diabetes mellitus, Diabetes mellitus, permanent neonatal, with neurologic features, Islet cell hyperplasia, Permanent neonatal diabetes mellitus, Persistent hyperinsulinemic hypoglycemia of infancy, Permanent neonatal diabetes mellitus, Hyperekplexia, Gnathodiaphyseal dysplasia, Limb-girdle muscular dystrophy, type 2L, Gnathodiaphyseal dysplasia, Limb-girdle muscular dystrophy, type 2L, Miyoshi muscular dystrophy, ANO5-Related Disorders, Limb-girdle muscular dystrophy, type 2L, Elevated serum creatine phosphokinase, Myopathy, Distal muscle weakness, Fatty replacement of skeletal muscle, Limb-girdle muscular dystrophy, type 2L, Follicle-stimulating hormone deficiency, isolated, Aniridia, Irido-corneo-trabecular dysgenesis, Foveal hypoplasia with cataract, Irido-corneo-trabecular dysgenesis, Anophthalmia-microphthalmia, Aniridia, Irido-corneo-trabecular dysgenesis, Wilms tumor, Combined cellular and humoral immune defects with granulomas, Severe combined immunodeficiency, B cell-negative, Histiocytic medullary reticulosis, Severe immunodeficiency, autosomal recessive, T-cell negative, B-cell negative, NK cell-positive, Combined cellular and humoral immune defects with granulomas, Multiple exostoses type 2, Parietal foramina, Congenital disorder of glycosylation type 2C, Thrombophilia, Hereditary factor II deficiency disease, Xeroderma pigmentosum, group E, Left ventricular noncompaction, Hypertrophic cardiomyopathy, Primary familial hypertrophic cardiomyopathy, Hypertrophic cardiomyopathy, Cardiovascular phenotype, Primary familial hypertrophic cardiomyopathy, Cardiovascular phenotype, Familial hypertrophic cardiomyopathy, Primary familial hypertrophic cardiomyopathy, Hypertrophic, Primary familial hypertrophic cardiomyopathy, Cardiovascular phenotype, Familial hypertrophic cardiomyopathy, Familial hypertrophic cardiomyopathy, Primary familial hypertrophic cardiomyopathy, Hypertrophic cardiomyopathy, Cardiovascular phenotype, Primary familial hypertrophic cardiomyopathy, Hypertrophic cardiomyopathy, Primary familial hypertrophic cardiomyopathy, Primary familial hypertrophic cardiomyopathy, Hypertrophic cardiomyopathy, Hypertrophic cardiomyopathy, Primary familial hypertrophic cardiomyopathy, Cardiovascular phenotype, Familial hypertrophic cardiomyopathy, Cardiovascular phenotype, Primary familial hypertrophic cardiomyopathy, Familial hypertrophic cardiomyopathy, Hypertrophic cardiomyopathy, Myasthenic syndrome, congenital, associated with acetylcholine receptor deficiency, Pena-Shokeir syndrome type I, Myasthenic syndrome, congenital, associated with acetylcholine receptor deficiency, Congenital myasthenic syndrome, Myopathy, Myasthenic syndrome, congenital, associated with acetylcholine receptor deficiency, Congenital Myasthenic Syndrome, Recessive, Myasthenic syndrome, congenital, associated with acetylcholine receptor deficiency, Hereditary angioedema type 1, Hereditary C1 esterase inhibitor deficiency—dysfunctional factor, Poikiloderma, hereditary fibrosing, with tendon contractures, myopathy, and pulmonary fibrosis, Gracile bone dysplasia, Joubert syndrome, Joubert syndrome, Meckel syndrome type, Retinal dystrophy, polycystic kidney disease with polycystic liver disease, Congenital generalized lipodystrophy type 2, Charcot-Marie-Tooth disease, type 2, Encephalopathy, progressive, with or without lipodystrophy, Familial renal hypouricemia, Platelet-type bleeding disorder, Glycogen storage disease, type V, Hereditary cancer-predisposing syndrome, Multiple endocrine neoplasia, type 1, Hereditary cancer-predisposing syndrome, Hereditary cancer-predisposing syndrome, Multiple endocrine neoplasia, type 1, Multiple endocrine neoplasia, type 1, Hereditary cancer-predisposing syndrome, Coffin-Siris syndrome, Calfan syndrome, Verloes Bourguignon syndrome, Bardet-Biedl syndrome, Bardet-Biedl syndrome, Spinocerebellar ataxia, autosomal recessive, Pyruvate carboxylase deficiency, Cold-induced sweating syndrome, Crisponi/Cold-induced sweating syndrome, Somatotroph adenoma, Pituitary adenoma predisposition, Mitochondrial complex I deficiency, Osteopetrosis autosomal recessive, Severe congenital neutropenia autosomal dominant, congenital neutropenia, High bone mass, Osteoporosis with pseudoglioma, Epilepsy, familial temporal lobe, Carnitine palmitoyltransferase I deficiency, Charcot-Marie-Tooth disease, Charcot-Marie-Tooth disease, axonal, type 2S, IGHMBP2-related condition, Spinal muscular atrophy, distal, autosomal recessive, Charcot-Marie-Tooth disease, axonal, type 2S, Werdnig-Hoffmann disease, Charcot-Marie-Tooth disease, axonal, type 2S, Deafness with labyrinthine aplasia microtia and microdontia (LAMM), Smith-Lemli-Opitz syndrome, Cerebral folate deficiency, Opsismodysplasia, 3-methylglutaconic aciduria with cataracts, neurologic involvement, and neutropenia, Joubert syndrome, Vitreoretinopathy, neovascular inflammatory, Usher syndrome, type 1, Usher syndrome, type 1, Usher syndrome, type 1B, Usher syndrome, type 1, MYO7A-Related Disorders, polycystic liver disease with or without kidney cysts, Tremor, hereditary essential, Mitochondrial complex I deficiency, Mitochondrial diseases, Tyrosinase-negative oculocutaneous albinism, Tyrosinase-negative oculocutaneous albinism, Oculocutaneous albinism type 1B, Albinism, ocular, with sensorineural deafness, Skin/hair/eye pigmentation, variation in, Oculocutaneous albinism, Hereditary cancer-predisposing syndrome, Ataxia-telangiectasia-like disorder, Charcot-Marie-Tooth disease, type 4B1, Focal segmental glomerulosclerosis, Coloboma, ocular, with or without hearing impairment, cleft lip/palate, and/or mental retardation, Metaphyseal chondrodysplasia, Spahr type, Short-rib polydactyly syndrome type III, Jeune thoracic dystrophy, Short-rib thoracic dysplasia with or without polydactyly, Short-rib polydactyly syndrome type I, Short-rib polydactyly syndrome type III, Deficiency of acetyl-CoA acetyltransferase, Hereditary cancer-predisposing syndrome, Ataxia-telangiectasia syndrome, Ataxia-telangiectasia syndrome, Ataxia-telangiectasia variant, Pyruvate dehydrogenase E2 deficiency, Pheochromocytoma, Paragangliomas, Hereditary cancer-predisposing syndrome, Paragangliomas, Hereditary Paraganglioma-Pheochromocytoma Syndromes, Paraganglioma and gastric stromal sarcoma, Pheochromocytoma, Paragangliomas, Hereditary Paraganglioma-Pheochromocytoma Syndromes, Cowden syndrome, Paraganglioma and gastric stromal sarcoma, Pheochromocytoma, Mitochondrial complex II deficiency, Paragangliomas, Hereditary Paraganglioma-Pheochromocytoma Syndromes, Cowden syndrome 3, Apolipoprotein A-IV polymorphism, APOA4*1/APOA4*2, Hyperalphalipoproteinemia, Coronary heart disease, Apolipoprotein A-I (Baltimore), Immunodeficiency, Kabuki syndrome, Wiedemann-Steiner syndrome, Short stature, rhizomelic, with microcephaly, micrognathia, and developmental delay, Glucose-6-phosphate transport defect, Acute intermittent porphyria, Congenital myasthenic syndrome, Noonan syndrome-like disorder with or without juvenile myelomonocytic leukemia, Microphthalmia, isolated, Gaze palsy, familial horizontal, with progressive scoliosis, Megalencephalic leukoencephalopathy with subcortical cysts 2a, Deficiency of isobutyryl-CoA dehydrogenase, Cone dystrophy, Retinal cone dystrophy, Megalencephaly-polymicrogyria-polydactyly-hydrocephalus syndrome, Tumoral calcinosis, familial, hyperphosphatemic, Episodic ataxia type 1, Myokymia, Atrial fibrillation, familial, von Willebrand disease type 3, von Willebrand disease type 2N, von Willebrand disease type 2N, TNF receptor-associated periodic fever syndrome (TRAPS), Sifrim-Hitz-Weiss syndrome, Triosephosphate isomerase deficiency, Ehlers-Danlos syndrome, type 8, Immunodeficiency with hyper IgM type 2, Aortic aneurysm, familial thoracic, Acute myeloid leukemia, Diarrhea, Brachydactyly with hypertension, Hypoglycemia with deficiency of glycogen synthetase in the liver, Lamb-shaffer syndrome, Non-small cell lung cancer, Colorectal Neoplasms, Neoplasm of the thyroid gland, Non-small cell lung cancer, Rasopathy, on-small cell lung cancer, Colorectal Neoplasms, Neoplasm of the thyroid gland, cetuximab response—Dosage, panitumumab response—Dosage, Non-small cell lung cancer, RAS-associated autoimmune leukoproliferative disorder, Colorectal Neoplasms, Cerebral arteriovenous malformation, Juvenile myelomonocytic leukemia, Carcinoma of pancreas, Non-small cell lung cancer, Acute myeloid leukemia, Nevus sebaceous, Nevus sebaceous, somatic, Ovarian Neoplasms, Colorectal Neoplasms, Neoplasm of the thyroid gland, Endometrial carcinoma, Lung cancer, Lung adenocarcinoma, Non-small cell lung cancer, Ovarian Neoplasms, Colorectal Neoplasms, Neoplasm of the thyroid gland, Charcot-Marie-Tooth disease, type 4H, Optic atrophy, Encephalopathy due to defective mitochondrial and peroxisomal fission, Arrhythmogenic right ventricular cardiomyopathy, Arrhythmogenic right ventricular cardiomyopathy, type 9, Arrhythmogenic right ventricular dysplasia/cardiomyopathy, Cardiovascular phenotype, Parkinson disease, late-onset, Parkinson disease, autosomal dominant, IRAK4 deficiency, Vitamin D-dependent rickets, type 2, Spondyloperipheral dysplasia, Short ribs, Absent vertebral body mineralization, Spondylometaphyseal dysplasia, Stickler syndrome type 1, Stickler syndrome, type I, nonsyndromic ocular, Achondrogenesis, type II, Stickler syndrome type 1, Spondylometaphyseal dysplasia, Spondylometaphyseal dysplasia, Stickler syndrome, type I, nonsyndromic ocular, Glycogen storage disease, type VII, Glycogen storage disease, type VII, Osteogenesis imperfecta, type xv, Osteogenesis imperfecta, type xv, Osteogenesis imperfecta, type xv, Kabuki syndrome, Smith-Magenis Syndrome-like, Lissencephaly, Diabetes insipidus, nephrogenic, autosomal recessive, Diffuse palmoplantar keratoderma, Bothnian type, Hypochromic microcytic anemia with iron overload, Early infantile epileptic encephalopathy, Hereditary hemorrhagic telangiectasia type 2, Pulmonary arterial hypertension related to hereditary hemorrhagic telangiectasia, Primary pulmonary hypertension, Beaded hair, Pachyonychia congenita, Epidermolysis bullosa simplex, Dowling-Meara type, with severe palmoplantar keratoderma, Epidermolysis bullosa simplex, Cockayne-Touraine type, Epidermolysis bullosa simplex, Koebner type, Dowling-Degos disease, Ichthyosis bullosa of Siemens, Bullous ichthyosiform erythroderma, Cirrhosis, cryptogenic, Cirrhosis, noncryptogenic, susceptibility to, Glucocorticoid deficiency with achalasia, Ectodermal dysplasia, hair/nail type, Pigmentary retinal dystrophy, Fundus albipunctatus, autosomal recessive, Sulfite oxidase deficiency, isolated, Immunodeficiency, Congenital cataract, axonal, type 2u, Nephrotic syndrome, type 11, Bardet-Biedl syndrome, Myopathy, centronuclear, Joubert syndrome, Leber congenital amaurosis, Meckel syndrome type 4, Senior-Loken syndrome, Bardet-Biedl syndrome, Joubert syndrome, Leber congenital amaurosis, Meckel-Gruber syndrome, Meckel syndrome type 4, Senior-Loken syndrome, Joubert syndrome, Bardet-Biedl syndrome, Nephronophthisis, Meckel-Gruber syndrome, Nephronophthisis, Leber congenital amaurosis, Meckel syndrome type 4, Senior-Loken syndrome, Meckel-Gruber syndrome, Nephronophthisis, CEP290-Related Disorders, Leber congenital amaurosis, Meckel syndrome type 4, Senior-Loken syndrome, Leber congenital amaurosis, Meckel syndrome type 4, Senior-Loken syndrome, Cone-rod dystrophy, Cornea plana, Nephronophthisis, I cell disease, Pseudo-Hurler polydystrophy, Phenylketonuria, Hyperphenylalaninemia, non-pku, Congenital central hypoventilation, Hypomyelinating leukodystrophy, with or without oligodontia and/or hypogonadotropic hypogonadism, Methylmalonic aciduria cblB type, Methylmalonic academia, Spondylometaphyseal dysplasia, Kozlowski type, Skeletal dysplasia, Charcot-Marie-Tooth disease type 2C, Skeletal dysplasia, Neuromuscular Diseases, Digital arthropathy-brachydactyly, familial, Metatrophic dysplasia, Spondylometaphyseal dysplasia, Distal spinal muscular atrophy, congenital nonprogressive, Scapuloperoneal spinal muscular atrophy, Charcot-Marie-Tooth disease type 2C, Skeletal dysplasia, Neuromuscular Diseases, Charcot-Marie-Tooth, Type 2, Brachyolmia, Metatrophic dysplasia, Skeletal dysplasia, Neuromuscular Diseases, Darier disease, acral hemorrhagic type, Darier disease, segmental, Familial hypertrophic cardiomyopathy, Primary familial hypertrophic cardiomyopathy, Familial hypertrophic cardiomyopathy, Death in infancy, Ventricular extrasystoles, Cardiovascular phenotype, Noonan syndrome, Noonan syndrome, Rasopathy, Juvenile myelomonocytic leukemia, Noonan syndrome, Leopard syndrome, Rasopathy, Metachondromatosis, Noonan syndrome with multiple lentigines, Noonan syndrome 1, LEOPARD syndrome, Scoliosis, Rasopathy, Abnormal facial shape, Cafe-au-lait spot, Specific learning disability, Intellectual disability, mild, Aortic valve disease, Holt-Oram syndrome, Mental retardation and distinctive facial features with or without cardiac defects, Charcot-Marie-Tooth disease, type 2L, Microcephaly, primary, autosomal recessive, Deficiency of butyryl-CoA dehydrogenase, Maturity-onset diabetes of the young, type 3, Immune dysfunction with T-cell inactivation due to calcium entry defect, Leukoencephalopathy with vanishing white matter, Joubert syndrome, Cutis laxa with osteodystrophy, Myopathy, lactic acidosis, and sideroblastic anemia, Knuckle pads, deafness and leukonychia syndrome, Keratitis-ichthyosis-deafness syndrome, autosomal dominant, Mutilating keratoderma, Hystrix-like ichthyosis with deafness, Keratitis-ichthyosis-deafness syndrome, autosomal dominant, Keratoderma palmoplantar deafness, Knuckle pads, deafness and leukonychia syndrome, Deafness, X-linked, Hearing impairment, Keratoderma palmoplantar deafness, Cardiomyopathy, Left ventricular noncompaction, Cardiomyopathy, Infantile muscular hypotonia, Combined oxidative phosphorylation deficiency, Pancreatic agenesis, congenital, Diabetes mellitus type 2, Acute lymphoid leukemia, Acute myeloid leukemia, Breast-ovarian cancer, familial, Hereditary breast and ovarian cancer syndrome, Breast-ovarian cancer, familial, Hereditary breast and ovarian cancer syndrome, Hereditary cancer-predisposing syndrome, Hereditary breast and ovarian cancer syndrome, Familial cancer of breast, Breast-ovarian cancer, familial, Hereditary breast and ovarian cancer syndrome, Hereditary cancer-predisposing syndrome, Familial cancer of breast, Breast-ovarian cancer, familial, Fanconi anemia, complementation group D1, Medulloblastoma, Wilms tumor, Malignant tumor of prostate, Tracheoesophageal fistula, Pancreatic cancer, Glioma susceptibility, Hereditary breast and ovarian cancer syndrome, Hereditary cancer-predisposing syndrome, Familial cancer of breast, Breast-ovarian cancer, familial, Hereditary breast and ovarian cancer syndrome, Neoplasm of the breast, Hereditary cancer-predisposing syndrome, Familial cancer of breast, Breast-ovarian cancer, familial, Fanconi anemia, complementation group D1, Medulloblastoma, Wilms tumor, Malignant tumor of prostate, Tracheoesophageal fistula, Pancreatic cancer, Glioma susceptibility, Hereditary breast and ovarian cancer syndrome, Hereditary cancer-predisposing syndrome, BRCA2-Related Disorders, Breast-ovarian cancer, familial, Fanconi anemia, complementation group D1, Fanconi anemia, Hereditary breast and ovarian cancer syndrome, Hereditary cancer-predisposing syndrome, Primary pulmonary hypertension, Congenital disorder of glycosylation type 2L, Hyperornithinemia-hyperammonemia-homocitrullinuria syndrome, Retinoblastoma, Retinoblastoma, Neoplasm, Small cell lung cancer, Neoplasm, Retinitis pigmentosa, Retinal dystrophy with or without extraocular anomalies, Retinitis pigmentosa, Retinal dystrophy with extraocular anomalies, Aicardi Goutieres syndrome, Wilson disease, Ceroid lipofuscinosis neuronal, Hirschsprung disease, Waardenburg syndrome type 4A, Deafness and myopia, Catel Manzke syndrome, Propionyl-CoA carboxylase deficiency, Hypotonia, infantile, with psychomotor retardation and characteristic facies, Congenital contractures of the limbs and face, hypotonia, and developmental delay, Xeroderma pigmentosum, group G, Xeroderma pigmentosum group g/Cockayne syndrome, Xeroderma pigmentosum, group G, Xeroderma pigmentosum, Schizencephaly, Angiopathy, hereditary, with nephropathy, aneurysms, and muscle cramps, Squamous cell carcinoma of the head and neck, Oguchi disease, Cone-rod dystrophy, Leber congenital amaurosis, Cone-Rod Dystrophy, Recessive, Autism, susceptibility to, Ocular coloboma, autosomal recessive, Lysinuric protein intolerance, Primary dilated cardiomyopathy, Wolff-Parkinson-White pattern, Dilated cardiomyopathy 1EE, Familial hypertrophic cardiomyopathy, Primary familial hypertrophic cardiomyopathy, Sudden cardiac death, Cardiovascular phenotype, Hypertrophic cardiomyopathy, Primary familial hypertrophic cardiomyopathy, Cardiovascular phenotype, Familial hypertrophic cardiomyopathy, Primary familial hypertrophic cardiomyopathy, Cardiovascular phenotype, Familial hypertrophic cardiomyopathy, Familial cardiomyopathy, Hypertrophic cardiomyopathy, Cardiomyopathy, Hypertrophic cardiomyopathy, Dyskeratosis congenita, Dyskeratosis congenita autosomal dominant, Dyskeratosis congenita autosomal dominant, Dyskeratosis congenita, autosomal dominant, Revesz syndrome, Dyskeratosis congenita autosomal dominant, Dyskeratosis congenita, Dyskeratosis Congenita, Dominant, Autosomal recessive congenital ichthyosis, Rett syndrome, congenital variant, Mitochondrial complex I deficiency, Ectodermal dysplasia, anhidrotic, with T-cell immunodeficiency, autosomal dominant, Benign hereditary chorea, Choreoathetosis, hypothyroidism, and neonatal respiratory distress, Partial congenital absence of teeth, Ciliary dyskinesia, primary, Kartagener syndrome, L-2-hydroxyglutaric aciduria, Penetrating foot ulcers, Distal sensory impairment, Osteomyelitis leading to amputation due to slow healing fractures, Distal lower limb muscle weakness, Glycogen storage disease, type VI, Dystonia, Dopa-responsive type, Microphthalmia syndromic, Anophthalmia, combined immunodeficiency and megaloblastic anemia, Hereditary cancer-predisposing syndrome, congenital disorder of glycosylation with defective fucosylation, Leber congenital amaurosis, Platelet-type bleeding disorder, Alzheimer disease, type 3, Alzheimer disease, type 3, Pick's disease, Alzheimer disease, type 3, Frontotemporal dementia, Pick's disease, Acne inversa, familial, Coenzyme Q10 deficiency, primary, Methylmalonate semialdehyde dehydrogenase deficiency, Niemann-Pick disease type C2, Niemann-Pick disease, type C, Leukoencephalopathy with vanishing white matter, Carcinoma of colon, Endometrial carcinoma, Hereditary nonpolyposis colorectal cancer type 7, Lynch syndrome, MLH3-Related Lynch Syndrome, Nevus comedonicus, Proliferative vasculopathy and hydranencephaly-hydrocephaly syndrome, Cone-rod dystrophy, Congenital muscular dystrophy-dystroglycanopathy with brain and eye anomalies, type A2, Congenital muscular dystrophy-dystroglycanopathy with mental retardation, type B2, Limb-girdle muscular dystrophy-dystroglycanopathy, type C2, Neuropathy, hereditary sensory, type IC, Hereditary sensory and autonomic neuropathy type IC, Thyroid adenoma, hyperfunctioning, somatic, Thyroid adenoma, hyperfunctioning, Hypothyroidism, congenital, nongoitrous, Hyperthyroidism, nonautoimmune, Thyroid adenoma, hyperfunctioning, somatic, Thyroid adenoma, hyperfunctioning, Galactosylceramide beta-galactosidase deficiency, Leber congenital amaurosis, Autosomal recessive cutis laxa type IA, TRIP11-related condition, Alpha-1-antitrypsin deficiency, Pineoblastoma, DICER1-related pleuropulmonary blastoma cancer predisposition syndrome, Hereditary cancer-predisposing syndrome, Gabriele-De Vries Syndrome, Spinal muscular atrophy, SMA, Spinal muscular atrophy, lower extremity predominant, autosomal dominant, Mental retardation, autosomal dominant, Mental retardation, autosomal dominant, Charcot-Marie-Tooth disease, dominant intermediate E, cerebellar-facial-dental syndrome, Cerebellofaciodental syndrome, Precocious puberty, central, Schaaf-yang syndrome, Angelman syndrome, Epileptic encephalopathy, early infantile, Tyrosinase-positive oculocutaneous albinism, Congenital stationary night blindness, type 1C, Andermann syndrome, Familial hypertrophic cardiomyopathy, Familial pulmonary capillary hemangiomatosis, Isovaleric acidemia, type I, Adams-Oliver syndrome, Limb-girdle muscular dystrophy, type 2A, Spherocytosis type 5, Peeling skin syndrome, Peeling skin syndrome, acral type, Microcephaly and chorioretinopathy, autosomal recessive, Hypoproteinemia, hypercatabolic, Arginine:glycine amidinotransferase deficiency, Bartter syndrome, type 1, antenatal, Marfan syndrome, Marfan lipodystrophy syndrome, Cardiovascular phenotype, Marfan syndrome, Thoracic aortic aneurysm and aortic dissection, Thoracic aortic Aneurysm and dissection (TAAD), Cardiovascular phenotype, Stiff skin syndrome, Marfan syndrome, Thoracic aortic aneurysm and aortic dissection, Thoracic aortic Aneurysm and dissection (TAAD), Marfan Syndrome/Loeys-Dietz Syndrome/Familial Thoracic Aortic Aneurysms and Dissections, Cardiovascular phenotype, Seckel syndrome, Aromatase deficiency, Lethal congenital contracture syndrome, Intellectual developmental disorder with cardiac arrhythmia, Primary ciliary dyskinesia, Craniosynostosis, Parkinson disease, age at onset, susceptibility to, Parkinson disease, Parkinson disease, autosomal recessive early-onset, Hyperchlorhidrosis, isolated, Nemaline myopathy, Congenital stationary night blindness, type 1D, Lung adenocarcinoma, Non-small cell lung cancer, Cutaneous melanoma, Cardio-facio-cutaneous syndrome, Cardiofaciocutaneous syndrome, Cardio-facio-cutaneous syndrome, Aortic valve disease, Thoracic aortic aneurysm and aortic dissection, Cardiovascular phenotype, Loeys-Dietz syndrome, Ceroid lipofuscinosis neuronal, Tay-Sachs disease, Bardet-Biedl syndrome, Sick sinus syndrome, autosomal dominant, Tyrosinemia type I, Tyrosinemia type I, Hypertyrosinemia, Osteochondritis dissecans, Autosomal dominant progressive external ophthalmoplegia with mitochondrial DNA deletions 1, Progressive sclerosing poliodystrophy, Progressive sclerosing poliodystrophy, Mitochondrial diseases, Camptocormia, Acrocallosal syndrome, Schinzel type, Spondylocostal dysostosis, Liver cancer, Acute myeloid leukemia, Neoplasm of brain, Hepatocellular carcinoma, Brainstem glioma, Colorectal Neoplasms, Multiple myeloma, Squamous cell carcinoma of the head and neck, Acute myeloid leukemia, Myelodysplastic syndrome, Colorectal Neoplasms, Bloom syndrome, Bloom syndrome, Hereditary cancer-predisposing syndrome, Arthrogryposis renal dysfunction cholestasis syndrome, Epileptic encephalopathy, childhood-onset, Congenital heart defects, multiple types, Weill-Marchesani-like syndrome, Autosomal recessive congenital ichthyosis, Microphthalmia, isolated, Osteosclerotic metaphyseal dysplasia, alpha Thalassemia, Hemoglobin Loire, Erythrocytosis, Hemoglobin Chesapeake, Erythrocytosis, Hemoglobin Legnano, Erythrocytosis, Spinocerebellar ataxia, autosomal recessive, Mucolipidosis III Gamma, You-Hoover-Fong syndrome, Renal dysplasia, retinal pigmentary dystrophy, cerebellar ataxia and skeletal dysplasia, Joubert syndrome with Jeune asphyxiating thoracic dystrophy, Renal dysplasia, retinal pigmentary dystrophy, cerebellar ataxia and skeletal dysplasia, Retinis pigmentosa, Leigh syndrome, Combined oxidative phosphorylation deficiency, Tuberous sclerosis, Tuberous sclerosis syndrome, Lymphangiomyomatosis, Tuberous sclerosis syndrome, Polycystic kidney disease, adult type, Digitorenocerebral syndrome, Early infantile epileptic encephalopathy, Myoclonic epilepsy, familial infantile, Digitorenocerebral syndrome, Progressive myoclonus epilepsy with ataxia, Familial Mediterranean fever, Rubinstein-Taybi syndrome, Nephronophthisis, Congenital disorder of glycosylation type 1K, Carbohydrate-deficient glycoprotein syndrome type I, Carbohydrate-deficient glycoprotein syndrome type I, Congenital disorder of glycosylation, Epilepsy, focal, with speech disorder and with or without mental retardation, Rolandic epilepsy, Bare lymphocyte syndrome type 2, complementation group A, Charcot-Marie-Tooth disease, type 1C, Fanconi anemia, complementation group Q, Dyskeratosis congenita, Dyskeratosis congenita, autosomal recessive, Lissencephaly, Aortic aneurysm, familial thoracic, Pseudoxanthoma elasticum, Pseudoxanthoma elasticum, Generalized arterial calcification of infancy, Familial juvenile gout, Uromodulin-associated kidney disease, Medullary cystic kidney disease, Bronchiectasis with or without elevated sweat chloride, Familial cancer of breast, Fanconi anemia, complementation group N, Tracheoesophageal fistula, Pancreatic cancer, Hereditary breast and ovarian cancer syndrome, Hereditary cancer-predisposing syndrome, Familial cancer of breast, Pancreatic cancer, Progressive sensorineural hearing impairment, IL21R immunodeficiency, Juvenile neuronal ceroid lipofuscinosis, Ceroid lipofuscinosis, neuronal, protracted, Brody myopathy, Spondyloepimetaphyseal dysplasia with multiple dislocations, Spondylocostal dysostosis, Bile acid synthesis defect, congenital, Generalized epilepsy with febrile seizures plus, type 9, Warfarin response, warfarin response—Dosage, Warfarin response, Familial renal glucosuria, Glycogen storage disease IXb, Behcet's syndrome, Cylindromatosis, familial, Townes-Brocks syndrome, Joubert syndrome, Hamamy syndrome, Multicentric osteolysis, nodulosis and arthropathy, Bardet-Biedl syndrome, Retinitis pigmentosa, Nephrotic syndrome, type 12, Familial hypokalemia-hypomagnesemia, Spondyloepimetaphyseal dysplasia, Faden-Alkuraya type, Polymicrogyria, bilateral frontoparietal, Lissencephaly, with microcephaly, Retinitis pigmentosa, Poikiloderma with neutropenia, Brachioskeletogenital syndrome, Mitochondrial DNA depletion syndrome, Lamellar cataract, Combined T and B cell immunodeficiency, Dyskeratosis congenita, autosomal dominant, Dyskeratosis congenita, autosomal recessive, Norum disease, Acanthosis nigricans, Skeletal dysplasia, Insulin resistance, Short stature, Self-injurious behavior, Abnormal facial shape, Brachydactyly, Renal hypoplasia, Abnormality of the dentition, Hepatic steatosis, Obesity, Lumbar hyperlordosis, Hyperlipidemia, Short metacarpal, Intellectual disability, severe, Short stature, brachydactyly, intellectual developmental disability, and seizures, Acanthosis nigricans, Skeletal dysplasia, Insulin resistance, Short stature, Self-injurious behavior, Abnormal facial shape, Brachydactyly, Renal hypoplasia, Abnormality of the dentition, Hepatic steatosis, Obesity, Lumbar hyperlordosis, Hyperlipidemia, Short metacarpal, Intellectual disability, severe, Hereditary diffuse gastric cancer, Hereditary cancer-predisposing syndrome, Ectropion inferior cleft lip and or palate, Breast cancer, lobular, Hereditary diffuse gastric cancer, Hereditary cancer-predisposing syndrome, Ectropion inferior cleft lip and or palate, Congenital disorder of glycosylation type 2J, Striatonigral degeneration, childhood-onset, Ciliary dyskinesia, primary, Kartagener syndrome, Tyrosinemia type 2, Macular corneal dystrophy Type I, Macular corneal dystrophy, type II, Microcomea, myopic chorioretinal atrophy, and telecanthus, Spinocerebellar ataxia, autosomal recessive, Cataract, multiple types, Ayme-gripp syndrome, Giant axonal neuropathy, Autoinflammation, antibody deficiency, and immune dysregulation, plcg2-associated, Ciliary dyskinesia, primary, Persistent fetal circulation, Keratoconus, Corneal fragility keratoglobus, blue sclerae AND joint hypermobility, Keratoconus, Granulomatous disease, chronic, autosomal recessive, cytochrome b-negative, Chronic granulomatous disease, Granulomatous disease, chronic, autosomal recessive, cytochrome b-negative, Lymphedema, hereditary, III, Adenine phosphoribosyltransferase deficiency, Mucopolysaccharidosis, MPS-IV-A, KBG syndrome, Astigmatism, Cryptorchidism, Hypertelorism, Esotropia, Retrognathia, Hypermetropia, Wide nasal bridge, Cryptorchidism, Epicanthus, Hypertelorism, Astigmatism, Intellectual disability, Global developmental delay, Fanconi anemia, complementation group A, Fanconi anemia, Cutaneous malignant melanoma, Malignant Melanoma Susceptibility, Ciliary dyskinesia, primary, Syndactyly type 9, Retinitis pigmentosa, Lissencephaly, Spongy degeneration of central nervous system, Spongy degeneration of central nervous system, Canavan Disease, Familial Form, Palmoplantar keratoderma, mutilating, with periorificial keratotic plaques, Nephropathic cystinosis, Cystinosis, atypical nephropathic, Myasthenic syndrome, congenital, 4a, slow-channel, Myasthenic syndrome, congenital, associated with acetylcholine receptor deficiency, Congenital myasthenic syndrome 1B, fast-channel, Pseudo von Willebrand disease, Amyotrophic lateral sclerosis, Combined oxidative phosphorylation deficiency, Leber congenital amaurosis, Orofaciodigital syndrome XV, Very long chain acyl-CoA dehydrogenase deficiency, Myasthenic syndrome, congenital, slow-channel, Li-Fraumeni syndrome, Hereditary cancer-predisposing syndrome, Familial colorectal cancer, Malignant lymphoma, non-Hodgkin, Liver cancer, Chronic lymphocytic leukemia, Medulloblastoma, Malignant melanoma of skin, Multiple myeloma, Squamous cell carcinoma of the head and neck, Small cell lung cancer, Lung adenocarcinoma, Squamous cell lung carcinoma, Acute myeloid leukemia, Neoplasm of brain, Neoplasm of the breast, Glioblastoma, Hepatocellular carcinoma, Hereditary cancer-predisposing syndrome, Pancreatic adenocarcinoma, Transitional cell carcinoma of the bladder, Brainstem glioma, Carcinoma of esophagus, Colorectal Neoplasms, Adrenocortical carcinoma, Adenocarcinoma of stomach, Ovarian Serous Cystadenocarcinoma, Malignant neoplasm of body of uterus, Adenocarcinoma of prostate, Uterine Carcinosarcoma, Metastatic pancreatic neuroendocrine tumours, Liver cancer, Chronic lymphocytic leukemia, Medulloblastoma, Malignant melanoma of skin, Multiple myeloma, Squamous cell carcinoma of the head and neck, Small cell lung cancer, Lung adenocarcinoma, Squamous cell lung carcinoma, Acute myeloid leukemia, Neoplasm of brain, Neoplasm of the breast, Glioblastoma, Hepatocellular carcinoma, Hereditary cancer-predisposing syndrome, Pancreatic adenocarcinoma, Transitional cell carcinoma of the bladder, Brainstem glioma, Carcinoma of esophagus, Colorectal Neoplasms, Adrenocortical carcinoma, Adenocarcinoma of stomach, Ovarian Serous Cystadenocarcinoma, Malignant neoplasm of body of uterus, Adenocarcinoma of prostate, Uterine Carcinosarcoma, Medulloblastoma, Multiple myeloma, Squamous cell carcinoma of the head and neck, Li-Fraumeni syndrome, Lung adenocarcinoma, Renal cell carcinoma, papillary, Neoplasm of the breast, Hereditary cancer-predisposing syndrome, Pancreatic adenocarcinoma, Squamous cell carcinoma of the skin, Transitional cell carcinoma of the bladder, Colorectal Neoplasms, Adenocarcinoma of stomach, Ovarian Serous Cystadenocarcinoma, Malignant neoplasm of body of uterus, Hereditary cancer-predisposing syndrome, Carcinoma of cervix, Liver cancer, Li-Fraumeni syndrome, Hepatocellular carcinoma, Hereditary cancer-predisposing syndrome, Liver cancer, Squamous cell carcinoma of the head and neck, Li-Fraumeni syndrome, Lung adenocarcinoma, Li-Fraumeni syndrome, Squamous cell lung carcinoma, Neoplasm of brain, Neoplasm of the breast, Glioblastoma, Hepatocellular carcinoma, Hereditary cancer-predisposing syndrome, Pancreatic adenocarcinoma, Transitional cell carcinoma of the bladder, Brainstem glioma, Carcinoma of esophagus, Colorectal Neoplasms, Adenocarcinoma of stomach, Ovarian Serous Cystadenocarcinoma, Adenocarcinoma of prostate, Uterine Carcinosarcoma, Liver cancer, Chronic lymphocytic leukemia, Multiple myeloma, Squamous cell carcinoma of the head and neck, Lung adenocarcinoma, Li-Fraumeni syndrome, Neoplasm of brain, Neoplasm of the breast, Glioblastoma, Hepatocellular carcinoma, Pancreatic adenocarcinoma, Transitional cell carcinoma of the bladder, Carcinoma of esophagus, Colorectal Neoplasms, Uterine cervical neoplasms, Adenocarcinoma of stomach, Ovarian Serous Cystadenocarcinoma, Malignant neoplasm of body of uterus, Uterine Carcinosarcoma, Li-Fraumeni syndrome, Liver cancer, Hepatocellular carcinoma, Hereditary cancer-predisposing syndrome, Liver cancer, Malignant melanoma of skin, Multiple myeloma, Squamous cell carcinoma of the head and neck, Lung adenocarcinoma, Breast cancer, somatic, Squamous cell lung carcinoma, Neoplasm of brain, Neoplasm of the breast, Hepatocellular carcinoma, Breast adenocarcinoma, Hereditary cancer-predisposing syndrome, Pancreatic adenocarcinoma, Transitional cell carcinoma of the bladder, Carcinoma of esophagus, Colorectal Neoplasms, Adenoid cystic carcinoma, Adenocarcinoma of stomach, Ovarian Serous Cystadenocarcinoma, Malignant neoplasm of body of uterus, Uterine Carcinosarcoma, Carcinoma of pancreas, Dyskeratosis congenita, autosomal recessive, Leber congenital amaurosis, Cone-rod dystrophy, Autosomal recessive congenital ichthyosis, Ichthyosis, Autosomal recessive congenital ichthyosis, Spondylocostal dysostosis, Inclusion Body Myopathy, Dominant, Hepatic failure, early-onset, and neurologic disorder due to cytochrome C oxidase deficiency, Charcot-Marie-Tooth disease and deafness, Dejerine-Sottas disease, Dejerine-Sottas disease, Dejerine-Sottas syndrome, autosomal dominant, Charcot-Marie-Tooth disease, type IA, Dejerine-Sottas syndrome, autosomal dominant, Charcot-Marie-Tooth disease, type I, Mitochondrial complex III deficiency, nuclear type 2, Common variable immunodeficiency, Immunoglobulin A deficiency, Common Variable Immune Deficiency, Dominant, Common variable immunodeficiency, Hereditary cancer-predisposing syndrome, Multiple fibrofolliculomas, Hereditary cancer-predisposing syndrome, Hereditary cancer-predisposing syndrome, Multiple fibrofolliculomas, Hereditary cancer-predisposing syndrome, Smith-Magenis syndrome, Joubert syndrome, Meckel-Gruber syndrome, Sjögren-Larsson syndrome, Congenital disorders of glycosylation type II, Congenital disorder of glycosylation IIp, Congenital defect of folate absorption, Immunodeficiency, Cone-Rod Dystrophy, Dominant, Neurofibromatosis, type 1, Hereditary cancer-predisposing syndrome, Breast-ovarian cancer, familial 4, Hereditary cancer-predisposing syndrome, Infantile Refsum's disease, Peroxisome biogenesis disorders, Zellweger syndrome spectrum, Peroxisome biogenesis disorder, Familial hypoplastic, glomerulocystic kidney, Limb-girdle muscular dystrophy, type 2G, Hyperphosphatasia with mental retardation syndrome, Neoplasm of the breast, Transitional cell carcinoma of the bladder, Carcinoma of esophagus, Uterine cervical neoplasms, Adenocarcinoma of stomach, Neoplasm of the breast, Colorectal Neoplasms, Adenocarcinoma of stomach, Hypothyroidism, congenital, nongoitrous, Autosomal recessive woolly hair, Autosomal Recessive Hypotrichosis with Woolly Hair, Bullous ichthyosiform erythroderma, Meesman's corneal dystrophy, Dermatopathia pigmentosa reticularis, Naxos disease, Ciliary dyskinesia, primary, Autoimmune disease, multisystem, infantile-onset, Mucopolysaccharidosis, MPS-III-B, Glycogen storage disease type 1A, Breast-ovarian cancer, familial, Breast-ovarian cancer, familial, Hereditary breast and ovarian cancer syndrome, Breast-ovarian cancer, familial 1, Hereditary breast and ovarian cancer syndrome, Hereditary cancer-predisposing syndrome, Familial cancer of breast, Hereditary cancer-predisposing syndrome, Breast-ovarian cancer, familial, Hereditary breast and ovarian cancer syndrome, Breast-ovarian cancer, familial, Familial cancer of breast, Breast-ovarian cancer, familial 1, Hereditary breast and ovarian cancer syndrome, Neoplasm of the breast, Hereditary cancer-predisposing syndrome, Breast-ovarian cancer, familial, Hereditary breast and ovarian cancer syndrome, Hereditary cancer-predisposing syndrome, Breast-ovarian cancer, familial, Hereditary breast and ovarian cancer syndrome, Hereditary breast and ovarian cancer syndrome, Hereditary cancer-predisposing syndrome, Familial cancer of breast, Breast-ovarian cancer, familial, Hereditary breast and ovarian cancer syndrome, Hereditary cancer-predisposing syndrome, Neoplasm of the breast, Renal tubular acidosis, autosomal dominant, Frontotemporal dementia, ubiquitin-positive, Growth and mental retardation, mandibulofacial dysostosis, microcephaly, and cleft palate, Alexander's disease, Progressive supranuclear ophthalmoplegia, Frontotemporal dementia, Progressive supranuclear ophthalmoplegia, Muscular dystrophy, Epilepsy, progressive myoclonic 6, Glanzmann thrombasthenia, Amelogenesis imperfecta, type IV, Tricho-dento-osseous syndrome, Osteogenesis imperfecta type I, Osteogenesis imperfecta type 2, thin-bone, Osteogenesis imperfecta with normal sclerae, dominant form, Osteogenesis imperfecta type I, Osteogenesis imperfecta type IIC, Osteogenesis imperfecta, recessive perinatal lethal, Osteogenesis imperfecta type I, Osteogenesis imperfecta with normal sclerae, dominant form, Osteogenesis imperfecta type III, Osteogenesis imperfecta, type III/iv, Osteogenesis imperfecta, recessive perinatal lethal, Osteogenesis imperfecta with normal sclerae, dominant form, Osteogenesis imperfecta type 1, mild, Proximal symphalangism, Tarsal carpal coalition syndrome, Joubert syndrome, Joubert syndrome, Fanconi anemia, complementation group O, Hereditary cancer-predisposing syndrome, Fanconi anemia, complementation group O, Retinitis pigmentosa, Ischiopatellar dysplasia, Familial cancer of breast, Fanconi anemia, complementation group J, Neoplasm of ovary, Hereditary breast and ovarian cancer syndrome, Hereditary cancer-predisposing syndrome, Hereditary cancer-predisposing syndrome, Familial cancer of breast, Fanconi anemia, complementation group J, Hereditary cancer-predisposing syndrome, Hereditary breast and ovarian cancer syndrome, Hereditary cancer-predisposing syndrome, Rolandic epilepsy, Isolated growth hormone deficiency type 1B, Hyperkalemic Periodic Paralysis Type 1, Potassium aggravated myotonia, Paramyotonia congenita of von Eulenburg, Paramyotonia congenita/hyperkalemic periodic paralysis, Hyperkalemic Periodic Paralysis Type 1, Hypokalemic periodic paralysis, Hypokalemic periodic paralysis, type 2, Hyperkalemic Periodic Paralysis Type 1, Carcinoma of colon, Oligodontia-colorectal cancer syndrome, Carney complex, type 1, Andersen Tawil syndrome, Familial periodic paralysis, Andersen Tawil syndrome, Andersen Tawil syndrome, Congenital long QT syndrome, Acampomelic campomelic dysplasia, Camptomelic dysplasia, Striatal necrosis, bilateral, and progressive polyneuropathy, Pontocerebellar hypoplasia type 4, Pontocerebellar hypoplasia type 2A, Pontocerebellar hypoplasia type 4, Pontocerebellar hypoplasia type 2A, Pontocerebellar hypoplasia type 5, Congenital cerebellar hypoplasia, Hypertonia, Microcephaly, Amblyopia, Global developmental delay, Olivopontocerebellar hypoplasia, Non-syndromic pontocerebellar hypoplasia, Olivopontocerebellar hypoplasia, Deficiency of galactokinase, Hemophagocytic lymphohistiocytosis, familial, Pseudoneonatal adrenoleukodystrophy, Epidermodysplasia verruciformis, Desbuquois dysplasia, Rolandic epilepsy, Ciliary dyskinesia, Ciliary dyskinesia, primary, Glycogen storage disease, type II, Glycogen storage disease type II, infantile, Glycogen storage disease, type II, Baraitser-Winter Syndrome, Nephrotic syndrome, type 8, Autosomal recessive cutis laxa type 2B, Encephalopathy, progressive, early-onset, with brain atrophy and thin corpus callosum, Arhinia choanal atresia microphthalmia, Oculomelic amyoplasia, Dystonia, Spinocerebellar ataxia, ACTH resistance, Glucocorticoid Deficiency, Renal hypodysplasia/aplasia, Left ventricular noncompaction, Pancreatic agenesis and congenital heart disease, Abnormality of cardiovascular system morphology, Congenital diaphragmatic hernia, Seckel syndrome, Niemann-Pick disease type C1, Niemann-Pick disease type C1, Niemann-Pick disease, type D, Scalp ear nipple syndrome, Arrhythmogenic right ventricular cardiomyopathy, type 10, Arrhythmogenic right ventricular cardiomyopathy, Cardiovascular phenotype, Arrhythmogenic right ventricular cardiomyopathy, type 10, Amyloidogenic transthyretin amyloidosis, Cardiovascular phenotype, Bainbridge-Ropers syndrome, Mental retardation, autosomal recessive, Vici syndrome, Carcinoma of pancreas, Juvenile polyposis syndrome, Colorectal Neoplasms, Juvenile polyposis/hereditary hemorrhagic telangiectasia syndrome, Juvenile polyposis syndrome, Colorectal Neoplasms, Mirror movements, Carcinoma of colon, Pitt-Hopkins syndrome, Erythropoietic protoporphyria, Progressive intrahepatic cholestasis, Periventricular nodular heterotopia with syndactyly, cleft palate and developmental delay, Periventricular nodular heterotopia, Immunodeficiency, Obesity, Schizophrenia, Obesity, Osteopetrosis autosomal recessive, Burn-McKeown syndrome, Severe congenital neutropenia autosomal dominant, Cyclical neutropenia, Complement factor d deficiency, Spondylometaphyseal dysplasia Sedaghatian type, Carcinoma of pancreas, Peutz-Jeghers syndrome, Hereditary cancer-predisposing syndrome, Hereditary cancer-predisposing syndrome, Cutaneous malignant melanoma, Cutaneous melanoma, Persistent mullerian duct syndrome, type I, Preimplantation embryonic lethality, Hypocalcemia, autosomal dominant, Cone-rod dystrophy, Age-related macular degeneration, Spinocerebellar ataxia, Cardiofaciocutaneous syndrome, Cardio-facio-cutaneous syndrome, CODAS syndrome, Leukodystrophy, hypomyelinating, Insulin-resistant diabetes mellitus and acanthosis nigricans, Pineal hyperplasia and diabetes mellitus syndrome, Insulin-resistant diabetes mellitus and acanthosis nigricans, Leprechaunism syndrome, Pineal hyperplasia and diabetes mellitus syndrome, Retinitis pigmentosa, Mucolipidosis type IV, Mucolipidosis type IV, Mucolipidosis type IV, Boucher Neuhauser syndrome, Weill-Marchesani syndrome, Cerebellar ataxia, deafness and narcolepsy, autosomal dominant, Tyrosine kinase 2 deficiency, Charcot-Marie-Tooth disease, type 2M, Familial hypercholesterolemia, Familial hypercholesterolemias, Kartagener syndrome, Ciliary dyskinesia, primary, Spondyloenchondrodysplasia with immune dysregulation, Deficiency of alpha-mannosidase, Aicardi Goutieres syndrome, Blood group—Lutheran inhibitor, Glutaric aciduria, type 1, Marshall-Smith syndrome, Epileptic encephalopathy, early infantile, Familial hemiplegic migraine type 1, Episodic ataxia type 2, Epileptic encephalopathy, early infantile, Familial hemiplegic migraine type 1, Autosomal recessive non-syndromic intellectual disability, Lehman syndrome, Cerebral autosomal dominant arteriopathy with subcortical infarcts and leukoencephalopathy, Combined oxidative phosphorylation deficiency, Severe combined immunodeficiency, autosomal recessive, T cell-negative, B cell-positive, NK cell-negative, Thyroid dyshormonogenesis, Cold-induced sweating syndrome, Pseudoachondroplastic spondyloepiphyseal dysplasia syndrome, Multiple epiphyseal dysplasia, Pseudoachondroplastic spondyloepiphyseal dysplasia syndrome, Epiphyseal dysplasia, multiple, severe, Bilateral right-sidedness sequence, Transposition of the great arteries, dextro-looped, Heterotaxia, Acute myeloid leukemia, Arthrogryposis multiplex congenita, neurogenic, with myelin defect, Hemochromatosis type 1, Dystonia 28, childhood-onset, Finnish congenital nephrotic syndrome, Central core disease, Central core disease, Malignant hyperthermia, susceptibility to, RYR1-Related Disorders, Congenital myopathy with fiber type disproportion, RYR1-Related Disorders, Myopathy, Congenital myopathy with fiber type disproportion, Central core disease, Malignant hyperthermia, susceptibility to, Central core disease, Congenital myopathy with fiber type disproportion, Central core disease, Cutis laxa with severe pulmonary, gastrointestinal, and urinary abnormalities, Nephrotic syndrome, type 9, Maple syrup urine disease, Diamond-Blackfan anemia, Alternating hemiplegia of childhood, Dystonia, Familial partial lipodystrophy 6, Ethylmalonic encephalopathy, Blood group—Lutheran Null, Familial type 3 hyperlipoproteinemia, Apolipoprotein C2 deficiency, Apolipoprotein C-II (Padova), Apolipoprotein C2 deficiency, Apolipoprotein C-II (Auckland), Immunodeficiency, Hermansky-Pudlak syndrome, Xeroderma pigmentosum, group D, Trichothiodystrophy, photosensitive, Congenital muscular dystrophy-dystroglycanopathy with mental retardation, type B5, Muscular dystrophy-dystroglycanopathy (congenital with brain and eye anomalies), type a, Congenital muscular dystrophy-dystroglycanopathy (with or without mental retardation) type B5, Limb-girdle muscular dystrophy-dystroglycanopathy, type C5, Limb-girdle muscular dystrophy, Congenital muscular dystrophy-dystroglycanopathy with brain and eye anomalies type A5, Muscle weakness, Headache, Gait imbalance, Difficulty walking, Paresthesia, Difficulty climbing stairs, Scapular winging, Difficulty standing, Muscular dystrophy-dystroglycanopathy, Walker-Warburg congenital muscular dystrophy, Congenital muscular dystrophy-dystroglycanopathy with mental retardation, type B5, Limb-girdle muscular dystrophy-dystroglycanopathy, type C5, Walker-Warburg congenital muscular dystrophy, Walker-Warburg congenital muscular dystrophy, Congenital muscular dystrophy-dystroglycanopathy without mental retardation, type B5, Walker-Warburg congenital muscular dystrophy, Congenital muscular dystrophy-dystroglycanopathy with mental retardation, type B5, Limb-girdle muscular dystrophy-dystroglycanopathy, type C5, Congenital muscular dystrophy-dystroglycanopathy with mental retardation, type B5, Congenital muscular dystrophy-dystroglycanopathy with brain and eye anomalies type A5, Walker-Warburg congenital muscular dystrophy, Hypocalciuric hypercalcemia, familial, type III, Mental retardation, autosomal recessive, Hyperferritinemia cataract syndrome, L-ferritin deficiency, autosomal recessive, Isolated lutropin deficiency, Autistic disorder of childhood onset, Motor delay, Iris coloboma, Autism, Delayed speech and language development, Abnormality of vision, Early infantile epileptic encephalopathy, Ataxia-oculomotor apraxia, Early infantile epileptic encephalopathy, Peripheral neuropathy, myopathy, hoarseness, and hearing loss, Spinocerebellar ataxia, Spinocerebellar ataxia, Retinitis pigmentosa, Nemaline myopathy, Polyglucosan body myopathy with or without immunodeficiency, Glycogen storage disease, type IV, Brown-Vialetto-Van Laere syndrome, Spinocerebellar ataxia, Cerebro-costo-mandibular syndrome, Neurohypophyseal diabetes insipidus, Pigmentary pallidal degeneration, Hypoprebetalipoproteinemia, acanthocytosis, retinitis pigmentosa, and pallidal degeneration, Pigmentary pallidal degeneration, Spongiform encephalopathy with neuropsychiatric features, Genetic prion diseases, Gerstmann-Straussler-Scheinker syndrome, Cerebral Amyloid Angiopathy, PRNP-related, Ataxia-telangiectasia-like disorder, Kindler's syndrome, Short stature, facial dysmorphism, and skeletal anomalies with or without cardiac anomalies, Auriculocondylar syndrome, McKusick Kaufman syndrome, Alagille syndrome, Mitochondrial complex I deficiency, Leigh syndrome, Congenital dyserythropoietic anemia, type II, Cowden syndrome, Congenital dyserythropoietic anemia, Retinitis pigmentosa, Otofaciocervical syndrome, Thrombophilia due to thrombomodulin defect, Thrombophilia due to thrombomodulin defect, Joint laxity, short stature, and myopia, Craniofacial anomalies and anterior segment dysgenesis syndrome, Familial hypertrophic cardiomyopathy, Cardiomyopathy, hypertrophic, midventricular, digenic, Dowling-Degos disease, C-like syndrome, Multiple synostoses syndrome, Symphalangism, proximal, Fibular hypoplasia and complex brachydactyly, schizophrenia, Aicardi Goutieres syndrome, Severe combined immunodeficiency due to ADA deficiency, Partial adenosine deaminase deficiency, Multiple congenital anomalies-hypotonia-seizures syndrome, Primary autosomal recessive microcephaly, Galloway-Mowat Syndrome, Arterial tortuosity syndrome, Epileptic encephalopathy, early infantile, Helsmoortel-van der aa syndrome, Congenital disorder of glycosylation type 1E, Idiopathic hypercalcemia of infancy, Cushing's syndrome, McCune-Albright syndrome, Polyostotic fibrous dysplasia, somatic, mosaic, Pituitary Tumor, Growth Hormone-Secreting, Somatic, Liver cancer, McCune-Albright syndrome, Malignant melanoma of skin, Squamous cell carcinoma of the head and neck, Lung adenocarcinoma, Neoplasm of the breast, Hepatocellular carcinoma, Pancreatic adenocarcinoma, Neoplasm, Colorectal Neoplasms, Uterine cervical neoplasms, Adrenocortical carcinoma, Adenocarcinoma of stomach, McCune-Albright syndrome, Pseudohypoparathyroidism, type IA, with testotoxicosis, Pseudohypoparathyroidism type 1C, Waardenburg syndrome type 4B, Early infantile epileptic encephalopathy, Benign familial neonatal seizures, Early infantile epileptic encephalopathy, Seizures, Generalized hypotonia, Early infantile epileptic encephalopathy, Benign familial neonatal seizures, Dyskeratosis congenita, autosomal recessive, Pulmonary fibrosis and/or bone marrow failure, telomere-related, Dyskeratosis congenita, Dyskeratosis congenita, autosomal recessive, Dyskeratosis congenita, autosomal recessive, Glomerulonephritis with sparse hair and telangiectases, Alzheimer disease, type 1, Amyotrophic lateral sclerosis type 1, Inflammatory bowel disease, autosomal recessive, Immunodeficiency, Familial platelet disorder with associated myeloid malignancy, Familial platelet disorder with associated myeloid malignancy, Transient myeloproliferative disorder of Down syndrome, Leukemia, acute myeloid, m0 subtype, Popliteal pterygium syndrome lethal type, Kartagener syndrome, Primary ciliary dyskinesia, Kartagener syndrome, Ciliary dyskinesia, Primary ciliary dyskinesia, Homocystinuria due to CBS deficiency, Epileptic encephalopathy, early infantile, Unverricht-Lundborg syndrome, Autoimmune polyglandular syndrome type 1, autosomal dominant, Leukocyte adhesion deficiency type 1, Bethlem myopathy, Ullrich congenital muscular dystrophy, Ullrich congenital muscular dystrophy, Microcephalic osteodysplastic primordial dwarfism type 2, Polyarteritis nodosa, childhood-onset, Peroxisome biogenesis disorder, Proline dehydrogenase deficiency, Schizophrenia, Autosomal recessive Noonan-like syndrome due to compound heterozygous variants in LZTR1, Spinal muscular atrophy, jokela type, Frontotemporal dementia and/or amyotrophic lateral sclerosis, Myopathy, isolated mitochondrial, autosomal dominant, Rhabdoid tumor predisposition syndrome, Schwannomatosis, Deficiency of beta-ureidopropionase, Congenital cataract, Klippel-feil syndrome, autosomal recessive, with nemaline myopathy and facial dysmorphism, Hermansky-Pudlak syndrome, Cataract, congenital nuclear, autosomal recessive, Cataract, multiple types, Familial cancer of breast, Hereditary cancer-predisposing syndrome, Hereditary cancer-predisposing syndrome, Familial cancer of breast, Prostate cancer, somatic, Hereditary cancer-predisposing syndrome, Osteosarcoma, Neurofibromatosis, type 2, Epilepsy, familial focal, with variable foci, Rolandic epilepsy, Parkinson disease, Sorsby fundus dystrophy, Macrothrombocytopenia and granulocyte inclusions with or without nephritis or sensorineural hearing loss, Microcytic anemia, Peripheral demyelinating neuropathy, central dysmyelination, Waardenburg syndrome, and Hirschsprung disease, Waardenburg syndrome type 4C, Parkinson disease, Infantile neuroaxonal dystrophy, Adenylosuccinate lyase deficiency, Nephronophthisis-like nephropathy, Carcinoma of colon, Rubinstein-Taybi syndrome, Carcinoma of colon, Kanzaki disease, Methemoglobinemia type 2, Autosomal recessive syndrome of syndactyly, undescended testes and central nervous system defects, Megalencephalic leukoencephalopathy with subcortical cysts, Microcephaly with chorioretinopathy, autosomal recessive, Mitochondrial DNA depletion syndrome (MNGIE type), Muscular dystrophy, congenital, megaconial type, Metachromatic leukodystrophy, juvenile type, Metachromatic leukodystrophy, late infantile, Metachromatic leukodystrophy, Metachromatic leukodystrophy, severe, Metachromatic leukodystrophy, Short stature, idiopathic, X-linked, Leri Weill dyschondrosteosis, Chondrodysplasia punctata, X-linked recessive, Kallmann syndrome, Ocular albinism, type I, Opitz-Frias syndrome, Amelogenesis imperfecta, type 1E, Spondyloepiphyseal dysplasia tarda, Oral-facial-digital syndrome, Joubert syndrome, Joubert syndrome, Oral-facial-digital syndrome, Paroxysmal nocturnal hemoglobinuria 1, Multiple congenital anomalies-hypotonia-seizures syndrome, Pettigrew syndrome, Nance-Horan syndrome, Congenital cataract, Early infantile epileptic encephalopathy, Early infantile epileptic encephalopathy, Atypical Rett syndrome, Early infantile epileptic encephalopathy, Angelman syndrome-like, Early infantile epileptic encephalopathy, Atypical Rett syndrome, Early infantile epileptic encephalopathy, Angelman syndrome-like, Early infantile epileptic encephalopathy, Atypical Rett syndrome, Early infantile epileptic encephalopathy, Angelman syndrome-like, Juvenile retinoschisis, Glycogen storage disease type IXal, Coffin-Lowry syndrome, Deafness, X-linked, IFAP syndrome with or without BRESHECK syndrome, Familial X-linked hypophosphatemic vitamin D refractory rickets, Hydranencephaly with abnormal genitalia, Proud Levine Carpenter syndrome, Lissencephaly, X-linked, epileptic encephalopathy, early infantile, Mental retardation, X-linked, Congenital adrenal hypoplasia, X-linked, Becker muscular dystrophy, Duchenne muscular dystrophy, Becker muscular dystrophy, Duchenne muscular dystrophy, Dilated cardiomyopathy, Granulomatous disease, chronic, X-linked, variant, Cone-rod dystrophy, X-linked, Retinitis pigmentosa, Ornithine carbamoyltransferase deficiency, Mental retardation, X-linked, Mental retardation, X-linked, Congenital stationary night blindness, type 1A, Mental retardation and microcephaly with pontine and cerebellar hypoplasia, FG syndrome, Monoamine oxidase A deficiency, Atrophia bulborum hereditaria, Familial exudative vitreoretinopathy, X-linked, Atrophia bulborum hereditaria, Kabuki syndrome, Retinitis pigmentosa, Arthrogryposis multiplex congenita, distal, X-linked, Properdin deficiency, X-linked, Chondrodysplasia punctata, X-linked dominant, atypical, Chondrodysplasia punctata X-linked dominant, MEND syndrome, Wiskott-Aldrich syndrome, GATA-1-related thrombocytopenia with dyserythropoiesis, Dyserythropoietic anemia with thrombocytopenia, GATA-1-related thrombocytopenia with dyserythropoiesis, Neurodegeneration with brain iron accumulation, Nephrolithiasis, X-linked recessive, Dent disease, Mental retardation, syndromic, Claes-Jensen type, X-linked, 2-methyl-3-hydroxybutyric aciduria, Aarskog syndrome, Hereditary sideroblastic anemia, Amyotrophic lateral sclerosis, with or without frontotemporal dementia, Androgen resistance syndrome, Partial androgen insensitivity syndrome, Prostate cancer susceptibility, Androgen resistance syndrome, Partial androgen insensitivity syndrome, Craniofrontonasal dysplasia, Hypohidrotic X-linked ectodermal dysplasia, Hypohidrotic ectodermal dysplasia, Tooth agenesis, selective, X-linked, Myopia, X-Linked, Female-Limited, X-linked severe combined immunodeficiency, Ohdo syndrome, X-linked, FG syndrome, Intellectual functioning disability, Cardiovascular phenotype, X-linked hereditary motor and sensory neuropathy, Mental retardation, X-linked, syndromic, Mental Retardation, X-Linked, Cornelia de Lange syndrome 5, Glycogen storage disease, Allan-Herndon-Dudley syndrome, Mental retardation, X-linked, Metacarpal 4-5 fusion, ATR-X syndrome, Menkes kinky-hair syndrome, Menkes kinky-hair syndrome, Cutis laxa, X-linked, Distal spinal muscular atrophy, X-linked, Phosphoglycerate kinase 1 deficiency, Cleft palate with ankyloglossia, Mental retardation, X-linked, Choroideremia, Early infantile epileptic encephalopathy, Mohr-Tranebjaerg syndrome, X-linked agammaglobulinemia, Agammaglobulinemia, non-Bruton type, Fabry disease, Fabry disease, Deoxygalactonojirimycin response, Pelizaeus-Merzbacher disease, Pelizaeus-Merzbacher disease, connatal, Thyroxine-binding globulin, variant P, Phosphoribosylpyrophosphate synthetase superactivity, Charcot-Marie-Tooth disease, X-linked recessive, type 5, Alport syndrome, X-linked recessive, Microscopic hematuria, Elevated mean arterial pressure, Chronic kidney disease, Mental retardation, X-linked, Megalocornea, Mental retardation, X-linked, Heterotopia, Lissencephaly, X-linked, Fucosidosis, Lissencephaly, X-linked, Subcortical laminar heterotopia, X-linked, Danon disease, Syndromic X-linked mental retardation, Cabezas type, Mental retardation, X-linked, syndromic, wu type, Lymphoproliferative syndrome, X-linked, Lymphoproliferative syndrome, X-linked, Simpson-Golabi-Behmel syndrome, Borjeson-Forssman-Lehmann syndrome, Lesch-Nyhan syndrome, Lesch-Nyhan syndrome, HPRT Flint, Partial hypoxanthine-guanine phosphoribosyltransferase deficiency, HPRT Munich, HPRT Milwaukee, Lesch-Nyhan syndrome, Christianson syndrome, Hypertrophic cardiomyopathy, Myopathy, reducing body, X-linked, early-onset, severe, Immunodeficiency with hyper IgM type 1, Pituitary adenoma, growth hormone-secreting, Heterotaxy, visceral, X-linked, VACTERL association with hydrocephaly, X-linked, Congenital heart defects, nonsyndromic, Heterotaxy, visceral, X-linked, Hereditary factor IX deficiency disease, Hereditary factor IX deficiency disease, Thrombophilia, X-linked, due to factor IX defect, Mucopolysaccharidosis, MPS-II, Mucopolysaccharidosis, type II, severe form, Mucopolysaccharidosis, MPS-II, Hypospadias, X-linked, Severe X-linked myotubular myopathy, Child syndrome, Spondyloepimetaphyseal dysplasia X-linked, Microcephaly, Carious teeth, Intellectual disability, Global developmental delay, Abnormality of the cerebral cortex, Skeletal muscle atrophy, Oral-pharyngeal dysphagia, Muscular hypotonia, Muscular hypotonia, Creatine deficiency, X-linked, Chromosome Xq28 deletion syndrome, Adrenoleukodystrophy, Nephrogenic diabetes insipidus, X-linked, Nephrogenic syndrome of inappropriate antidiuresis, N-terminal acetyltransferase deficiency, Rett syndrome, Mental retardation, X-linked, syndromic, Rett syndrome, Rett syndrome, Stereotypy, Delayed speech and language development, Delayed gross motor development, Bruxism, Deuteranopia, Otopalatodigital spectrum disorder, Melnick-Needles syndrome, Periventricular nodular heterotopia, Melnick-Needles syndrome, Oto-palato-digital syndrome, type II, Frontometaphyseal dysplasia, Cardiac valvular dysplasia, X-linked, Periventricular nodular heterotopia, Oto-palato-digital syndrome, type II, Oto-palato-digital syndrome, type I, Emery-Dreifuss muscular dystrophy, X-linked, 3-Methylglutaconic aciduria type 2, Galloway-Mowat Syndrome, X-Linked, Glucose 6 phosphate dehydrogenase deficiency, G6pd a-, G6PD Canton, G6PD GIFU, G6PD Agrigento, G6PD Taiwan-Hakka, Anemia, nonspherocytic hemolytic, due to G6PD deficiency, G6PD LOMA Linda, Anemia, nonspherocytic hemolytic, due to G6PD deficiency, Glucose phosphate dehydrogenase deficiency, G6pd a-G6PD Gastonia, G6PD Marion, G6PD Minnesota, Anemia, nonspherocytic hemolytic, due to G6PD deficiency, Hypohidrotic ectodermal dysplasia with immune deficiency, Dyskeratosis congenita X-linked, Hereditary factor VIII deficiency disease, Parkinsonism, early onset with mental retardation, Mental retardation, X-linked, Leri Weill dyschondrosteosis, XY sex reversal, type 1, Leigh syndrome, Chloramphenicol resistance, nonsyndromic sensorineural, mitochondrial, Leber's optic atrophy, Cytochrome c oxidase i deficiency, Leigh syndrome, Mitochondrial complex I deficiency, Leigh syndrome, Retinitis pigmentosa-deafness syndrome, Cerebellar ataxia, cataract, and diabetes mellitus.

Pathogenic T to G or A to C mutations may be corrected using the methods and compositions provided herein, for example by mutating the C to a T, and/or the G to an A, and thereby restoring gene function. Guide RNA (gRNA) sequences, which encode RNA that can direct a napDNAbp, or any of the base editors provided herein, to a target site may be cloned into an expression vector, such as Addgene pFYF1320 (which targets EGFP), to encode a gRNA that targets a napDNAbp, or any of the base editors provided herein, to a target site in order to correct a disease-related mutation.

In some aspects, the present disclosure provides uses of any one of the fusion proteins (e.g., base editors) described herein, and a guide RNA targeting this base editor to a target C:G base pair in a nucleic acid molecule, in the manufacture of a kit for base editing, wherein the base editing comprises contacting the nucleic acid molecule with the base editor and guide RNA under conditions suitable for the substitution of the cytosine (C) of the C:G nucleobase pair with a thymine (T). In some embodiments of these uses, the nucleic acid molecule is a double-stranded DNA molecule. In some embodiments, the step of contacting induces separation of the double-stranded DNA at a target region. In some embodiments, the step of contacting further comprises nicking one strand of the double-stranded DNA, wherein the one strand comprises an unmutated strand that comprises the G of the target C:G nucleobase pair.

In some embodiments of the described uses, the step of contacting is performed in vitro. In other embodiments, the step of contacting is performed in vivo. In some embodiments, the step of contacting is performed in a subject (e.g., a human subject or a non-human animal subject). In some embodiments, the step of contacting is performed in a cell, such as a human or non-human animal cell.

The present disclosure also provides uses of any one of the fusion proteins (e.g., base editors, prime editors, or other fusion proteins provided herein) described herein as a medicament. The present disclosure also provides uses of any one of the complexes of fusion proteins and guide RNAs described herein as a medicament.

### Pharmaceutical Compositions

Other aspects of the present disclosure relate to pharmaceutical compositions comprising any of the fusion proteins, guide RNAs, complexes, systems, polynucleotides, vectors, and/or cells described herein. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” (or “pharmaceutically acceptable excipient”) means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservatives, and antioxidants can also be present in the formulation. Terms such as “excipient”, “carrier”, “pharmaceutically acceptable carrier,” “pharmaceutically acceptable excipient,” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105). Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical compositions for administration by injection are solutions in sterile isotonic aqueous buffer. Where necessary, the pharmaceutical composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical composition is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical compositions described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease and may have a sterile access port. For example, the container may be an intravenous solution bag or a vial having a stopper pierce-able by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Delivery Methods

In some aspects, the disclosure provides methods comprising delivering any of the Cas14a1 variants, fusion proteins (e.g., base editors and prime editors), gRNAs, and/or complexes described herein. In other embodiments, the disclosure provides methods comprising delivery of one or more vectors as described herein, one or more transcripts thereof, and/or one or proteins transcribed therefrom, to a host cell. In some embodiments, the invention further provides cells produced by such methods, and organisms (such as animals, plants, or fungi) comprising or produced from such cells. In some embodiments, a fusion protein (e.g., base editor) as described herein in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a base editor to cells in culture, or in a host organism. Non-viral vector delivery systems include ribonucleoprotein (RNP) complexes, DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

In some embodiments, the Cas14a1 variant or fusion protein (e.g., base editor) and gRNA are delivered or administered as a protein:RNA complex. In certain embodiments, the method of delivery and vector provided herein is an RNP complex. For example, RNP delivery of base editors markedly increases the DNA specificity of base editing. RNP delivery of base editors leads to decoupling of on- and off-target editing. RNP delivery ablated off-target editing at non-repetitive sites while maintaining on-target editing comparable to plasmid delivery, and greatly reduced off-target editing even at the highly repetitive VEGFA site 2. See Rees, H. A. et al., Improving the DNA specificity and applicability of base editing through protein engineering and protein delivery, Nat. Commun. 8, 15790 (2017), which is incorporated by reference herein in its entirety.

Methods of non-viral delivery of nucleic acids include RNP complexes, lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. Nos. 5,049,386, 4,946,787, and 4,897,355, and lipofection reagents are sold commercially (e.g., Lipofectamine, Lipofectamine 2000, Lipofectamine 3000, Transfectam™ and Lipofectin™). In certain embodiments of the disclosed methods of editing, a cationic lipid comprising Lipofectamine 2000 is used for delivery of nucleic acids to cells. Cationic and neutral lipids that are suitable for efficient receptor-recognition lipofection of polynucleotides include those of Feigner (see WO 1991/17424 and WO 1991/16024). Delivery can be to cells (e.g., in vitro or ex vivo administration) or target tissues (e.g., in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, 4,946,787, 9,526,784, and 9,737,604).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo), or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); Samulski et al., J. Virol. 63:03822-3828 (1989); and International Patent Application No. PCT/US2023/066389, filed Apr. 28, 2023.

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. Reference is made to US 2003-0087817, published May 8, 2003, International Patent Application No. WO 2016/205764, published Dec. 22, 2016, International Patent Application No. WO 2018/071868, published Apr. 19, 2018, U.S. Patent Publication No. 2018/0127780, published May 10, 2018, and International Patent Application No. PCT/US2020/033873, the disclosures of each of which are incorporated herein by reference.

In various embodiments, the disclosed expression constructs may be engineered for delivery in one or more rAAV vectors. An rAAV as related to any of the methods and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). An rAAV may comprise a genetic load (i.e., a recombinant nucleic acid vector that expresses a gene of interest, such as a base editor that is carried by the rAAV into a cell) that is to be delivered to a cell. An rAAV may be chimeric.

As used herein, the serotype of an rAAV refers to the serotype of the capsid proteins of the recombinant virus. Non-limiting examples of derivatives and pseudotypes include rAAV2/1, rAAV2/5, rAAV2/8, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6 (Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. A non-limiting example of derivatives and pseudotypes that have chimeric VP1 proteins is rAAV2/5-1VPlu, which has the genome of AAV2, capsid backbone of AAV5 and VPlu of AAV1. Other non-limiting examples of derivatives and pseudotypes that have chimeric VP1 proteins are rAAV2/5-8VPlu, rAAV2/9-1VPlu, and rAAV2/9-8VPlu.

AAV derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Methods of making or packaging rAAV particles are known in the art, and reagents for doing so are commercially available (see, e.g., Zolotukhin et al., Production and purification of serotype 1, 2, and 5 recombinant adeno-associated viral vectors. Methods 28 (2002) 158-167; and U.S. Patent Publication Numbers US-2007-0015238 and US-2012-0322861; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into recombinant cells such that the rAAV particle can be packaged and subsequently purified.

Additional methods for the delivery of nucleic acids to cells are known to those skilled in the art. See, for example, U.S. Publication No. 2003/0087817, incorporated herein by reference.

It should be appreciated that any fusion protein, e.g., any of the base editors provided herein, may be introduced into the cell in any suitable way, either stably or transiently. In some embodiments, a fusion protein may be transfected into the cell. In some embodiments, the cell may be transduced or transfected with a nucleic acid construct that encodes a fusion protein described herein. For example, a cell may be transduced (e.g., with a virus encoding a fusion protein such as a base editor), or transfected (e.g., with a plasmid encoding a fusion protein such as a base editor) with a nucleic acid that encodes a fusion protein, or the translated fusion protein. Such transduction may be a stable or transient transduction. In some embodiments, cells expressing a fusion protein or containing a fusion protein may be transduced or transfected with one or more gRNA molecules, for example when the fusion protein comprises a Cas protein (e.g., any of the Cas14a1 variants provided herein) domain. In some embodiments, a plasmid expressing a fusion protein may be introduced into cells through electroporation, transient (e.g., lipofection) and stable genome integration (e.g., piggybac) and viral transduction or other methods known to those of skill in the art.

### Vectors

Some aspects of this disclosure relate to polynucleotides and vector constructs for producing the disclosed Cas14a1 variants, fusion proteins (e.g., base editors and prime editors), gRNAs, and complexes. Some aspects of this disclosure relate to cells (e.g., host cells) comprising the Cas14a1 variants or fusion proteins disclosed herein, cells comprising the disclosed polynucleotides, and cells comprising the disclosed vectors.

In certain embodiments, methods of manufacturing the base editors for use in the methods of DNA editing, methods of treatment, pharmaceutical compositions, and kits disclosed herein comprise the use of recombinant protein expression methodologies and techniques known to those of skill in the art.

Several embodiments of the making and using of the fusion proteins of the invention relate to vector systems comprising one or more vectors, or vectors as such. Vectors may be designed to clone and/or express the fusion proteins as disclosed herein. Vectors may also be designed to clone and/or express one or more gRNAs having complementarity to the target sequence, as disclosed herein. Vectors may also be designed to transfect the fusion proteins and gRNAs of the disclosure into one or more cells, e.g., a target diseased eukaryotic cell for treatment with the fusion proteins methods disclosed herein.

Vectors can be designed for expression of fusion protein transcripts (e.g., nucleic acid transcripts, proteins, or enzymes) in prokaryotic or eukaryotic cells. For example, fusion protein transcripts can be expressed in bacterial cells such as Escherichia coli, insect cells (using baculovirus expression vectors), yeast cells, plant cells, or mammalian cells. Suitable host cells are discussed further in Goeddel, Gene Expression Technology: Methods In Enzymology 185, Academic Press. San Diego, Calif. (1990). Alternatively, expression vectors encoding one or more fusion proteins described herein can be transcribed and translated in vitro, for example, using T7 promoter regulatory sequences and T7 polymerase.

Vectors may be introduced and propagated in a prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion proteins or non-fusion proteins.

Fusion expression vectors also may be used to express the fusion proteins (e.g., base editors and prime editors) of the disclosure. Such vectors generally add a number of amino acids to a protein encoded therein, such as to the amino terminus of the recombinant protein. Such fusion vectors may serve one or more purposes, such as: (i) to increase expression of a recombinant protein; (ii) to increase the solubility of a recombinant protein; and (iii) to aid in the purification of a recombinant protein by acting as a ligand in affinity purification. Often, in fusion expression vectors, a proteolytic cleavage site is introduced at the junction of the fusion domain and the recombinant protein to enable separation of the recombinant protein from the fusion domain subsequent to purification of the base editor. Such enzymes, and their cognate recognition sequences, include Factor Xa, thrombin, and enterokinase. Exemplary fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., Gene Expression Technology: Methods In Enzymology 185, Academic Press, San Diego, Calif. (1990) 60-89).

In some embodiments, a vector is a yeast expression vector for expressing the fusion proteins, such as base editors, described herein. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter, U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

### Kits and Cells

The Cas proteins, fusion proteins, gRNAs, complexes, polynucleotides, systems, vectors, and cells of the present disclosure may be assembled into kits. In some embodiments, a kit comprises any of the Cas14a1 variants disclosed herein. In some embodiments, a kit comprises any of the fusion proteins (e.g., base editors and prime editors comprising Cas14a1 variants) provided herein. In some embodiments, a kit comprises any of the gRNAs provided herein. In some embodiments, a kit comprises any of the complexes provided herein. In some embodiments, a kit comprises any of the polynucleotides provided herein. In some embodiments, a kit comprises any of the vectors provided herein. In some embodiments, a kit comprises any of the cells provided herein.

The kit described herein may include one or more containers housing components for performing the methods described herein, and optionally instructions for use. Any of the kits described herein may further comprise components needed for performing the genome editing methods described herein. Each component of the kits, where applicable, may be provided in liquid form (e.g., in solution) or in solid form, (e.g., a dry powder). In certain cases, some of the components may be reconstitutable or otherwise processible (e.g., to an active form), for example, by the addition of a suitable solvent or other species (for example, water), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral, and electronic communication of any form, associated with the disclosure. Additionally, the kits may include other components depending on the specific application, as described herein.

The kits may contain any one or more of the components described herein in one or more containers. The components may be prepared sterilely, packaged in a syringe, and shipped refrigerated. Alternatively, they may be housed in a vial or other container for storage. A second container may have other components prepared sterilely. Alternatively, the kits may include the active agents premixed and shipped in a vial, tube, or other container.

The kits may have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box, or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc. Some aspects of this disclosure provide kits comprising a nucleic acid construct comprising a nucleotide sequence encoding the Cas proteins, fusion proteins, gRNAs, and/or complexes described herein (e.g., including, but not limited to, the napDNAbps, deaminase domains, and reverse transcriptases). In some embodiments, the nucleotide sequence(s) comprises a heterologous promoter (or more than a single promoter) that drives expression of the components encoded by the polynucleotide. In some aspects, the present disclosure provides vectors (e.g., expression vectors) comprising any of the polynucleotides described herein.

Cells that may contain any of the Cas proteins, fusion proteins, gRNAs, complexes, polynucleotides, and/or vectors described herein include prokaryotic cells and eukaryotic cells. In some embodiments, a cell comprises any of the Cas14a1 variants described herein. In some embodiments, a cell comprises any of the fusion proteins provided herein. In some embodiments, a cell comprises any of the gRNAs provided herein. In some embodiments, a cell comprises any of the complexes provided herein. In some embodiments, a cell comprises any of the polynucleotides provided herein. In some embodiments, a cell comprises any of the vectors provided herein.

Typically, the eukaryotic cell is a mammalian cell, such as a human cell, a chicken cell, or an insect cell. Examples of suitable mammalian cells are, but are not limited to, HEK-293T cells, COS7 cells, Hela cells and HEK-293 cells. Examples of suitable insect cells include, but are not limited to, High5 cells and Sf9 cells. In some embodiment, the cells are insect cells as they are devoid of undesirable human proteins, and their culture does not require animal serum.

Mammalian cells of the present disclosure include human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, the Cas proteins, fusion proteins, gRNAs and/or complexes described herein are delivered into human embryonic kidney (HEK) cells (e.g., HEK 293 or HEK 293T cells). In some embodiments, the Cas proteins, fusion proteins, gRNAs and/or complexes described herein are delivered into stem cells (e.g., human stem cells) such as, for example, pluripotent stem cells (e.g., human pluripotent stem cells including human induced pluripotent stem cells (hiPSCs)). A stem cell refers to a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1, and YAR cells.

Some aspects of this disclosure provide cells comprising any of the constructs disclosed herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof.

Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells, are used in assessing one or more test compounds.

### EXAMPLES

### Example 1. Evolution of Improved Cas14a1 Variants

A suite of evolved Cas14a1 enzymes was developed, and sgRNAs were engineered that exhibit improved gene editing efficiencies compared to wild-type Cas14a1/sgRNA in bacterial and human cells. Cas14a1, also known as Cas12f1, is one of the smallest known Cas enzymes discovered to date. However, wild-type Cas14a1 and its sgRNA exhibit virtually no gene editing activity above background in human cells. In bacteria, wild-type Cas14a1/sgRNA were weakly active, and phage-assisted continuous and non-continuous evolution (PACE and PANCE) were therefore used to improve its activity. In this evolution system, either Cas14a1 DNA binding activity or TadA8e-Cas14a1 base editing activity was coupled to successful phage propagation, and Cas14a1 enzymes with improved activity were thus evolved, as assayed in bacterial cells.

In parallel, it was observed that the wild-type Cas14a1 sgRNA contains a polyuridine tract, which prevents complete expression from the U6 promoter in human cells (a promoter that is commonly used to express sgRNAs in human cells). To overcome this, a variety of Cas14a1 sgRNAs were engineered that lack this polyuridine tract and are therefore compatible with expression from the U6 promoter. These engineered sgRNAs were screened, and one construct (engineered sgRNA 4) that enabled the most efficient DNA binding in bacteria was identified.

This newly engineered sgRNA 4 was then combined with PACE and PANCE-evolved Cas14a1 proteins. These evolved Cas14a1/engineered sgRNA pairs exhibited substantial improvements compared to wild-type Cas14a1/sgRNA in adenine base editing efficiencies across four genomic loci in HEK293T cells. Higher-stringency DNA-binding PACE and ABE-PACE were performed to further improve the activity of the evolved Cas14a1 variants. The evolved mutations tend to cluster around Cas protein:DNA interfaces, which is consistent with a model proposing that the mutations help to improve DNA-binding activity. After PACE campaigns and characterization of the evolved Cas14a1 proteins as base editors in HEK293T cells, the P24-L4.7-4/sgRNA4 and P28-L2.5-2A/sgRNA4 variants were identified as the ones that exhibit the greatest improvements in editing efficiencies.

### REFERENCES

Harrington, L. B. et al. Programmed DNA destruction by miniature CRISPR-Cas14 enzymes. Science 362, 839-842 (2018).
Karvelis, T. et al. PAM recognition by miniature CRISPR-Cas12f nucleases triggers programmable double-stranded DNA target cleavage. Nucleic Acids Res. 48, 5016-5023 (2020).
Miller, S. M. et al. Continuous evolution of SpCas9 variants compatible with non-G PAMs. Nat. Biotechnol. 38, 471-481 (2020).
Gao, Z. et al. Delineation of the Exact Transcription Termination Signal for Type 3 Polymerase III. Mol. Ther. Nucleic Acids 10, 36-44 (2017).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A Cas protein comprising an amino acid sequence that is at least 70%, at least 75%, at least 80%, at least 85%, at least 90%, at least 95%, or at least 99% identical to the amino acid sequence of a Cas protein of SEQ ID NO: 2, wherein the amino acid sequence of the Cas protein comprises at least 1, at least 2, at least 3, at least 4, at least 5, at least 6, at least 7, at least 8, at least 9, at least 10, at least 11, or at least 12 substitutions at positions selected from the group consisting of amino acid residues 1, 2, 11, 25, 32, 37, 41, 43, 44, 46, 58, 66, 76, 87, 118, 131, 134, 137, 138, 148, 157, 179, 201, 203, 206, 209, 210, 228, 260, 266, 268, 274, 282, 284, 296, 297, 298, 301, 303, 305, 309, 313, 320, 330, 341, 349, 352, 353, 366, 367, 372, 378, 392, 423, 425, 430, 461, 471, 477, 483, 486, 507, 508, 510, 513, 519, 528, and 529 of the amino acid sequence provided in SEQ ID NO: 2.

2. The Cas protein of claim 1, wherein the amino acid sequence of the Cas protein comprises at least 1, at least 2, at least 3, at least 4, at least 5, at least 6, at least 7, at least 8, at least 9, at least 10, at least 11, or at least 12 substitutions selected from the group consisting of M1X, A2X, K11X, K25X, N32X, I37X, K41X, K43X, D44X, V46X, A58X, R66X, K76X, G87X, I118X, I131X, A134X, V137X, E138X, R148X, A157X, K179X, Q201X, T203X, E206X, N209X, H210X, E228X, K260X, S266X, D268X, E274X, D282X, Q284X, I296X, C297X, E298X, A301X, M303X, N305X, D309X, I313X, S320X, K330X, F341X, N349X, F352X, H353X, L366X, K367X, K372X, A378X, S392X, N423X, E425X, K430X, I461X, T471X, K477X, N483X, N486X, E507X, N508X, A510X, A513X, N519X, E528X, and P529X, relative to the amino acid sequence provided in SEQ ID NO: 2, wherein X represents any amino acid other than the wild type amino acid.

3. The Cas protein of claim 1 or 2, wherein the amino acid sequence of the Cas protein comprises at least 1, at least 2, at least 3, at least 4, at least 5, at least 6, at least 7, at least 8, at least 9, at least 10, at least 11, or at least 12 substitutions selected from the group consisting of M1R, A2S, K11T, K25R, N32D, I37V, K41E, K43R, D44G, V46G, A58T, R66S, K76E, K76T, G87E, I118F, I131T, A134T, V137A, E138A, R148K, A157T, K179T, Q201R, T203R, E206K, N209K, H210Y, E228D, K260R, S266I, D268A, E274D, D282E, Q284R, I296N, I296F, C297G, E298G, A301T, M303V, N305H, D309A, I313V, S320N, K330T, F341S, F341C, N349S, F352Y, H353Y, L366M, K367E, K372M, A378V, S392I, N423T, N423S, N423D, E425K, K430R, I461V, T471I, K477E, N483D, N486D, E507D, N508D, A510D, A513S, N519I, E528K, and P529S, relative to the amino acid sequence of SEQ ID NO: 2.

4. The Cas protein of any one of claims 1-3, wherein the Cas protein comprises a combination of substitutions of any one of the Cas clones listed in Table 1.

5. The Cas protein of any one of claims 1-4, wherein the Cas protein comprises a combination of substitutions of any one of the clones selected from the group consisting of P21-L1.7-1, P21-L1.7-2, P21-L1.7-3, P21-L1.7-4, P21-L1.7-5, P21-L1.7-6, P21-L1.7-7, P21-L1.7-8, P21-L2.7-1, P21-L2.7-2, P21-L2.7-3, P21-L2.7-4, P21-L2.7-5, P21-L2.7-6, P21-L2.7-7, P21-L2.7-8, P21-L3.7-1, P21-L3.7-2, P21-L3.7-3, P21-L3.7-4, P21-L3.7-5, P21-L3.7-6, P21-L3.7-7, P21-L3.7-8, P21-L4.7-1, P21-L4.7-2, P21-L4.7-3, P21-L4.7-4, P21-L4.7-5, P21-L4.7-6, P21-L4.7-7, P21-L4.7-8, P24-L4.7-2, P24-L4.7-4, P24-L4.7-5, and P24-L4.7-6.

6. The Cas protein of any one of claims 1-5, wherein the Cas protein comprises substitutions at any of the following groups of positions:
K76, Q201, H210, E274, A301, F341, E425, and N486;
A58, K76, E206, N209, S266, F352, S392, N483, and E507;
E206, N209, D268, E298, I313, F341, and P529;
I131, E206, N209, D268, E298, S392, N423, and P529; and
T203, N209, D268, and C297.

7. The Cas protein of claim 6, wherein the Cas protein comprises any of the following groups of substitutions:
K76E, Q201R, H210Y, E274D, A301T, F341C, E425K, and N486D;
A58T, K76T, E206K, N209K, S266I, F352Y, S392I, N483D, and E507D;
E206K, N209K, D268A, E298G, I313V, F341S, and P529S;
I131T, E206K, N209K, D268A, E298G, S392I, N423D, and P529S; and
T203R, N209K, D268A, and C297G.

8. The Cas protein of any one of claims 1-5, wherein the Cas protein comprises substitutions at any of the following groups of positions:
K76, Q201, H210, E274, A301, I313, F341, E425, N486, and S524;
A58, K76, Q201, H210, E274, A301, F341, E425, N486, and S524;
Q201, H210, S246, E274, A301, F341, N369, N423, E425, N486, and S524; and
K76, Q201, H210, E274, A301, F341, E425, N486, K506, and N508.

9. The Cas protein of claim 8, wherein the Cas protein comprises any of the following groups of substitutions:
K76E, Q201R, H210Y, E274D, A301T, I313V, F341C, E425K, N486D, and S524A;
A58T, K76E, Q201R, H210Y, E274D, A301T, F341C, E425K, N486D, and S524P;
Q201R, H210Y, S246F, E274D, A301T, F341C, N369S, N423T, E425K, N486D, and S524P; and
K76E, Q201R, H210Y, E274D, A301T, F341C, E425K, N486D, K506E, and N508D.

10. The Cas protein of any one of claims 1-9, wherein the Cas protein comprises at least 2, at least 3, at least 4, at least 5, at least 6, at least 7, at least 8, at least 9, at least 10, at least 11, or at least 12 substitutions at positions selected from the group consisting of amino acid residues 1, 79, 111, 121, 133, 135, 151, 179, 202, 213, 228, 232, 236, 244, 260, 261, 280, 285, 313, 344, 369, 374, 388, 392, 393, 423, 425, 429, 430, 448, 459, 460, 464, 497, 513, 516, 525, and 526 of the amino acid sequence provided in SEQ ID NO: 2.

11. The Cas protein of claim 10, wherein the Cas protein comprises at least 2, at least 3, at least 4, at least 5, at least 6, at least 7, at least 8, at least 9, at least 10, at least 11, or at least 12 substitutions selected from the group consisting of M1X, D79X, E111X, Y121X, N133X, S135X, E151X, K179X, Y202X, D213X, E228X, Y232X, E236X, Q244X, K260X, R261X, N280X, T285X, I313X, Y344X, N369X, A374X, L388X, S392X, E393X, N423X, K425X, R429X, K430X, M448X, Y459X, G460X, R464X, H497X, A513X, N516X, T525X, and K526X, relative to the amino acid sequence provided in SEQ ID NO: 2, wherein X represents any amino acid other than the wild type amino acid.

12. The Cas protein of claim 10 or 11, wherein the Cas protein comprises at least 2, at least 3, at least 4, at least 5, at least 6, at least 7, at least 8, at least 9, at least 10, at least 11, or at least 12 substitutions selected from the group consisting of M1K, M1I, D79Y, E111K, Y121H, N133T, N133K, S135R, E151K, E151A, K179E, Y202D, Y202C, D213A, D213N, E228G, Y232C, Y232F, E236D, Q244K, Q244R, K260R, R261K, N280S, T285I, I313V, I313T, Y344C, N369D, A374V, L388R, S392I, E393K, N423T, N423D, K425E, R429L, K430R, M448I, Y459S, G460A, R464I, H497P, A513V, N516S, T525A, and K526R, relative to the amino acid sequence provided in SEQ ID NO: 2.

13. The Cas protein of any one of claims 10-12, wherein the Cas protein comprises a combination of substitutions of any one of the Cas clones listed in Table 2.

14. The Cas protein of any one of claims 10-13, wherein the Cas protein comprises a combination of substitutions of any one of the clones selected from the group consisting of P28L1.5-1, P28L1.5-2, P28L1.5-3A, P28L1.5-4, P28L1.5-4A, P28L1.5-5, P28L1.5-5A, P28L1.5-6, P28L1.5-6A, P28L1.5-7, P28L2.5-1A, P28L2.5-2, P28L2.5-2A, P28L2.5-3, P28L2.5-3A, P28L2.5-4A, P28L2.5-5A, P28L2.5-6, P28L2.5-6A, P28L2.5-7, P28L3.5-1, P28L3.5-2, P28L3.5-3, P28L3.5-4, P28L3.5-5, P28L3.5-6, P28L3.5-7, P28L3.5-8, P28L4.5-2, P28L4.5-3, P28L4.5-4, P28L4.5-5, and P28L4.5-6.

15. The Cas protein of any one of claims 10-14, wherein the Cas protein comprises substitutions at any of the following groups of positions:
A58, K76, Q201, H210, E274, A301, F341, E425, N486, and S524;
A58, K76, N133, Q201, H210, E228, E236, Q244, K260, E274, T285, A301, F341, A374, N486, and S524;
A58, K76, N133, K179, Q201, H210, D213, E228, E274, T285, A301, F341, S392, E425, N486, and S524; and
A58, K76, D79, D91, K179, Q201, H210, D213, Q244, E274, N280, T285, E298, A301, F341, E393, E425, N486, A510, A513, and S524.

16. The Cas protein of claim 15, wherein the Cas protein comprises any of the following groups of substitutions:
A58T, K76E, Q201R, H210Y, E274D, A301T, F341C, E425K, N486D, and S524P;
A58T, K76E, N133K, Q201R, H210Y, E228G, E236D, Q244K, K260R, E274D, T285I, A301T, F341C, A374V, N486D, and S524P;
A58T, K76E, N133K, K179E, Q201R, H210Y, D213A, E228G, E274D, T285I, A301T, F341C, S392I, E425K, N486D, and S524P; and
A58T, K76E, D79Y, D91A, K179E, Q201R, H210Y, D213N, Q244R, E274D, N280S, T285I, E298D, A301T, F341C, E393K, E425K, N486D, A510D, A513V, and S524P.

17. The Cas protein of any one of claims 1-16, wherein the Cas protein is fused to one or more nuclear localization sequences (NLS).

18. The Cas protein of claim 17, wherein the NLS is fused to the N-terminus of the Cas protein.

19. A Cas protein comprising the amino acid substitutions A58T, K76E, Q201R, H210Y, E274D, A301T, F341C, E425K, N486D, and S524P relative to SEQ ID NO: 2.

20. A Cas protein comprising the amino acid substitutions N133K, E228G, E236D, Q244K, K260R, T285I, A374V, and K425E relative to SEQ ID NO: 2.

21. A fusion protein comprising:
(i) the Cas protein of any one of claims 1-20; and
(ii) an effector domain.

22. The fusion protein of claim 21, wherein the effector domain comprises nuclease activity, nickase activity, recombinase activity, deaminase activity, methyltransferase activity, methylase activity, acetylase activity, acetyltransferase activity, transcriptional activation activity, transcriptional repression activity, or polymerase activity.

23. The fusion protein of claim 21 or 22, wherein the effector domain is a nucleic acid editing domain.

24. The fusion protein of claim 23, wherein the nucleic acid editing domain comprises a deaminase domain.

25. The fusion protein of claim 24, wherein the deaminase domain is an adenosine deaminase domain.

26. The fusion protein of claim 25, wherein the adenosine deaminase domain is an E. coli Tad A (ecTadA) deaminase domain.

27. The fusion protein of claim 26, wherein the adenosine deaminase domain is ecTadA(8e).

28. The fusion protein of claim 24, wherein the deaminase domain is a cytosine deaminase domain.

29. The fusion protein of claim 28, wherein the cytosine deaminase domain is an apolipoprotein B mRNA-editing complex (APOBEC) family deaminase domain.

30. The fusion protein of any one of claims 21-29, wherein the fusion protein exhibits increased base editing activity on a target sequence as compared to a fusion protein comprising a wild-type Cas14a1 protein as provided by SEQ ID NO: 2.

31. The fusion protein of claim 30, wherein the activity is increased by at least 2-fold, at least 3-fold, at least 4-fold, at least 5-fold, at least 6-fold, at least 7-fold, at least 8-fold, at least 9-fold, or at least 10-fold as compared to a fusion protein comprising a wild-type Cas14a1 protein as provided by SEQ ID NO: 2.

32. A fusion protein comprising:
(i) the Cas protein of any one of claims 1-20; and
(ii) a domain comprising an RNA-dependent DNA polymerase activity.

33. The fusion protein of claim 32, wherein the domain comprising an RNA-dependent DNA polymerase activity is a reverse transcriptase.

34. A guide RNA (gRNA) comprising a nucleic acid sequence of any one of SEQ ID NOs: 173-176, or a nucleic acid sequence that is at least at least 70%, at least 75%, at least 80%, at least 85%, at least 90%, at least 95%, or at least 99% identical to the nucleic acid sequence of any one of SEQ ID NOs: 173-176.

35. The gRNA of claim 34, wherein the gRNA comprises a nucleic acid sequence that is 100% identical to the nucleic acid sequence of any one of SEQ ID NOs: 173-176.

36. The gRNA of claim 34 or 35, wherein the gRNA comprises the nucleic acid sequence 5′-CUUCACUGAUAAAGUGGAGAACCGCUUCACCAAAAGCUGUCCCUUAGGGGAUUA GAACUUGAGUGAAGGUGGGCUGCUUGCAUCAGCCUAAUGUCGAGAAGUGCUUUC UUCGGAAAGUAACCCUCGAAACAAAUUCAUUUCAAGAAAGUGAAUGAAGGAAUG CAAC-3′ (SEQ ID NO: 176).

37. The gRNA of any one of claims 34-36, wherein the gRNA exhibits increased expression from a U6 promoter compared to a wild-type Cas14a1 gRNA.

38. The gRNA of any one of claims 34-37, wherein the backbone sequence of the gRNA comprises one or more substitutions relative to a wild-type Cas14a1 gRNA.

39. The gRNA of claim 38, wherein the portions of the gRNA besides the backbone sequence do not comprise any substitutions relative to a wild-type Cas14a1 gRNA.

40. A complex comprising a fusion protein of any one of claims 21-33 and a guide RNA.

41. A complex comprising a fusion protein and a guide RNA of any one of claims 34-39.

42. A complex comprising a fusion protein of any one of claims 21-33 and a guide RNA of any one of claims 34-39.

43. A method for modifying a target nucleic acid molecule comprising contacting the target nucleic acid molecule with the fusion protein of any one of claims 21-33 and a guide RNA.

44. The method of claim 43, wherein the guide RNA comprises a nucleic acid sequence of any one of SEQ ID NOs: 172-176, or a nucleic acid sequence that is at least at least 70%, at least 75%, at least 80%, at least 85%, at least 90%, at least 95%, or at least 99% identical to the nucleic acid sequence of any one of SEQ ID NOs: 172-176.

45. A method for modifying a target nucleic acid molecule comprising contacting the target nucleic acid molecule with the complex of any one of claims 40-42.

46. The method of any one of claims 43-45, wherein the contacting is performed in vitro.

47. The method of any one of claims 43-45, wherein the contacting is performed in vivo.

48. The method of claim 47, wherein the contacting is performed in a subject.

49. The method of claim 48, wherein the subject has been diagnosed with a disease or disorder.

50. The method of any one of claims 43-49, wherein the target nucleic acid molecule comprises a sequence associated with a disease or disorder.

51. The method of claim 50, wherein the target nucleic acid molecule comprises a point mutation associated with a disease or disorder.

52. The method of claim 51, wherein the point mutation comprises a T→C point mutation associated with a disease or disorder.

53. The method of claim 51, wherein the point mutation comprises an A→G point mutation associated with a disease or disorder.

54. The method of any one of claims 51-53, wherein the step of editing the target nucleic acid molecule results in correction of the point mutation.

55. A polynucleotide encoding a Cas protein of any one of claims 1-20, a fusion protein of any one of claims 21-33, a guide RNA of any one of claims 34-39, or a complex of any one of claims 40-42.

56. A vector comprising a polynucleotide of claim 55.

57. A cell comprising a Cas protein of any one of claims 1-20, a fusion protein of any one of claims 21-33, a guide RNA of any one of claims 34-39, a complex of any one of claims 40-42, a polynucleotide of claim 55, or a vector of claim 56.

58. A kit comprising a Cas protein of any one of claims 1-20, a fusion protein of any one of claims 21-33, a guide RNA of any one of claims 34-39, a complex of any one of claims 40-42, a polynucleotide of claim 55, a vector of claim 56, or a cell of claim 57.

59. A pharmaceutical composition comprising a Cas protein of any one of claims 1-20, a fusion protein of any one of claims 21-33, a guide RNA of any one of claims 34-39, a complex of any one of claims 40-42, a polynucleotide of claim 55, a vector of claim 56, or a cell of claim 57, and a pharmaceutically acceptable excipient.

60. An AAV comprising a Cas protein of any one of claims 1-20, a fusion protein of any one or claims 21-33, a guide RNA of any one of claims 34-39, a complex of any one of claims 40-42, a polynucleotide of claim 55, a vector of claim 56, or a pharmaceutical composition of claim 59.

61. A Cas protein of any one of claims 1-20, a fusion protein of any one of claims 21-33, a guide RNA of any one of claims 34-39, a complex of any one of claims 40-42, a polynucleotide of claim 55, a vector of claim 56, or a pharmaceutical composition of claim 59 for use in medicine.

62. Use of a Cas protein of any one of claims 1-20, a fusion protein of any one of claims 21-33, a guide RNA of any one of claims 34-39, a complex of any one of claims 40-42, a polynucleotide of claim 55, a vector of claim 56, or a pharmaceutical composition of claim 59 in the manufacture of a medicament for the treatment of a disease or disorder.
